(19) United States
(12) Patent Application Publication
(10) Pub. No.: US 2025/0171454 A1
(43) Pub. Date: May 29, 2025

(54) HETEROBIFUNCTIONAL PYRIDINYLMETHYL AMINOPURINE AND RELATED COMPOUNDS AND THEIR USE IN NSD2 DEGRADATION AND TREATING MEDICAL CONDITIONS

(71) Applicant: K36 Therapeutics, Inc., Cambridge, MA (US)

(72) Inventors: Chad Arthur Lewis, Boylston, MA (US); Terrence Joseph Connolly, Concord, MA (US)

(21) Appl. No.: 18/961,883

(22) Filed: Nov. 27, 2024

Related U.S. Application Data
(60) Provisional application No. 63/653,524, filed on May 30, 2024.
(60) Provisional application No. 63/604,014, filed on Nov. 29, 2023.

(51) Int. Cl. C07D 487/04; A61K 47/55

(52) U.S. Cl. CPC C07D 487/04; A61K 47/55

(57) ABSTRACT

The disclosure provides heterobifunctional substituted pyridinylmethyl aminopurine and related compounds, pharmaceutical compositions, their use for inhibiting NSD2 protein and/or inducing NSD2 protein degradation, and their use in the treatment of medical disorders, such as cancer.

### CROSS-REFERENCE TO RELATED APPLICATIONS

This application claims the benefit of and priority to U.S. Provisional Patent Application Ser. No. 63/653,524, filed May 30, 2024, and U.S. Provisional Patent Application Ser. No. 63/604,014, filed Nov. 29, 2023; the contents of each of which are hereby incorporated by reference in their entirety.

### FIELD OF THE DISCLOSURE

The present disclosure provides heterobifunctional substituted pyridinylmethyl aminopurine and related compounds, pharmaceutical compositions, their use for inhibiting NSD2 protein and/or inducing NSD2 protein degradation, and their use in the treatment of medical disorders, such as cancer.

### BACKGROUND

Cancer continues to be a significant health problem despite the substantial research efforts and scientific advances reported in the literature for treating this disease. Solid tumors, including breast cancer, lung cancer, and pancreatic cancer remain prevalent among the world population. Current treatment options for these cancers are not effective for all patients and/or can have substantial adverse side effects. New therapies are needed to address this unmet need in cancer therapy.

The nuclear receptor-binding SET domain protein 2 (NSD2), also known as multiple myeloma SET domain (MMSET) or Wolf-Hirschhorn syndrome candidate 1 (WHSC1), is an epigenetic modifier having a role in oncogenesis. Several human cancers are associated with NSD2 overexpression and/or activating point mutations. (Coussens et al., J. Biol. Chem. 293 (2018) 13750-13654.) For example, high expression of NSD2 has been reported in human cancers including bladder, brain, gastrointestinal, lung, liver, ovary, skin, uterus, breast, prostrate, and glioblastoma. Additionally, pediatric cancer genomes appear to be particularly likely to contain NSD2 mutations. Finally, upregulation of NSD2 has been linked with aggressive tumor behavior and poor clinical outcomes. Certain compounds that inhibit NSD2 are described in international patent application publication WO 2021/028854. Additional compounds that inhibit and/or degrade NSD2 would be beneficial to patients suffering from an NSD2-related disease or condition.

Ubiquitin-Proteasome Pathway (UPP) is a critical pathway that regulates key regulator proteins and degrades misfolded or abnormal proteins. UPP is central to multiple cellular processes, and if defective or imbalanced, it leads to pathogenesis of a variety of diseases. The covalent attachment of ubiquitin to specific protein substrates is achieved through the action of E3 ubiquitin ligases.

There are over 600 E3 ubiquitin ligases which facilitate the ubiquitination of different proteins in vivo, which can be divided into four families: HECT-domain E3s, U-box E3s, monomeric RING E3s and multi-subunit E3s. See generally Li et al. (PLOS One, 2008, 3, 1487) titled “Genome-wide and functional annotation of human E3 ubiquitin ligases identifies MULAN, a mitochondrial E3 that regulates the organelle's dynamics and signaling.”; Berndsen et al. (Nat. Struct. Mol. Biol., 2014, 21, 301-307) titled “New insights into ubiquitin E3 ligase mechanism”; Deshaies et al. (Ann. Rev. Biochem., 2009, 78, 399-434) titled “RING domain E3 ubiquitin ligases.”; Spratt et al. (Biochem. 2014, 458, 421-437) titled “RBR E3 ubiquitin ligases: new structures, new insights, new questions.”; and Wang et al. (Nat. Rev. Cancer., 2014, 14, 233-347) titled “Roles of F-box proteins in cancer.”

The UPP is used to induce selective protein degradation, including use of fusion proteins to artificially ubiquitinate target proteins and synthetic small-molecule probes to induce proteasome-dependent degradation. Bifunctional compounds composed of a target protein-binding ligand and an E3 ubiquitin ligase ligand, induce proteasome-mediated degradation of selected proteins via their recruitment to E3 ubiquitin ligase and subsequent ubiquitination. These drug-like molecules offer the possibility of temporal control over protein expression. Such compounds are capable of inducing the inactivation of a protein of interest upon addition to cells or administration to an animal or human, and are useful for treating diseases by removing pathogenic or oncogenic proteins (Crews C, Chemistry & Biology, 2010, 17(6):551-555; Schnnekloth J S Jr., Chembiochem, 2005, 6(1):40-46).

New compounds that are inhibitors of NSD2 and/or induce degradation of NSD2 are therefore needed as therapeutic agents for the treatment of medical disorders, such as cancer. The present disclosure addresses the foregoing needs and provides other related advantages.

### SUMMARY

The disclosure provides heterobifunctional substituted pyridinylmethyl aminopurine and related compounds, pharmaceutical compositions, their use for inhibiting NSD2 protein and/or inducing NSD2 protein degradation, and their use in the treatment of medical disorders, such as cancer. In particular, one aspect of the disclosure provides a collection of heterobifunctional substituted pyridinylmethyl aminopurine and related compounds, such as a compound represented by Formula I:

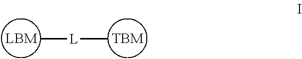

or a pharmaceutically acceptable salt thereof, where the variables are as defined in the detailed description. Further description of additional collections of heterobifunctional substituted pyridinylmethyl aminopurine and related compounds are described in the detailed description. The compounds may be part of a pharmaceutical composition comprising a pharmaceutically acceptable carrier.

Another aspect of the disclosure provides a collection of heterobifunctional substituted pyridinylmethyl aminopurine and related compounds, such as a compound represented by Formula II:

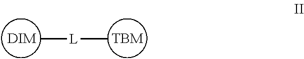

or a pharmaceutically acceptable salt thereof, where the variables are as defined in the detailed description. Further description of additional collections of heterobifunctional substituted pyridinylmethyl aminopurine and related compounds are described in the detailed description. The compounds may be part of a pharmaceutical composition comprising a pharmaceutically acceptable carrier.

Another aspect of the disclosure provides a method of treating a disorder mediated by NSD2 in a subject. The method comprises administering a therapeutically effective amount of a compound described herein, such as a compound of Formula I or Formula II, to a subject in need thereof to treat the disorder, as further described in the detailed description.

Another aspect of the disclosure provides a method of degrading an NSD2 protein in a cell. The method comprises administering to the cell an effective amount of a compound described herein, such as a compound of Formula I or Formula II, resulting in degradation of the NSD2 protein in the cell, as further described in the detailed description.

Another aspect of the disclosure provides a method of inhibiting NSD2 activity. The method comprises contacting NSD2 with an effective amount of a compound described herein, such as a compound of Formula I or Formula II, to inhibit NSD2 activity, as further described in the detailed description.

### DETAILED DESCRIPTION

The present disclosure provides heterobifunctional substituted pyridinylmethyl aminopurine and related compounds, pharmaceutical compositions, their use for inhibiting NSD2 protein and/or inducing NSD2 protein degradation, and their use in the treatment of medical disorders, such as cancer. The practice of the present disclosure employs, unless otherwise indicated, conventional techniques of organic chemistry, pharmacology, molecular biology (including recombinant techniques), cell biology, biochemistry, and immunology. Such techniques are explained in the literature, such as in “Comprehensive Organic Synthesis” (B. M. Trost & I. Fleming, eds., 1991-1992); “Handbook of experimental immunology” (D. M. Weir & C. C. Blackwell, eds.); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety.

Various aspects of the disclosure are set forth below in sections; however, aspects of the disclosure described in one particular section are not to be limited to any particular section. Further, when a variable is not accompanied by a definition, the previous definition of the variable controls.

### Definitions

Compounds of the present disclosure include those described generally herein, and are further illustrated by the classes, subclasses, and species disclosed herein. As used herein, the following definitions shall apply unless otherwise indicated. These definitions apply regardless of whether a term is used by itself or in combination with other terms, unless otherwise indicated. Hence, the definition of “alkyl” applies to “alkyl” as well as the “alkyl” portions of “—O-alkyl” etc. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”), that has a single point of attachment to the rest of the molecule. Unless otherwise specified, aliphatic groups contain 1-6 aliphatic carbon atoms. In certain embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In certain embodiments, “cycloaliphatic” refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e. carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In certain embodiments, a bicyclic group has 7-12 ring members and 0-4 heteroatoms independently selected from nitrogen, oxygen, or sulfur. As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In certain embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

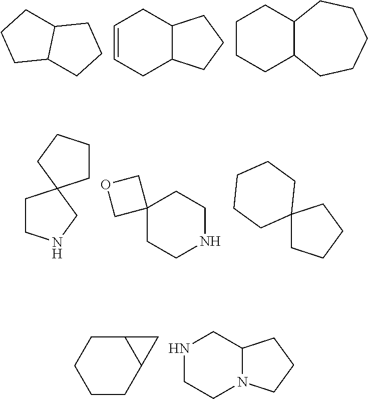

Exemplary bridged bicyclics include:

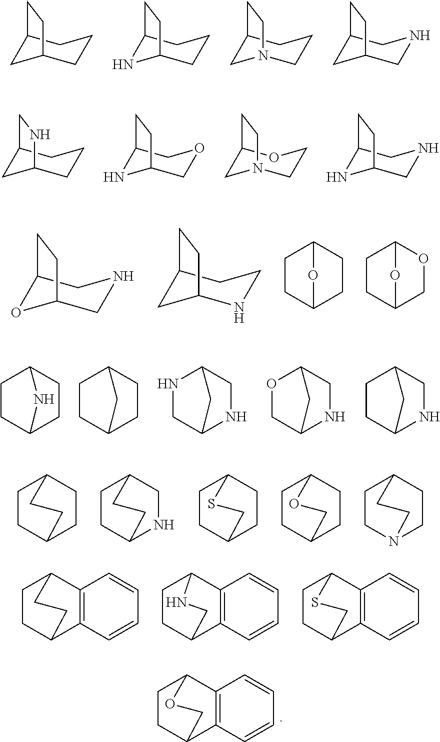

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “—(C₀ alkylene)-” refers to a bond. Accordingly, the term “—(C₀₋₃ alkylene)-” encompasses a bond (i.e., C₀) and a —(C₁₋₃ alkylene)- group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like. The term “phenylene” refers to a multivalent phenyl group having the appropriate number of open valences to account for groups attached to it. For example, “phenylene” is a bivalent phenyl group when it has two groups attached to it (e.g.,

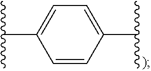 [SMILES: *c1ccc(*)cc1]

“phenylene” is a trivalent phenyl group when it has three groups attached to it (e.g.,

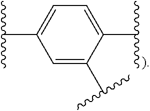 [SMILES: *c1ccc(*)c(*)c1]

The term “arylene” refers to a bivalent aryl group.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

The term “heteroarylene” refers to a multivalent heteroaryl group having the appropriate number of open valences to account for groups attached to it. For example, “heteroarylene” is a bivalent heteroaryl group when it has two groups attached to it; “heteroarylene” is a trivalent heteroaryl group when it has three groups attached to it. The term “pyridinylene” refers to a multivalent pyridine radical having the appropriate number of open valences to account for groups attached to it. For example, “pyridinylene” is a bivalent pyridine radical when it has two groups attached to it (e.g.,

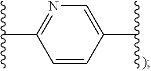 [SMILES: *c1ccc(*)nc1]

“pyridinylene” is a trivalent pyridine radical when it has three groups attached to it (e.g.,

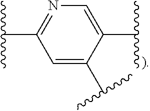 [SMILES: *c1cc(*)c(*)cn1]

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted. The term “oxo-heterocyclyl” refers to a heterocyclyl substituted by an oxo group. The term “heterocyclylene” refers to a multivalent heterocyclyl group having the appropriate number of open valences to account for groups attached to it. For example, “heterocyclylene” is a bivalent heterocyclyl group when it has two groups attached to it; “heterocyclylene” is a trivalent heterocyclyl group when it has three groups attached to it.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the disclosure may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Each optional substituent on a substitutable carbon is a monovalent substituent independently selected from halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄₀S(O)₂R^{∘}; —S(O)₂NR^{∘}₂; —S(O)(NR^{∘})R^{∘}; —S(O)₂N═C(NR^{∘}₂)₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂.

Each R^{∘} is independently hydrogen, C₁₋₆ aliphatic, —CH₂Ph, —O(CH₂)₀₋₁Ph, —CH₂-(5- or 6-membered heteroaryl ring), or a 5- or 6-membered saturated, partially unsaturated, or aryl ring having 0, 1, 2, 3, or 4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0, 1, 2, 3, or 4 heteroatoms independently selected from nitrogen, oxygen, or sulfur, which may be substituted by a divalent substituent on a saturated carbon atom of R^{∘} selected from ═O and ═S; or each R^{∘} is optionally substituted with a monovalent substituent independently selected from halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●}.

Each R^{●} is independently selected from C₁₋₄ aliphatic, —CH₂Ph, —O(CH₂)₀₋₁Ph, or a 5- or 6-membered saturated, partially unsaturated, or aryl ring having 0, 1, 2, 3, or 4 heteroatoms independently selected from nitrogen, oxygen, or sulfur, and wherein each R^{●} is unsubstituted or where preceded by halo is substituted only with one or more halogens; or wherein an optional substituent on a saturated carbon is a divalent substituent independently selected from ═O, ═S, ═NNR^{●}₂, ═NNHC(O)R^{●}, ═NNHC(O)OR^{●}, ═NNHS(O)₂R^{●}, ═NR^{●}, ═NOR^{●}, —O(C(R^{●}₂))₂₋₃O—, or —S(C(R^{●}₂))₂₋₃S—, or a divalent substituent bound to vicinal substitutable carbons of an “optionally substituted” group is —O(CR^{●}₂)₂₋₃O—, wherein each independent occurrence of R^{●} is selected from hydrogen, C₁₋₆ aliphatic or an unsubstituted 5- or 6-membered saturated, partially unsaturated, or aryl ring having 0, 1, 2, 3, or 4 heteroatoms independently selected from nitrogen, oxygen, or sulfur.

When R^{●} is C₁₋₆ aliphatic, R^{●} is optionally substituted with halogen, —R^{●}, -(haloR^{●}), —OH, —OR^{●}, —O(haloR^{●}), —CN, —C(O)OH, —C(O)OR^{●}, —NH₂, —NHR, —NR^{●}₂, or —NO₂, wherein each R^{●} is independently selected from C₁₋₄ aliphatic, —CH₂Ph, —O(CH₂)₀₋₁Ph, or a 5- or 6-membered saturated, partially unsaturated, or aryl ring having 0, 1, 2, 3, or 4 heteroatoms independently selected from nitrogen, oxygen, or sulfur, and wherein each R^{●} is unsubstituted or where preceded by halo is substituted only with one or more halogens.

An optional substituent on a substitutable nitrogen is independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic, unsubstituted —OPh, or an unsubstituted 5- or 6-membered saturated, partially unsaturated, or aryl ring having 0, 1, 2, 3, or 4 heteroatoms independently selected from nitrogen, oxygen, or sulfur, or, two independent occurrences of R^{†}, taken together with their intervening atom(s) form an unsubstituted 3-12-membered saturated, partially unsaturated, or aryl mono- or bicyclic ring having 0, 1, 2, 3, or 4 heteroatoms independently selected from nitrogen, oxygen, or sulfur; wherein when R^{†} is C₁₋₆ aliphatic, R^{†} is optionally substituted with halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is independently selected from C₁₋₄ aliphatic, —CH₂Ph, —O(CH₂)₀₋₁Ph, or a 5- or 6-membered saturated, partially unsaturated, or aryl ring having 0, 1, 2, 3, or 4 heteroatoms independently selected from nitrogen, oxygen, or sulfur, and wherein each R^{●} is unsubstituted or where preceded by halo is substituted only with one or more halogens.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Further, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al., Journal of Pharmaceutical Sciences (1977) 66(1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C. on their website). These disclosures are incorporated herein by reference.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the disclosure. Unless otherwise stated, all tautomeric forms of the compounds of the disclosure are within the scope of the disclosure. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this disclosure. Such compounds are useful, for example, as analytical tools, as probes in biological assays, or as therapeutic agents in accordance with the present disclosure.

Diastereomeric mixtures can be separated into their individual diastereomers on the basis of their physical chemical differences by methods known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Alternatively, a particular enantiomer of a compound of the present disclosure may be prepared by asymmetric synthesis. Still further, where the molecule contains a basic functional group (such as amino) or an acidic functional group (such as carboxylic acid) diastereomeric salts are formed with an appropriate optically-active acid or base, followed by resolution of the diastereomers thus formed by fractional crystallization or chromatographic means known in the art, and subsequent recovery of the pure enantiomers.

Individual stereoisomers of the compounds of the disclosure may, for example, be substantially free of other isomers, or may be admixed, for example, as racemates or with all other, or other selected, stereoisomers. Chiral center(s) in a compound of the present disclosure can have the S or R configuration as defined by the IUPAC 1974 Recommendations. Further, to the extent a compound described herein may exist as a atropisomer (e.g., substituted biaryls), all forms of such atropisomer are considered part of this disclosure.

Chemical names, common names, and chemical structures may be used interchangeably to describe the same structure. If a chemical compound is referred to using both a chemical structure and a chemical name, and an ambiguity exists between the structure and the name, the structure predominates. It should also be noted that any carbon as well as heteroatom with unsatisfied valences in the text, schemes, examples and tables herein is assumed to have the sufficient number of hydrogen atom(s) to satisfy the valences.

The terms “a” and “an” as used herein mean “one or more” and include the plural unless the context is inappropriate.

The term “alkyl” refers to a saturated straight or branched hydrocarbon, such as a straight or branched group of 1-12, 1-10, or 1-6 carbon atoms, referred to herein as C₁-C₁₂ alkyl, C₁-C₁₀ alkyl, and C₁-C₆ alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₃-C₆ cycloalkyl,” derived from a cycloalkane. Exemplary cycloalkyl groups include cyclohexyl, cyclopentyl, cyclobutyl, and cyclopropyl. The term “cycloalkylene” refers to a bivalent cycloalkyl group.

The term “haloalkyl” refers to an alkyl group that is substituted with at least one halogen. Exemplary haloalkyl groups include —CH₂F, —CHF₂, —CF₃, —CH₂CF₃, —CF₂CF₃, and the like. The term “haloalkylene” refers to a bivalent haloalkyl group.

The term “hydroxyalkyl” refers to an alkyl group that is substituted with at least one hydroxyl. Exemplary hydroxyalkyl groups include —CH₂CH₂OH, —C(H)(OH)CH₃, —CH₂C(H)(OH)CH₂CH₂OH, and the like.

The terms “alkenyl” and “alkynyl” are art-recognized and refer to unsaturated aliphatic groups analogous in length and possible substitution to the alkyls described above, but that contain at least one double or triple bond respectively.

The term “carbocyclylene” refers to a multivalent carbocyclyl group having the appropriate number of open valences to account for groups attached to it. For example, “carbocyclylene” is a bivalent carbocyclyl group when it has two groups attached to it; “carbocyclylene” is a trivalent carbocyclyl group when it has three groups attached to it.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. The term “haloalkoxyl” refers to an alkoxyl group that is substituted with at least one halogen. Exemplary haloalkoxyl groups include —OCH₂F, —OCHF₂, —OCF₃, —OCH₂CF₃, —OCF₂CF₃, and the like. The term “hydroxyalkoxyl” refers to an alkoxyl group that is substituted with at least one hydroxyl. Exemplary hydroxyalkoxyl groups include —OCH₂CH₂OH, —OCH₂C(H)(OH)CH₂CH₂OH, and the like. The term “alkoxylene” refers to a bivalent alkoxyl group.

The term “oxo” is art-recognized and refers to a “═O” substituent. For example, a cyclopentane substituted with an oxo group is cyclopentanone.

The symbol “ [symbol] ” indicates a point of attachment.

When a point of attachment is depicted with a bond that crosses square brackets, the point of attachment may replace any single hydrogen atom of the chemical structure within the square brackets. For example, the chemical structure

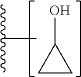

encompasses

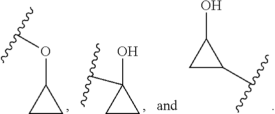

When the chemical structure within square brackets contains one or more variables, the point of attachment may replace a hydrogen atom on a variable. To illustrate, the chemical structure

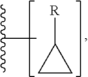

where R is hydroxyl or C₁₋₄ alkyl, encompasses, for example,

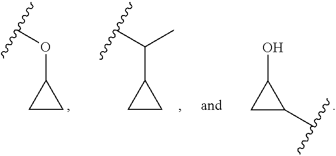

When a chemical structure containing a ring is depicted with a substituent having a bond that crosses a ring bond, the substituent may be attached at any available position on the ring. For example, the chemical structure

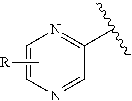

encompasses

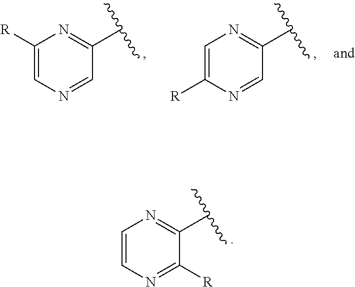

In the context of a polycyclic fused ring, when a chemical structure containing a polycyclic fused ring is depicted with one or more substituent(s) having a bond that crosses multiple rings, the one or more substituent(s) may be independently attached to any of the rings crossed by the bond. To illustrate, the chemical structure

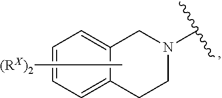

encompasses, for example,

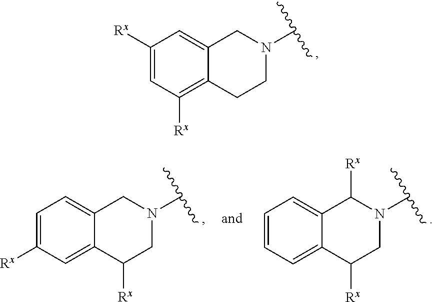

When any substituent or variable occurs more than one time in any constituent or the compound of the disclosure, its definition on each occurrence is independent of its definition at every other occurrence, unless otherwise indicated.

One or more compounds of the disclosure may exist in unsolvated as well as solvated forms with pharmaceutically acceptable solvents such as water, ethanol, and the like, and it is intended that the disclosure embrace both solvated and unsolvated forms. “Solvate” means a physical association of a compound of this disclosure with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

As used herein, the terms “subject” and “patient” are used interchangeably and refer to organisms to be treated by the methods of the present disclosure. Such organisms preferably include, but are not limited to, mammals (e.g., murines, simians, equines, bovines, porcines, canines, felines, and the like), and, most preferably, includes humans.

The term “IC₅₀” is art-recognized and refers to the concentration of a compound that is required to achieve 50% inhibition of the target. The potency of an inhibitor is usually defined by its IC₅₀ value. The lower the IC₅₀ value the greater the potency of the antagonist and the lower the concentration that is required to inhibit the maximum biological response. In certain embodiments, an inhibitor has an IC₅₀ and/or binding constant of less than about 100 μM, less than about 50 μM, less than about 1 μM, less than about 500 nM, less than about 100 nM, less than about 10 nM, or less than about 1 nM.

As used herein, the terms “NSD2 inhibitor” or “NSD2 antagonist” are defined as a compound that binds to and/or inhibits NSD2 with measurable affinity. In certain embodiments, inhibition in the presence of the inhibitor is observed in a dose-dependent manner. In certain embodiments, the measured signal (e.g., signaling activity or biological activity) is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or at least about 100% lower than the signal measured with a negative control under comparable conditions.

The terms “measurable affinity” and “measurably inhibit,” as used herein, means a measurable change or inhibition in enzyme activity between a sample comprising a compound of the present disclosure, or composition thereof, and the enzyme, and an equivalent sample comprising the enzyme, in the absence of said compound, or composition thereof.

As used herein, the term “effective amount” refers to the amount of a compound sufficient to effect beneficial or desired results (e.g., a therapeutic, ameliorative, inhibitory, or preventative result). An effective amount can be administered in one or more administrations, applications, or dosages and is not intended to be limited to a particular formulation or administration route.

As used herein, the term “treating” includes any effect, e.g., lessening, reducing, modulating, ameliorating or eliminating, that results in the improvement of the condition, disease, disorder, and the like, or ameliorating a symptom thereof. In certain embodiments, treatment can be administered after one or more symptoms have developed. In other embodiments, treatment can be administered in the absence of symptoms. For example, treatment can be administered to a susceptible individual prior to the onset of symptoms (e.g., in light of a history of symptoms and/or in light of genetic or other susceptibility factors). Treatment can also be continued after symptoms have resolved, for example, to prevent or delay their recurrence.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

For therapeutic use, salts of the compounds of the present disclosure are contemplated as being pharmaceutically acceptable. However, salts of acids and bases that are non-pharmaceutically acceptable may also find use, for example, in the preparation or purification of a pharmaceutically acceptable compound.

In addition, when a compound of the disclosure contains both a basic moiety (such as, but not limited to, a pyridine or imidazole) and an acidic moiety (such as, but not limited to, a carboxylic acid) zwitterions (“inner salts”) may be formed. Such acidic and basic salts used within the scope of the disclosure are pharmaceutically acceptable (i.e., non-toxic, physiologically acceptable) salts. Such salts of the compounds of the disclosure may be formed, for example, by reacting a compound of the disclosure with an amount of acid or base, such as an equivalent amount, in a medium such as one in which the salt precipitates or in an aqueous medium followed by lyophilization.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

As a general matter, compositions specifying a percentage are by weight unless otherwise specified.

### I. Heterobifunctional Substituted Pyridinylmethyl Aminopurine and Related Compounds

The disclosure provides heterobifunctional substituted pyridinylmethyl aminopurine and related compounds. The compounds may be used in the pharmaceutical compositions and therapeutic methods described herein. Exemplary compounds are described in the following sections, along with exemplary procedures for making the compounds.

One aspect of the disclosure provides a compound represented by Formula I:

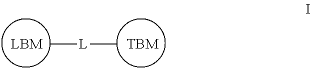

or a pharmaceutically acceptable salt thereof, wherein:
LBM is a ligase binding moiety;
L is a bivalent linker; and
TBM is a radical of Formula A:

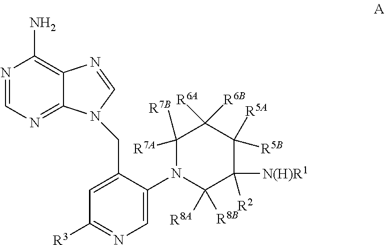

wherein:
R¹ is hydrogen or C₁₋₄ alkyl;
R² is —(C₁₋₄ haloalkyl substituted with 0 or 1 occurrences of hydroxyl) or a 5-6 membered heteroaryl containing 1 nitrogen atom, wherein the heteroaryl is substituted with 0 or 1 occurrences of halo;
R³ is phenyl or pyridinyl, each of which are substituted with p occurrences of R⁴;
R⁴ represents independently for each occurrence halo, hydroxyl, C₁₋₄ haloalkyl, —O—(C₁₋₄ haloalkyl), or C₁₋₄ alkoxyl;
R^{5A}, R^{5B}, R^{6A}, R^{6B}, R^{7A}, R^{7B}, R^{8A}, and R^{8B} are independently hydrogen, halo, cyano, aryl, —(C₀₋₆ alkylene)-(hydroxyl), C₁₋₆ alkyl, —(C₁₋₆ haloalkyl substituted with 0 or 1 occurrences of hydroxyl), —(C₀₋₆ alkylene)-(C₁₋₆ alkoxyl substituted with 0 or 1 occurrences of halo), —C(O)—OR⁹, or —(C(R¹⁰)(R¹¹))ₛ—C(O)—N(R¹²)(R¹³); R⁹, R¹⁰, R¹¹, R¹², and R¹³ are independently hydrogen or C₁₋₄ alkyl; and
p is 0, 1, 2, or 3; and
s is 0, 1, 2, 3, or 4.

The definitions of variables in Formula I above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I.

The compound may be further characterized according to, for example, the identity of TBM, L, and/or LBM, as described below.

The description above describes multiple embodiments relating to compounds of Formula I. The patent application specifically contemplates all combinations of the embodiments.

Another aspect provides a compound of Formula II:

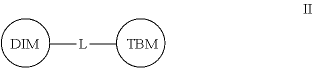

or a pharmaceutically acceptable salt thereof, wherein DIM is a degradation inducing moiety, L is a linker, and TBM is a target binding moiety. In certain embodiments, the compound is a compound of Formula II. In certain embodiments, the TBM is a moiety described in Part D below. The compound may be further characterized according to, for example, the identity of DIM, L, and/or TBM, as described below.

### Part A: Ligase Binding Moiety (LBM)

As defined generally above, LBM is a ligase binding moiety. In certain embodiments, LBM is an E3 ligase ligand. Such E3 ligase ligands are well known to one of ordinary skill in the art and include those described in M. Toure, C. M. Crews, Angew. Chem. Int. Ed. 2016, 55, 1966, T. Uehara et al. Nature Chemical Biology 2017, 13, 675, and the following U.S. and international patent publications: WO 2017/176708, US 2017/0281784, WO 2017/161119, WO 2017/176957, WO 2017/176958, WO 2015/160845, US 2015/0291562, WO 2016/197032, WO 2016/105518, US 2018/0009779, WO 2017/007612, 2018/0134684, WO 2013/106643, US 2014/0356322, WO 2002/020740, US 2002/0068063, WO 2012/078559, US 2014/0302523, WO 2012/003281, US 2013/0190340, US 2016/0022642, WO 2014/063061, US 2015/0274738, WO 2016/118666, US 2016/0214972, WO 2016/149668, US 2016/0272639, WO 2016/169989, US 2018/0118733, WO 2016/197114, US 2018/0147202, WO 2017/011371, US 2017/0008904, WO 2017/011590, US 2017/0037004, WO 2017/079267, US 2017/0121321, WO 2017/117473, WO 2017/117474, WO 2013/106646, WO 2014/108452, WO 2017/197036, WO 2017/197046, WO 2017/197051, WO 2017/197055, WO 2017/197056, WO 2019/060693, WO 2019/140387, and WO 2020/010177, the contents of each of which are herein incorporated by reference in their entireties.

In certain embodiments, LBM is a cereblon E3 ubiquitin ligase binding moiety, a VHL E3 ubiquitin ligase binding moiety, an IAP E3 ubiquitin ligase binding moiety, or an MDM2 E3 ubiquitin ligase binding moiety. In certain embodiments, LBM is a cereblon E3 ubiquitin ligase binding moiety. In certain embodiments, LBM is a VHL E3 ubiquitin ligase binding moiety. In certain embodiments, LBM is an IAP E3 ubiquitin ligase binding moiety. In certain embodiments, LBM is an MDM2 E3 ubiquitin ligase binding moiety.

In certain embodiments, the present disclosure provides a compound of Formula I, wherein LBM is an E3 ubiquitin ligase (cereblon) binding moiety thereby forming a compound of Formulae I-a-1, I-a-2, I-a-3, I-a-4, I-a-5, I-a-6, I-a-7, I-a-8, I-a-9, or I-a-10 respectively:

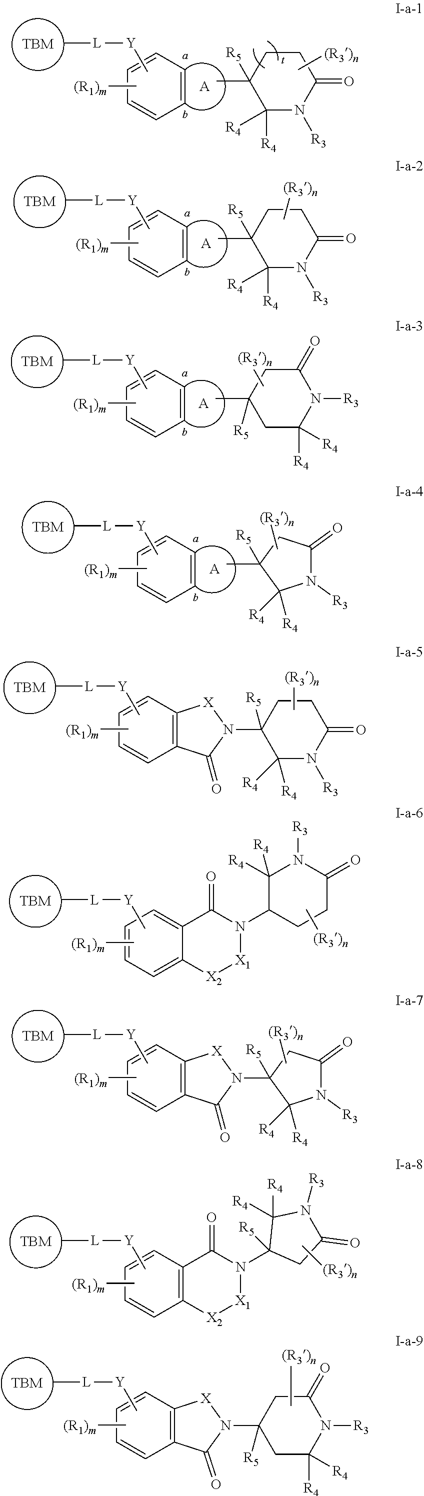

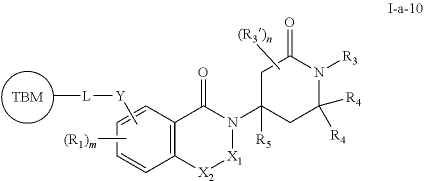

or a compound of Formulae I-a′-1, I-a′-2, I-a′-3, I-a′-4, I-a′-5, I-a′-6, I-a′-7, I-a′-8, I-a′-9, or I-a′-10 respectively:

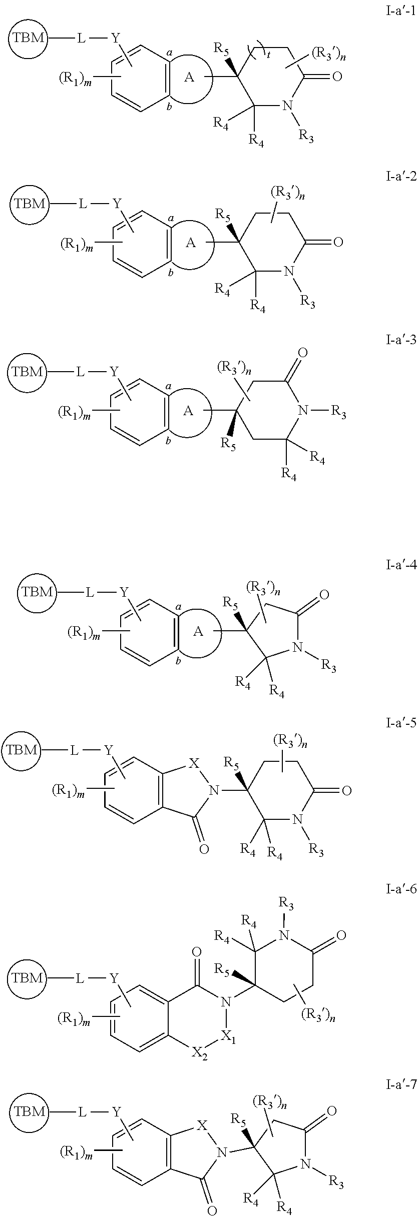

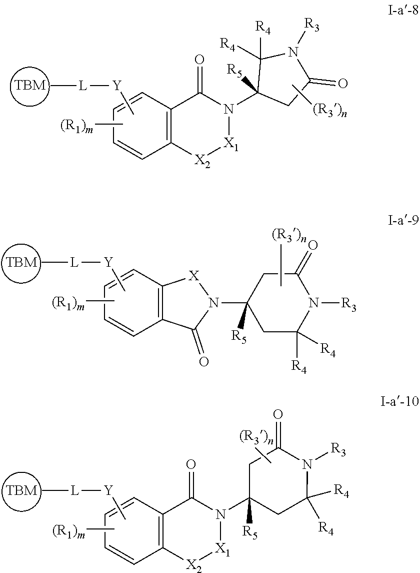

or a compound of Formulae I-a″-1, I-a″-2, I-a″-3, I-a″-4, I-a″-5, I-a″-6, I-a″-7, I-a″-8, I-a″-9, or I-a″-10 respectively:

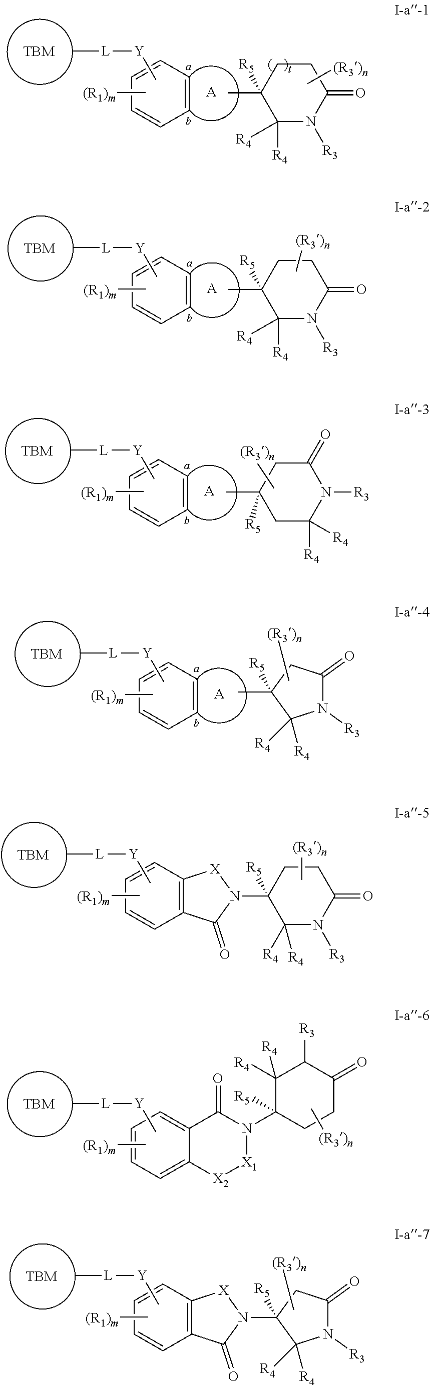

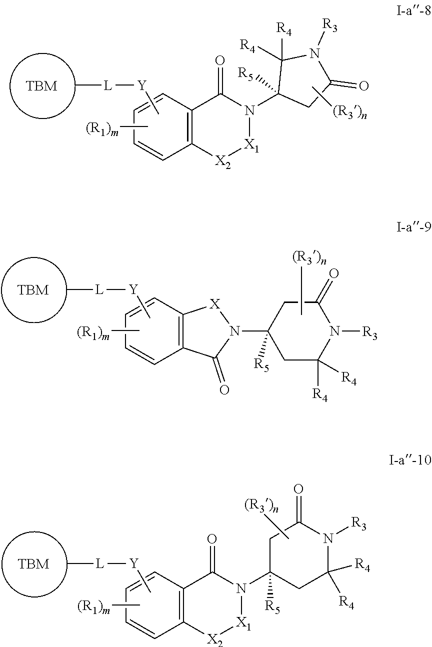

or a pharmaceutically acceptable salt thereof, wherein each of L and TBM is independently as defined above and described in embodiments herein, and wherein each of the variables

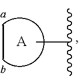

X, X₁, X₂, Y, R₁, R₃, R₃′, R₄, R₅, t, m, and n is independently as defined and described in international and U.S. patent publications, WO 2017/007612 and US 2018/0134684, the contents of each of which are herein incorporated by reference in their entireties.

For example, in certain embodiments:

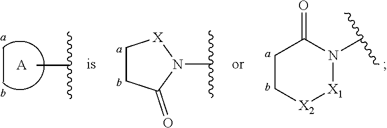

Y is a bond, Y₁, O, NH, NR₂, C(O)O, OC(O), C(O)NR₂′, NR₂′C(O), Y₁—O, Y₁—NH, Y₁—NR₂, Y₁—C(O), Y₁—C(O)O, Y₁—OC(O), Y₁—C(O)NR₂′, or Y₁—NR₂′C(O), wherein Y₁ is C₁-C₆ alkylene, C₂-C₆ alkenylene, or C₂-C₆ alkynylene;
X is C(O) or C(R₃)₂;
X₁-X₂ is C(R₃)═N or C(R₃)₂—C(R₃)₂;
each R₁ is independently halogen, nitro, NH₂, OH, C(O)OH, C₁-C₆ alkyl, or C₁-C₆ alkoxy; R₂ is C₁-C₆ alkyl, C₂-C₆ alkenyl, C₃-C₈ cycloalkyl, 3- to 8-membered heterocycloalkyl, C(O)—C₁-C₆ alkyl, C(O)—C₂-C₆ alkenyl, C(O)—C₃-C₈ cycloalkyl, or C(O)-3- to 8-membered heterocycloalkyl, and R₂ is optionally substituted with one or more of halogen, N(Rₐ)₂, NHC(O)Rₐ, NHC(O)ORₐ, OR_{b}, C₃-C₈ cycloalkyl, 3- to 8-membered heterocycloalkyl, C₆-C₁₀ aryl, or 5- to 10-membered heteroaryl, wherein each of the C₃-C₈ cycloalkyl, 3- to 8-membered heterocycloalkyl, C₆-C₁₀ aryl or 5- to 10-membered heteroaryl is optionally further substituted with one or more of halogen, NH₂, CN, nitro, OH, C(O)OH, C₁-C₆ alkyl, C₁-C₆ haloalkyl, C₁-C₆ alkoxy, or C₁-C₆ haloalkoxy;
R₂′ is H, C₁-C₆ alkyl, C₂-C₆ alkenyl, C₃-C₈ cycloalkyl, or 3- to 8-membered heterocycloalkyl, and R₂′, when not being H, is optionally substituted with one or more of halogen, N(Rₐ)₂, NHC(O)Rₐ, NHC(O)ORₐ, OR_{b}, C₃-C₈ cycloalkyl, 3- to 8-membered heterocycloalkyl, C₆-C₁₀ aryl, or 5- to 10-membered heteroaryl, wherein each of the C₃-C₈ cycloalkyl, 3- to 8-membered heterocycloalkyl, C₆-C₁₀ aryl or 5- to 10-membered heteroaryl is optionally further substituted with one or more of halogen, NH₂, CN, nitro, OH, C(O)OH, C₁-C₆ alkyl, C₁-C₆ haloalkyl, C₁-C₆ alkoxy, or C₁-C₆ haloalkoxy;
each R₃ is independently H or C₁-C₃ alkyl optionally substituted with C₆-C₁₀ aryl or 5- to 10-membered heteroaryl;
each R₃′ is independently C₁-C₃ alkyl;
each R₄ is independently H or C₁-C₃ alkyl; or two R₄, together with the carbon atom to which they are attached, form C(O), a C₃-C₆ carbocycle, or a 4-, 5-, or 6-membered heterocycle comprising 1 or 2 heteroatoms selected from N and O;
R₅ is H, C₁-C₃ alkyl, F, or Cl;
each Rₐ independently is H or C₁-C₆ alkyl;
R_{b} is H or tosyl;
t is 0 or 1;
m is 0, 1, 2 or 3; and
n is 0, 1 or 2.

The definitions of variables in the Formulae above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the present disclosure provides a compound of Formula I, wherein LBM is an E3 ubiquitin ligase (cereblon) binding moiety thereby forming a compound of Formulae I-b-1, I-b-2, I-b-3, I-b-4, I-b-5, or I-b-6 respectively:

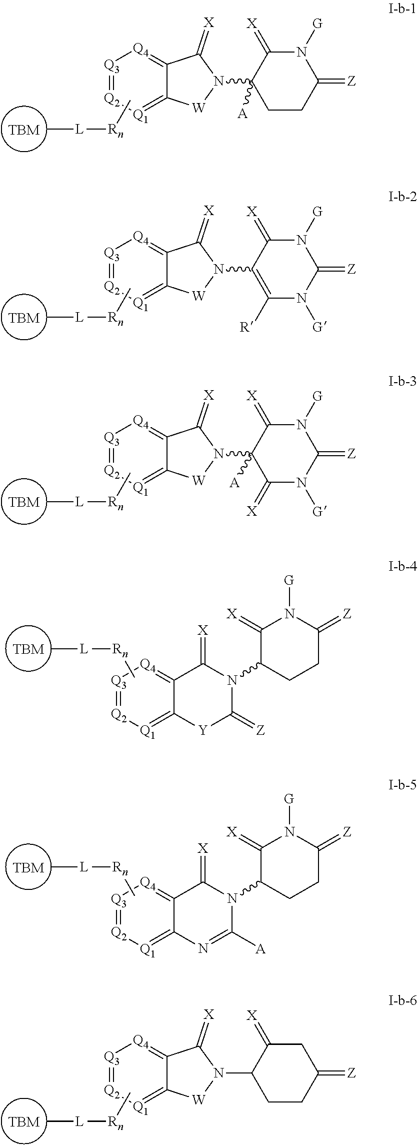

or a pharmaceutically acceptable salt thereof, wherein each of L and TBM is independently as defined above and described in embodiments herein, and wherein each of the variables A, G, G′, Q₁, Q₂, Q₃, Q₄, R, R′, W, X, Y, Z, [symbol] , and n is independently as defined and described in international and U.S. patent publications WO 2016/197114 and US 2018/0147202, the contents of each of which are herein incorporated by reference in their entireties.

For example, in certain embodiments:
W is independently selected from the group CH₂, CHR, C═O, SO₂, NH, and N-alkyl;
X is independently selected from the group O, S, and H₂;
Y is independently selected from the group NH, N-alkyl, N-aryl, N-hetaryl, N-cycloalkyl, N-heterocyclyl, O, and S;
Z is independently selected from the group O, S, and H₂, provided that both X and Z cannot both be H₂;
G and G′ are independently selected from the group H, alkyl, OH, CH₂-heterocyclyl optionally substituted with R′, and benzyl optionally substituted with R′;
Q₁-Q₄ each represent independently CR′, N, or N-oxide;
A is independently selected from the group alkyl, cycloalkyl, Cl and F;
R is selected from the group —CONR′R″, —OR′, —NR′R″, —SR, —SO₂R′, —SO₂NR′R″, —CR′R″—, —CR′NR′R″—, -aryl, -hetaryl, -alkyl, -cycloalkyl, -heterocyclyl, —P(O)(OR′)R″, —P(O)R′R″, —OP(O)(OR′)R″, —OP(O)R′R″, —Cl, —F, —Br, —I, —CF₃, —CN, —NR′SO₂NR′R″, —NR′CONR′R″, —CONR′COR″, —NR′C(═N—CN)NR′R″, —C(═N—CN)NR′R″, —NR′C(═N—CN)R″, —NR′C(═C—NO₂)NR′R″, —SO₂NR′COR″, —NO₂, —CO₂R′, —C(C═N—OR′)R″, —CR′═CR′R″, —CCR′, —S(C═O)(C═N—R′)R″, —SF₅, and —OCF₃;
R′ and R″ are independently selected from a bond, H, alkyl, cycloalkyl, aryl, heteroaryl, and heterocyclyl;
n is an integer from 1-4;
[symbol] represents a bond that may be stereospecific ((R) or (S)) or non-stereospecific; and
Rₙ comprises 1-4 independent functional groups or atoms.

The definitions of variables in the Formulae above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, LBM is

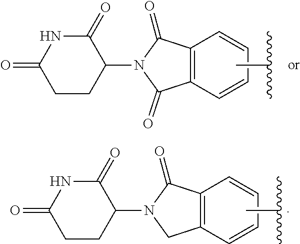

In certain embodiments, LBM is

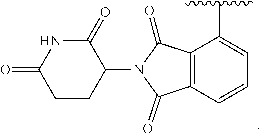 [SMILES: *c1cccc2c1C(=O)N(C1CCC(=O)NC1=O)C2=O]

In certain embodiments, LBM is

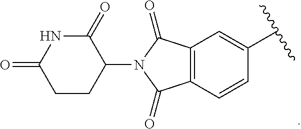 [SMILES: *c1ccc2c(c1)C(=O)N(C1CCC(=O)NC1=O)C2=O]

In certain embodiments, LBM is

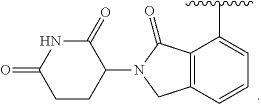 [SMILES: *c1cccc2c1C(=O)N(C1CCC(=O)NC1=O)C2]

In certain embodiments, LBM is

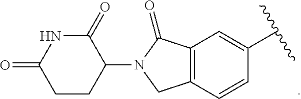 [SMILES: *c1ccc2c(c1)C(=O)N(C1CCC(=O)NC1=O)C2]

In certain embodiments, LBM is

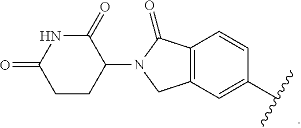 [SMILES: *c1ccc2c(c1)CN(C1CCC(=O)NC1=O)C2=O]

In certain embodiments, LBM is

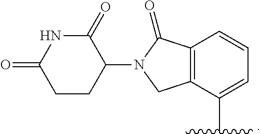 [SMILES: *c1cccc2c1CN(C1CCC(=O)NC1=O)C2=O]

In certain embodiments, LBM is

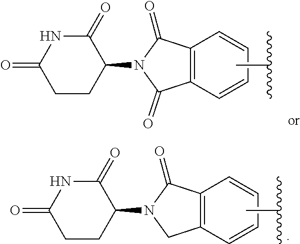

In certain embodiments, LBM is

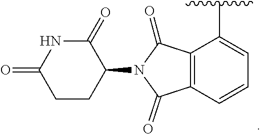 [SMILES: *c1cccc2c1C(=O)N([C@H]1CCC(=O)NC1=O)C2=O]

In certain embodiments, LBM is

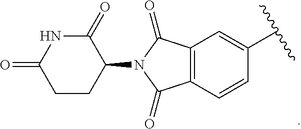 [SMILES: *c1ccc2c(c1)C(=O)N([C@H]1CCC(=O)NC1=O)C2=O]

In certain embodiments, LBM is

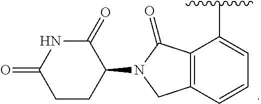 [SMILES: *c1cccc2c1C(=O)N([C@H]1CCC(=O)NC1=O)C2]

In certain embodiments, LBM is

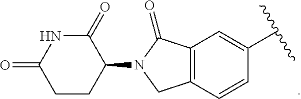 [SMILES: *c1ccc2c(c1)C(=O)N([C@H]1CCC(=O)NC1=O)C2]

In certain embodiments, LBM is

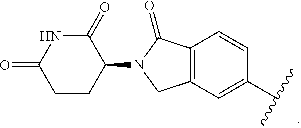 [SMILES: *c1ccc2c(c1)CN([C@H]1CCC(=O)NC1=O)C2=O]

In certain embodiments, LBM is

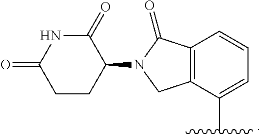 [SMILES: *c1cccc2c1CN([C@H]1CCC(=O)NC1=O)C2=O]

In certain embodiments, LBM is

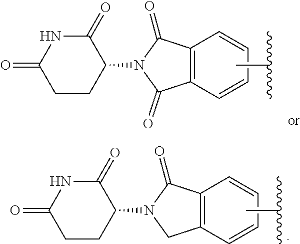

In certain embodiments, LBM is

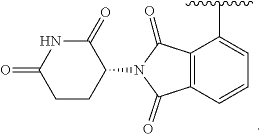 [SMILES: *c1cccc2c1C(=O)N([C@@H]1CCC(=O)NC1=O)C2=O]

In certain embodiments, LBM is

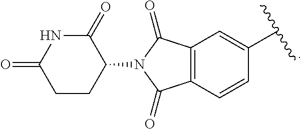 [SMILES: *c1ccc2c(c1)C(=O)N([C@@H]1CCC(=O)NC1=O)C2=O]

In certain embodiments, LBM is

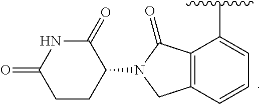 [SMILES: *c1cccc2c1C(=O)N([C@@H]1CCC(=O)NC1=O)C2]

In certain embodiments, LBM is

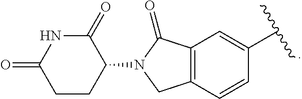 [SMILES: *c1ccc2c(c1)C(=O)N([C@@H]1CCC(=O)NC1=O)C2]

In certain embodiments, LBM is

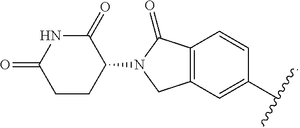 [SMILES: *c1ccc2c(c1)CN([C@@H]1CCC(=O)NC1=O)C2=O]

In certain embodiments, LBM is

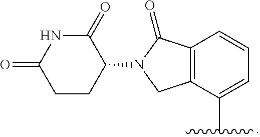 [SMILES: *c1cccc2c1CN([C@@H]1CCC(=O)NC1=O)C2=O]

In certain embodiments, the present disclosure provides a compound of Formula I, wherein LBM is an E3 ubiquitin ligase (cereblon) binding moiety thereby forming a compound of Formulae I-c-1, I-c-2, or I-c-3 respectively:

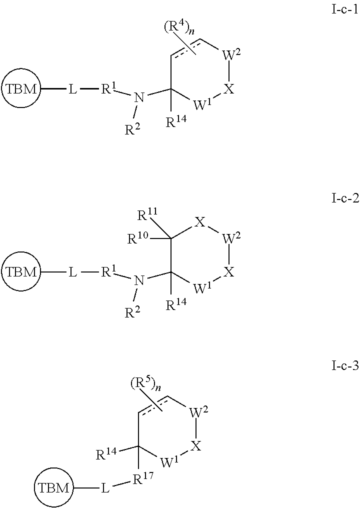

or a pharmaceutically acceptable salt thereof, wherein each of L and TBM is independently as defined above and described herein, and wherein each of the variables R¹, R², R⁴, R⁵, R¹⁰, R¹¹, R¹⁴, R¹⁷, W¹, W², X, [symbol] , and n is independently as defined in international patent publication WO 2017/197051, the content of which is herein incorporated by reference in its entirety, and wherein

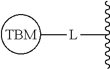

is attached to R¹, the ring formed by combining R¹ and R², or R¹⁷ at the site of attachment of R¹² as defined in international patent publication WO 2017/197051 such that

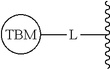

takes the place of the R¹² substituent.

For example, in certain embodiments:
W¹ is CR⁶R⁷, C═O, C═S, C═CH₂, SO₂, S(O), P(O)Oalkyl, P(O)NHalkyl, P(O)N(alkyl)₂, P(O)alkyl, P(O)OH, P(O)NH₂;
W² is CR′R⁹, C═O, C═S, C═CH₂, SO₂, S(O), P(O)Oalkyl, P(O)NHalkyl, P(O)N(alkyl)₂, P(O)alkyl, P(O)OH, P(O)NH₂;
X is independently selected from NH, NR³, CH₂, CHR³, C(R³)₂, O, and S;
n is 0, 1, 2, or 3;
[symbol] is a single or double bond; wherein when [symbol] represents a single bond, n is 0, 1, 2, or 3, and when [symbol] represents a double bond, n is 0, 1, or 2;
R¹ is selected from:

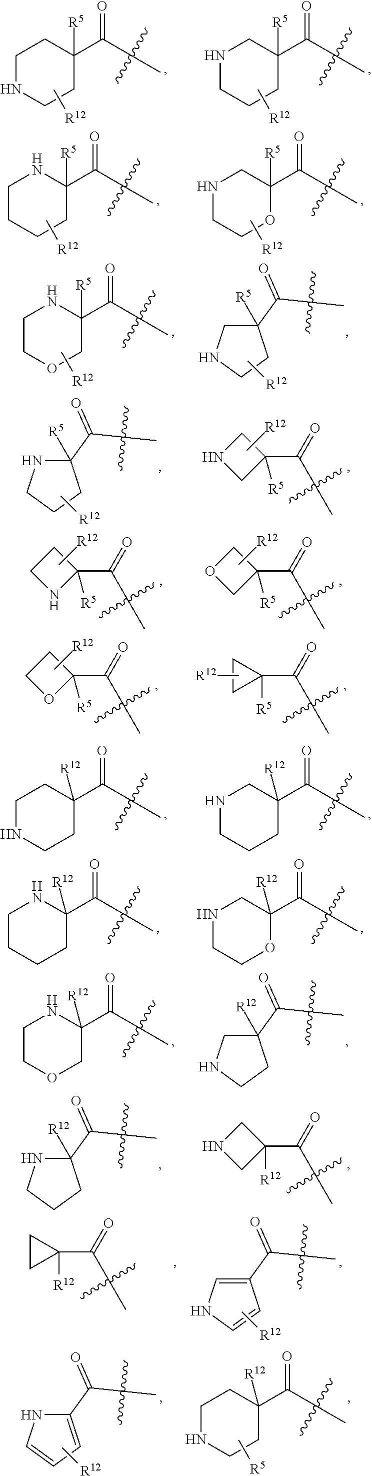

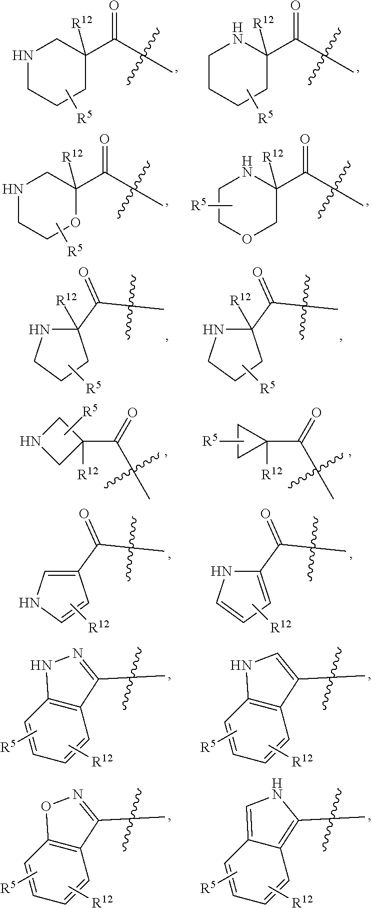

### and R¹*;

or R¹ is selected from:

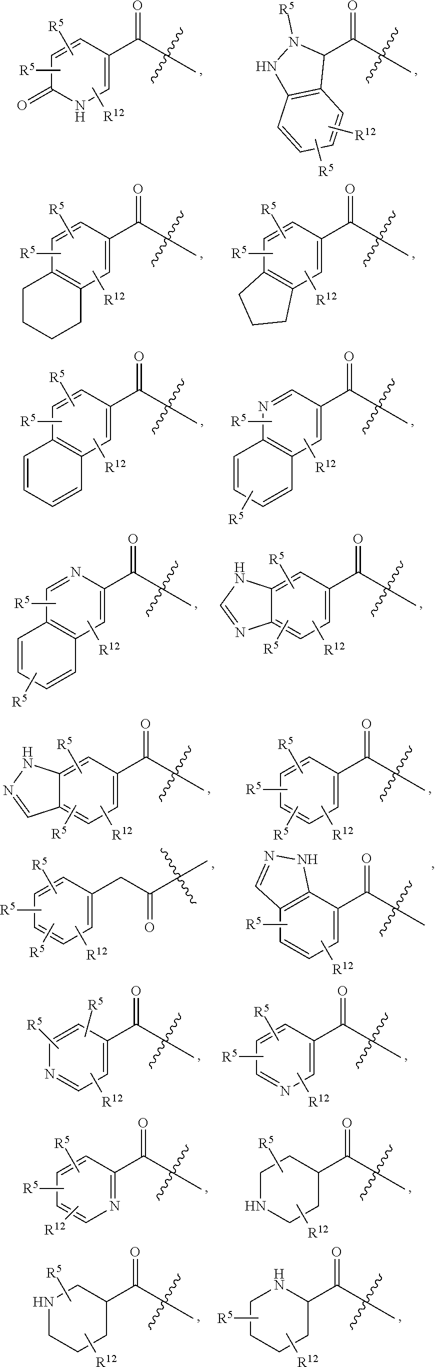

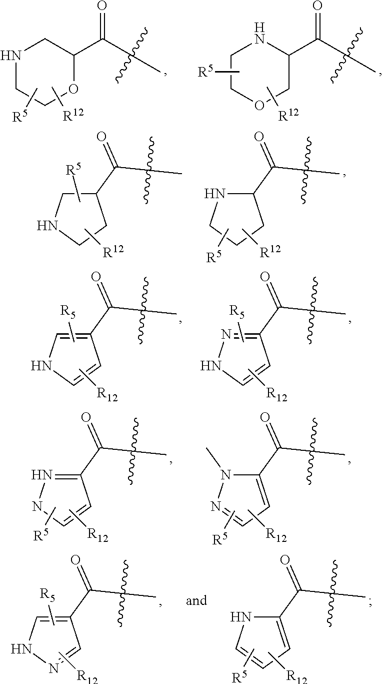

R² is alkyl, hydrogen, aliphatic, heteroaliphatic, aryl, heteroaryl or heterocyclic;
or R¹ and R² are combined to form a 4, 5, 6, 7, 8, 9, or 10 membered heterocyclo or heteroaryl species, wherein the heterocyclo or heteroaryl species is substituted with R¹² at any desired position, wherein the heterocyclo or heteroaryl species is optionally further substituted with one or more substituents selected from R⁵;
R¹* is selected from:

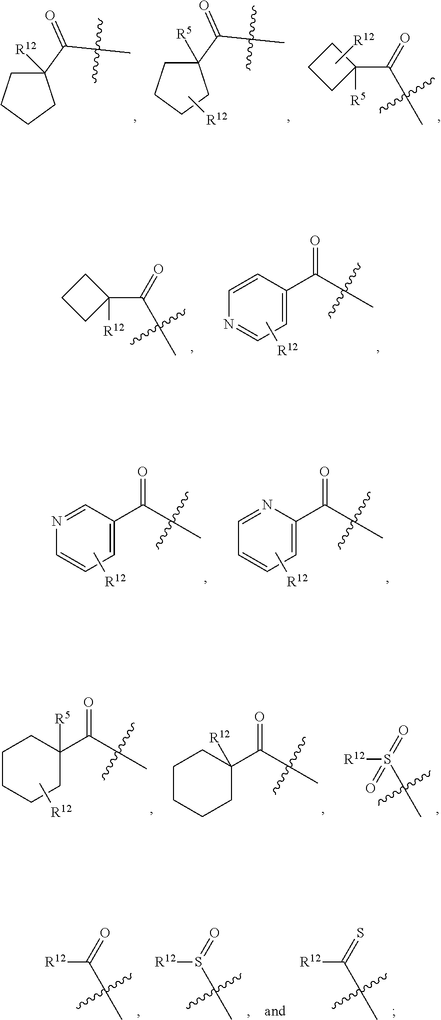

R³ is selected at each instance from: alkyl, —C(O)H, —C(O)OH, —C(O)alkyl, —C(O)Oalkyl, alkene, and alkyne, aliphatic, heteroaliphatic, aryl, heteroaryl and heteroalkyl;
R⁴ is selected at each instance from: alkyl, alkene, alkyne, halogen, hydroxyl, alkoxy, azide, amino, cyano, —NH(aliphatic), —N(aliphatic)₂, —NHSO₂(aliphatic), —N(aliphatic)SO₂alkyl, —NHSO₂(aryl, heteroaryl or heterocyclic), —N(alkyl)SO₂(aryl, heteroaryl or heterocyclic) —NHSO₂alkenyl, —N(alkyl)SO₂alkenyl, —NHSO₂alkynyl, —N(alkyl)SO₂alkynyl, and haloalkyl; aliphatic, heteroaliphatic, aryl, heteroaryl, heteroalkyl and carbocyclic;
or two R⁴ substituents together with the carbon atom(s) to which they are bound can form a 3, 4, 5 or 6 membered ring;
R⁵ and R¹⁴ are selected at each instance from: hydrogen, alkyl, alkene, alkyne, halogen, hydroxyl, alkoxy, azide, amino, cyano, —NH(aliphatic), —N(aliphatic)₂, —NHSO₂(aliphatic), —N(aliphatic)SO₂alkyl, —NHSO₂(aryl, heteroaryl or heterocyclic), —N(alkyl)SO₂(aryl, heteroaryl or heterocyclic) —NHSO₂alkenyl, —N(alkyl)SO₂alkenyl, —NHSO₂alkynyl, —N(alkyl)SO₂alkynyl, and haloalkyl; aliphatic, heteroaliphatic, aryl, heteroaryl, heteroalkyl and carbocyclic;
or R⁵ is independently selected from C(O)R⁴, cyano, aryl, aryloxy, heterocyclo, heteroaryl, arylalkyl, alkoxy, hydroxyl, O-arylalkyl, or cycloalkyl;
R⁶, R⁷, R⁸, R⁹, R¹⁰, and R¹¹, are independently selected from hydrogen, alkyl, aliphatic, heteroaliphatic, hydroxyl, alkoxy, amine, —NH(aliphatic), and —N(aliphatic)₂;
or R⁶ and R⁷ together with the carbon to which they are bound form a 3-, 4-, 5-, or 6-membered spirocarbocycle, or a 4-, 5-, or 6-membered spiroheterocycle comprising 1 or 2 heteroatoms selected from N and 0;
or R⁸ and R⁹ together with the carbon to which they are bound form a 3-, 4-, 5-, or 6-membered spirocarbocycle, or a 4-, 5-, or 6-membered spiro heterocycle comprising 1 or 2 heteroatoms selected from N and 0;
or R¹⁰ and R¹¹ together with the carbon to which they are bound form a 3-, 4-, 5-, or 6-membered spirocarbocycle, or a 4-, 5-, or 6-membered spiro heterocycle comprising 1 or 2 heteroatoms selected from N and 0;
or R⁶ and R⁸ form a 1 or 2 carbon bridged ring;
or R⁶ and R¹⁰ form a 1 or 2 carbon bridged ring;
or R⁸ and R¹⁰ form a 1 or 2 carbon bridged ring;
or R¹⁴ and R⁶ form a 3, 4, 5, or 6 carbon fused ring;
or R¹⁴ and R¹⁰ form a 3, 4, 5, or 6 carbon fused ring;
or R¹⁴ and R⁸ form a 1 or 2 carbon bridged ring;
or R¹⁴ and R⁴ form a 3, 4, 5, or 6 carbon fused ring wherein R⁵ is on the carbon alpha to R¹⁴ or a 1, 2, 3, or 4 carbon bridged ring wherein R⁵ is not on the carbon alpha to R¹⁴;
R¹² is a bond to L;
R¹⁷ is selected from:

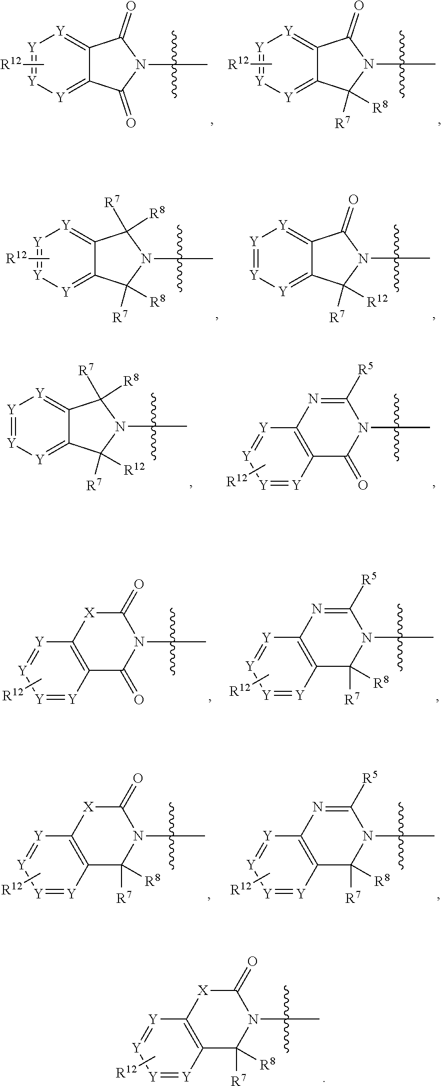

Y is independently selected from N, CH, or CR¹⁰¹, wherein 0, 1, 2, or 3 instances of Y are selected to be N; and
R¹⁰¹ is independently selected at each occurrence from hydrogen, alkyl, alkenyl, alkynyl, haloalkyl, alkoxyl, hydroxyl, aryl, heteroaryl, heterocycle, arylalkyl, heteroarylalkyl, heterocycloalkyl, aryloxy, heteroaryloxy, CN, —COOalkyl, COOH, NO₂, F, Cl, Br, I, CF₃, NH₂, NHalkyl, N(alkyl)₂, aliphatic, and heteroaliphatic.

The definitions of variables in the Formulae above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the present disclosure provides a compound of Formula I, wherein LBM is an E3 ubiquitin ligase (cereblon) binding moiety thereby forming a compound of Formulae I-d-1, I-d-2, I-d-3, or I-d-4, respectively:

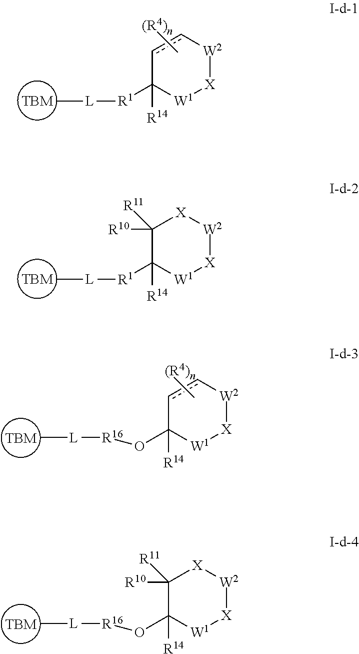

or a pharmaceutically acceptable salt thereof, wherein each of L and TBM is independently as defined above and described herein, and wherein each of the variables R¹, R⁴, R¹⁰, R¹¹, R¹⁴, R¹⁶, W¹, W², X, [symbol] , and n is independently as defined in international patent publication WO 2018/237026, the content of which is herein incorporated by reference in its entirety, and wherein

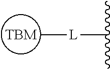

is attached to R¹ or R¹⁶ at the site of attachment of R¹² as defined in international patent publication WO 2018/237026, such that

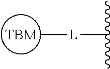

takes the place of the R¹² substituent.

For example, in certain embodiments, the present disclosure provides a compound of Formulae I-d-1 or I-d-2, wherein:
W¹ is CR⁶R⁷, C═O, C═S, C═CH₂, SO₂, S(O), P(O)Oalkyl, P(O)NHalkyl, P(O)N(alkyl)₂, P(O)alkyl, P(O)OH, P(O)NH₂;
W² is CR⁸R⁹, C═O, C═S, C═CH₂, SO₂, S(O), P(O)Oalkyl, P(O)NHalkyl, P(O)N(alkyl)₂, P(O)alkyl, P(O)OH, P(O)NH₂;
X is independently selected from NH, NR³, CH₂, CHR³, C(R³)₂, O, and S;
n is 0, 1, 2, or 3;
[symbol] is a single or double bond; wherein when [symbol] represents a single bond, n is 0, 1, 2, or 3, and when [symbol] represents a double bond, n is 0, 1, or 2;
R¹ is selected from —N(R²)(R¹²), —OC(O)N(R²)(R¹²), and —OR¹²;
R² is independently alkyl, hydrogen, aliphatic, heteroaliphatic, aryl, heteroaryl or heterocyclic;
R³ is selected at each instance from alkyl, —C(O)H, —C(O)OH, —C(O)alkyl, —C(O)Oalkyl, alkene, and alkyne;
R⁴ is selected at each instance from alkyl, alkene, alkyne, halogen, hydroxyl, alkoxy, azide, amino, —NH(alkyl), —N(alkyl)₂, —NHSO₂(alkyl), —N(alkyl)SO₂alkyl, —NHSO₂(aryl), —N(alkyl)SO₂(aryl), —NHSO₂alkenyl, —N(alkyl)SO₂alkenyl, —NHSO₂alkynyl, —N(alkyl)SO₂alkynyl, and haloalkyl;
or two R⁴ substituents together with the carbon atom(s) to which they are bound can form a 3, 4, 5 or 6 membered ring;
R¹⁴ is selected from hydrogen, alkyl, alkene, alkyne, halogen, hydroxyl, alkoxy, azide, amino, cyano, —NH(aliphatic, including alkyl), —N(aliphatic, including alkyl)₂, —NHSO₂(aliphatic, including alkyl), —N(aliphatic, including alkyl)SO₂alkyl, —NHSO₂(aryl, heteroaryl or heterocyclic), —N(alkyl)SO₂(aryl, heteroaryl or heterocyclic) —NHSO₂alkenyl, —N(alkyl)SO₂alkenyl, —NHSO₂alkynyl, —N(alkyl)SO₂alkynyl, haloalkyl, aliphatic, heteroaliphatic, aryl, heteroaryl, heteroalkyl, and carbocyclic;
R⁶, R⁷, R⁸, R⁹, R¹⁰, and R¹¹, are independently selected from hydrogen, alkyl, aliphatic, heteroaliphatic, hydroxyl, alkoxy, amine, —NH(aliphatic), and —N(aliphatic)₂; or R⁶ and R⁷ together with the carbon to which they are bound form a 3-, 4-, 5-, or 6-membered spirocarbocycle, or a 4-, 5-, or 6-membered spiroheterocycle comprising 1 or 2 heteroatoms selected from N and 0;
or R⁸ and R⁹ together with the carbon to which they are bound form a 3-, 4-, 5-, or 6-membered spirocarbocycle, or a 4-, 5-, or 6-membered spiro heterocycle comprising 1 or 2 heteroatoms selected from N and 0;
or R¹⁰ and R¹¹ together with the carbon to which they are bound form a 3-, 4-, 5-, or 6-membered spirocarbocycle, or a 4-, 5-, or 6-membered spiro heterocycle comprising 1 or 2 heteroatoms selected from N and 0;
or R⁶ and R⁸ form a 1 or 2 carbon bridged ring;
or R⁶ and R¹⁰ form a 1 or 2 carbon bridged ring;
or R⁸ and R¹⁰ form a 1 or 2 carbon bridged ring;
or R¹⁴ and R⁶ form a 3, 4, 5, or 6 carbon fused ring;
or R¹⁴ and R¹⁰ form a 3, 4, 5, or 6 carbon fused ring;
or R¹⁴ and R⁸ form a 1 or 2 carbon bridged ring;
or R¹⁴ and R⁴ form a 3, 4, 5, or 6 carbon fused ring wherein R⁵ is on the carbon alpha to R¹⁴ or a 1, 2, 3, or 4 carbon bridged ring wherein R⁵ is not on the carbon alpha to R¹⁴; and
R¹² is a bond to L.

In certain embodiments, the present disclosure provides a compound of Formulae I-d-3 or I-d-4, wherein R¹⁶ is selected from:

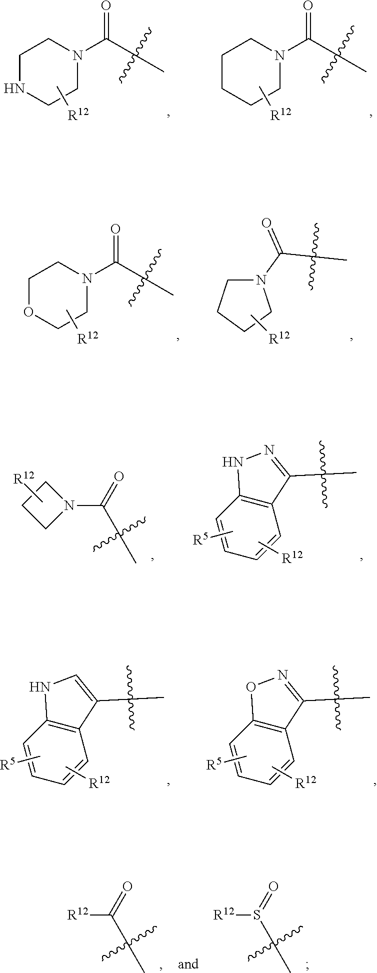

wherein each R is optionally substituted with one or more substituents independently selected from R⁵; and all other variables are as defined in Formulae I-d-1 and I-d-2, above.

The definitions of variables in the Formulae above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the present disclosure provides a compound of Formula I, wherein LBM is an E3 ubiquitin ligase (cereblon) binding moiety thereby forming a compound of Formulae I-e-1 or I-e-3, respectively:

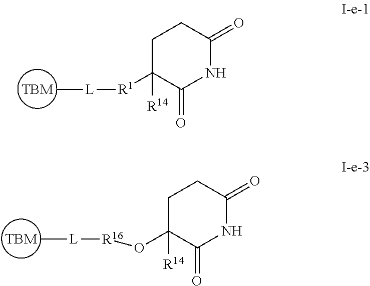

or a pharmaceutically acceptable salt thereof, wherein each of L and TBM is independently as defined above and described herein, and wherein each of the variables R¹, R¹⁴, and R¹⁶ is independently as defined in international patent publication WO 2018/237026, the contents of which are herein incorporated by reference in their entirety, and wherein

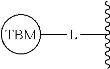

is attached to R¹ or R¹⁶ at the site of attachment of R¹² as defined in international patent publication WO 2018/237026, such that

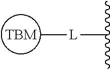

takes the place of the R¹² substituent.

For example, in certain embodiments, the present disclosure provides a compound of Formulae I-e-1 or I-e-3, wherein R¹, R¹⁴, and R¹⁶ are as defined for Formulae I-d-1, I-d-2, I-d-3, and I-d-4.

In certain embodiments, the present disclosure provides a compound of Formula I, wherein LBM is an E3 ubiquitin ligase (cereblon) binding moiety thereby forming a compound of Formulae I-f-1, I-f-2, I-f-3, I-f-4, I-f-5, I-f-6, I-f-7, or I-f-8:

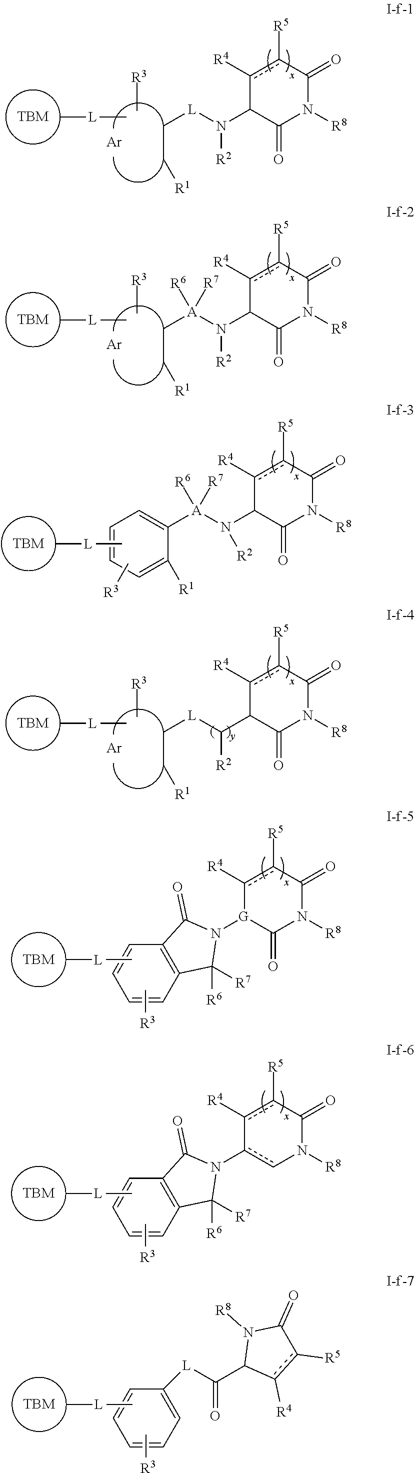

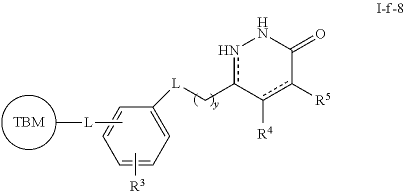

or a pharmaceutically acceptable salt thereof, wherein each of L and TBM is independently as defined above and described in embodiments herein, and wherein each of the variables Ar, R¹, R², R³, R⁴, R⁵, R, R⁷, R⁸, A, L, x, y, and [symbol] is independently as described and defined in international patent publication WO 2017/161119, the contents of which are herein incorporated by reference in their entirety.

For example, in certain embodiments, the present disclosure provides a compound of Formula I-f-1, wherein:
Ar is aryl, heteroaryl, cycloalkyl, or heterocyclyl;
the L connecting Ar and N(R²) is: absent or a linker selected from the group consisting of —SO₂, —SO₂R; SO₂R′R″, —SO₂NR′R″; —SO₂NR′R″C(═O); —NR′SO₂R″; —R′SO₂NR′R″; —C(═O); —C(═O)R′; —OC(═O)R′; —C(═O)NR′R″; —NR′C(═O)R″; —NR′C(═O)R″C(═O); —OR′; —NR′R″; —SR′; —N₃—C(═O)OR′; —O(CR′R″)rC(═O)R′; —O(CR′R″)rNR″C(═O)R′; —O(CR′R″)rNR″SO₂R′; —OC(═O)NR′R″; —NR′C(═O)OR″; and substituted or unsubstituted C₁-C₆ aliphatic alkyl;
wherein R′, R″, and R′″ are individually selected from hydrogen; substituted or unsubstituted alkyl; substituted or unsubstituted alkenyl; substituted or unsubstituted ether; substituted or unsubstituted cycloalkyl; substituted or unsubstituted heterocyclyl; substituted or unsubstituted cycloalkenyl, substituted or unsubstituted aryl, substituted or unsubstituted heteroaryl, substituted or unsubstituted arylalkyl, substituted or unsubstituted heteroalkyl, substituted or unsubstituted alkylheteroaryl, or substituted or unsubstituted amine; and r is an integer from 1 to 6;
R¹, R², and R³ are individually selected from the group consisting of hydrogen, halogen, hydroxyl, azide, alkoxyl, sulfhydryl, imino, amido, phosphonate, phosphinate, carbonyl, carboxyl, cyano, silyl, ether, alkylthio, sulfonyl, sulfonamido, ketone, aldehyde, substituted or unsubstituted amine, substituted or unsubstituted amide, nitro, ester, morpholino, dioxolane, substituted or unsubstituted alkyl, substituted or unsubstituted alkylhalide, substituted or unsubstituted aralkyl, substituted or unsubstituted alkenyl, substituted or unsubstituted alkynyl, substituted or unsubstituted cycloalkyl, substituted or unsubstituted cycloalkyl; substituted or unsubstituted heterocyclyl; substituted or unsubstituted cycloalkenyl, substituted or unsubstituted aryl, substituted or unsubstituted heteroaryl, substituted or unsubstituted arylalkyl, substituted or unsubstituted heteroalkyl, substituted or unsubstituted alkylheteroaryl, and combinations thereof; or
wherein R¹ and R² combine to form a 5-7 membered heterocyclic ring; and wherein when R¹ and R² combine to form a 5-7 membered heterocyclic ring, Ar is optionally not fused to the 5-7 membered heterocyclic ring but is a substituent of the 5-7 membered heterocyclic ring;
R⁴ and R⁵ are individually selected from the group consisting of hydrogen, halogen, hydroxyl, azide, ether, alkoxyl, sulfhydryl, alkylthio, sulfonyl, sulfonamido, ketone, aldehyde, ester, substituted or unsubstituted alkyl, substituted or unsubstituted alkylhalide, substituted or unsubstituted aralkyl, substituted or unsubstituted alkenyl, substituted or unsubstituted alkynyl, substituted or unsubstituted cycloalkyl, heterocyclyl, and combinations thereof,
R⁸ is selected from the group consisting of hydrogen, substituted or unsubstituted alkyl, substituted or unsubstituted alkylhalide, substituted or unsubstituted aralkyl, substituted or unsubstituted alkenyl, substituted or unsubstituted alkynyl, substituted or unsubstituted cycloalkyl, heterocyclyl, substituted or unsubstituted aryl, substituted or unsubstituted alkylaryl, and combinations thereof;
x is 0, 1, or 2; and
[symbol] is a single or double bond.

In certain embodiments, the present disclosure provides a compound of Formulae I-f-2 and/or I-f-3, wherein A comprises C, S, substituted or unsubstituted C₁-C₈ alkyl, or combinations thereof, and R⁶ and R⁷ are individually ═O, hydrogen, C₁-C₈ alkyl, or R⁶ and R⁷ combine to form ═O; and all other variables in Formulae I-f-2 and/or I-f-3 are as defined in Formulae I-f-1, above.

In certain embodiments, the present disclosure provides a compound of Formula I-f-4, wherein y is from 1 to 6; and all other variables in Formula I-f-4 are as defined in Formulae I-f-1, above.

In certain embodiments, the present disclosure provides a compound of Formulae I-f-5 and/or I-f-6 wherein:
G comprises C, S, N, substituted of unsubstituted C1-C8 alkyl, or combinations thereof;
R³ is selected from the group consisting of hydrogen, halogen, hydroxyl, azide, alkoxyl, sulfhydryl, imino, amido, phosphonate, phosphinate, carbonyl, carboxyl, cyano, silyl, ether, alkylthio, sulfonyl, sulfonamido, ketone, aldehyde, substituted or unsubstituted amine, substituted or unsubstituted amide, nitro, ester, morpholino, dioxolane, substituted or unsubstituted alkyl, substituted or unsubstituted alkylhalide, substituted or unsubstituted aralkyl, substituted or unsubstituted alkenyl, substituted or unsubstituted alkynyl, substituted or unsubstituted cycloalkyl; substituted or unsubstituted heterocyclyl; substituted or unsubstituted cycloalkenyl, substituted or unsubstituted aryl, substituted or unsubstituted heteroaryl, substituted or unsubstituted arylalkyl, substituted or unsubstituted heteroalkyl, substituted or unsubstituted alkylheteroaryl, substituted or unsubstituted cycloalkyl, and combinations thereof,
R⁴, R⁵, and R⁸ are individually selected from the group consisting of hydrogen, halogen, hydroxyl, azide, ether, alkoxyl, sulfhydryl, alkylthio, sulfonyl, sulfonamido, ketone, aldehyde, ester, substituted or unsubstituted alkyl, substituted or unsubstituted alkylhalide, substituted or unsubstituted aralkyl, substituted or unsubstituted aryl, substituted or unsubstituted alkylaryl, substituted or unsubstituted alkenyl, substituted or unsubstituted alkynyl, substituted or unsubstituted cycloalkyl, heterocyclyl, and combinations thereof; and
R⁶ and R⁷ are individually ═O, hydrogen, C1-C8 alkyl, or R⁶ and R⁷ combine to form ═O;
x is 0, 1, or 2; and
[symbol] is a single or double bond.

In certain embodiments, the present disclosure provides a compound of Formulae I-f-7 and/or I-f-8 wherein R¹ and the occurrence of L that is not attached to TBM are as defined in Formula I-f-1, y is 0, 1, or 2, and R³, R⁴, R⁵, and R⁸ are as defined in Formulae I-f-5 and/or I-f-6.

The definitions of variables in the Formulae above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the present disclosure provides a compound of Formula I, wherein LBM is an E3 ubiquitin ligase (cereblon) binding moiety thereby forming a compound of Formula I-g:

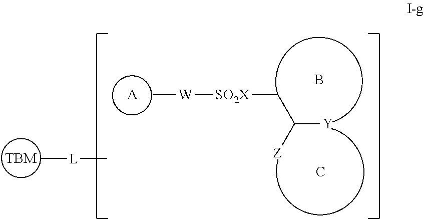

or a pharmaceutically acceptable salt thereof, wherein each of L and TBM is independently as defined above and described in embodiments herein, and wherein each of the variables A, B, C, W, X, Y, and Z is independently as described and defined in U.S. Pat. No. 5,721,246, the contents of which are herein incorporated by reference in their entirety.

For example, in certain embodiments:
A represents a monocyclic or bicyclic aromatic ring which may be substituted;
B represents a six-membered unsaturated hydrocarbon ring or a six-membered unsaturated heterocycle containing one nitrogen atom as the heteroatom, each of which may be substituted;
C represents a five-membered heterocycle containing one or two nitrogen atoms which may be substituted with halogen, cyano, lower alkyl, lower alkoxy, hydroxyl, oxo, groups represented by formula —C(O)-r (wherein r represents hydrogen, amino which may be substituted with lower alkyl, lower alkyl, lower alkoxy or hydroxyl), amino substituted with lower alkyl, and trifluoromethyl;
W represents a single bond or a group represented by formula —CH═CH—;
X represents the formula —N(R′)— or oxygen;
Y represents carbon or nitrogen;
Z represents a group represented by formula —N(R²)— or nitrogen; and
R¹ and R² may be the same or different from each other and each represent hydrogen or lower alkyl.

In certain embodiments, the variables in Formula I-g are as defined above with the provisos that (1) the case wherein A is 4-methyl-benzene, W is a single bond, X is a group represented by formula —NH—, B is methoxybenzene and C is unsubstituted imidazole and (2) the case wherein A is 4-(acetamido)benzene or 4-aminobenzene, W is a single bond, X is a group represented by formula —NH—, B is unsubstituted benzene and C is unsubstituted pyrazole, and (3) the case wherein A is a lower alkyl-substituted phenyl, nitro-substituted phenyl or halogen-substituted phenyl, and X is oxygen, are excepted.

The definitions of variables in Formula I-g above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the present disclosure provides a compound of Formula I, wherein LBM is an E3 ubiquitin ligase (cereblon) binding moiety thereby forming a compound of Formula I-h:

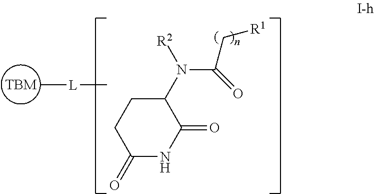

or a pharmaceutically acceptable salt thereof, wherein each of L and TBM is independently as defined above and described in embodiments herein, and wherein each of the variables R₁, R₂, and n is independently as described and defined in international patent publication WO 2019/043214, the contents of which are herein incorporated by reference in their entireties.

For example, in certain embodiments:
R¹ is selected from the group consisting of:
   i) aryl, optionally substituted by one or more R³,
   ii) heterocycloalkyl, optionally substituted by one or more R⁴,
   iii) cycloalkyl, optionally substituted by one or more R⁵, and
   iv) heteroaryl, optionally substituted by one or more R⁶,
R² is H, C₁₋₆alkyl or —NC(O)—C₁₋₆alkyl,
R³ is H or C₁₋₆alkyl,
R⁴ is H or C₁₋₆alkyl,
R⁵ is H or C₁₋₆alkyl,
R⁶ is H, C₁₋₆alkyl, —C(O)O—C₁₋₆alkyl or oxo, and
n is 0 or 1.

The definitions of variables in Formula I-h above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, LBM is an IAP E3 Ubiquitin ligase binding moiety recited in Varfolomeev, E. et al., IAP Antagonists Induce Autoubiquitination of c-IAPs, NF-κB activation, and TNFα-Dependent Apoptosis, Cell, 2007, 131(4): 669-81, such as, for example:

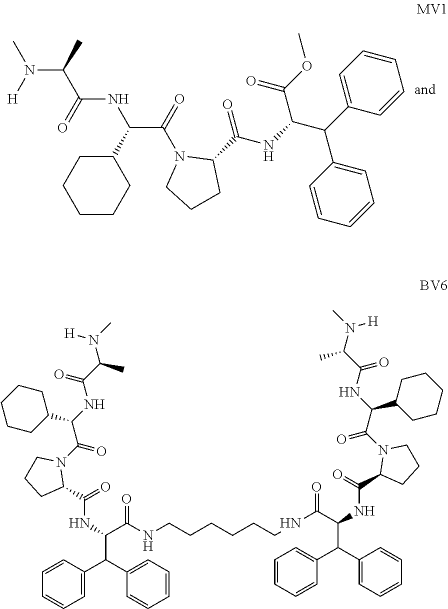 [SMILES: CN[C@@H](C)C(=O)N[C@H](C(=O)N1CCC[C@H]1C(=O)N[C@H](C(=O)OC)C(c1ccccc1)c1ccccc1)C1CCCCC1 ; CN[C@@H](C)C(=O)N[C@H](C(=O)N1CCC[C@H]1C(=O)N[C@H](C(=O)NCCCCCCNC(=O)[C@@H](NC(=O)[C@@H]1CCCN1C(=O)[C@@H](NC(=O)[C@H](C)NC)C1CCCCC1)C(c1ccccc1)c1ccccc1)C(c1ccccc1)c1ccccc1)C1CCCCC1]

wherein

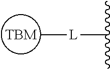

is attached to a modifiable carbon, oxygen, nitrogen or sulfur atom.

In certain embodiments, the present disclosure provides a compound of Formula I, wherein LBM is a VHL E3 ubiquitin ligase binding moiety thereby forming a compound of Formulae I-i-1, I-i-2, I-i-3, I-i-4, or I-i-5 respectively:

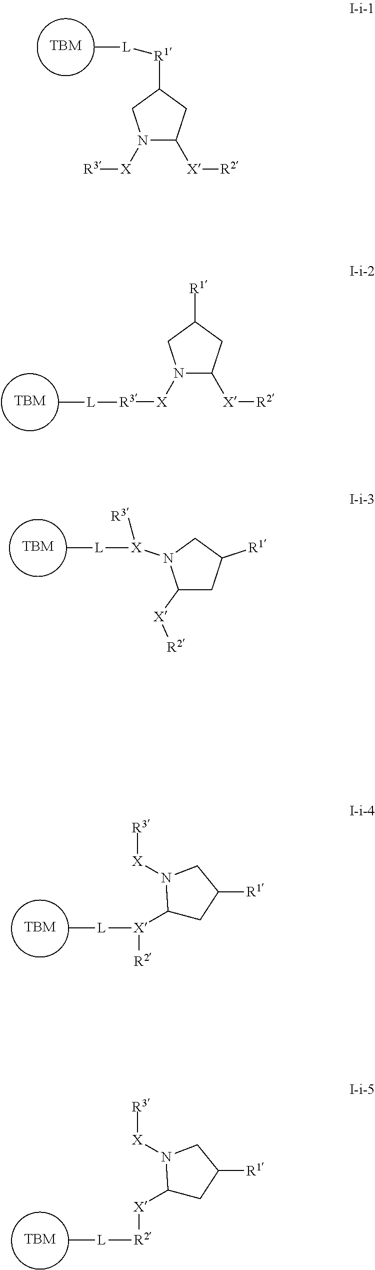

or a pharmaceutically acceptable salt thereof, wherein each of L and TBM is independently as defined above and described in embodiments herein, and wherein each of the variables R^{1′}, R^{2′}, R^{3′}, X, and X′ is as independently defined and described in international and U.S. patent publications WO 2013/106643 and US 2014/0356322, the contents of each of which are herein incorporated by reference in their entireties.

For example, in certain embodiments, the present disclosure provides a compound of Formulae I-i-1, I-i-2, I-i-3, I-i-4, or I-i-5 wherein:
R^{1′} is an optionally substituted C₁-C₆ alkyl group, an optionally substituted —(CH₂)ₙOH, an optionally substituted —(CH₂)ₙSH, an optionally substituted (CH₂)ₙ—O—(C₁-C₆)alkyl group, an optionally substituted (CH₂)ₙ—WCOCW—(C₀-C₆)alkyl group containing an epoxide moiety WCOCW where each W is independently H or a C₁-C₃ alkyl group, an optionally substituted —(CH₂)ₙCOOH, an optionally substituted —(CH₂)ₙC(O)—(C₁-C₆ alkyl), an optionally substituted —(CH₂)ₙNHC(O)—R₁, an optionally substituted —(CH₂)ₙC(O)—NR₁R², an optionally substituted —(CH₂)ₙOC(O)—NR₁R², —(CH₂O)ₙH, an optionally substituted —(CH₂)ₙOC(O)—(C₁-C₆ alkyl), an optionally substituted —(CH₂)ₙC(O)—O—(C₁-C₆ alkyl), an optionally substituted —(CH₂O)ₙCOOH, an optionally substituted —(OCH₂)ₙO—(C₁-C₆ alkyl), an optionally substituted —(CH₂O)ₙC(O)—(C₁-C₆ alkyl), an optionally substituted —(OCH₂)ₙNHC(O)—R₁, an optionally substituted —(CH₂O)ₙC(O)—NR₁R², —(CH₂CH₂O)ₙH, an optionally substituted —(CH₂CH₂O)ₙCOOH, an optionally substituted —(OCH₂CH₂)ₙO—(C₁-C₆ alkyl), an optionally substituted —(CH₂CH₂O)ₙC(O)—(C₁-C₆ alkyl), an optionally substituted —(OCH₂CH₂)ₙNHC(O)—R₁, an optionally substituted —(CH₂CH₂O)ₙC(O)—NR₁R², an optionally substituted —SO₂R_{S}, an optionally substituted S(O)R_{S}, NO₂, CN or halogen (F, Cl, Br, I, preferably F or Cl);
R₁ and R₂ are each independently H or a C₁-C₆ alkyl group which may be optionally substituted with one or two hydroxyl groups or up to three halogen groups (preferably fluorine);
R_{S} is a C₁-C₆ alkyl group, an optionally substituted aryl, heteroaryl or heterocycle group or a —(CH₂)ₘNR₁R₂ group,
X and X′ are each independently C═O, C═S, —S(O), S(O)₂, (preferably X and X′ are both C═O);
R^{2′} is an optionally substituted —(CH₂)ₙ—(C═O)ᵤ(NR₁)ᵥ(SO₂)_{w}alkyl group, an optionally substituted —(CH₂)ₙ—(C═O)ᵤ(NR₁)ᵥ(SO₂)_{w}NR_{1N}R_{2N} group, an optionally substituted —(CH₂)ₙ—(C═O)ᵤ(NR₁)ᵥ(SO₂)_{w}-Aryl, an optionally substituted —(CH₂)ₙ—(C═O)ᵤ(NR₁)ᵥ(SO₂)_{w}-Heteroaryl, an optionally substituted —(CH₂)ₙ—(C═O)ᵤNR₁)ᵥ(SO₂)_{w}-Heterocycle, an optionally substituted —NR¹—(CH₂)ₙ—C(O)ᵤ(NR₁)ᵥ(SO₂)_{w}-alkyl, an optionally substituted —NR′—(CH₂)ₙ—C(O)ᵤ(NR₁)ᵥ(SO₂)_{w}—NR_{1N}R_{2N}, an optionally substituted —NR′—(CH₂)ₙ—C(O)ᵤ(NR₁)ᵥ(SO₂)_{w}—NR¹C(O)R_{1N}, an optionally substituted —NR¹—(CH₂)ₙ—(C═O)ᵤ(NR₁)ᵥ(SO₂)_{w}-Aryl, an optionally substituted —NR¹—(CH₂)ₙ—(C═O)ᵤ(NR₁)ᵥ(SO₂)_{w}-Heteroaryl or an optionally substituted —NR′—(CH₂)ₙ—(C═O)ᵥNR₁—(SO₂)_{w}-Heterocycle, an optionally substituted —X^{R2′}-alkyl group; an optionally substituted —X^{R2′}-Aryl group; an optionally substituted —X^{R2′}-Heteroaryl group; or an optionally substituted —XR^{2′}-Heterocycle group;
R^{3′} is an optionally substituted alkyl, an optionally substituted —(CH₂)ₙ—C(O)ᵤ(NR₁)ᵥ(SO₂)_{w}-alkyl, an optionally substituted —(CH₂)ₙ—C(O)ᵤ(NR₁)ᵥ(SO₂)_{w}—NR_{1N}R_{2N}, an optionally substituted —(CH₂)ₙ—C(O)ᵤ(NR₁)ᵥ(SO₂)_{w}—NR₁C(O)R_{1N}, an optionally substituted —(CH₂)ₙ—C(O)ᵤ(NR₁)ᵥ(SO₂)_{w}—C(O)NR_{1N}R_{2N}, an optionally substituted —(CH₂)ₙ—C(O)ᵤ(NR₁)ᵥ(SO₂)_{w}-Aryl, an optionally substituted —(CH₂)ₙ—C(O)ᵤ(NR₁)ᵥ(SO₂)_{w}-Heteroaryl, an optionally substituted —(CH₂)ₙ—C(O)ᵤ(NR₁)ᵥ(SO₂)_{w}-Heterocycle, an optionally substituted —NR′—(CH₂)ₙ—C(O)ᵤ—(NR₁)ᵥ(SO₂)_{w}-alkyl, an optionally substituted —NR′—(CH₂)ₙ—C(O)ᵤ(NR₁)ᵥ(SO₂)_{w}—NR_{1N}R_{2N}, an optionally substituted —NR¹—(CH₂)ₙ—C(O)ᵤ(NR₁)ᵥ(SO₂)_{w}—NR₁C(O)R_{1N}, an optionally substituted —NR¹—(CH₂)ₙ—C(O)ᵤ(NR₁)ᵥ(SO₂)_{w}-Aryl, an optionally substituted —NR′—(CH₂)ₙ—C(O)ᵤ(NR₁)ᵥ—(SO₂)_{w}-Heteroaryl, an optionally substituted —NR¹—(CH₂)ₙ—C(O)ᵤ(NR₁)ᵥ(SO₂)_{w}-Heterocycle, an optionally substituted —O—(CH₂)ₙ—(C═O)ᵤ(NR₁)ᵥ(SO₂)_{w}-alkyl, an optionally substituted —O—(CH₂)ₙ—(C═O)ᵤ(NR₁)ᵥ(SO₂)_{w}—NR_{1N}R_{2N}, an optionally substituted —O—(CH₂)ₙ—(C═O)ᵤ(NR₁)ᵥ(SO₂)_{w}—NR₁C(O)R_{1N}, an optionally substituted —O—(CH₂)ₙ—(C═O)ᵤ(NR₁)ᵥ(SO₂)_{w}-Aryl, an optionally substituted —O—(CH₂)ₙ—(C═O)ᵤ(NR₁)ᵥ(SO₂)_{w}-Heteroaryl or an optionally substituted —O—(CH₂)ₙ—(C═O)ᵤ(NR₁)ᵥ(SO₂)_{w}-Heterocycle; —(CH₂)ₙ—(V)_{n′}—(CH₂)ₙ—(V)_{n′}-alkyl group, an optionally substituted —(CH₂)ₙ—(V)_{n′}—(CH₂)ₙ—(V)ₙ-Aryl group, an optionally substituted —(CH₂)ₙ—(V)_{n′}—(CH₂)ₙ—(V)ₙ-Heteroaryl group, an optionally substituted —(CH₂)ₙ—(V)_{n′}—(CH₂)ₙ—(V)ₙ-Heterocycle group, an optionally substituted —(CH₂)ₙ—N(R_{1′})(C═O)_{m′}—(V)ₙ-alkyl group, an optionally substituted —(CH₂)ₙ—N(R_{1′})(C═O)_{m′}—(V)ₙ-Aryl group, an optionally substituted —(CH₂)ₙ—N(R_{1′})(C═O)ₘ—(V)ₙ-Heteroaryl group, an optionally substituted —(CH₂)ₙ—N(R_{1′})(C═O)_{m′}—(V)ₙ-Heterocycle group, an optionally substituted —XR^{3′}-alkyl group; an optionally substituted —X^{R3′}-Aryl group; an optionally substituted —X^{R3′}-Heteroaryl group; an optionally substituted —XR^{3′}-Heterocycle group; an optionally substituted;
where R_{1N} and R_{2N} are each independently H, C₁-C₆ alkyl which is optionally substituted with one or two hydroxyl groups and up to three halogen groups or an optionally substituted —(CH₂)ₙ-Aryl, —(CH₂)ₙ-Heteroaryl or —(CH₂)ₙ-Heterocycle group;
V is O, S or NR₁;
R¹ and R_{1′} are each independently H or a C₁-C₃ alkyl group;
X^{R2′} and X^{R3′} are each independently an optionally substituted —CH₂)ₙ—, —CH₂)ₙ—CH(Xᵥ)═CH(Xᵥ)— (cis or trans), —CH₂)ₙ—CH≡CH—, —(CH₂CH₂O)ₙ— or a C₃-C₆ cycloalkyl group, where Xᵥ is H, a halo or a C₁-C₃ alkyl group which is optionally substituted;
each m is independently 0, 1, 2, 3, 4, 5, 6;
each m′ is independently 0 or 1;
each n is independently 0, 1, 2, 3, 4, 5, 6;
each n′ is independently 0 or 1;
each u is independently 0 or 1;
each v is independently 0 or 1;
each w is independently 0 or 1; and
wherein the appropriate one of R^{1′}, R^{2′}, R^{3′}, X, and X′ is modified to be covalently bonded to L.

For example, in Formulae I-i-1, R^{1′} is modified to be covalently bonded to L; in Formula I-i-2, R^{3′} is modified to be covalently bonded to L; etc.

The definitions of variables in the Formulae above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the present disclosure provides a compound of Formula I, wherein LBM is a VHL E3 ubiquitin ligase binding moiety thereby forming a compound of Formulae I-j-1, I-j-2, I-j-3, I-j-4, I-j-5 or I-j-6 respectively:

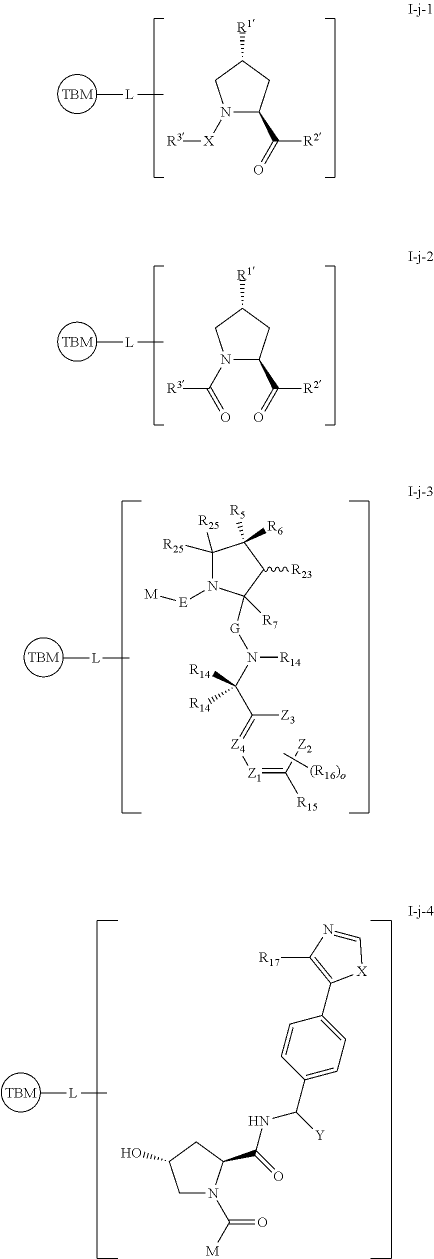

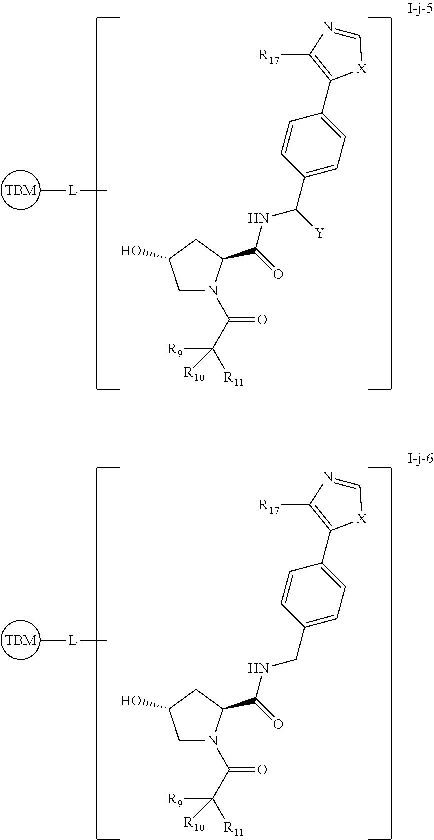

or a pharmaceutically acceptable salt thereof, wherein each of L and TBM is independently as defined above and described in embodiments herein, and wherein each of the variables R^{1′}, R^{2′}, R^{3′}, R₅, R₆, R₇, R₉, R₁₀, R₁₁, R₁₄, R₁₅, R₁₆, R₁₇, R₂₃, R₂₅, E, G, M, X, X′, Y, Z₁, Z₂, Z₃, Z₄, and o is independently as defined and described in international and U.S. patent publications: WO 2016/149668 and US 2016/0272639, the contents of each of which are herein incorporated by reference in their entireties.

For example, in certain embodiments, the present disclosure provides a compound of Formulae I-j-1, wherein X is C═O, C═S, —S(═O)—, or —S(═O)₂—, and R^{1′}, R^{2′}, and R^{3′} are as defined for Formulae I-i-1, I-i-2, I-i-3, I-i-4, and I-i-5, above. In certain embodiments, the present disclosure provides a compound of Formulae I-j-2, wherein R^{1′}, R^{2′}, and R^{3′} are as defined for Formulae I-i-1, I-i-2, I-i-3, I-i-4, and I-i-5, above.

In certain embodiments, the present disclosure provides a compound of Formulae I-j-3, wherein:
each R₅ and R₆ is independently —OH, —SH, or optionally substituted alkyl; or R₅, R₆, and the carbon atom to which they are attached form a carbonyl;
R₇ is H or optionally substituted alkyl;
E is a bond, C═O, or C═S;
G is a bond, optionally substituted alkyl, —COOH, or C=J;
J is O or N—R⁸;
R₈ is H, CN, optionally substituted alkyl, or optionally substituted alkoxy;
M is optionally substituted aryl, optionally substituted heteroaryl, optionally substituted heterocyclic, or —C(R₉)(R₁₀)(R₁₁);
each R₉ and R₁₀ is independently H; optionally substituted alkyl, optionally substituted cycloalkyl, optionally substituted hydroxyalkyl, optionally substituted thioalkyl, optionally substituted heteroaryl, or haloalkyl; or R₉, R₁₀, and the carbon atom to which they are attached form an optionally substituted cycloalkyl;
R₁₁ is optionally substituted heterocyclic, optionally substituted alkoxy, optionally substituted heteroaryl, optionally substituted aryl, or —N(R¹²)(R¹³);
R¹² is H or optionally substituted alkyl;
R¹³ is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, optionally substituted aralkyl, or optionally substituted (oxoalkyl)carbamate;
each R₁₄ is independently H, haloalkyl, optionally substituted cycloalkyl, optionally substituted alkyl, or optionally substituted heterocycloalkyl;
R₁₅ is H, optionally substituted heteroaryl, haloalkyl, optionally substituted aryl, optionally substituted alkoxy, or optionally substituted heterocyclyl;
each R₁₆ is independently halo, optionally substituted alkyl, optionally substituted haloalkyl, CN, or optionally substituted haloalkoxy;
each R₂₅ is independently H or optionally substituted alkyl; or both R₂₅ groups can be taken together to form an oxo or optionally substituted cycloalkyl group;
R²³ is H or OH;
Z₁, Z₂, Z₃, and Z₄ are independently C or N; and
o is 0, 1, 2, 3, or 4.

In certain embodiments, the present disclosure provides a compound of Formulae I-j-4, wherein:
X is O or S;
Y is H, methyl, or ethyl;
R₁₇ is H, methyl, ethyl, hydoxymethyl, or cyclopropyl; and
M is optionally substituted heteroaryl or optionally substituted aryl.

In certain embodiments, the present disclosure provides a compound of Formulae I-j-5, wherein:
X is O or S;
Y is H, methyl, or ethyl;
R₁₇ is H, methyl, ethyl, hydoxymethyl, or cyclopropyl;
R₉ is H;
R₁₀ is H, optionally substituted alkyl, optionally substituted haloalkyl, optionally substituted heteroaryl, optionally substituted aryl, optionally substituted hydroxyalkyl, optionally substituted thioalkyl or cycloalkyl;
R₁₁ is optionally substituted heteroaromatic, optionally substituted heterocyclic, optionally substituted aryl, or —N(R₁₂)(R₁₃);
R¹² is H or optionally substituted alkyl; and
R¹³ is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl; optionally substituted (oxoalkyl)carbamate.

In certain embodiments, the present disclosure provides a compound of Formulae I-j-6, wherein X, R₁₇, R₉, R₁₀, R₁₁, R₁₂, and R₁₃, are as defined for Formulae I-j-5, above.

The definitions of variables in the Formulae above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, LBM is represented by Formula L-1:

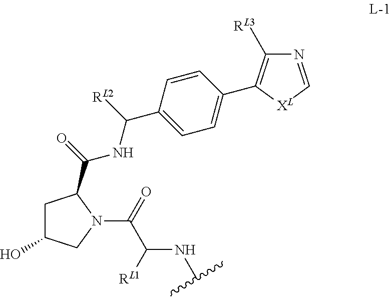

wherein:
R^{L1} is C₁₋₆ alkyl or C₃₋₇ cycloalkyl;
R^{L2} is C₁₋₄ alkyl or hydrogen;
R^{L3} is C₁₋₄ alkyl, C₃₋₅ cycloalkyl, C₁₋₄ hydroxyalkyl, or hydrogen; and
X^{L} is sulfur or oxygen.

The definitions of variables in Formula L-1 above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

As defined generally above, R^{L1} is C₁₋₆ alkyl or C₃₋₇ cycloalkyl. In certain embodiments, R^{L1} is C₃₋₅ alkyl or C₅₋₆ cycloalkyl. In certain embodiments, R^{L1} is C₁₋₆ alkyl. In certain embodiments, R^{L1} is C₃₋₅ alkyl. In certain embodiments, R^{L1} is isopropyl, sec-butyl, or tert-butyl. In certain embodiments, R^{L1} is tert-butyl. In certain embodiments, R^{L1} is C₃₋₇ cycloalkyl. In certain embodiments, R^{L1} is C₅₋₆ cycloalkyl.

As defined generally above, R^{L2} is C₁₋₄ alkyl or hydrogen. In certain embodiments, R^{L2} is C₁₋₄ alkyl. In certain embodiments, R^{L2} is methyl or ethyl. In certain embodiments, R^{L2} is methyl. In certain embodiments, R^{L2} is hydrogen.

As defined generally above, R^{L3} is C₁₋₄ alkyl, C₃₋₅ cycloalkyl, C₁₋₄ hydroxyalkyl, or hydrogen. In certain embodiments, R^{L3} is methyl, ethyl, cyclopropyl, hydroxymethyl, or hydrogen. In certain embodiments, R^{L3} is C₁₋₄ alkyl. In certain embodiments, R^{L3} is methyl or ethyl. In certain embodiments, R^{L3} is methyl. In certain embodiments, R^{L3} is C₃₋₅ cycloalkyl. In certain embodiments, R^{L3} is cyclopropyl. In certain embodiments, R^{L3} is C₁₋₄ hydroxyalkyl. In certain embodiments, R^{L3} is hydroxymethyl. In certain embodiments, R^{L3} is hydrogen.

As defined generally above, X^{L} is sulfur or oxygen. In certain embodiments, X^{L} is sulfur. In certain embodiments, X^{L} is oxygen.

In certain embodiments, LBM is represented by Formula L-la:

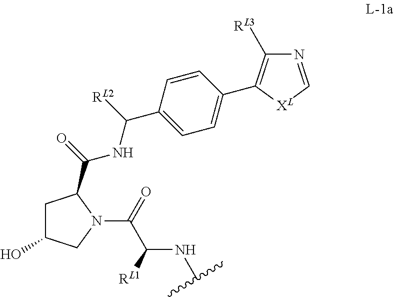

wherein:
R^{L1} is C₁₋₆ alkyl or C₃₋₇ cycloalkyl;
R^{L2} is C₁₋₄ alkyl or hydrogen;
R^{L3} is C₁₋₄ alkyl, C₃₋₅ cycloalkyl, C₁₋₄ hydroxyalkyl, or hydrogen; and
X^{L} is sulfur or oxygen.

The definitions of variables in Formula L-la above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

As defined generally above, R^{L1} is C₁₋₆ alkyl or C₃₋₇ cycloalkyl. In certain embodiments, R^{L1} is C₃₋₅ alkyl or C₅₋₆ cycloalkyl. In certain embodiments, R^{L1} is C₁₋₆ alkyl. In certain embodiments, R^{L1} is C₃₋₅ alkyl. In certain embodiments, R^{L1} is isopropyl, sec-butyl, or tert-butyl. In certain embodiments, R^{L1} is tert-butyl. In certain embodiments, R^{L1} is C₃₋₇ cycloalkyl. In certain embodiments, R^{L1} is C₅₋₆ cycloalkyl.

As defined generally above, R^{L2} is C₁₋₄ alkyl or hydrogen. In certain embodiments, R^{L2} is C₁₋₄ alkyl. In certain embodiments, R^{L2} is methyl or ethyl. In certain embodiments, R^{L2} is methyl. In certain embodiments, R^{L2} is hydrogen.

As defined generally above, R^{L3} is C₁₋₄ alkyl, C₃₋₅ cycloalkyl, C₁₋₄ hydroxyalkyl, or hydrogen. In certain embodiments, R^{L3} is methyl, ethyl, cyclopropyl, hydroxymethyl, or hydrogen. In certain embodiments, R^{L3} is C₁₋₄ alkyl. In certain embodiments, R^{L3} is methyl or ethyl. In certain embodiments, R^{L3} is methyl. In certain embodiments, R^{L3} is C₃₋₅ cycloalkyl. In certain embodiments, R^{L3} is cyclopropyl. In certain embodiments, R^{L3} is C₁₋₄ hydroxyalkyl. In certain embodiments, R^{L3} is hydroxymethyl. In certain embodiments, R^{L3} is hydrogen.

As defined generally above, X^{L} is sulfur or oxygen. In certain embodiments, X^{L} is sulfur. In certain embodiments, X^{L} is oxygen.

The description above describes multiple embodiments relating to LBM groups of Formula L-1. The patent application specifically contemplates all combinations of the embodiments.

In certain embodiments, LBM is or

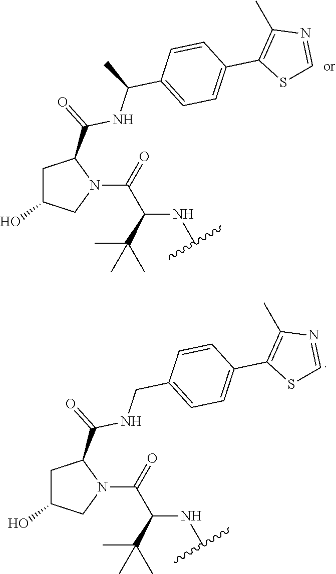 [SMILES: *N[C@H](C(=O)N1C[C@H](O)C[C@H]1C(=O)N[C@@H](C)c1ccc(-c2scnc2C)cc1)C(C)(C)C ; *N[C@H](C(=O)N1C[C@H](O)C[C@H]1C(=O)NCc1ccc(-c2scnc2C)cc1)C(C)(C)C]

In certain embodiments, LBM is

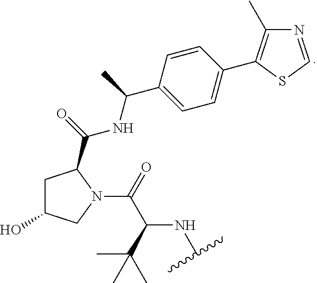 [SMILES: *N[C@H](C(=O)N1C[C@H](O)C[C@H]1C(=O)N[C@@H](C)c1ccc(-c2scnc2C)cc1)C(C)(C)C]

In certain embodiments, LBM is

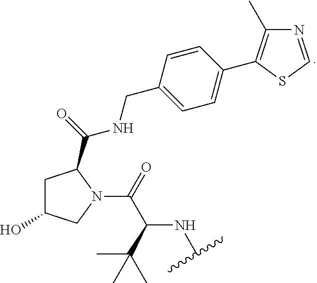 [SMILES: *N[C@H](C(=O)N1C[C@H](O)C[C@H]1C(=O)NCc1ccc(-c2scnc2C)cc1)C(C)(C)C]

In certain embodiments, the present disclosure provides a compound of Formula I, wherein LBM is a VHL E3 ubiquitin ligase binding moiety thereby forming a compound of Formulae I-k-1, I-k-2, or I-k-3 respectively:

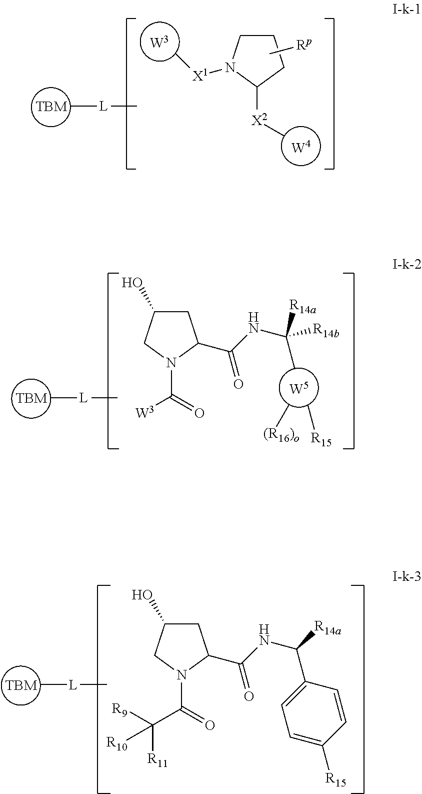

or a pharmaceutically acceptable salt thereof, wherein each of L and TBM is independently as defined above and described in embodiments herein, and wherein each of the variables R^{p}, R₉, R₁₀, R₁₁, R₁₄ₐ, R_{14b}, R₁₅, R₁₆, W³, W⁴, W⁵, X¹, X², and o is independently as defined and described in international and U.S. patent publications WO 2016/118666 and US 2016/0214972, the contents of each of which are herein incorporated by reference in their entireties.

In certain embodiments, the present disclosure provides a compound of Formula I, wherein LBM is a CRBN or VHL E3 ubiquitin ligase binding moiety thereby forming a compound of Formulae I-1-1, I-1-2, I-1-3, I-1-4, I-1-5, I-1-6, or I-1-7 respectively:

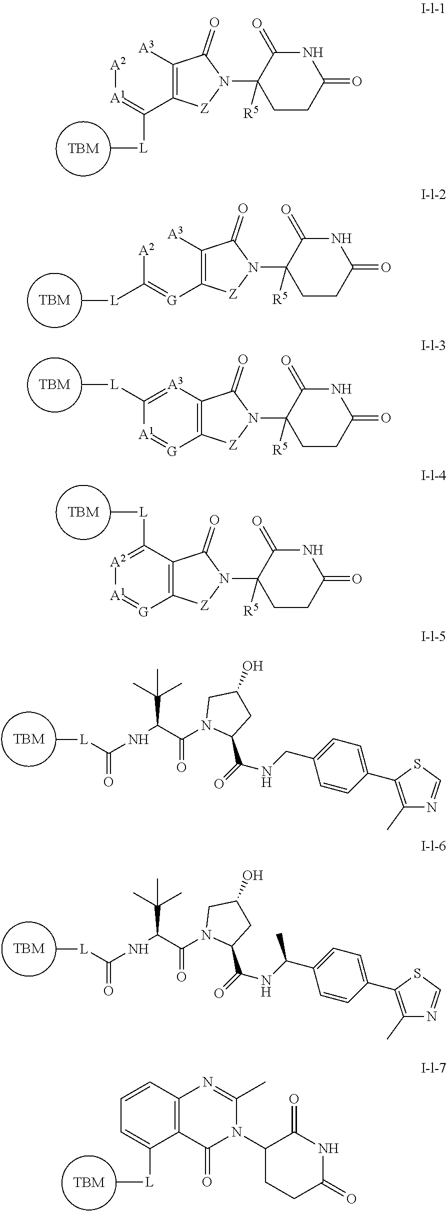

or a pharmaceutically acceptable salt thereof, wherein each of L and TBM is independently as defined above and described in embodiments herein, and wherein each of the variables A¹, A², A³, R⁵, G and Z is independently as defined and described in international publication WO 2017/176958, the contents of which are herein incorporated by reference in their entirety.

In certain embodiments, the present disclosure provides a compound of Formula I, wherein LBM is a CRBN E3 ubiquitin ligase binding moiety thereby forming a compound of Formulae I-1′-1, I-1″-1, I-1′-2, I-1″-2, I-1′-3, I-1″-3, I-1′-4, I-1″-4, I-1′-7 or I-1″-7 respectively:

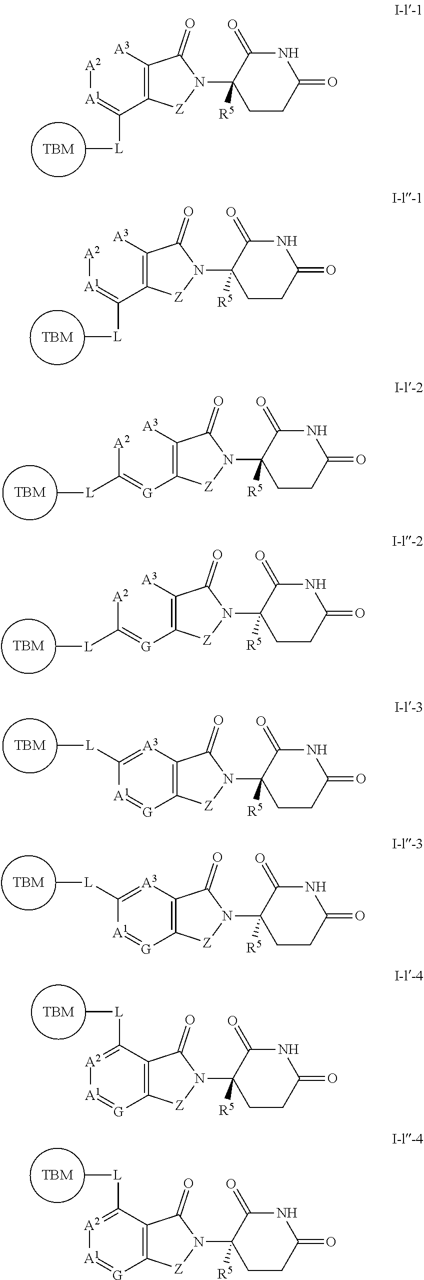

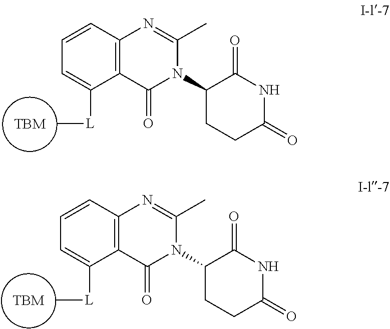

or a pharmaceutically acceptable salt thereof, wherein each of L and TBM is independently as defined above and described in embodiments herein, and wherein each of the variables A¹, A², A³, R⁵, G and Z is independently as defined and described in international patent publication WO 2017/176958, the contents of which are herein incorporated by reference in their entireties.

In certain embodiments, LBM is represented by Formula L-2:

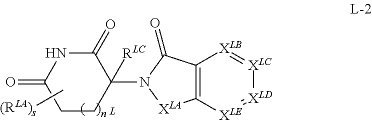

wherein:
R^{LA} and R^{LB} each represents independently for each occurrence hydrogen, halo, C₁₋₆ alkyl, C₁₋₆ haloalkyl, C₃₋₅ cycloalkyl, C₁₋₆ alkoxyl, hydroxyl, or cyano; or, alternatively, two occurrences of R^{LA} on adjacent carbon atoms are taken together to represent a double bond;
R^{LC} is hydrogen, C₁₋₄ alkyl, or halo;
X^{LA} is —C(O)—, —CH₂—, —CH(R^{LA})—, —(CH₂)₂—, ϕ-CH₂CH(R^{LA})—, —CH₂CH(R^{LA})-ϕ, —CH₂CH(O)-ϕ, —S(O)₂—, or —C(R^{LA})═N-ϕ; wherein ϕ is a bond to the aromatic ring;
X^{LB}, X^{LC}, X^{LD}, and X^{LE} each represent independently for each occurrence C(R^{LB}), N, or C(ψ), wherein ψ is a bond to L; provided that one of X^{LB}, X^{LC}, X^{LD}, and X^{LE} is C(ψ), and at least one of X^{LB}, X^{LC}, X^{LD}, and X^{LE} is C(R^{LB});
s is 0, 1, 2, 3, or 4; and
n^{L} is 0 or 1.

The definitions of variables in Formula L-2 above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

As defined generally above, R^{LA} represents independently for each occurrence hydrogen, halo, C₁₋₆ alkyl, C₁₋₆ haloalkyl, C₃₋₅ cycloalkyl, C₁₋₆ alkoxyl, hydroxyl, or cyano; or two occurrences of R^{LA} on adjacent carbon atoms are taken together to represent a double bond.

In certain embodiments, two occurrences of R^{LA} on adjacent carbon atoms are taken together to represent a double bond.

In certain embodiments, R^{LA} represents independently for each occurrence hydrogen, halo, C₁₋₆ alkyl, C₁₋₆ haloalkyl, C₃₋₅ cycloalkyl, C₁₋₆ alkoxyl, hydroxyl, or cyano. In certain embodiments, R^{LA} represents independently for each occurrence hydrogen, halo, C₁₋₆ alkyl, C₁₋₆ haloalkyl, C₃₋₅ cycloalkyl, or cyano. In certain embodiments, R^{LA} represents independently for each occurrence C₁₋₆ alkyl, C₁₋₆ haloalkyl, C₃₋₅ cycloalkyl, or cyano. In certain embodiments, R^{LA} represents independently for each occurrence hydrogen, halo, C₁₋₆ alkoxyl, hydroxyl, or cyano. In certain embodiments, R^{LA} represents independently for each occurrence halo, C₁₋₆ alkoxyl, hydroxyl, or cyano. In certain embodiments, R^{LA} represents independently for each occurrence C₁₋₆ alkoxyl or hydroxyl.

In certain embodiments, R^{LA} represents independently for each occurrence hydrogen, halo, C₁₋₆ alkyl, or C₁₋₆ haloalkyl. In certain embodiments, R^{LA} represents independently for each occurrence hydrogen or halo. In certain embodiments, R^{LA} represents independently for each occurrence C₁₋₆ alkyl or C₁₋₆ haloalkyl.

In certain embodiments, R^{LA} is hydrogen. In certain embodiments, R^{LA} represents independently for each occurrence halo. In certain embodiments, R^{LA} represents independently for each occurrence fluoro or chloro. In certain embodiments, R^{LA} represents independently for each occurrence C₁₋₆ alkyl. In certain embodiments, R^{LA} is methyl. In certain embodiments, R^{LA} represents independently for each occurrence C₁₋₆ haloalkyl. In certain embodiments, R^{LA} represents independently for each occurrence C₃₋₅ cycloalkyl. In certain embodiments, R^{LA} represents independently for each occurrence C₁₋₆ alkoxyl. In certain embodiments, R^{LA} is hydroxyl. In certain embodiments, R^{LA} is cyano.

As defined generally above, R^{LB} represents independently for each occurrence hydrogen, halo, C₁₋₆ alkyl, C₁₋₆ haloalkyl, C₃₋₅ cycloalkyl, C₁₋₆ alkoxyl, hydroxyl, or cyano; or two occurrences of R^{LB} on adjacent carbon atoms are taken together to represent a double bond.

In certain embodiments, two occurrences of R^{LB} on adjacent carbon atoms are taken together to represent a double bond.

In certain embodiments, R^{LB} represents independently for each occurrence hydrogen, halo, C₁₋₆ alkyl, C₁₋₆ haloalkyl, C₃₋₅ cycloalkyl, C₁₋₆ alkoxyl, hydroxyl, or cyano. In certain embodiments, R^{LB} represents independently for each occurrence hydrogen, halo, C₁₋₆ alkyl, C₁₋₆ haloalkyl, C₃₋₅ cycloalkyl, or cyano. In certain embodiments, R^{LB} represents independently for each occurrence C₁₋₆ alkyl, C₁₋₆ haloalkyl, C₃₋₅ cycloalkyl, or cyano. In certain embodiments, R^{LB} represents independently for each occurrence hydrogen, halo, C₁₋₆ alkoxyl, hydroxyl, or cyano. In certain embodiments, R^{LB} represents independently for each occurrence halo, C₁₋₆ alkoxyl, hydroxyl, or cyano. In certain embodiments, R^{LB} represents independently for each occurrence C₁₋₆ alkoxyl or hydroxyl.

In certain embodiments, R^{LB} represents independently for each occurrence hydrogen, halo, C₁₋₆ alkyl, or C₁₋₆ haloalkyl. In certain embodiments, R^{LB} represents independently for each occurrence hydrogen or halo. In certain embodiments, R^{LB} represents independently for each occurrence C₁₋₆ alkyl or C₁₋₆ haloalkyl.

In certain embodiments, R^{LB} is hydrogen. In certain embodiments, R^{LB} represents independently for each occurrence halo. In certain embodiments, R^{LB} represents independently for each occurrence fluoro or chloro. In certain embodiments, R^{LB} represents independently for each occurrence C₁₋₆ alkyl. In certain embodiments, R^{LB} is methyl. In certain embodiments, R^{LB} represents independently for each occurrence C₁₋₆ haloalkyl. In certain embodiments, R^{LB} represents independently for each occurrence C₃₋₅ cycloalkyl. In certain embodiments, R^{LB} represents independently for each occurrence C₁₋₆ alkoxyl. In certain embodiments, R^{LB} is hydroxyl. In certain embodiments, R^{LB} is cyano.

In certain embodiments, R^{LA} and R^{LB} are hydrogen.

As defined generally above, R^{LC} is hydrogen, C₁₋₄ alkyl, or halo. In certain embodiments, R^{LC} is hydrogen. In certain embodiments, R^{LC} is C₁₋₄ alkyl. In certain embodiments, R^{LC} is methyl. In certain embodiments, R^{LC} is halo. In certain embodiments, R^{LC} is fluoro or chloro.

In certain embodiments, the stereocenter bearing R^{LC} is in the (S) stereochemical configuration. In certain embodiments, the stereocenter bearing R^{LC} is in the (R) stereochemical configuration.

As defined generally above, X^{LA} is —C(O)—, —CH₂—, —CH(R^{LA})—, —(CH₂)₂—, ϕ-CH₂CH(R^{LA})—, —CH₂CH(R^{LA})-ϕ, —CH₂CH(O)-ϕ, —S(O)₂—, or —C(R^{LA})═N-ϕ; wherein ϕ is a bond to the aromatic ring.

In certain embodiments, X^{LA} is —C(O)—, —CH₂—, —CH₂CH(O)-ϕ, or —C(R^{LA})═N-ϕ; wherein ϕ is a bond to the aromatic ring. In certain embodiments, X^{LA} is —C(O)—, —CH₂—, or —C(R^{LA})═N-ϕ; wherein ϕ is a bond to the aromatic ring. In certain embodiments, X^{LA} is —C(O)— or —CH₂—.

In certain embodiments, X^{LA} is —C(O)—, —CH₂—, —CH(R^{LA})—, or —S(O)₂—. In certain embodiments, X^{LA} is —C(O)—, —CH₂—, or —CH(R^{LA})—. In certain embodiments, X^{LA} is —(CH₂)₂—, ϕ-CH₂CH(R^{LA})—, —CH₂CH(R^{LA})-ϕ, —CH₂CH(O)-ϕ, or —C(R^{LA})═N-ϕ; wherein ϕ is a bond to the aromatic ring. In certain embodiments, X^{LA} is —(CH₂)₂—, ϕ-CH₂CH(R^{LA})—, or —CH₂CH(R^{LA})-ϕ; wherein ϕ is a bond to the aromatic ring. In certain embodiments, X^{LA} is —CH₂CH(O)-ϕ or —C(R^{LA})═N-ϕ; wherein φ is a bond to the aromatic ring.

In certain embodiments, X^{LA} is —C(O)—. In certain embodiments, X^{LA} is —CH₂—. In certain embodiments, X^{LA} is —CH(R^{LA})—. In certain embodiments, X^{LA} is —(CH₂)₂—. In certain embodiments, X^{LA} is ϕ-CH₂CH(R^{LA})—; wherein ϕ is a bond to the aromatic ring. In certain embodiments, X^{LA} is —CH₂CH(R^{LA})-ϕ; wherein ϕ is a bond to the aromatic ring. In certain embodiments, X^{LA} is —CH₂CH(O)-ϕ; wherein ϕ is a bond to the aromatic ring. In certain embodiments, X^{LA} is —S(O)₂—. In certain embodiments, X^{LA} is —C(R^{LA})═N-ϕ; wherein φ is a bond to the aromatic ring.

As defined generally above, X^{LB}, X^{LC}, X^{LD}, and X^{LE} each represent independently for each occurrence C(R^{LB}), N, or C(ψ), wherein ψ is a bond to L; provided that one of X^{LB}, X^{LC}, X^{LD}, and X^{LE} is C(ψ), and at least one of X^{LB}, X^{LC}, X^{LD}, and X^{LE} is C(R^{LB}).

In certain embodiments, X^{LB} is C(ψ), wherein ψ is a bond to L, and X^{LC}, X^{LD}, and X^{LE} are each C(R^{LB}). In certain embodiments, X^{LB} is C(ψ), wherein ψ is a bond to L, X^{LC} is N, and X^{LD} and X^{LE} are each C(R^{LB}). In certain embodiments, X^{LB} is C(ψ), wherein ψ is a bond to L, X^{LD} is N, and X^{LC} and X^{LE} are each C(R^{LB}). In certain embodiments, X^{LB} is C(ψ), wherein ψ is a bond to L, X^{LE} is N, and X^{LC} and X^{LD} are each C(R^{LB}).

In certain embodiments, X^{LC} is C(ψ), wherein ψ is a bond to L, and X^{LB}, X^{LD}, and X^{LE} are each C(R^{LB}). In certain embodiments, X^{LC} is C(ψ), wherein ψ is a bond to L, X^{LB} is N, and X^{LD} and X^{LE} are each C(R^{LB}). In certain embodiments, X^{LC} is C(ψ), wherein ψ is a bond to L, X^{LD} is N, and X^{LB} and X^{LE} are each C(R^{LB}). In certain embodiments, X^{LC} is C(ψ), wherein ψ is a bond to L, X^{LE} is N, and X^{LB} and X^{LD} are each C(R^{LB}).

In certain embodiments, X^{LD} is C(ψ), wherein ψ is a bond to L, and X^{LB}, X^{LC}, and X^{LE} are each C(R^{LB}). In certain embodiments, X^{LD} is C(ψ), wherein ψ is a bond to L, X^{LB} is N, and X^{LC} and X^{LE} are each C(R^{LB}). In certain embodiments, X^{LD} is C(ψ), wherein ψ is a bond to L, X^{LC} is N, and X^{LB} and X^{LE} are each C(R^{LB}). In certain embodiments, X^{LD} is C(ψ), wherein ψ is a bond to L, X^{LE} is N, and X^{LB} and X^{LC} are each C(R^{LB}).

In certain embodiments, X^{LE} is C(ψ), wherein ψ is a bond to L, and X^{LB}, X^{LC}, and X^{LD} are each C(R^{LB}). In certain embodiments, X^{LE} is C(ψ), wherein ψ is a bond to L, X^{LB} is N, and X^{LC} and X^{LD} are each C(R^{LB}). In certain embodiments, X^{LE} is C(ψ), wherein ψ is a bond to L, X^{LC} is N, and X^{LB} and X^{LD} are each C(R^{LB}). In certain embodiments, X^{LE} is C(ψ), wherein ψ is a bond to L, X^{LD} is N, and X^{LB} and X^{LC} are each C(R^{LB}).

As defined generally above, s is 0, 1, 2, 3, or 4. In certain embodiments, s is 0. In certain embodiments, s is 1. In certain embodiments, s is 2. In certain embodiments, s is 3. In certain embodiments, s is 4. In certain embodiments, s is 0 or 1. In certain embodiments, s is 1 or 2. In certain embodiments, s is 2 or 3. In certain embodiments, s is 3 or 4. In certain embodiments, s is 0, 1, or 2. In certain embodiments, s is 1, 2, or 3. In certain embodiments, s is 2, 3, or 4. In certain embodiments, s is 0, 1, 2, or 3. In certain embodiments, s is 1, 2, 3, or 4.

As defined generally above, n^{L} is 0 or 1. In certain embodiments, n^{L} is 0. In certain embodiments, n^{L} is 1.

The description above describes multiple embodiments relating to LBM groups of Formula L-2. The patent application specifically contemplates all combinations of the embodiments.

In certain embodiments, LBM is represented by Formula L-3:

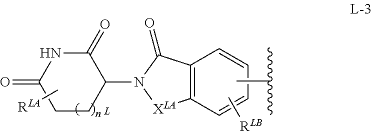

wherein:
R^{LA} and R^{LB} are each independently hydrogen, halo, C₁₋₆ alkyl, or C₁₋₆ haloalkyl;
X^{LA} is —C(O)— or —CH₂—; and
n^{L} is 0 or 1.

The definitions of variables in Formula L-3 above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

As defined generally above, R^{LA} is hydrogen, halo, C₁₋₆ alkyl, or C₁₋₆ haloalkyl. In certain embodiments, R^{LA} is hydrogen or halo. In certain embodiments, R^{LA} is C₁₋₆ alkyl or C₁₋₆ haloalkyl. In certain embodiments, R^{LA} is hydrogen. In certain embodiments, R^{LA} is halo. In certain embodiments, R^{LA} is fluoro or chloro. In certain embodiments, R^{LA} is C₁₋₆ alkyl. In certain embodiments, R^{LA} is methyl. In certain embodiments, R^{LA} is C₁₋₆ haloalkyl.

As defined generally above, R^{LB} is hydrogen, halo, C₁₋₆ alkyl, or C₁₋₆ haloalkyl. In certain embodiments, R^{LB} is hydrogen or halo. In certain embodiments, R^{LB} is C₁₋₆ alkyl or C₁₋₆ haloalkyl. In certain embodiments, R^{LB} is hydrogen. In certain embodiments, R^{LB} is halo. In certain embodiments, R^{LB} is fluoro or chloro. In certain embodiments, R^{LB} is C₁₋₆ alkyl. In certain embodiments, R^{LB} is methyl. In certain embodiments, R^{LB} is C₁₋₆ haloalkyl.

In certain embodiments, R^{LA} and R^{LB} are hydrogen.

In certain embodiments, the stereocenter bearing the phthalimide or phthalamide substituent is in the (S) stereochemical configuration. In certain embodiments, the stereocenter bearing the phthalimide or phthalamide substituent is in the (R) stereochemical configuration.

As defined generally above, X^{LA} is —C(O)— or —CH₂—. In certain embodiments, X^{LA} is —C(O)—. In certain embodiments, X^{LA} is —CH₂—.

As defined generally above, n^{L} is 0 or 1. In certain embodiments, n^{L} is 0. In certain embodiments, n^{L} is 1.

The definitions of variables in Formula L-3 above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

The description above describes multiple embodiments relating to LBM groups of Formula L-3. The patent application specifically contemplates all combinations of the embodiments.

In certain embodiments, the present disclosure provides a compound of Formula I, wherein LBM is a MDM2 (i.e., human double minute 2 or HDM2) E3 ligase binding moiety thereby forming a compound of Formulae I-m-1, I-m-2, I-m-3, I-m-4, I-m-5, I-m-6, I-m-7, I-m-8, I-m-9, I-m-10, I-m-11, I-m-12, I-m-13, I-m-14, I-m-15, I-m-16, I-m-17, or I-m-18 respectively:

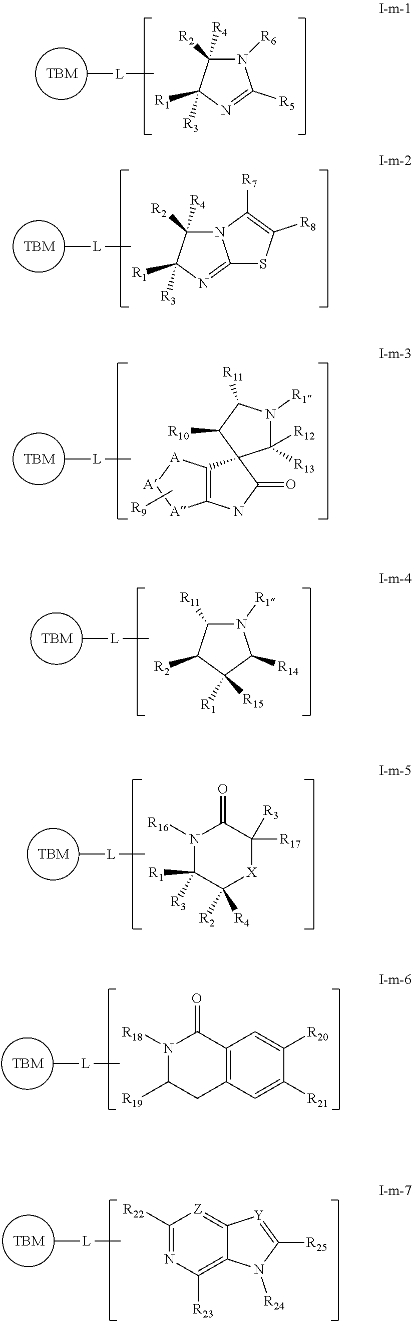

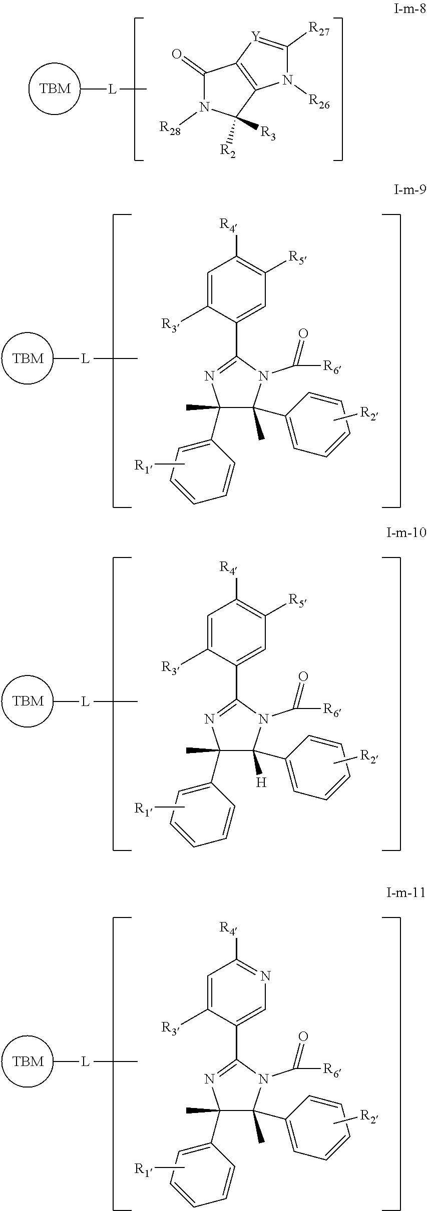

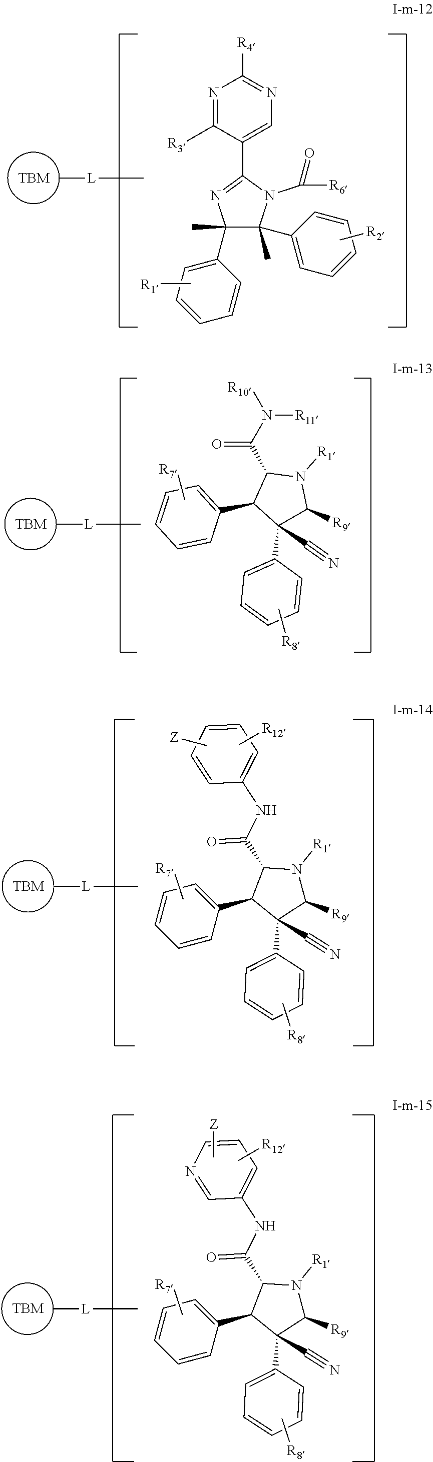

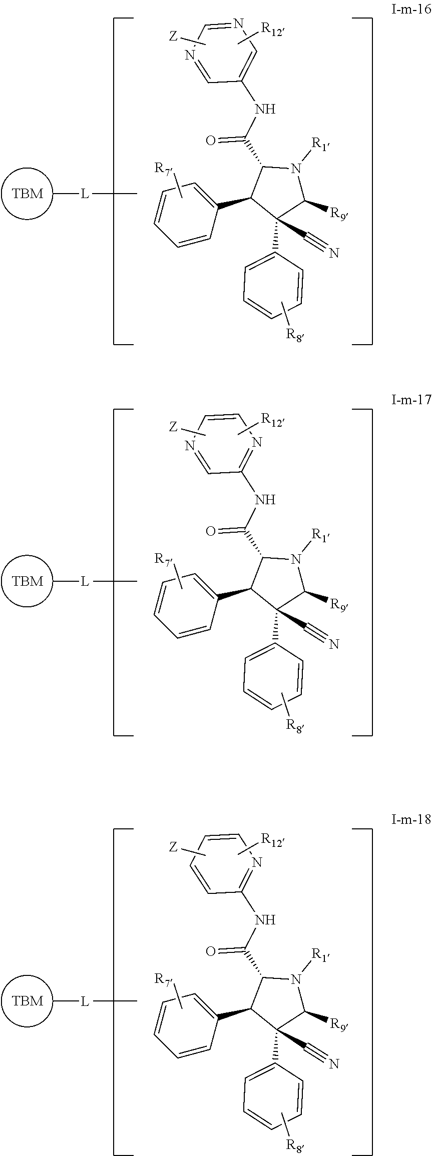

or a pharmaceutically acceptable salt thereof, wherein each of L and TBM is independently as defined above and described in embodiments herein, and wherein each of the variables R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R_{1′}, R_{2′}, R_{3′}, R_{4′}, R_{5′}, R_{6′}, R_{7′}, R_{8′}, R_{9′}, R_{10′}, R_{11′}, R₁₂′, R_{1″}, A, A′, A″, X, Y, and Z is independently as defined and described in international and U.S. patent publications WO 2017/011371 and US 2017/0008904, the contents of each of which are herein incorporated by reference in their entireties.

In certain embodiments, the present disclosure provides a compound of Formula I, wherein LBM is an IAP E3 ubiquitin ligase binding moiety thereby forming a compound of Formulae I-n-1, I-n-2, I-n-3, or I-n-4 respectively:

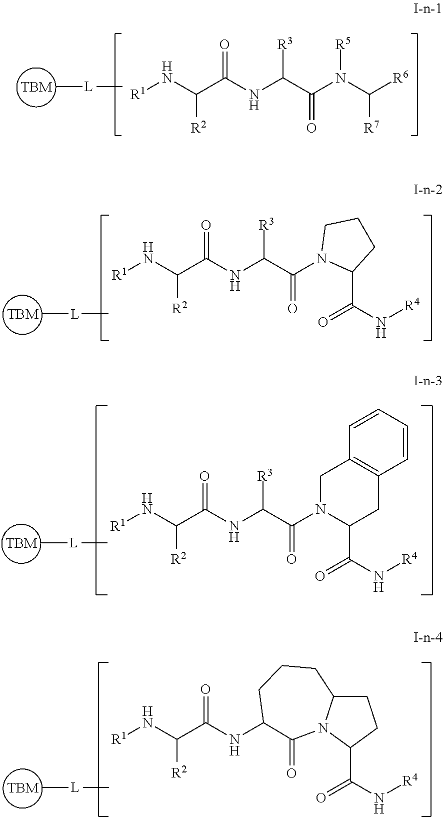

or a pharmaceutically acceptable salt thereof, wherein each of L and TBM is independently as defined above and described in embodiments herein, and wherein each of the variables R¹, R², R³, R⁴, R⁵, R⁶, and R⁷, is independently as defined and described in international and U.S. patent publications WO 2017/011590 and US 2017/0037004, the contents of each of which are herein incorporated by reference in their entireties.

In certain embodiments, the present disclosure provides a compound of Formula I, wherein LBM is a VHL binding moiety thereby forming a compound of Formulae I-o-1, I-o-2, or I-o-3:

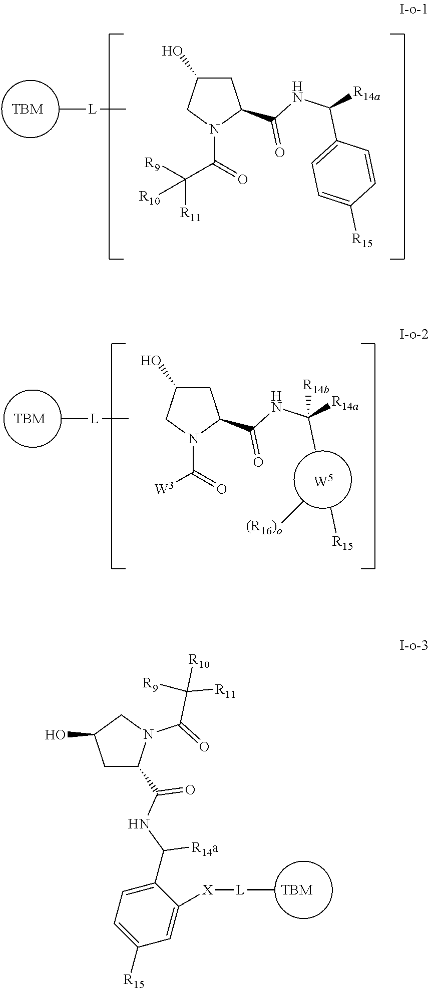

or a pharmaceutically acceptable salt thereof, wherein each of L and TBM is independently as defined above and described in embodiments herein, and wherein each of the variables X, W³, W⁵, R₉, R₁₀, R₁₁, R₁₄ₐ, R_{14b}, R₁₅, R₁₆, and o is independently as described and defined in international and U.S. patent publications WO 2017/030814, WO 2016/118666, and US 2017/0327469, the contents of each of which are herein incorporated by reference in their entireties.

In certain embodiments, the present disclosure provides a compound of Formula I, wherein LBM is an IAP binding moiety thereby forming a compound of Formula I-p:

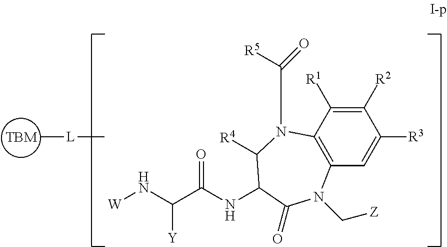

or a pharmaceutically acceptable salt thereof, wherein each of L and TBM is independently as defined above and described in embodiments herein, and wherein each of the variables W, Y, Z, R¹, R², R³, R⁴, and R⁵ is independently as described and defined in international and U.S. patent publications WO 2014/044622, US 2015/0225449, WO 2015/071393, and US 2016/0272596, the contents of each of which are herein incorporated by reference in their entireties.

In certain embodiments, the present disclosure provides a compound of Formula I, wherein LBM is a MDM2 binding moiety thereby forming a compound of Formula I-q:

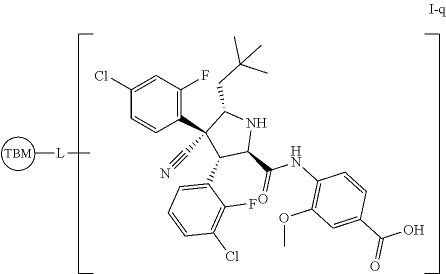

or a pharmaceutically acceptable salt thereof, as described and defined in Hines, J. et al., Cancer Res. (DOI: 10.1158/0008-5472.CAN-18-2918), the contents of which are herein incorporated by reference in their entirety.

In certain embodiments, the present disclosure provides a compound of Formula I, wherein LBM is a DCAF16 binding moiety thereby forming a compound of Formula I-r:

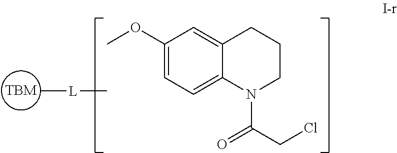

or a pharmaceutically acceptable salt thereof, as described and defined in Zhang, X. et al., bioRxiv (doi: https://doi.org/10.1101/443804), the contents of which are herein incorporated by reference in their entirety.

In certain embodiments, the present disclosure provides a compound of Formula I, wherein LBM is a RNF114 binding moiety thereby forming a compound of Formula I-s:

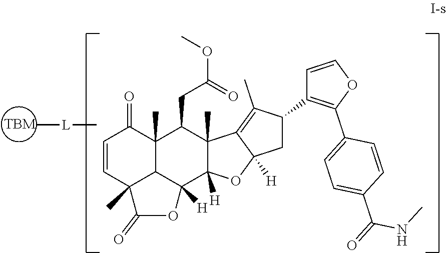

or a pharmaceutically acceptable salt thereof, as described and defined in Spradin, J. N. et al., bioRxiv (doi: https://doi.org/10.1101/436998), the contents of which are herein incorporated by reference in their entirety.

In certain embodiments, the present disclosure provides a compound of Formula I, wherein LBM is a RNF4 binding moiety thereby forming a compound of Formula I-t:

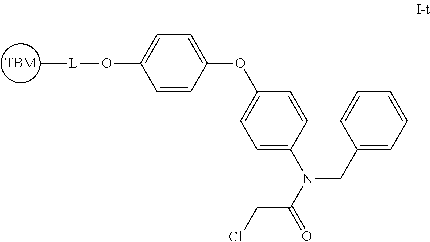

or a pharmaceutically acceptable salt thereof, as described and defined in Ward, C. C., et al., bioRxiv (doi: https://doi.org/10.1101/439125), the contents of which are herein incorporated by reference in their entirety.

In certain embodiments, the present disclosure provides a compound of Formula I, wherein LBM is a VHL binding moiety thereby forming a compound of Formula I-u-1 or I-u-2:

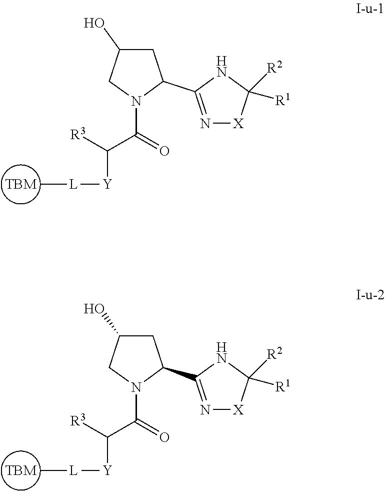

or a pharmaceutically acceptable salt thereof, wherein each of L and TBM is independently as defined above and described in embodiments herein, and wherein each of the variables R¹, R², R³, X, and Y is independently as defined and described in international patent publication WO 2019/084026, the contents of which are herein incorporated by reference in their entirety.

In certain embodiments, the present disclosure provides a compound of Formula I, wherein LBM is a VHL binding moiety thereby forming a compound of Formulae I-v-1 or I-v-2:

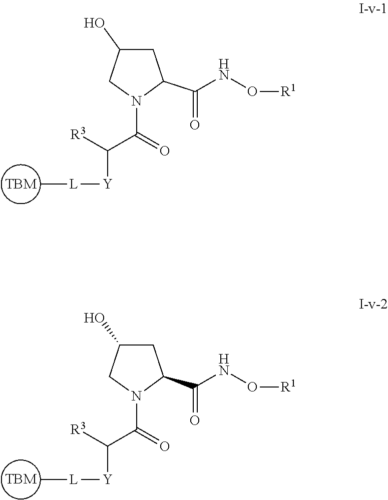

or a pharmaceutically acceptable salt thereof, wherein each of L and TBM is independently as defined above and described in embodiments herein, and wherein each of the variables R¹, R³, and Y is independently as defined and described in international patent publication WO 2019/084030, the contents of which are herein incorporated by reference in their entirety.

In certain embodiments, the present disclosure provides a compound of Formula I, wherein LBM is a E3 ubiquitin ligase (cereblon) binding moiety thereby forming a compound of Formulae I-w-1, I-w-2, I-w-3, or I-w-4:

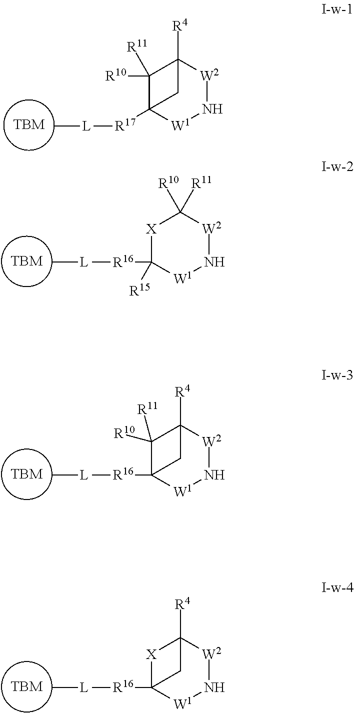

or a pharmaceutically acceptable salt thereof, wherein L and TBM are as defined above and described herein, and wherein each of the variables R⁴, R¹⁰, R¹¹, R¹⁵, R¹⁶, R¹⁷, W¹, W², and X is as defined in international patent publication WO 2019/099868, the contents of which are herein incorporated by reference in their entirety, and wherein

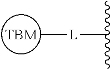

is attached to R¹⁷ or R¹⁶ at the site of attachment of R¹² as defined in international patent publication WO 2018/237026, such that

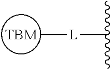

takes the place of the R¹² substituent.

In certain embodiments, the present disclosure provides a compound of Formula I, wherein LBM is a RPN13 binding moiety thereby forming a compound of Formula I-x:

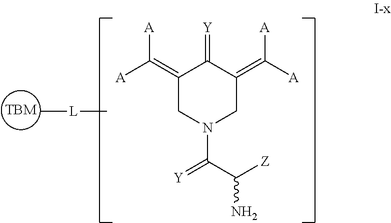

or a pharmaceutically acceptable salt thereof, wherein L and TBM are as defined above and described in embodiments herein, and wherein each of the variables A, Y, and Z is as described and defined in international patent publication WO 2019/165229, the contents of which are herein incorporated by reference in their entirety.

In certain embodiments, the present disclosure provides a compound of Formula I, wherein LBM is a Ubr1 binding moiety as described in Shanmugasundaram, K. et al, J. Bio. Chem. 2019, doi: 10.1074/jbc.AC119.010790, the contents of which are herein incorporated by reference in their entirety, thereby forming a compound of Formula I-y-1 or I-y-2:

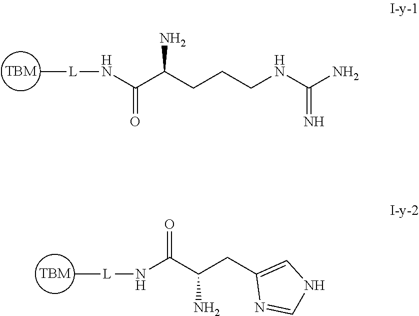

or a pharmaceutically acceptable salt thereof, wherein each of L and TBM is independently as defined above and described in embodiments herein.

In certain embodiments, the present disclosure provides a compound of Formula I, wherein LBM is a CRBN binding moiety thereby forming a compound of Formula I-z:

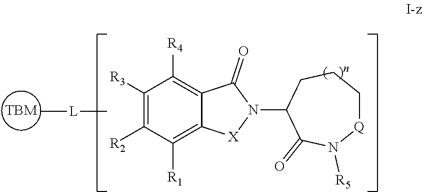

or a pharmaceutically acceptable salt thereof, wherein each of L and TBM is independently as defined above and described in embodiments herein, and wherein each of the variables R₁, R₂, R₃, R₄, R₅, Q, X, and n is independently as described and defined in U.S. patent publication 2019/276474, the contents of which are herein incorporated by reference in their entirety.

In certain embodiments, the present disclosure provides a compound of Formula I, wherein LBM is a CRBN E3 ubiquitin ligase binding moiety thereby forming a compound of Formulae I-aa-1, I-aa-2, I-aa-3, or I-aa-4:

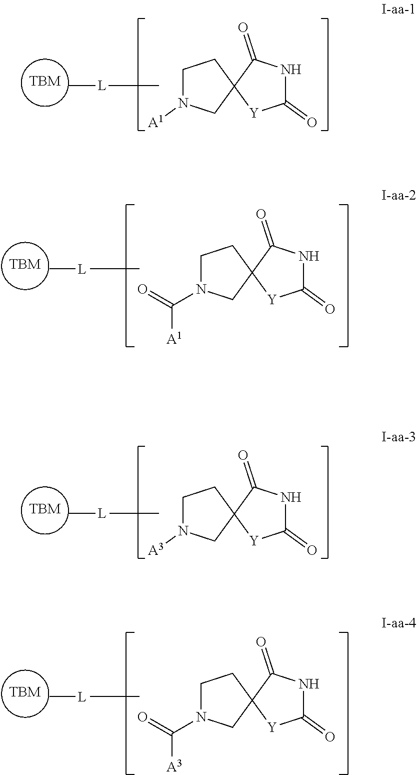

or a pharmaceutically acceptable salt thereof, wherein each of L and TBM is independently as defined above and described in embodiments herein, and wherein each of the variables Y, A¹, and A³ is independently as described and defined in international patent publication WO 2019/236483, the contents of which are herein incorporated by reference in their entirety.

In certain embodiments, the present disclosure provides a compound of Formula I, wherein LBM is a CRBN E3 ubiquitin ligase binding moiety thereby forming a compound of Formulae I-bb-1, I-bb-2, I-bb-3, or I-bb-4:

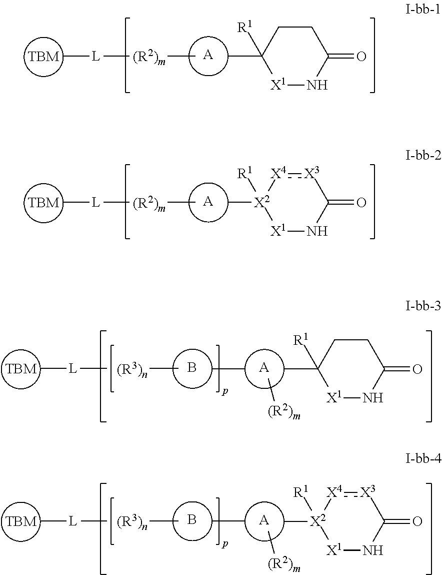

or a pharmaceutically acceptable salt thereof, wherein each of L and TBM is independently as defined above and described in embodiments herein, and wherein each of the variables Ring A, Ring B, R¹, R², R³, X¹, X², X³, X⁴, m, n, and p is independently as described and defined in international patent publication WO 2019/060693, the contents of which are herein incorporated by reference in their entirety.

In certain embodiments, the present disclosure provides a compound of Formula I, wherein LBM is a CRBN E3 ubiquitin ligase binding moiety thereby forming a compound of Formula I-cc-1:

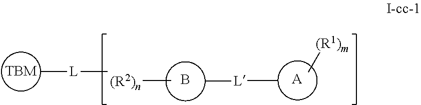

or a pharmaceutically acceptable salt thereof, wherein each of L and TBM is independently as defined above and described in embodiments herein, wherein each of the variables Ring A, Ring B, R¹, R², m, and n is independently as described and defined in international patent publication WO 2019/140387, and wherein variable L′ corresponds to variable L in WO 2019/140387 and is selected from the embodiments for variable L as described and defined in WO 2019/140387. The contents of WO 2019/140387 are herein incorporated by reference in their entirety.

In certain embodiments, the present disclosure provides a compound of Formula I, wherein LBM is a CRBN E3 ubiquitin ligase binding moiety thereby forming a compound of Formula I-dd-1:

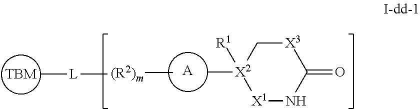

or a pharmaceutically acceptable salt thereof, wherein each of L and TBM is independently as defined above and described in embodiments herein, wherein each of the variables Ring A, R¹, R², X¹, X², X³, and m is independently as described and defined in international patent publication WO 2020/010177, the contents of which are herein incorporated by reference in their entirety.

In certain embodiments, LBM is represented by Formula L-1 or Formula L-2. In certain embodiments, LBM is represented by Formula L-1 or Formula L-2; wherein each of the variables R^{L1}, R^{L2}, R^{L3}, X^{L}, R^{LA}, R^{LB}, R^{LC}, X^{LA}, X^{LB}, X^{LC}, X^{LD}, X^{LE}, s, and n^{L} is independently as described and defined in embodiments above, both singly and in combination. In certain embodiments, LBM is represented by Formula L-1 or Formula L-3. In certain embodiments, LBM is represented by Formula L-1 or Formula L-3; wherein each of the variables R^{L1}, R^{L2}, R^{L3}, X^{L}, R^{LA}, R^{LB}, X^{LA}, and n^{L} is independently as described and defined in embodiments above, both singly and in combination.

In certain embodiments, LBM is

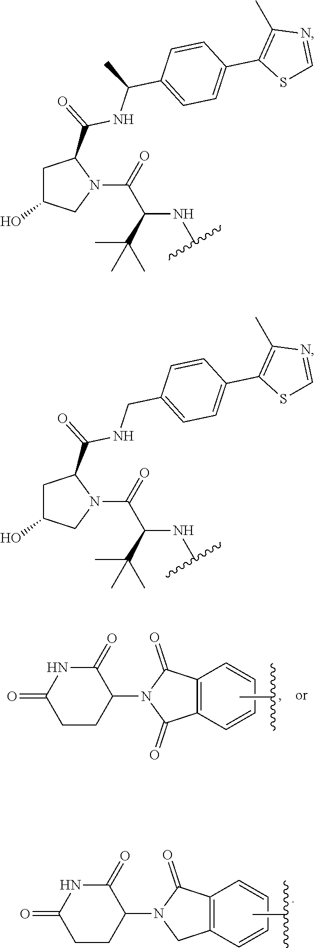

In certain embodiments, LBM is

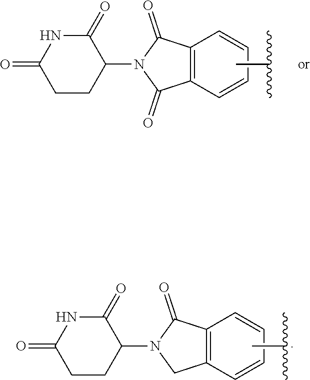

In certain embodiments, LBM is

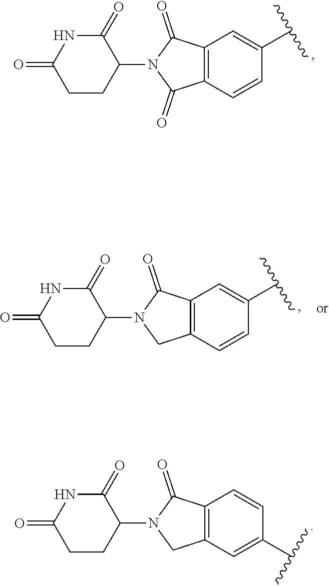 [SMILES: *c1ccc2c(c1)C(=O)N(C1CCC(=O)NC1=O)C2=O ; *c1ccc2c(c1)C(=O)N(C1CCC(=O)NC1=O)C2 ; *c1ccc2c(c1)CN(C1CCC(=O)NC1=O)C2=O]

In certain embodiments, LBM is

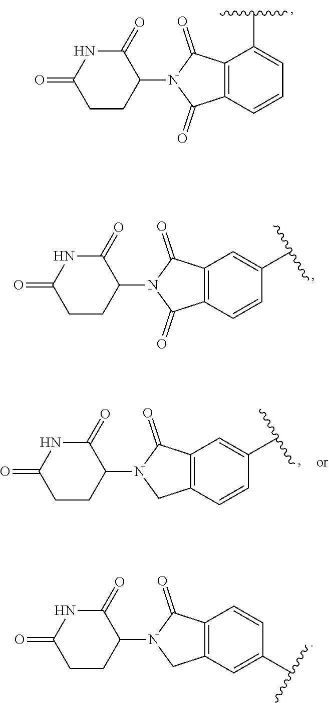 [SMILES: *c1cccc2c1C(=O)N(C1CCC(=O)NC1=O)C2=O ; *c1ccc2c(c1)C(=O)N(C1CCC(=O)NC1=O)C2=O ; *c1ccc2c(c1)C(=O)N(C1CCC(=O)NC1=O)C2 ; *c1ccc2c(c1)CN(C1CCC(=O)NC1=O)C2=O]

In certain embodiments, LBM is

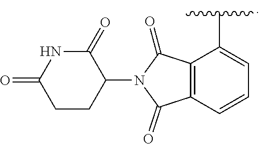 [SMILES: *c1cccc2c1C(=O)N(C1CCC(=O)NC1=O)C2=O]

In certain embodiments, LBM is

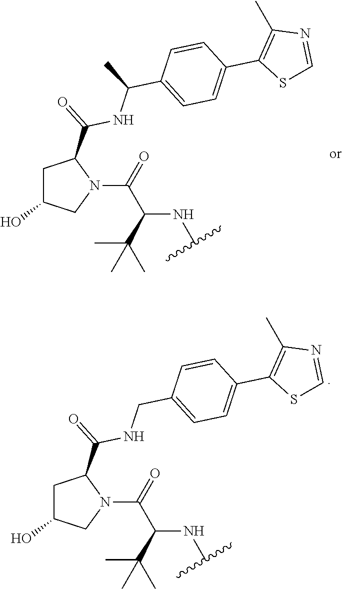 [SMILES: *N[C@H](C(=O)N1C[C@H](O)C[C@H]1C(=O)N[C@@H](C)c1ccc(-c2scnc2C)cc1)C(C)(C)C ; *N[C@H](C(=O)N1C[C@H](O)C[C@H]1C(=O)NCc1ccc(-c2scnc2C)cc1)C(C)(C)C]

In certain embodiments, LBM is selected from the groups depicted in the compounds in Table 1, below.

### Part B: Degradation Inducing Moiety (DIM)

As defined generally above, DIM is a degradation inducing moiety. In certain embodiments, DIM is LBM as described above and herein. In certain embodiments, DIM is a lysine mimetic. In certain embodiments, the covalent attachment of ubiquitin to an NSD2 protein is achieved through the action of a lysine mimetic. In certain embodiments, upon the binding of a compound of Formula II to NSD2, the moiety that mimics a lysine undergoes ubiquitination thereby marking NSD2 for degradation via the Ubiquitin-Proteasome Pathway (UPP).

In certain embodiments, DIM is

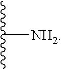 [SMILES: *N]

In certain embodiments, DIM is

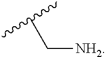 [SMILES: *CN]

In certain embodiments, DIM is

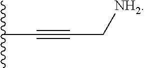 [SMILES: *C#CCN]

In certain embodiments, the present disclosure provides the compound of Formula II as a compound of Formula II-a:

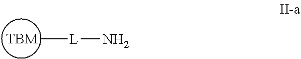

or a pharmaceutically acceptable salt thereof, wherein each of TBM and L is as defined above and described in embodiments herein, both singly and in combination.

In certain embodiments, the present disclosure provides the compound of Formula II as a compound of Formula II-b:

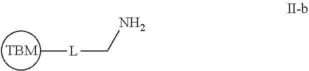

or a pharmaceutically acceptable salt thereof, wherein each of TBM and L is as defined above and described in embodiments herein, both singly and in combination.

In certain embodiments, the present disclosure provides the compound of Formula II as a compound of Formula II-c:

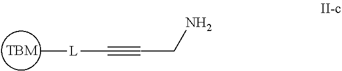

or a pharmaceutically acceptable salt thereof, wherein each of TBM and L is as defined above and described in embodiments herein, both singly and in combination.

In certain embodiments, the present disclosure provides a compound of Formula II, wherein DIM is a lysine mimetic

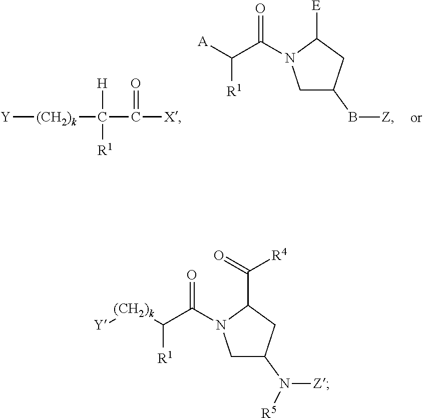

thereby forming a compound of Formulae II-d-1, II-d-2, or II-d-3, respectively:

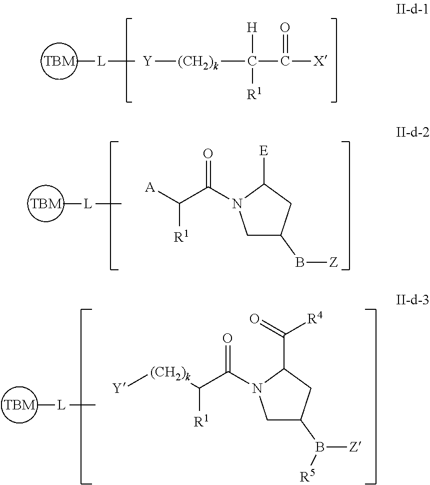

or a pharmaceutically acceptable salt thereof, wherein each of L and TBM is independently as defined above and described in embodiments herein, and wherein each of the variables R¹, R⁴, R⁵, A, B, E, Y, Y′, Z, Z′, and k is independently as defined and described in U.S. Pat. No. 7,622,496, the contents of which are herein incorporated by reference in their entirety.

In certain embodiments, DIM is a hydrogen atom. In certain embodiments, the covalent attachment of ubiquitin to an NSD2 protein is achieved through a provided compound wherein DIM is a hydrogen atom. In certain embodiments, upon the binding of a compound of Formula II to NSD2, the moiety being hydrogen effectuates ubiquitination thereby marking NSD2 for degradation via the Ubiquitin-Proteasome Pathway (UPP).

In certain embodiments, the present disclosure provides the compound of Formula II wherein DIM is a hydrogen atom, thereby forming a compound of Formula II-d-4:

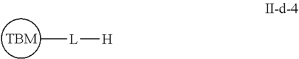

or a pharmaceutically acceptable salt thereof, wherein each of TBM and L is as defined above and described in embodiments herein, both singly and in combination.

In certain embodiments, DIM is selected from the groups depicted in the compounds in Table 1, below.

Another aspect of the disclosure provides a collection of heterobifunctional substituted pyridinylmethyl aminopurine and related compounds, such as a compound represented by Formula II:

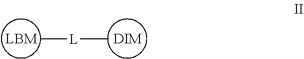

or a pharmaceutically acceptable salt thereof, where the variables are as defined in the detailed description.

### Part C: Linker (L)

As defined generally above, L is a bivalent linker. A variety of linker are known to one of skill in the art and may be used in the compounds described herein. In certain embodiments, L is a bivalent linker as described in international patent publications WO 2018/237026, WO 2019/099926, WO 2019/199816, WO 2019/160915, WO 2019/060693, WO 2019/140387, or WO 2020/010177, the contents of each of which are herein incorporated by reference in their entireties.

In certain embodiments, L is a covalent bond, or a bivalent, saturated or partially unsaturated, straight or branched C₁₋₆₀ hydrocarbon chain, wherein 1-20 methylene units of L are independently and optionally replaced by -Cy-, —O—, —N(R′)—, —S—, —C(O)—, —S(O)—, —S(O)₂—, or

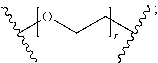

wherein each -Cy- is independently a 3-7 membered saturated or partially unsaturated carbocyclylene, a 3-7 membered saturated or partially unsaturated heterocyclylene containing 1 or 2 heteroatoms independently selected from nitrogen, oxygen, and sulfur, or a 5-6 membered heteroarylene containing 1, 2, 3, or 4 heteroatoms independently selected from nitrogen, oxygen, and sulfur, wherein the carbocyclene, heterocyclylene, and heteroarylene are substituted with 0-2 occurrences of R^{J}; wherein R^{I} represents independently for each occurrence hydrogen or C₁₋₆ alkyl; wherein R^{J} represents independently for each occurrence C₁₋₆ alkyl, C₁₋₆ haloalkyl, or C₁₋₆ alkoxyl; and wherein r is 0, 1, 2, 3, 4, 5, 6, 7, 8, 9, or 10.

In certain embodiments, each -Cy- is independently a 3-7 membered saturated or partially unsaturated carbocyclylene, wherein the carbocyclene is substituted with 0-2 occurrences of R^{J}. In certain embodiments, -Cy- is a 3 membered saturated or partially unsaturated carbocyclylene, wherein the carbocyclene is substituted with 0-2 occurrences of R^{J}. In certain embodiments, -Cy- is a 4 membered saturated or partially unsaturated carbocyclylene, wherein the carbocyclene is substituted with 0-2 occurrences of R^{J}. In certain embodiments, -Cy- is a 5 membered saturated or partially unsaturated carbocyclylene, wherein the carbocyclene is substituted with 0-2 occurrences of R^{J}. In certain embodiments, -Cy- is a 6 membered saturated or partially unsaturated carbocyclylene, wherein the carbocyclene is substituted with 0-2 occurrences of R^{J}. In certain embodiments, -Cy- is a 7 membered saturated or partially unsaturated carbocyclylene, wherein the carbocyclene is substituted with 0-2 occurrences of R^{J}.

In certain embodiments, each -Cy- is independently a 3-7 membered saturated or partially unsaturated heterocyclylene containing 1 or 2 heteroatoms independently selected from nitrogen, oxygen, and sulfur, wherein the heterocyclylene is substituted with 0-2 occurrences of R^{J}. In certain embodiments, -Cy- is a 3 membered saturated or partially unsaturated heterocyclylene containing 1 or 2 heteroatoms independently selected from nitrogen, oxygen, and sulfur, wherein the heterocyclylene is substituted with 0-2 occurrences of R^{J}. In certain embodiments, -Cy- is a 4 membered saturated or partially unsaturated heterocyclylene containing 1 or 2 heteroatoms independently selected from nitrogen, oxygen, and sulfur, wherein the heterocyclylene is substituted with 0-2 occurrences of R^{J}. In certain embodiments, -Cy- is a 5 membered saturated or partially unsaturated heterocyclylene containing 1 or 2 heteroatoms independently selected from nitrogen, oxygen, and sulfur, wherein the heterocyclylene is substituted with 0-2 occurrences of R^{J}. In certain embodiments, -Cy- is a 6 membered saturated or partially unsaturated heterocyclylene containing 1 or 2 heteroatoms independently selected from nitrogen, oxygen, and sulfur, wherein the heterocyclylene is substituted with 0-2 occurrences of R^{J}. In certain embodiments, -Cy- is a 7 membered saturated or partially unsaturated heterocyclylene containing 1 or 2 heteroatoms independently selected from nitrogen, oxygen, and sulfur, wherein the heterocyclylene is substituted with 0-2 occurrences of R^{J}.

In certain embodiments, each -Cy- is independently a 5-6 membered heteroarylene containing 1, 2, 3, or 4 heteroatoms independently selected from nitrogen, oxygen, and sulfur, wherein the heteroarylene is substituted with 0-2 occurrences of R^{J}. In certain embodiments, -Cy- is a 5 membered heteroarylene containing 1, 2, 3, or 4 heteroatoms independently selected from nitrogen, oxygen, and sulfur, wherein the heteroarylene is substituted with 0-2 occurrences of R^{J}. In certain embodiments, -Cy- is a 6 membered heteroarylene containing 1, 2, 3, or 4 heteroatoms independently selected from nitrogen, oxygen, and sulfur, wherein the heteroarylene is substituted with 0-2 occurrences of R^{J}.

In certain embodiments, -Cy- is

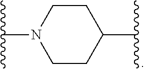 [SMILES: *C1CCN(*)CC1]

In certain embodiments, -Cy- is

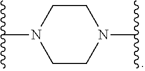 [SMILES: *N1CCN(*)CC1]

In certain embodiments, -Cy- is

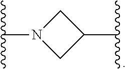 [SMILES: *C1CN(*)C1]

In certain embodiments, -Cy- is

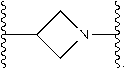 [SMILES: *C1CN(*)C1]

In certain embodiments, -Cy- is

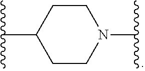 [SMILES: *C1CCN(*)CC1]

In certain embodiments, -Cy- is

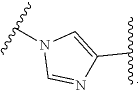 [SMILES: *c1cn(*)cn1]

In certain embodiments, R^{I} is hydrogen. In certain embodiments, R^{I} is C₁₋₆ alkyl. In certain embodiments, R^{I} is C₁₋₆ alkyl. In certain embodiments, R^{I} is C₂ alkyl. In certain embodiments, R^{I} is C₃ alkyl. In certain embodiments, R^{I} is C₄ alkyl. In certain embodiments, R^{I} is C₅ alkyl. In certain embodiments, R^{I} is C₆ alkyl.

In certain embodiments, R^{J} is C₁₋₆ alkyl. In certain embodiments, R^{J} is C₁ alkyl. In certain embodiments, R^{J} is C₂ alkyl. In certain embodiments, R^{J} is C₃ alkyl. In certain embodiments, R^{J} is C₁₋₆ haloalkyl. In certain embodiments, R^{J} is C₁ haloalkyl. In certain embodiments, R^{J} is C₂ haloalkyl. In certain embodiments, R^{J} is C₃ haloalkyl. In certain embodiments, R^{J} is C₁₋₆ fluoroalkyl. In certain embodiments, R^{J} is —CF₃. In certain embodiments, R^{J} is —CHF₂. In certain embodiments, R^{J} is C₁₋₆ alkoxyl. In certain embodiments, R^{J} is C₁ alkoxyl. In certain embodiments, R^{J} is C₂ alkoxyl. In certain embodiments, R^{J} is C₃ alkoxyl. In certain embodiments, R^{J} is C₄ alkoxyl.

In certain embodiments, r is 0. In certain embodiments, r is 1. In certain embodiments, r is 2. In certain embodiments, r is 3. In certain embodiments, r is 4. In certain embodiments, r is 5. In certain embodiments, r is 6. In certain embodiments, r is 7. In certain embodiments, r is 8. In certain embodiments, r is 9. In certain embodiments, r is 10.

In certain embodiments, L is a covalent bond.

In certain embodiments, L is a bivalent, saturated, straight or branched C₁₋₂₀ hydrocarbon chain, wherein 1, 2, 3, 4, 5, 6, 7, or 8 methylene units of L are independently and optionally replaced by -Cy-, —O—, —N(H)—, —N(C₁₋₄ alkyl)-, —S—, —C(O)—, —S(O)—, or —S(O)₂—, wherein each -Cy- is independently a 3-7 membered saturated carbocyclylene, a 3-7 membered saturated heterocyclylene containing 1 or 2 heteroatoms independently selected from nitrogen, oxygen, and sulfur, or a 5-6 membered heteroarylene containing 1, 2, 3, or 4 heteroatoms independently selected from nitrogen, oxygen, and sulfur, wherein the carbocyclene, heterocyclylene, and heteroarylene are substituted with 0-2 occurrences of R^{J}, and wherein R^{J} represents independently for each occurrence C₁₋₆ alkyl, C₁₋₆ haloalkyl, or C₁₋₆ alkoxyl.

In certain embodiments, each -Cy- is independently a 3-7 membered saturated carbocyclylene, wherein the carbocyclene is substituted with 0-2 occurrences of R^{J}. In certain embodiments, -Cy- is a 3 membered saturated carbocyclylene, wherein the carbocyclene is substituted with 0-2 occurrences of R^{J}. In certain embodiments, -Cy- is a 4 membered saturated carbocyclylene, wherein the carbocyclene is substituted with 0-2 occurrences of R^{J}. In certain embodiments, -Cy- is a 5 membered saturated carbocyclylene, wherein the carbocyclene is substituted with 0-2 occurrences of R^{J}. In certain embodiments, -Cy- is a 6 membered saturated carbocyclylene, wherein the carbocyclene is substituted with 0-2 occurrences of R^{J}. In certain embodiments, -Cy- is a 7 membered saturated carbocyclylene, wherein the carbocyclene is substituted with 0-2 occurrences of R^{J}.

In certain embodiments, each -Cy- is independently a 3-7 membered saturated heterocyclylene containing 1 or 2 heteroatoms independently selected from nitrogen, oxygen, and sulfur, wherein the heterocyclylene is substituted with 0-2 occurrences of R^{J}. In certain embodiments, -Cy- is a 3 membered saturated heterocyclylene containing 1 or 2 heteroatoms independently selected from nitrogen, oxygen, and sulfur, wherein the heterocyclylene is substituted with 0-2 occurrences of R^{J}. In certain embodiments, -Cy- is a 4 membered saturated heterocyclylene containing 1 or 2 heteroatoms independently selected from nitrogen, oxygen, and sulfur, wherein the heterocyclylene is substituted with 0-2 occurrences of R^{J}. In certain embodiments, -Cy- is a 5 membered saturated heterocyclylene containing 1 or 2 heteroatoms independently selected from nitrogen, oxygen, and sulfur, wherein the heterocyclylene is substituted with 0-2 occurrences of R^{J}. In certain embodiments, -Cy- is a 6 membered saturated heterocyclylene containing 1 or 2 heteroatoms independently selected from nitrogen, oxygen, and sulfur, wherein the heterocyclylene is substituted with 0-2 occurrences of R^{J}. In certain embodiments, -Cy- is a 7 membered saturated heterocyclylene containing 1 or 2 heteroatoms independently selected from nitrogen, oxygen, and sulfur, wherein the heterocyclylene is substituted with 0-2 occurrences of R^{J}.

In certain embodiments, each -Cy- is independently a 5-6 membered heteroarylene containing 1, 2, 3, or 4 heteroatoms independently selected from nitrogen, oxygen, and sulfur, wherein the heteroarylene is substituted with 0-2 occurrences of R^{J}. In certain embodiments, -Cy- is a 5 membered heteroarylene containing 1, 2, 3, or 4 heteroatoms independently selected from nitrogen, oxygen, and sulfur, wherein the heteroarylene is substituted with 0-2 occurrences of R^{J}. In certain embodiments, -Cy- is a 5 membered heteroarylene containing 1 or 2 nitrogen atoms, wherein the heteroarylene is substituted with 0-2 occurrences of R^{J}. In certain embodiments, -Cy- is a 5 membered heteroarylene containing 1 or 2 nitrogen atoms. In certain embodiments, -Cy- is a 6 membered heteroarylene containing 1, 2, 3, or 4 heteroatoms independently selected from nitrogen, oxygen, and sulfur, wherein the heteroarylene is substituted with 0-2 occurrences of R^{J}. In certain embodiments, -Cy- is a 6 membered heteroarylene containing 1 or 2 nitrogen atoms, wherein the heteroarylene is substituted with 0-2 occurrences of R^{J}. In certain embodiments, -Cy- is a 6 membered heteroarylene containing 1 or 2 nitrogen atoms.

In certain embodiments, -Cy- is

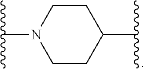 [SMILES: *C1CCN(*)CC1]

In certain embodiments, -Cy- is

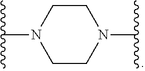 [SMILES: *N1CCN(*)CC1]

In certain embodiments, -Cy- is

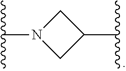 [SMILES: *C1CN(*)C1]

In certain embodiments, -Cy- is

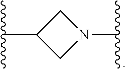 [SMILES: *C1CN(*)C1]

In certain embodiments, -Cy- is

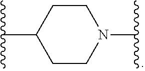 [SMILES: *C1CCN(*)CC1]

In certain embodiments, -Cy- is

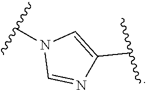 [SMILES: *c1cn(*)cn1]

In certain embodiments, L is a bivalent, saturated, straight or branched C₁₋₂₀ hydrocarbon chain, wherein 1, 2, 3, 4, or 5 methylene units of L are independently and optionally replaced by —O—, —N(H)—, —N(C₁₋₆ aliphatic)-, —S—, —C(O)—, —S(O)—, or —S(O)₂—, and one methylene unit of L is optionally replaced by a bivalent ring selected from a 3-, 4-, 5-, 6-, or 7-membered saturated carbocyclylene, a 3-, 4-, 5-, 6-, or 7-membered saturated heterocyclylene having 1 or 2 heteroatoms independently selected from nitrogen, oxygen, and sulfur, or a 5- or 6-membered heteroarylene having 1, 2, 3, or 4 heteroatoms independently selected from nitrogen, oxygen, and sulfur.

In certain embodiments, L is a bivalent, saturated, straight or branched C₁₋₂₀ hydrocarbon chain, wherein 1, 2, 3, 4, or 5 methylene units of L are independently replaced by —O—, —N(H)—, —N(C₁₋₄ alkyl)-, —S—, —C(O)—, —S(O)—, or —S(O)₂—.

In certain embodiments, L is a bivalent, saturated, straight or branched C₅₋₂₀ hydrocarbon chain, wherein 1, 2, 3, 4, or 5 methylene units of L are independently replaced by —O—, —N(H)—, —N(C₁₋₄ alkyl)-, —S—, —C(O)—, —S(O)—, or —S(O)₂—.

In certain embodiments, L is —C(O)—(CH₂)₁₋₁₀—C(O)—, —C(O)—(CH₂)₂—(OCH₂CH₂)₁₋₅—C(O)—, —C(O)—CH₂—(OCH₂CH₂)₁₋₅—, —C(O)—(CH₂)₁₋₁₀—, —C(O)—(CH₂)₂—(OCH₂CH₂)₁₋₅—, —O—(CH₂)₂—(OCH₂CH₂)₁₋₅—C(O)—, or —O—(CH₂)₂—(OCH₂CH₂)₁₋₅—. In certain embodiments, L is —C(O)—(CH₂)₂—(OCH₂CH₂)₁₋₅—, —C(O)—CH₂—(OCH₂CH₂)₁₋₅—, —C(O)—(CH₂)₁₋₁₀—, —O—(CH₂)₂—(OCH₂CH₂)₁₋₅—, —C(O)—(CH₂)₂—(OCH₂CH₂)₁₋₅—C(O)—, or —C(O)—(CH₂)₁₋₁₀—C(O)—.

In certain embodiments, L is —C(O)—(CH₂)₂—(OCH₂CH₂)₁₋₅—, —C(O)—CH₂—(OCH₂CH₂)₁₋₅—, —C(O)—(CH₂)₁₋₁₀—, or —O—(CH₂)₂—(OCH₂CH₂)₁₋₅—, wherein the carbon atom of the terminal CH₂ group is attached to TBM. In certain embodiments, L is —C(O)—(CH₂)₂—(OCH₂CH₂)₁₋₅—, wherein the carbon atom of the terminal CH₂ group is attached to TBM. In certain embodiments, L is —C(O)—CH₂—(OCH₂CH₂)₁₋₅—, wherein the carbon atom of the terminal CH₂ group is attached to TBM. In certain embodiments, L is —C(O)—(CH₂)₁₋₁₀—, wherein the carbon atom of the terminal CH₂ group is attached to TBM. In certain embodiments, L is —O—(CH₂)₂—(OCH₂CH₂)₁₋₅—, wherein the carbon atom of the terminal CH₂ group is attached to TBM.

In certain embodiments, L is —C(O)—(CH₂)₂—(OCH₂CH₂)₁₋₅—C(O)—, —C(O)—(CH₂)₁₋₁₀—C(O)—, or —O—(CH₂)₂—(OCH₂CH₂)₁₋₅—C(O)—. In certain embodiments, L is —C(O)—(CH₂)₂—(OCH₂CH₂)₁₋₅—C(O)— or —C(O)—(CH₂)₁₋₁₀—C(O)—. In certain embodiments, L is —C(O)—(CH₂)₂—(OCH₂CH₂)₁₋₅—C(O)—. In certain embodiments, L is —C(O)—(CH₂)₁₋₁₀—C(O)—. In certain embodiments, L is —O—(CH₂)₂—(OCH₂CH₂)₁₋₅—C(O)—.

In certain embodiments, L is

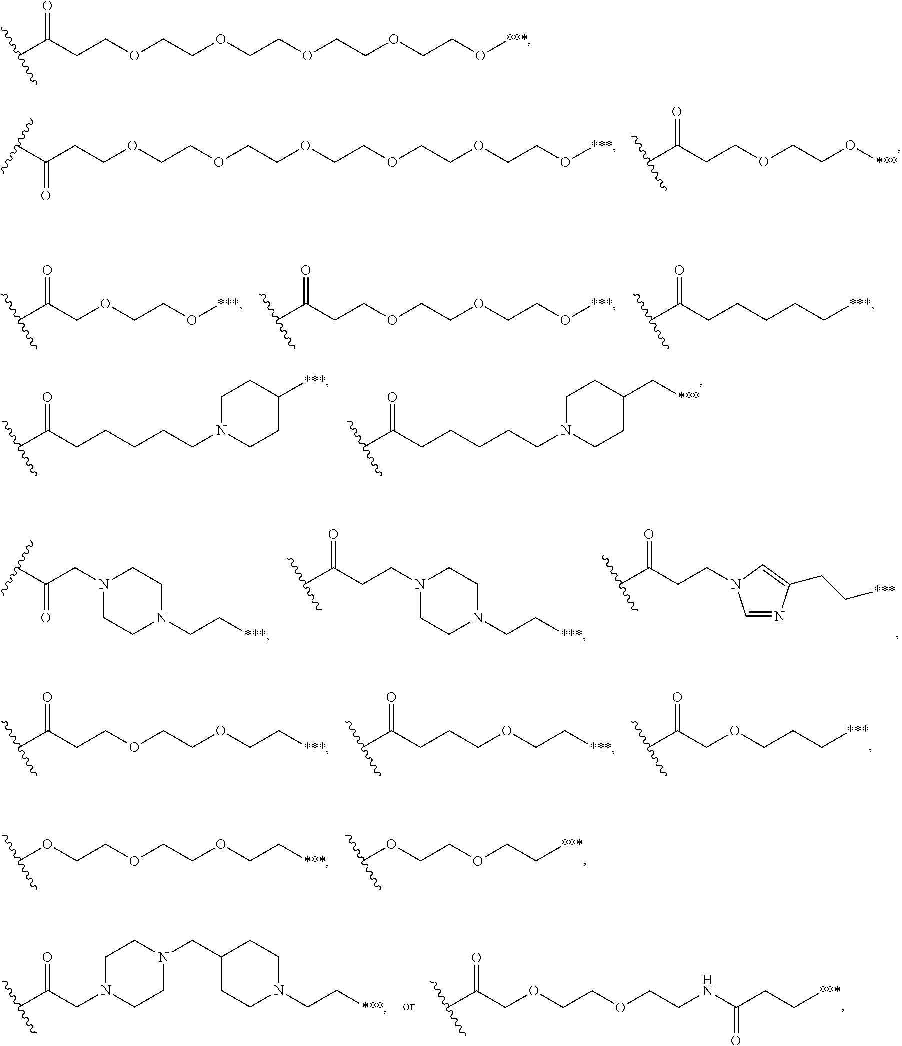

wherein *** is a point of attachment to TBM. In certain embodiments, L is

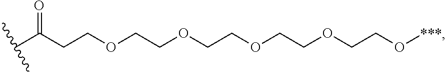

wherein *** is a point of attachment to TBM. In certain embodiments, L is

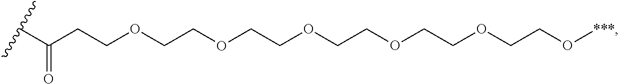

wherein *** is a point of attachment to TBM. In certain embodiments, L is

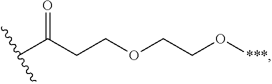

wherein *** is a point of attachment to TBM. In certain embodiments, L is

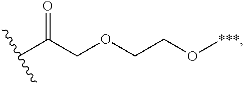

wherein *** is a point of attachment to TBM In certain embodiments, L is

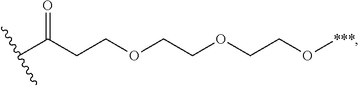

wherein *** is a point of attachment to TBM. In certain embodiments, L is

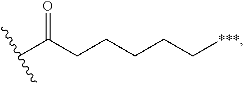

wherein *** is a point of attachment to TBM. In certain embodiments, L is

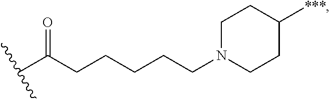

wherein *** is a point of attachment to TBM. In certain embodiments, L is

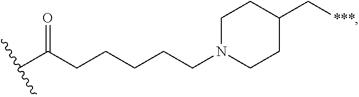

wherein *** is a point of attachment to TBM. In certain embodiments, L is

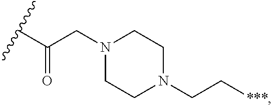

wherein *** is a point of attachment to TBM. In certain embodiments, L is

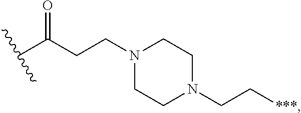

wherein *** is a point of attachment to TBM. In certain embodiments, L is

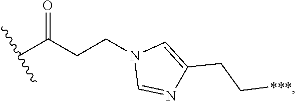

wherein *** is a point of attachment to TBM. In certain embodiments, L is

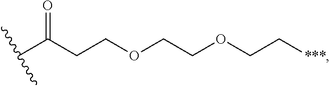

wherein *** is a point of attachment to TBM. In certain embodiments, L is

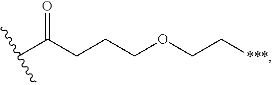

**wherein *** is a point of attachment to TBM. In certain embodiments, L is

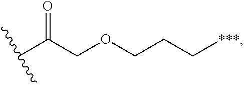

wherein *** is a point of attachment to TBM. In certain embodiments, L is

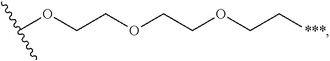

wherein *** is a point of attachment to TBM. In certain embodiments, L is

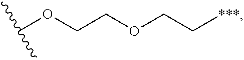

wherein *** is a point of attachment to TBM. In certain embodiments, L is

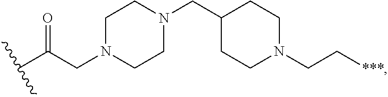

wherein *** is a point of attachment to TBM. In certain embodiments, L is

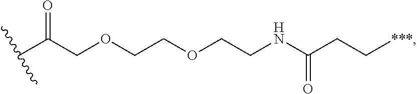

wherein *** is a point of attachment to TBM.

In certain embodiments, L is

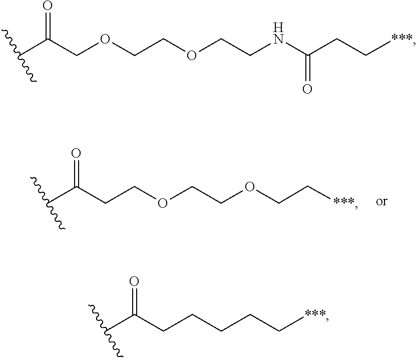

or wherein *** is a point of attachment to TBM.

In certain embodiments, L is

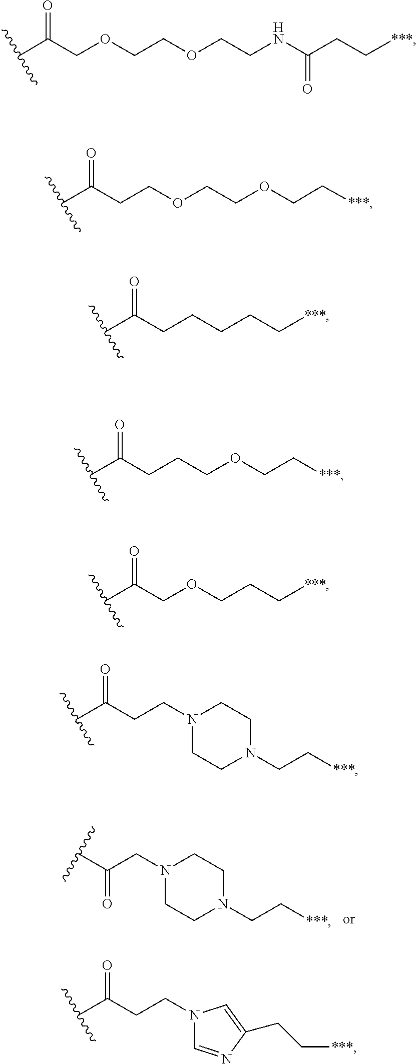

wherein *** is a point of attachment to TBM. In certain embodiments, L is

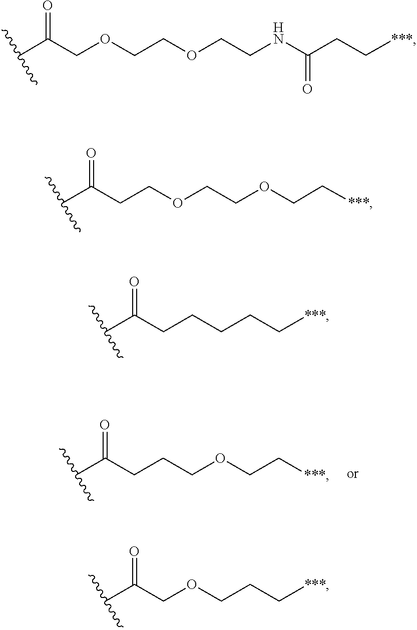

wherein *** is a point of attachment to TBM. In certain embodiments, L is

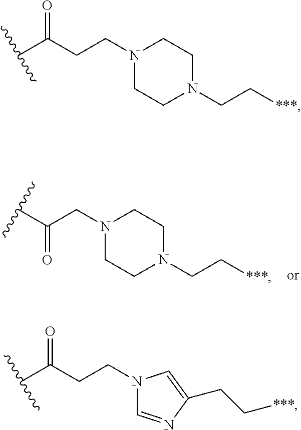

wherein *** is a point of attachment to TBM. In certain embodiments, L is

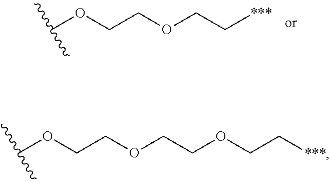

wherein *** is a point of attachment to TBM.

In certain embodiments, L is

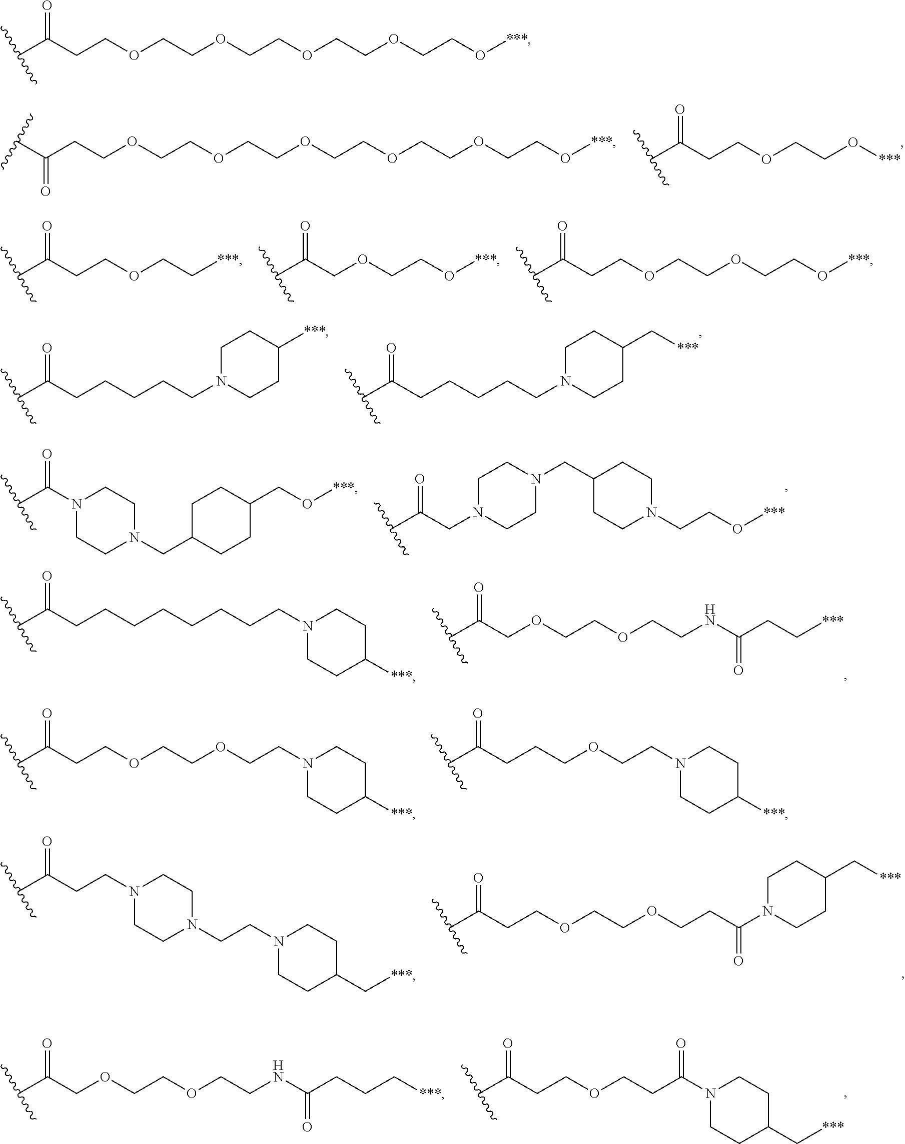

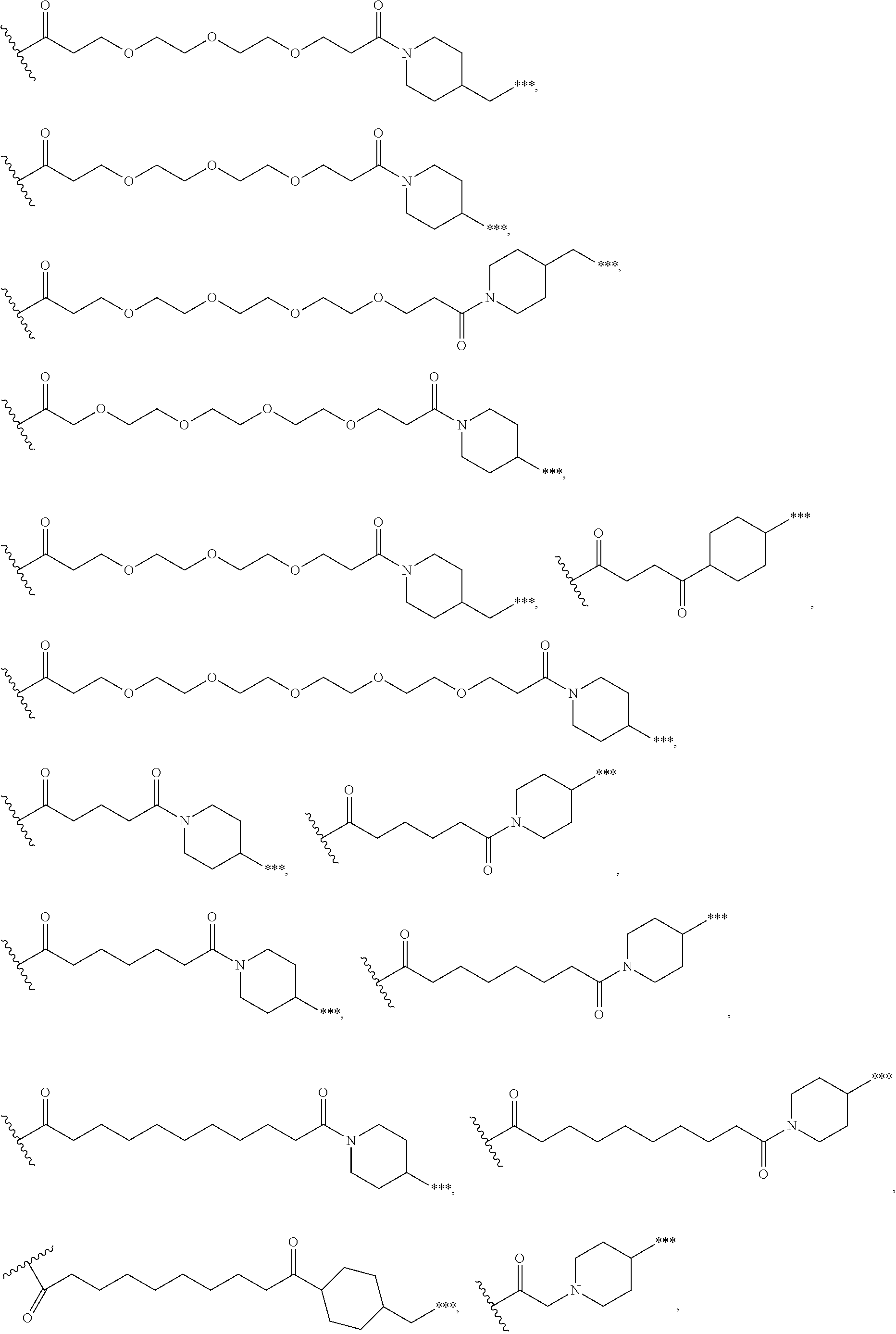

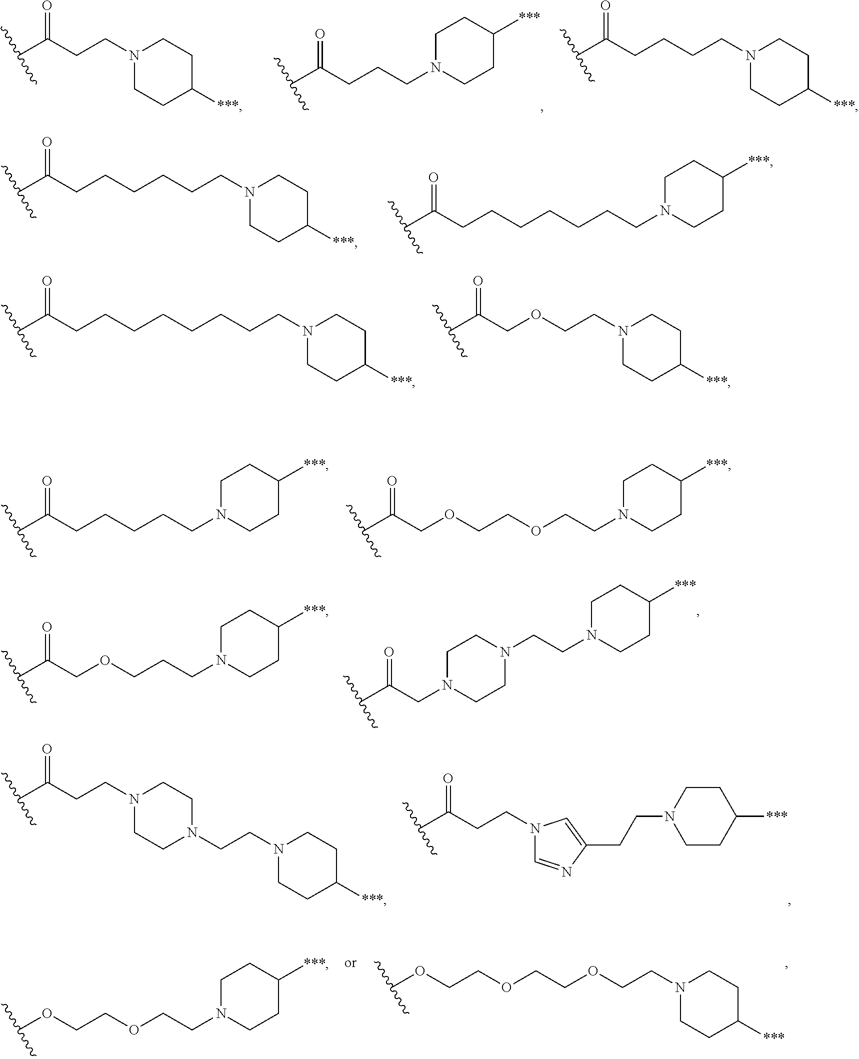

wherein *** is a point of attachment to TBM. In certain embodiments, L is

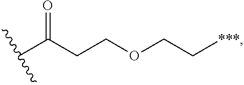

wherein *** is a point of attachment to TBM. In certain embodiments, L is

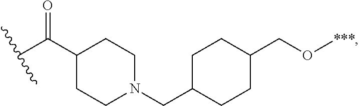

wherein *** is a point of attachment to TBM. In certain embodiments, L is

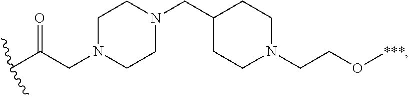

wherein *** is a point of attachment to TBM. In certain embodiments, L is

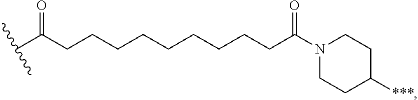

wherein *** is a point of attachment to TBM. In certain embodiments, L is

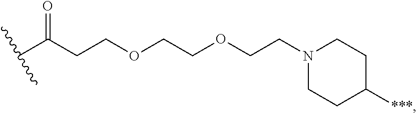

wherein *** is a point of attachment to TBM. In certain embodiments, L is

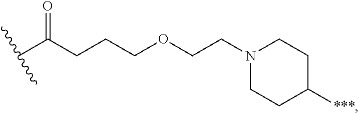

wherein *** is a point of attachment to TBM. In certain embodiments, L is

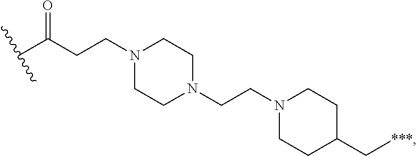

wherein *** is a point of attachment to TBM. In certain embodiments, L is

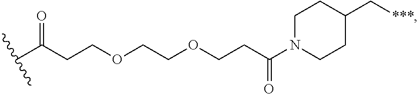

wherein *** is a point of attachment to TBM. In certain embodiments, L is

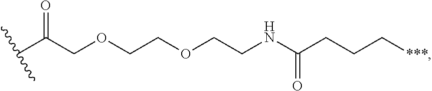

wherein *** is a point of attachment to TBM. In certain embodiments, L is

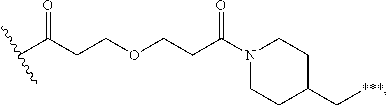

wherein *** is a point of attachment to TBM. In certain embodiments, L is

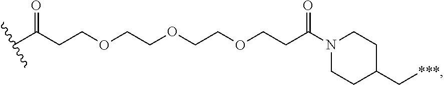

wherein *** is a point of attachment to TBM. In certain embodiments, L is

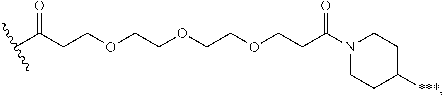

wherein *** is a point of attachment to TBM. In certain embodiments, L is

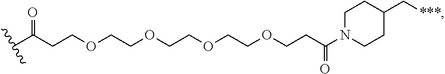

wherein *** is a point of attachment to TBM. In certain embodiments, L is

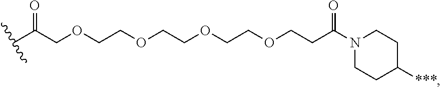

wherein *** is a point of attachment to TBM. In certain embodiments, L is

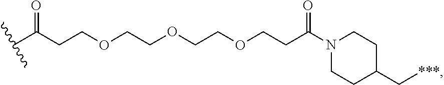

wherein *** is a point of attachment to TBM. In certain embodiments, L is

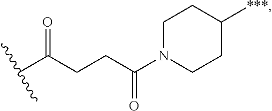

wherein *** is a point of attachment to TBM. In certain embodiments, L is

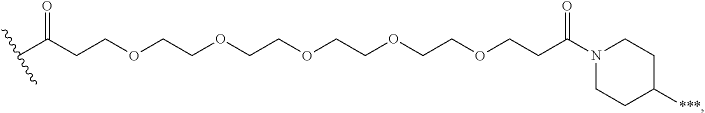

wherein *** is a point of attachment to TBM. In certain embodiments, L is

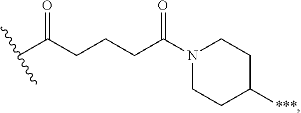

wherein *** is a point of attachment to TBM. In certain embodiments, L is

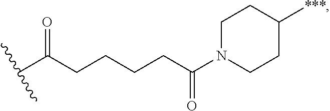

wherein *** is a point of attachment to TBM. In certain embodiments, L is

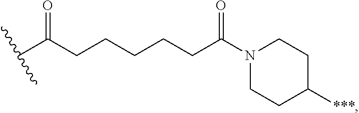

wherein *** is a point of attachment to TBM. In certain embodiments, L is

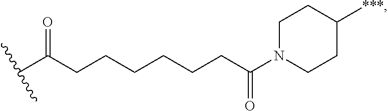

wherein *** is a point of attachment to TBM. In certain embodiments, L is

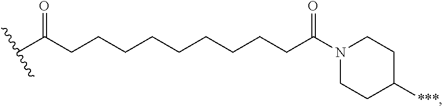

wherein *** is a point of attachment to TBM. In certain embodiments, L is

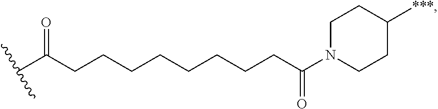

wherein *** is a point of attachment to TBM. In certain embodiments, L is

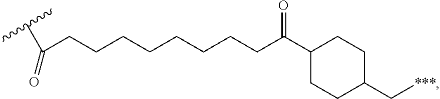

wherein *** is a point of attachment to TBM. In certain embodiments, L is

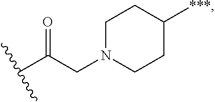

wherein *** is a point of attachment to TBM. In certain embodiments, L is

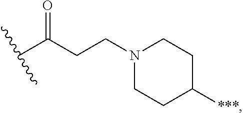

wherein *** is a point of attachment to TBM. In certain embodiments, L is

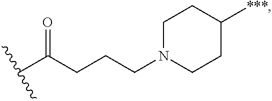

wherein *** is a point of attachment to TBM. In certain embodiments, L is

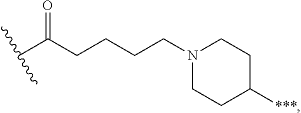

wherein *** is a point of attachment to TBM. In certain embodiments, L is

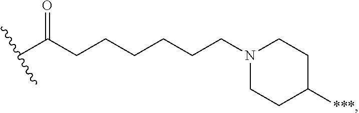

wherein *** is a point of attachment to TBM. In certain embodiments, L is

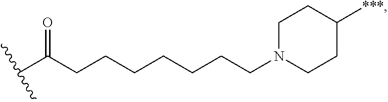

wherein *** is a point of attachment to TBM. In certain embodiments, L is

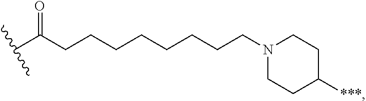

wherein *** is a point of attachment to TBM. In certain embodiments, L is

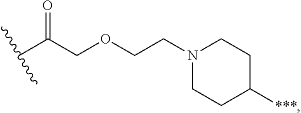

wherein *** is a point of attachment to TBM. In certain embodiments, L is

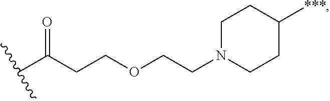

wherein *** is a point of attachment to TBM. In certain embodiments, L is

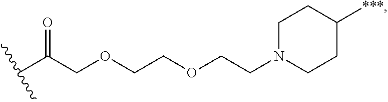

wherein *** is a point of attachment to TBM. In certain embodiments, L is

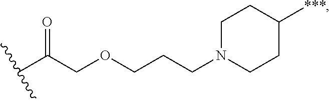

wherein *** is a point of attachment to TBM. In certain embodiments, L is

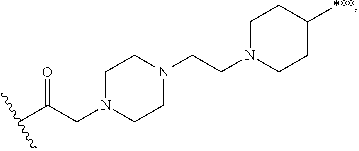

wherein *** is a point of attachment to TBM. In certain embodiments, L is

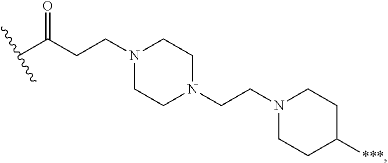

wherein *** is a point of attachment to TBM. In certain embodiments, L is

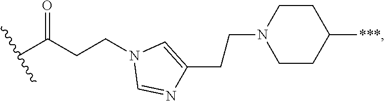

wherein *** is a point of attachment to TBM. In certain embodiments, L is

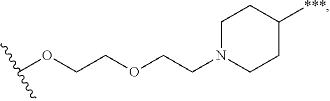

wherein *** is a point of attachment to TBM. In certain embodiments, L is

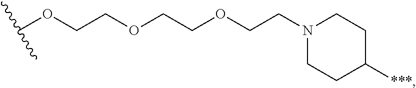

wherein *** is a point of attachment to TBM.

In certain embodiments, L is

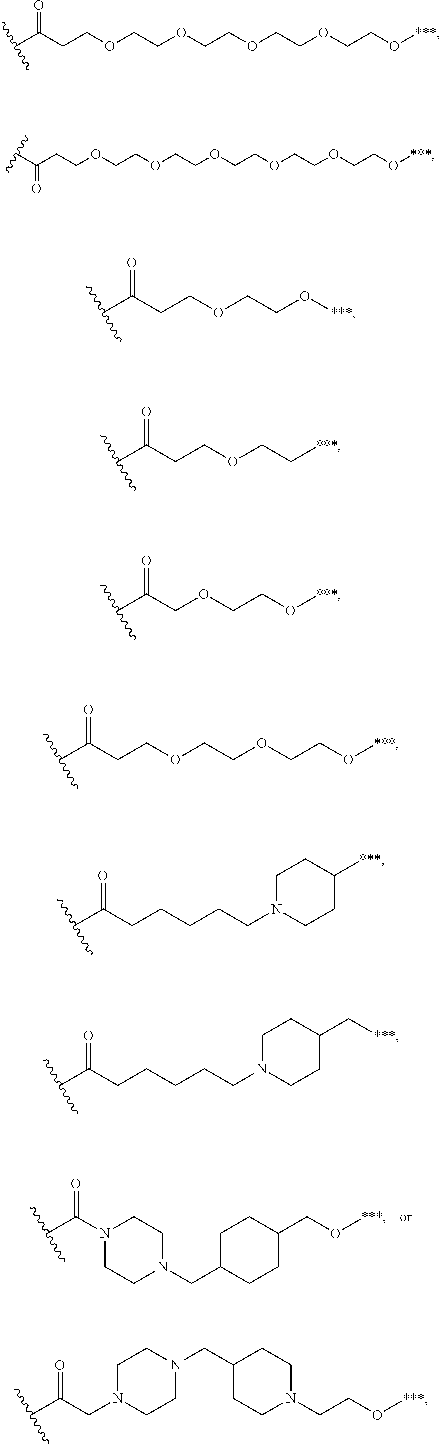

wherein *** is a point of attachment to TBM. In certain embodiments, L is

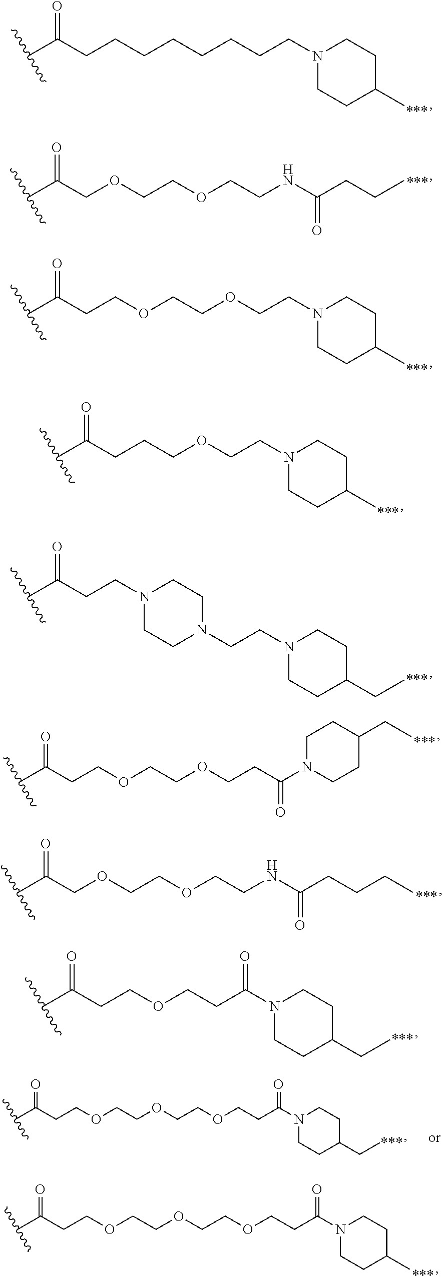

wherein *** is a point of attachment to TBM. In certain embodiments, L is

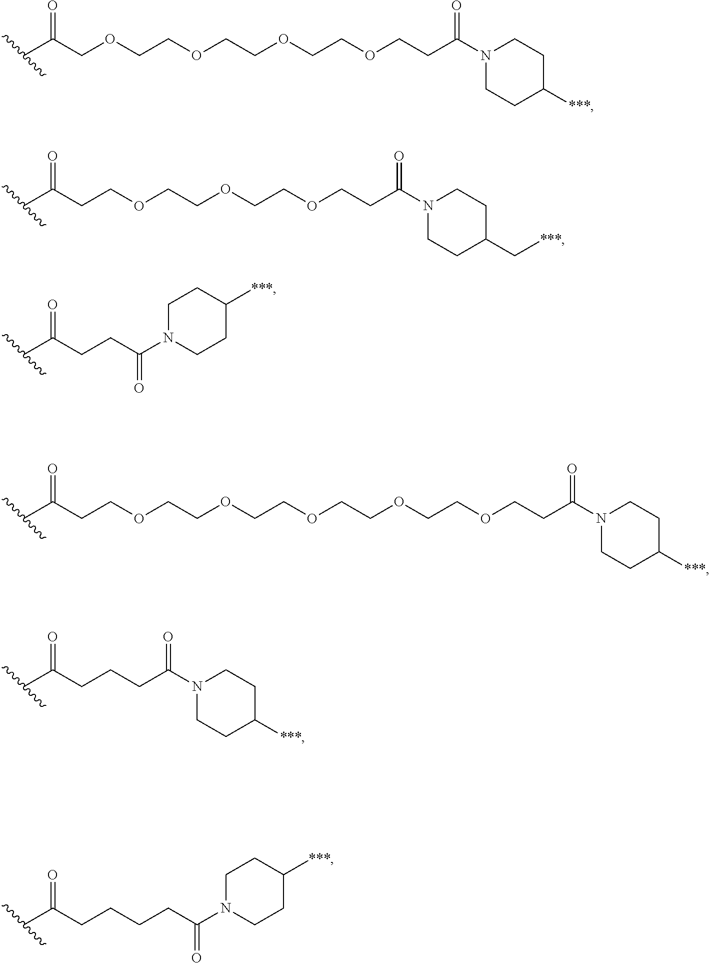

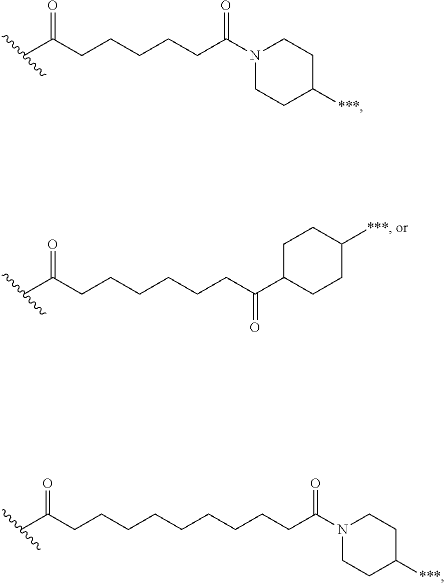

wherein *** is a point of attachment to TBM. In certain embodiments, L is

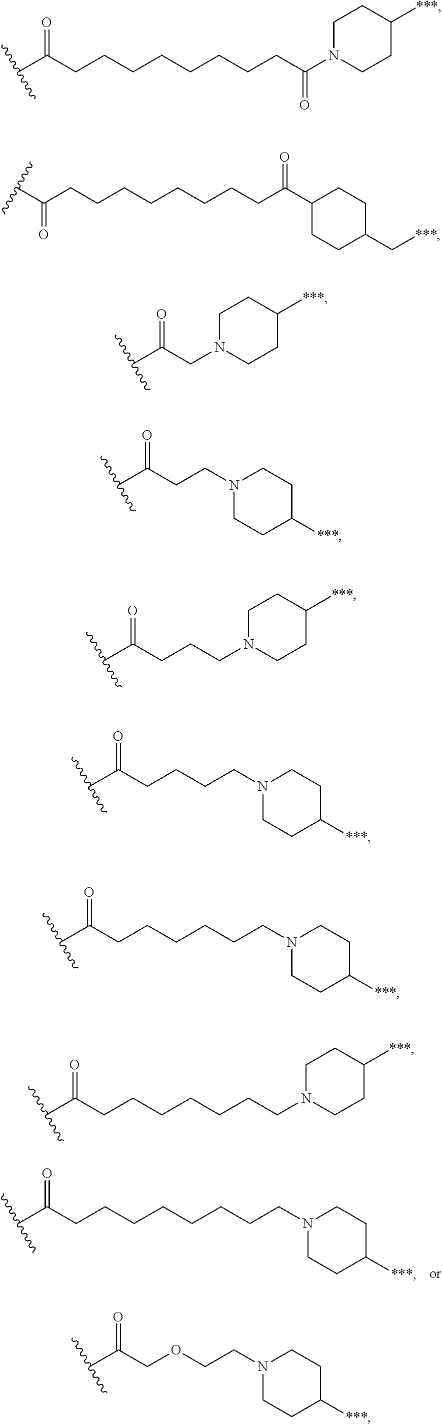

wherein *** is a point of attachment to TBM. In certain embodiments, L is

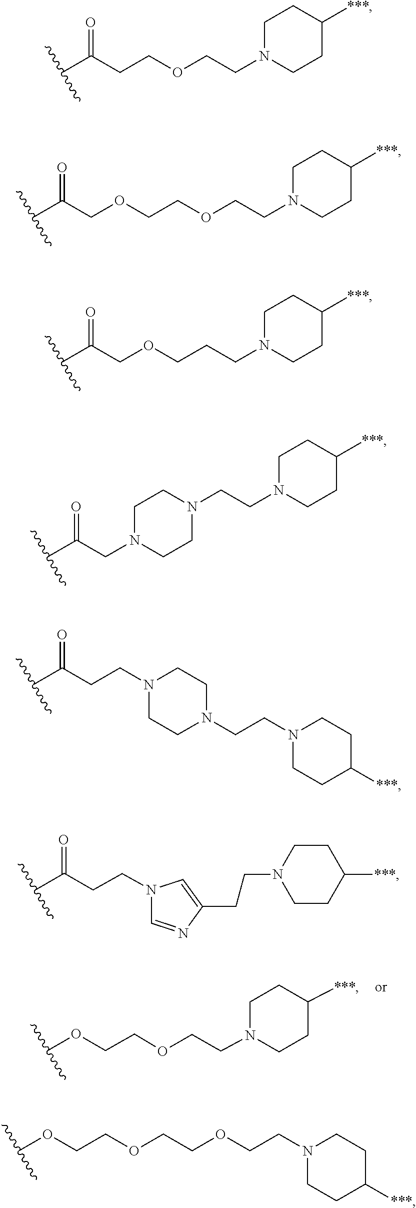

wherein *** is a point of attachment to TBM.

In certain embodiments, L is

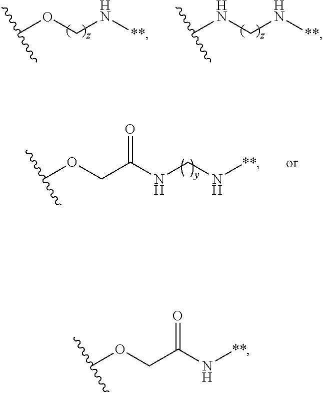

wherein y is 1, 2, or 3, wherein z is 4, 5, 6, or 7, and wherein ** is a point of attachment to LBM. In certain embodiments, L is

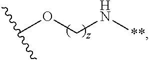

wherein z is 4, 5, 6, or 7, and wherein ** is a point of attachment to LBM. In certain embodiments, L is

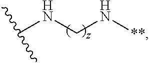

wherein z is 4, 5, 6, or 7, and wherein ** is a point of attachment to LBM. In certain embodiments, L is

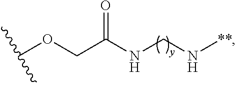

wherein y is 1, 2, or 3, and wherein ** is a point of attachment to LBM. In certain embodiments, L is

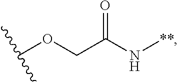

wherein ** is a point of attachment to LBM.

In certain embodiments, L is H

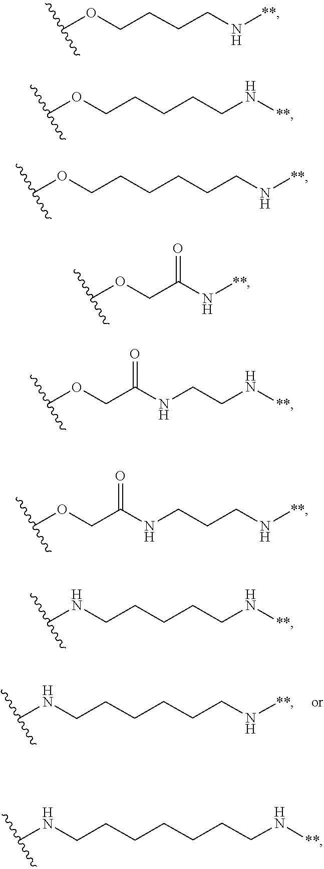

wherein ** is a point of attachment to LBM. In certain embodiments, L is

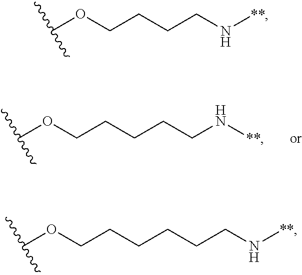

wherein ** is a point of attachment to LBM. In certain embodiments L is

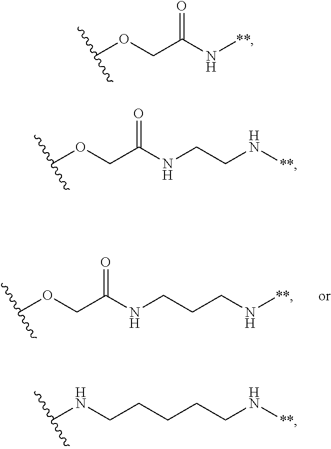

wherein ** is a point of attachment to LBM. In certain embodiments, L is

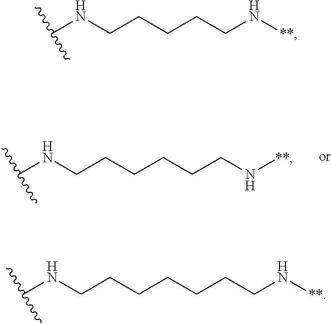

wherein ** is a point of attachment to LBM.

In certain embodiments, L is

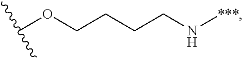

wherein ** is a point of attachment to LBM. In certain embodiments, L is

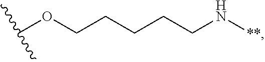

wherein ** is a point of attachment to LBM. In certain embodiments, L is

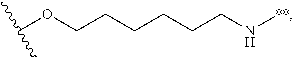

wherein ** is a point of attachment to LBM. In certain embodiments, L is

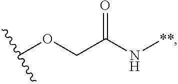

wherein ** is a point of attachment to LBM. In certain embodiments, L is

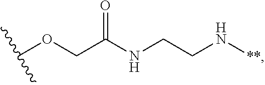

wherein ** is a point of attachment to LBM. In certain embodiments, L is

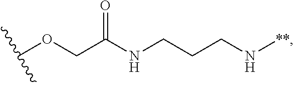

wherein ** is a point of attachment to LBM. In certain embodiments, L is

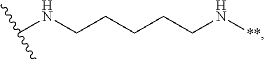

wherein ** is a point of attachment to LBM. In certain embodiments, L is

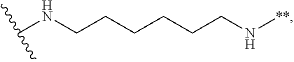

wherein ** is a point of attachment to LBM. In certain embodiments, L is

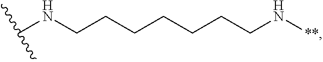

wherein ** is a point of attachment to LBM.

In certain embodiments, L is selected from the groups depicted in the compounds in Table 1, below.

### Part D: Target Binding Moiety (TBM) of Formula A

As defined generally above, TBM can be a radical of Formula A:

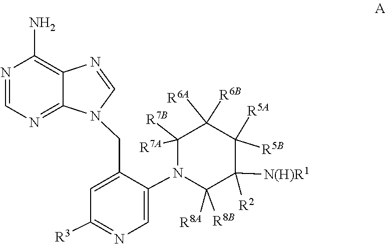

wherein:
R¹ is hydrogen or C₁₋₄ alkyl;
R² is —(C₁₋₄ haloalkyl substituted with 0 or 1 occurrences of hydroxyl) or a 5-6 membered heteroaryl containing 1 nitrogen atom, wherein the heteroaryl is substituted with 0 or 1 occurrences of halo;
R³ is phenyl or pyridinyl, each of which are substituted with p occurrences of R⁴;
R⁴ represents independently for each occurrence halo, hydroxyl, C₁₋₄ haloalkyl, —O—(C₁₋₄ haloalkyl), or C₁₋₄ alkoxyl;
R^{5A}, R^{5B}, R^{6A}, R^{6B}, R^{7A}, R^{7B}, R^{8A}, and R^{8B} are independently hydrogen, halo, cyano, aryl, —(C₀₋₆ alkylene)-(hydroxyl), C₁₋₆ alkyl, —(C₁₋₆ haloalkyl substituted with 0 or 1 occurrences of hydroxyl), —(C₀₋₆ alkylene)-(C₁₋₆ alkoxyl substituted with 0 or 1 occurrences of halo), —C(O)—OR⁹, or —(C(R¹⁰)(R¹¹))ₛ—C(O)—N(R¹²)(R¹³);
R⁹, R¹⁰, R¹¹, R¹², and R¹³ are independently hydrogen or C₁₋₄ alkyl;
p is 0, 1, 2, or 3; and
s is 0, 1, 2, 3, or 4.

The definitions of variables in Formula A above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

As defined generally above, R¹ is hydrogen or C₁₋₄ alkyl. In certain embodiments, R¹ is hydrogen. In certain embodiments, R¹ is C₁₋₄ alkyl. In certain embodiments, R¹ is C₁ alkyl. In certain embodiments, R¹ is C₂ alkyl. In certain embodiments, R¹ is C₃ alkyl. In certain embodiments, R¹ is C₄ alkyl. In certain embodiments, R¹ is selected from the groups depicted in the compounds in Table A below.

As defined generally above, R² is —(C₁₋₄ haloalkyl substituted with 0 or 1 occurrences of hydroxyl) or a 5-6 membered heteroaryl containing 1 nitrogen atom, wherein the heteroaryl is substituted with 0 or 1 occurrences of halo. In certain embodiments, R² is C₁₋₄ haloalkyl substituted with 0 or 1 occurrences of hydroxyl. In certain embodiments, R² is C₁ haloalkyl substituted with 0 or 1 occurrences of hydroxyl. In certain embodiments, R² is C₂ haloalkyl substituted with 0 or 1 occurrences of hydroxyl. In certain embodiments, R² is C₃ haloalkyl substituted with 0 or 1 occurrences of hydroxyl. In certain embodiments, R² is C₄ haloalkyl substituted with 0 or 1 occurrences of hydroxyl. In certain embodiments, R² is C₁₋₄ haloalkyl. In certain embodiments, R² is C₁₋₄ haloalkyl substituted with 1 occurrence of hydroxyl. In certain embodiments, R² is a 5-6 membered heteroaryl containing 1 nitrogen atom, wherein the heteroaryl is substituted with 0 or 1 occurrences of halo. In certain embodiments, R² is a 5 membered heteroaryl containing 1 nitrogen atom, wherein the heteroaryl is substituted with 0 or 1 occurrences of halo. In certain embodiments, R² is a 6 membered heteroaryl containing 1 nitrogen atom, wherein the heteroaryl is substituted with 0 or 1 occurrences of halo. In certain embodiments, R² is C₁₋₄ fluoroalkyl substituted with 0 or 1 occurrences of hydroxyl. In certain embodiments, R² is pyridinyl substituted with 0 or 1 occurrences of halo. In certain embodiments, R² is pyridinyl substituted with 1 occurrence of halo. In certain embodiments, R² is pyridinyl substituted with 1 occurrence of chloro.

In certain embodiments, R² is selected from

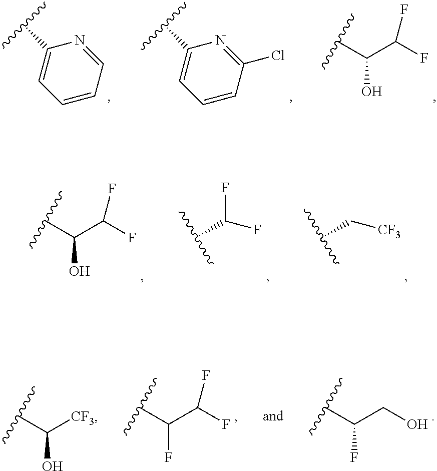

In certain embodiments R² is selected from

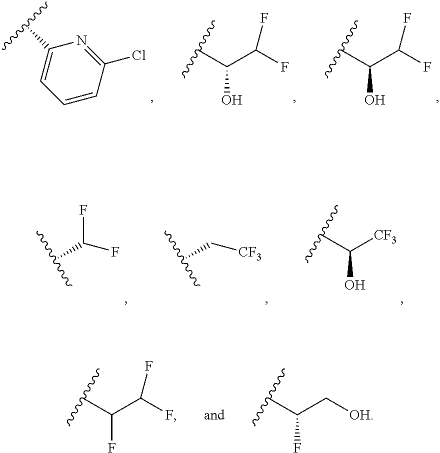

In certain embodiments, R² is selected from

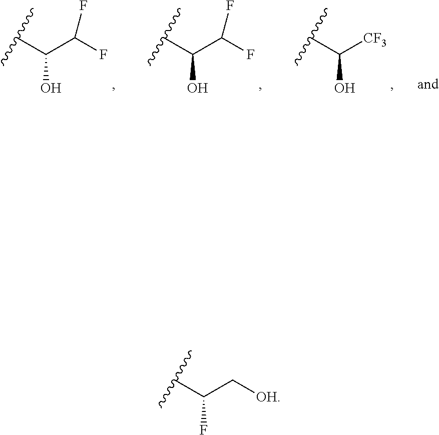 [SMILES: *[C@@H](O)C(F)F ; *[C@H](O)C(F)F ; *[C@H](O)C(F)(F)F ; *[C@@H](F)CO]

In certain embodiments, R² is selected from

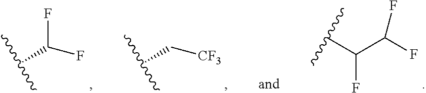

In certain embodiments, R² is

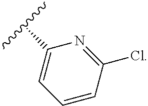

In certain embodiments, R² is

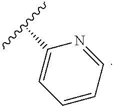

In certain embodiments, R² is

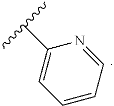 [SMILES: *c1ccccn1]

In certain embodiments, R² is

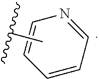

In certain embodiments, R² is

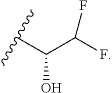 [SMILES: *[C@@H](O)C(F)F]

In certain embodiments, R² is

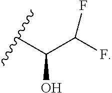 [SMILES: *[C@H](O)C(F)F]

In certain embodiments, R² is

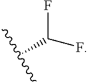

In certain embodiments, R² is

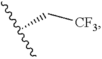

In certain embodiments, R² is

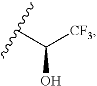 [SMILES: *[C@H](O)C(F)(F)F]

In certain embodiments, R² is

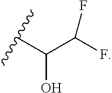 [SMILES: *C(O)C(F)F]

In certain embodiments, R² is

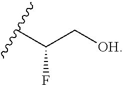 [SMILES: *[C@@H](F)CO]

In certain embodiments, R² is selected from the groups depicted in the compounds in Table A below.

As defined generally above, R³ is phenyl or pyridinyl, each of which are substituted with p occurrences of R⁴. In certain embodiments, R³ is phenyl substituted with p occurrences of R⁴. In certain embodiments, R³ is pyridinyl substituted with p occurrences of R⁴. In certain embodiments, R³ is selected from

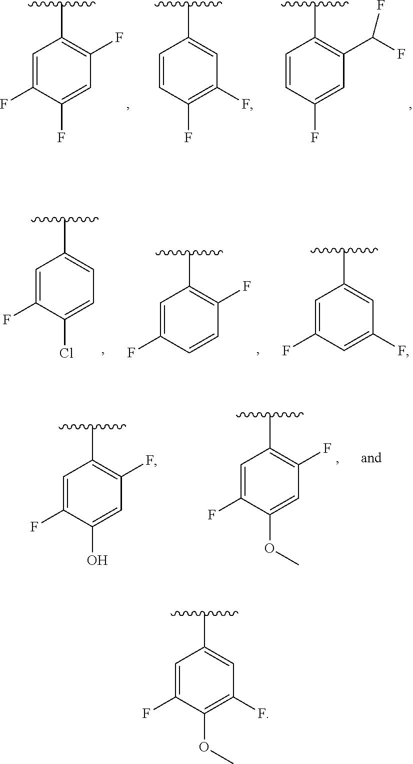

In certain embodiments, R³ is a phenyl substituted with 2 or 3 occurrences of R⁴. In certain embodiments, R³ is selected from

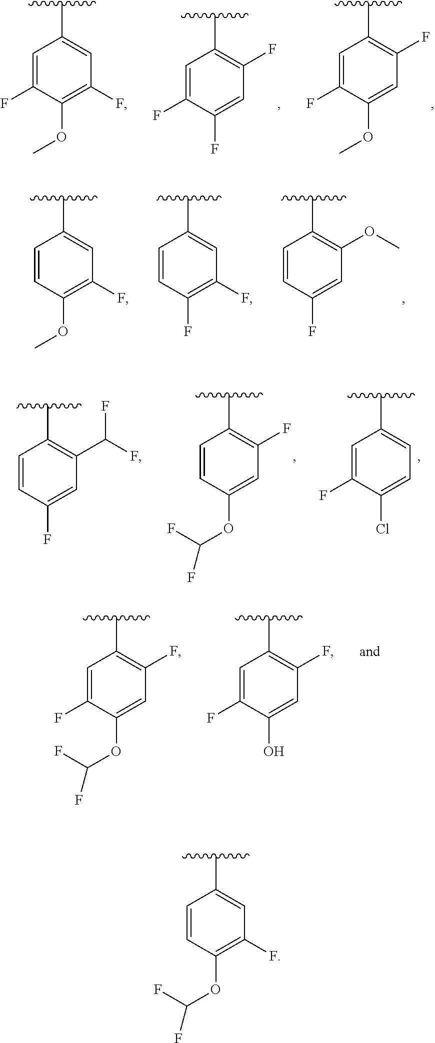

In certain embodiments, R³ is

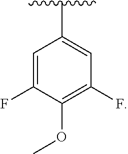 [SMILES: *c1cc(F)c(OC)c(F)c1]

In certain embodiments, R³ is

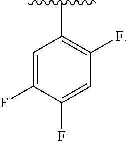 [SMILES: *c1cc(F)c(F)cc1F]

In certain embodiments, R³ is

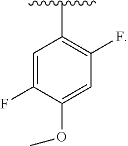 [SMILES: *c1cc(F)c(OC)cc1F]

In certain embodiments, R³ is

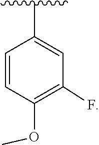 [SMILES: *c1ccc(OC)c(F)c1]

In certain embodiments, R³ is

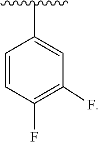 [SMILES: *c1ccc(F)c(F)c1]

In certain embodiments, R³ is

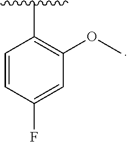 [SMILES: *c1ccc(F)cc1OC]

In certain embodiments, R³ is

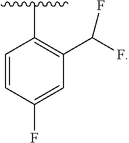 [SMILES: *c1ccc(F)cc1C(F)F]

In certain embodiments, R³ is

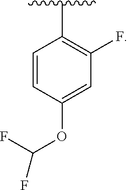 [SMILES: *c1ccc(OC(F)F)cc1F]

In certain embodiments, R³ is

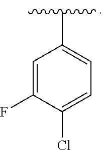 [SMILES: *c1ccc(Cl)c(F)c1]

In certain embodiments, R³ is

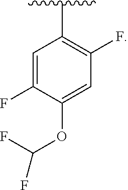 [SMILES: *c1cc(F)c(OC(F)F)cc1F]

In certain embodiments, R³ is

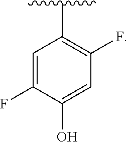 [SMILES: *c1cc(F)c(O)cc1F]

In certain embodiments, R³ is

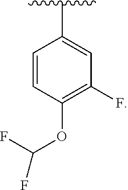 [SMILES: *c1ccc(OC(F)F)c(F)c1]

In certain embodiments, R³ is

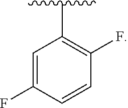 [SMILES: *c1cc(F)ccc1F]

In certain embodiments, R³ is

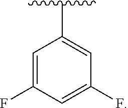 [SMILES: *c1cc(F)cc(F)c1]

In certain embodiments, R³ is

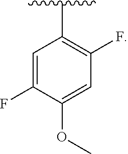 [SMILES: *c1cc(F)c(OC)cc1F]

In certain embodiments, R³ is

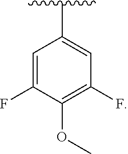 [SMILES: *c1cc(F)c(OC)c(F)c1]

In certain embodiments, R³ is selected from

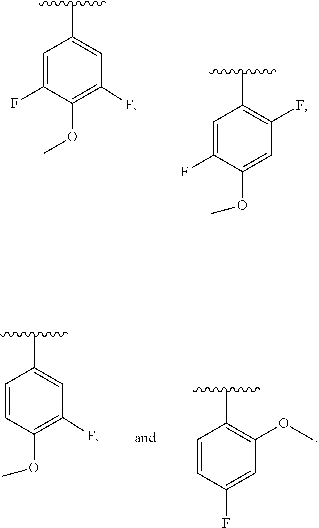

In certain embodiments, R³ is selected from

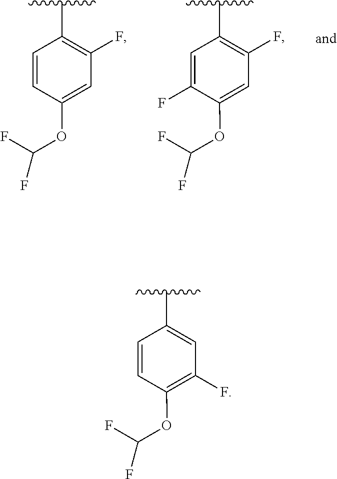 [SMILES: *c1ccc(OC(F)F)cc1F ; *c1cc(F)c(OC(F)F)cc1F ; *c1ccc(OC(F)F)c(F)c1]

In certain embodiments, R³ is selected from

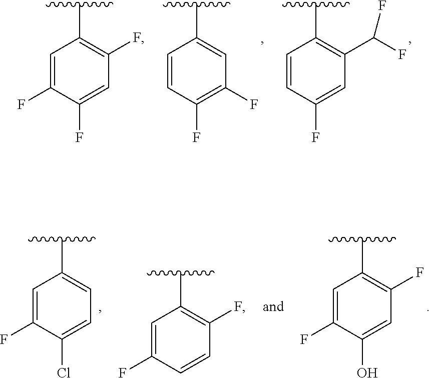

In certain embodiments, R³ is selected from the groups depicted in the compounds in Table A below.

As defined generally above, R⁴ represents independently for each occurrence halo, hydroxyl, C₁₋₄ haloalkyl, —O—(C₁₋₄ haloalkyl), or C₁₋₄ alkoxyl. In certain embodiments, R⁴ is halo. In certain embodiments, R⁴ is chloro. In certain embodiments, R⁴ is fluoro. In certain embodiments, R⁴ is hydroxyl. In certain embodiments, R⁴ is C₁₋₄ haloalkyl. In certain embodiments, R⁴ is C₁ haloalkyl. In certain embodiments, R⁴ is C₂ haloalkyl. In certain embodiments, R⁴ is C₃ haloalkyl. In certain embodiments, R⁴ is C₄ haloalkyl. In certain embodiments, R⁴ is —O—(C₁₋₄ haloalkyl). In certain embodiments, R⁴ is —O—(C₁ haloalkyl). In certain embodiments, R⁴ is —O—(C₂ haloalkyl). In certain embodiments, R⁴ is —O—(C₃ haloalkyl). In certain embodiments, R⁴ is —O—(C₄ haloalkyl). In certain embodiments, R⁴ is C₁₋₄ alkoxyl. In certain embodiments, R⁴ is C₁ alkoxyl. In certain embodiments, R⁴ is C₂ alkoxyl. In certain embodiments, R⁴ is C₃ alkoxyl. In certain embodiments, R⁴ is C₄ alkoxyl.

In certain embodiments, R⁴ represents independently for each occurrence fluoro, chloro, hydroxyl, C₁₋₄ fluoroalkyl, —O—(C₁₋₄ fluoroalkyl), or C₁₋₄ alkoxyl. In certain embodiments, R⁴ is C₁₋₄ fluoroalkyl. In certain embodiments, R⁴ is —O—(C₁₋₄ fluoroalkyl). In certain embodiments, R⁴ represents independently for each occurrence fluoro, chloro, methoxy, —OCHF₂, or —CHF₂. In certain embodiments, R⁴ is methoxy. In certain embodiments, R⁴ is —OCHF₂. In certain embodiments, R⁴ is —CHF₂. In certain embodiments, R⁴ is selected from the groups depicted in the compounds in Table A below.

As defined generally above, R^{5A}, R^{5B}, R^{6A}, R^{6B}, R^{7A}, R^{7B}, R^{8A}, and R^{8B} are independently hydrogen, halo, cyano, aryl, —(C₀₋₆ alkylene)-(hydroxyl), C₁₋₆ alkyl, —(C₁₋₆ haloalkyl substituted with 0 or 1 occurrences of hydroxyl), —(C₀₋₆ alkylene)-(C₁₋₆ alkoxyl substituted with 0 or 1 occurrences of halo), —C(O)—OR⁹, or —(C(R¹⁰)(R¹¹))ₛ—C(O)—N(R¹²)(R¹³). In certain embodiments, each of R^{5A}, R^{5B}, R^{6A}, R^{6B}, R^{7A}, R^{7B}, R^{8A}, and R^{8B} is hydrogen.

In certain embodiments, R^{5A} is hydrogen. In certain embodiments, R^{5A} is halo. In certain embodiments, R^{5A} is cyano. In certain embodiments, R^{5A} is aryl. In certain embodiments, R^{5A} is —(C₀₋₆ alkylene)-(hydroxyl). In certain embodiments, R^{5A} is C₁₋₆ alkyl. In certain embodiments, R^{5A} is C₁₋₆ haloalkyl substituted with 0 or 1 occurrences of hydroxyl. In certain embodiments, R^{5A} is —(C₀₋₆ alkylene)-(C₁₋₆ alkoxyl substituted with 0 or 1 occurrences of halo). In certain embodiments, R^{5A} is —C(O)—OR⁹. In certain embodiments, R^{5A} is —(C(R¹⁰)(R¹¹))ₛ—C(O)—N(R¹²)(R¹³). In certain embodiments, R^{5B} is hydrogen. In certain embodiments, R^{5B} is halo. In certain embodiments, R^{5B} is cyano. In certain embodiments, R^{5B} is aryl. In certain embodiments, R^{5B} is —(C₀₋₆ alkylene)-(hydroxyl). In certain embodiments, R^{5B} is C₁₋₆ alkyl. In certain embodiments, R^{5B} is C₁₋₆ haloalkyl substituted with 0 or 1 occurrences of hydroxyl. In certain embodiments, R^{5B} is —(C₀₋₆ alkylene)-(C₁₋₆ alkoxyl substituted with 0 or 1 occurrences of halo). In certain embodiments, R^{5B} is —C(O)—OR⁹. In certain embodiments, R^{5B} is —(C(R¹⁰)(R¹¹))ₛ—C(O)—N(R¹²)(R¹³).

In certain embodiments, R^{6A} is hydrogen. In certain embodiments, R^{6A} is halo. In certain embodiments, R^{6A} is cyano. In certain embodiments, R^{6A} is aryl. In certain embodiments, R^{6A} is —(C₀₋₆ alkylene)-(hydroxyl). In certain embodiments, R^{6A} is C₁₋₆ alkyl. In certain embodiments, R^{6A} is C₁₋₆ haloalkyl substituted with 0 or 1 occurrences of hydroxyl. In certain embodiments, R^{6A} is —(C₀₋₆ alkylene)-(C₁₋₆ alkoxyl substituted with 0 or 1 occurrences of halo). In certain embodiments, R^{6A} is —C(O)—OR⁹. In certain embodiments, R^{6A} is —(C(R¹⁰)(R¹¹))ₛ—C(O)—N(R¹²)(R¹³). In certain embodiments, R^{6B} is hydrogen. In certain embodiments, R^{6B} is halo. In certain embodiments, R^{6B} is cyano. In certain embodiments, R^{6B} is aryl. In certain embodiments, R^{6B} is —(C₀₋₆ alkylene)-(hydroxyl). In certain embodiments, R^{6B} is C₁₋₆ alkyl. In certain embodiments, R^{6B} is C₁₋₆ haloalkyl substituted with 0 or 1 occurrences of hydroxyl. In certain embodiments, R^{6B} is —(C₀₋₆ alkylene)-(C₁₋₆ alkoxyl substituted with 0 or 1 occurrences of halo). In certain embodiments, R^{6B} is —C(O)—OR⁹. In certain embodiments, R^{6B} is —(C(R¹⁰)(R¹¹))ₛ—C(O)—N(R¹²)(R¹³).

In certain embodiments, R^{7A} is hydrogen. In certain embodiments, R^{7A} is halo. In certain embodiments, R^{7A} is cyano. In certain embodiments, R^{7A} is aryl. In certain embodiments, R^{7A} is —(C₀₋₆ alkylene)-(hydroxyl). In certain embodiments, R^{7A} is C₁₋₆ alkyl. In certain embodiments, R^{7A} is C₁₋₆ haloalkyl substituted with 0 or 1 occurrences of hydroxyl. In certain embodiments, R^{7A} is —(C₀₋₆ alkylene)-(C₁₋₆ alkoxyl substituted with 0 or 1 occurrences of halo). In certain embodiments, R^{7A} is —C(O)—OR⁹. In certain embodiments, R^{7A} is —(C(R¹⁰)(R¹¹))ₛ—C(O)—N(R¹²)(R¹³). In certain embodiments, R^{7B} is hydrogen. In certain embodiments, R^{7B} is halo. In certain embodiments, R^{7B} is cyano. In certain embodiments, R^{7B} is aryl. In certain embodiments, R^{7B} is —(C₀₋₆ alkylene)-(hydroxyl). In certain embodiments, R^{7B} is C₁₋₆ alkyl. In certain embodiments, R^{7B} is C₁₋₆ haloalkyl substituted with 0 or 1 occurrences of hydroxyl. In certain embodiments, R^{7B} is —(C₀₋₆ alkylene)-(C₁₋₆ alkoxyl substituted with 0 or 1 occurrences of halo). In certain embodiments, R^{7B} is —C(O)—OR⁹. In certain embodiments, R^{7B} is —(C(R¹⁰)(R¹¹))ₛ—C(O)—N(R¹²)(R¹³).

In certain embodiments, R^{8A} is hydrogen. In certain embodiments, R^{8A} is halo. In certain embodiments, R^{8A} is cyano. In certain embodiments, R^{8A} is aryl. In certain embodiments, R^{8A} is —(C₀₋₆ alkylene)-(hydroxyl). In certain embodiments, R^{8A} is C₁₋₆ alkyl. In certain embodiments, R^{8A} is C₁₋₆ haloalkyl substituted with 0 or 1 occurrences of hydroxyl. In certain embodiments, R^{8A} is —(C₀₋₆ alkylene)-(C₁₋₆ alkoxyl substituted with 0 or 1 occurrences of halo). In certain embodiments, R^{8A} is —C(O)—OR⁹. In certain embodiments, R^{8A} is —(C(R¹⁰)(R¹¹))ₛ—C(O)—N(R¹²)(R¹³). In certain embodiments, R^{8B} is hydrogen. In certain embodiments, R^{8B} is halo. In certain embodiments, R^{8B} is cyano. In certain embodiments, R^{8B} is aryl. In certain embodiments, R^{8B} is —(C₀₋₆ alkylene)-(hydroxyl). In certain embodiments, R^{8B} is C₁₋₆ alkyl. In certain embodiments, R^{8B} is C₁₋₆ haloalkyl substituted with 0 or 1 occurrences of hydroxyl. In certain embodiments, R^{8B} is —(C₀₋₆ alkylene)-(C₁₋₆ alkoxyl substituted with 0 or 1 occurrences of halo). In certain embodiments, R^{8B} is —C(O)—OR⁹. In certain embodiments, R^{8B} is —(C(R¹⁰)(R¹¹))ₛ—C(O)—N(R¹²)(R¹³).

In certain embodiments, R⁵ is selected from the groups depicted in the compounds in Table A below. In certain embodiments, R^{5B} is selected from the groups depicted in the compounds in Table A below. In certain embodiments, R^{6A} is selected from the groups depicted in the compounds in Table A below. In certain embodiments, R^{6B} is selected from the groups depicted in the compounds in Table A below. In certain embodiments, R^{7A} is selected from the groups depicted in the compounds in Table A below. In certain embodiments, R^{7B} is selected from the groups depicted in the compounds in Table A below. In certain embodiments, R^{8A} selected from the groups depicted in the compounds in Table A below. In certain embodiments, R^{8B} is selected from the groups depicted in the compounds in Table A below.

As defined generally above, R⁹, R¹⁰, R¹¹, R¹², and R¹³ are independently hydrogen or C₁₋₄ alkyl. In certain embodiments, R⁹ is hydrogen. In certain embodiments, R⁹ is C₁₋₄ alkyl. In certain embodiments, R⁹ is C₁ alkyl. In certain embodiments, R⁹ is C₂ alkyl. In certain embodiments, R⁹ is C₃ alkyl. In certain embodiments, R⁹ is C₄ alkyl. In certain embodiments, R¹⁰ is hydrogen. In certain embodiments, R¹⁰ is C₁₋₄ alkyl. In certain embodiments, R¹⁰ is C₁ alkyl. In certain embodiments, R¹⁰ is C₂ alkyl. In certain embodiments, R¹⁰ is C₃ alkyl. In certain embodiments, R¹⁰ is C₄ alkyl. In certain embodiments, R¹¹ is hydrogen. In certain embodiments, R¹¹ is C₁₋₄ alkyl. In certain embodiments, R¹¹ is C₁ alkyl. In certain embodiments, R¹¹ is C₂ alkyl. In certain embodiments, R¹¹ is C₃ alkyl. In certain embodiments, R¹¹ is C₄ alkyl. In certain embodiments, R¹² is hydrogen. In certain embodiments, R¹² is C₁₋₄ alkyl. In certain embodiments, R¹² is C₁ alkyl. In certain embodiments, R¹² is C₂ alkyl. In certain embodiments, R¹² is C₃ alkyl. In certain embodiments, R¹² is C₄ alkyl. In certain embodiments, R¹³ is hydrogen. In certain embodiments, R¹³ is C₁₋₄ alkyl. In certain embodiments, R¹³ is C₁ alkyl. In certain embodiments, R¹³ is C₂ alkyl. In certain embodiments, R¹³ is C₃ alkyl. In certain embodiments, R¹³ is C₄ alkyl. In certain embodiments, R⁹ is selected from the groups depicted in the compounds in Table A below. In certain embodiments, R¹⁰ is selected from the groups depicted in the compounds in Table A below. In certain embodiments, R¹¹ is selected from the groups depicted in the compounds in Table A below. In certain embodiments, R¹² is selected from the groups depicted in the compounds in Table A below. In certain embodiments, R¹³ is selected from the groups depicted in the compounds in Table A below.

As defined generally above, p is 0, 1, 2, or 3. In certain embodiments p is 0. In certain embodiments p is 1. In certain embodiments p is 2. In certain embodiments p is 3.

As defined generally above, s is 0, 1, 2, 3, or 4. In certain embodiments s is 0. In certain embodiments s is 1. In certain embodiments s is 2. In certain embodiments s is 3. In certain embodiments s is 4.

In certain embodiments, the TBM is of Formula A*:

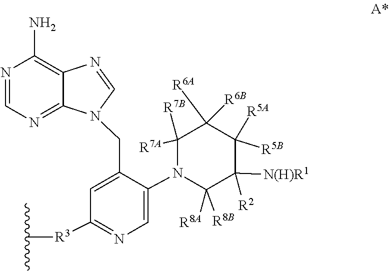

wherein:
R¹ is hydrogen or C₁₋₄ alkyl;
R² is —(C₁₋₄ haloalkyl substituted with 0 or 1 occurrences of hydroxyl) or a 5-6 membered heteroaryl containing 1 nitrogen atom, wherein the heteroaryl is substituted with 0 or 1 occurrences of halo;
R³ is phenylene or pyridinylene, each of which are substituted with p occurrences of R⁴;
R⁴ represents independently for each occurrence halo, hydroxyl, C₁₋₄ haloalkyl, —O—(C₁₋₄ haloalkyl), or C₁₋₄ alkoxyl;
R^{5A}, R^{5B}, R^{6A}, R^{6B}, R^{7A}, R^{7B}, R^{8A}, and R^{8B} are independently hydrogen, halo, cyano, aryl, —(C₀₋₆ alkylene)-(hydroxyl), C₁₋₆ alkyl, —(C₁₋₆ haloalkyl substituted with 0 or 1 occurrences of hydroxyl), —(C₀₋₆ alkylene)-(C₁₋₆ alkoxyl substituted with 0 or 1 occurrences of halo), —C(O)—OR⁹, or —(C(R¹⁰)(R¹¹))ₛ—C(O)—N(R¹²)(R¹³);
R⁹, R¹⁰, R¹¹, R¹², and R¹³ are independently hydrogen or C₁₋₄ alkyl;
p is 0, 1, 2, or 3; and
s is 0, 1, 2, 3, or 4.

In certain embodiments, one or more of the variables in Formula A* is as defined by one or more of the embodiments set foth above in connection with Formula A, except for variable R³.

In certain embodiments, the TBM of Formula A is further defined by Formula A-1:

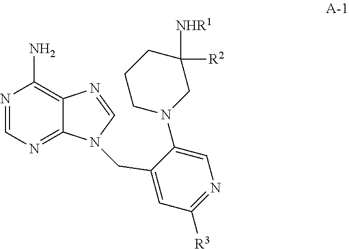

or a pharmaceutically acceptable salt thereof. In certain embodiments, the definition of variables R¹, R², and R³ is one of the embodiments described above in connection with Formula A.

The description above describes multiple embodiments relating to compounds of Formula A-1. The patent application specifically contemplates all combinations of the embodiments.

In certain embodiments, the TBM of Formula A-1 is:

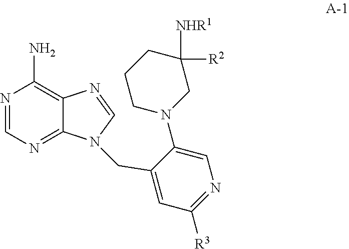

wherein:
   R¹ is hydrogen or C₁₋₄ alkyl;
   R² is —(C₁₋₄ haloalkyl substituted with 0 or 1 occurrences of hydroxyl) or a 5-6 membered heteroaryl containing 1 nitrogen atom, wherein the heteroaryl is substituted with 0 or 1 occurrences of halo;
   R³ is phenyl substituted with p occurrences of R⁴; and
   R⁴ represents independently for each occurrence halo, hydroxyl, C₁₋₄ haloalkyl, —O—(C₁₋₄ haloalkyl), or C₁₋₄ alkoxyl.

In certain embodiments, the TBM of Formula A is further defined by Formula A-2:

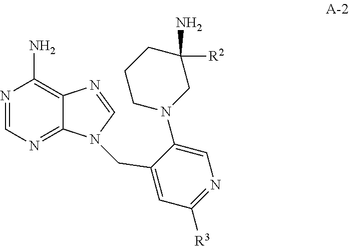

or a pharmaceutically acceptable salt thereof. In certain embodiments, the definition of variables R² and R³ is one of the embodiments described above in connection with Formula A.

The description above describes multiple embodiments relating to compounds of Formula A-2. The patent application specifically contemplates all combinations of the embodiments.

In certain embodiments, the TBM of Formula A-2 is:

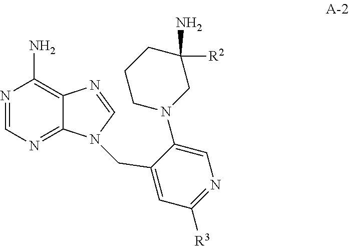

wherein:
   R² is —(C₁₋₄ haloalkyl substituted with 0 or 1 occurrences of hydroxyl) or a 5-6 membered heteroaryl containing 1 nitrogen atom, wherein the heteroaryl is substituted with 0 or 1 occurrences of halo;
   R³ is phenyl or pyridinyl, each of which is substituted with p occurrences of R⁴; and
   R⁴ represents independently for each occurrence halo, hydroxyl, C₁₋₄ haloalkyl, —O—(C₁₋₄ haloalkyl), or C₁₋₄ alkoxyl.

In certain embodiments, the TBM of Formula A is further defined by Formula A-3:

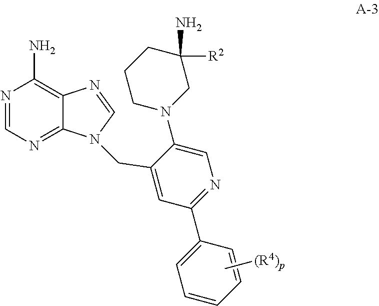

or a pharmaceutically acceptable salt thereof. In certain embodiments, the definition of variables R², R⁴, and p is one of the embodiments described above in connection with Formula A.

The description above describes multiple embodiments relating to compounds of Formula A-3. The patent application specifically contemplates all combinations of the embodiments.

In certain embodiments, the TBM is of Formula A-4:

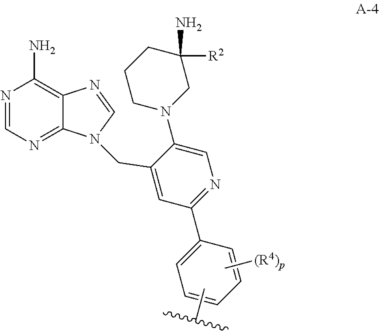

wherein:
   R² is —(C₁₋₄ haloalkyl substituted with 0 or 1 occurrences of hydroxyl) or a 5-6 membered heteroaryl containing 1 nitrogen atom, wherein the heteroaryl is substituted with 0 or 1 occurrences of halo;
   R⁴ represents independently for each occurrence halo, hydroxyl, C₁₋₄ haloalkyl, —O—(C₁₋₄ haloalkyl), or C₁₋₄ alkoxyl; and
   p is 0, 1, 2, or 3.

In certain embodiments, TBM is a radical of a compound described in embodiments of international patent application PCT/US2021/048973, the contents of which are herein incorporated by reference in their entirety.

In certain embodiments, TBM is a radical of

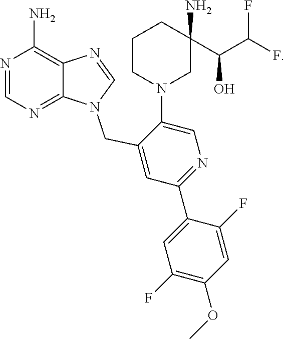

In certain embodiments, TBM is

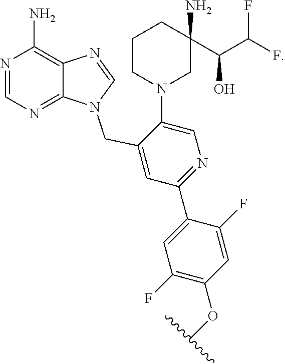 [SMILES: *Oc1cc(F)c(-c2cc(Cn3cnc4c(N)ncnc43)c(N3CCC[C@](N)([C@H](O)C(F)F)C3)cn2)cc1F]

In certain embodiments, TBM is

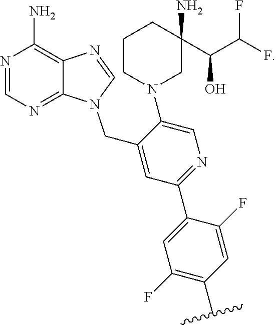 [SMILES: *c1cc(F)c(-c2cc(Cn3cnc4c(N)ncnc43)c(N3CCC[C@](N)([C@H](O)C(F)F)C3)cn2)cc1F]

In certain embodiments, TBM is

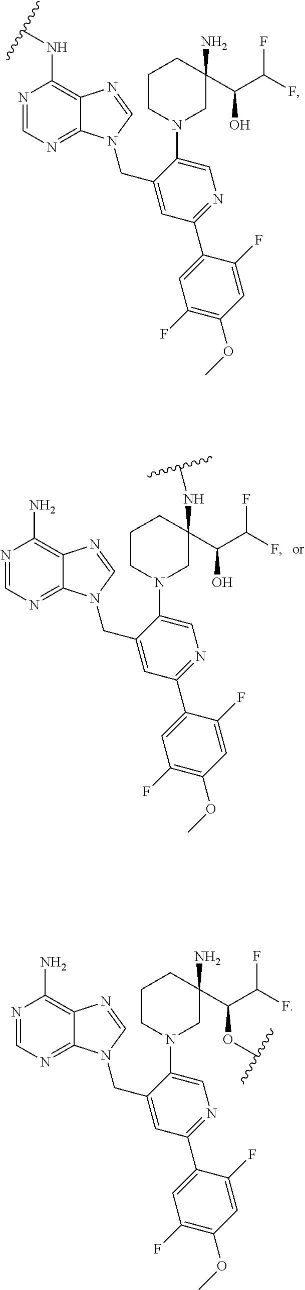 [SMILES: *Nc1ncnc2c1ncn2Cc1cc(-c2cc(F)c(OC)cc2F)ncc1N1CCC[C@](N)([C@H](O)C(F)F)C1 ; *N[C@]1([C@H](O)C(F)F)CCCN(c2cnc(-c3cc(F)c(OC)cc3F)cc2Cn2cnc3c(N)ncnc32)C1 ; *O[C@H](C(F)F)[C@@]1(N)CCCN(c2cnc(-c3cc(F)c(OC)cc3F)cc2Cn2cnc3c(N)ncnc32)C1]

In certain embodiments, TBM is

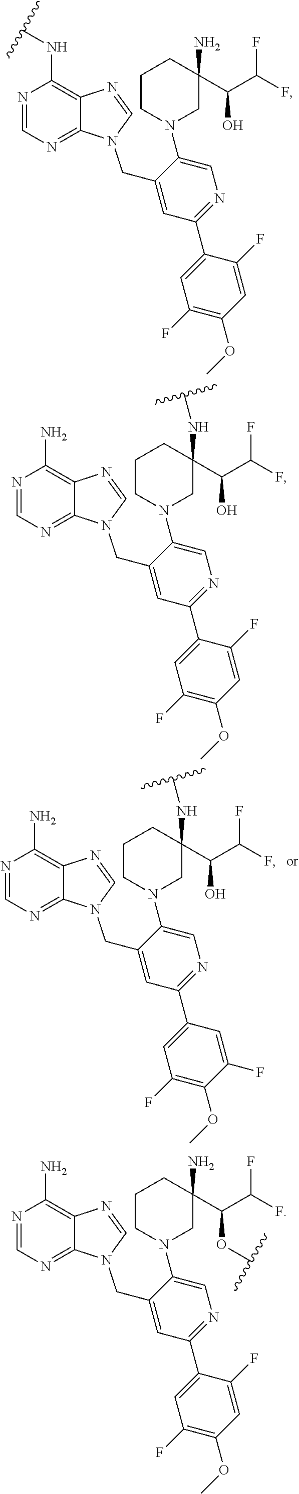

In certain embodiments, TBM is

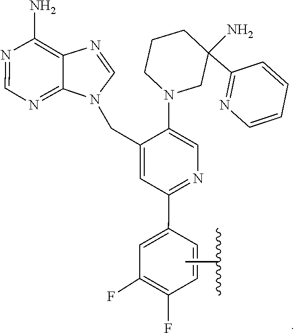

In certain embodiments, TBM is

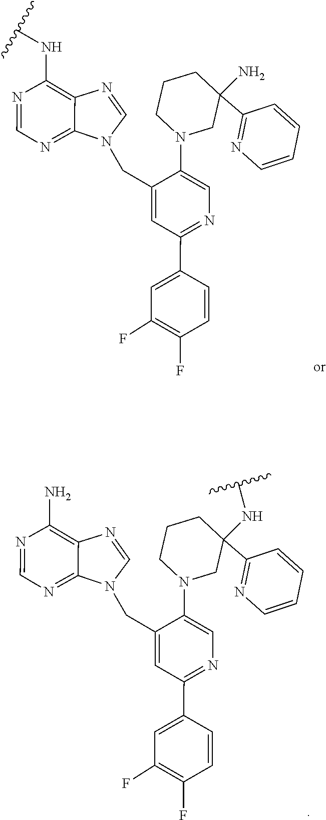 [SMILES: *Nc1ncnc2c1ncn2Cc1cc(-c2ccc(F)c(F)c2)ncc1N1CCCC(N)(c2ccccn2)C1 ; *NC1(c2ccccn2)CCCN(c2cnc(-c3ccc(F)c(F)c3)cc2Cn2cnc3c(N)ncnc32)C1]

In certain embodiments, TBM is

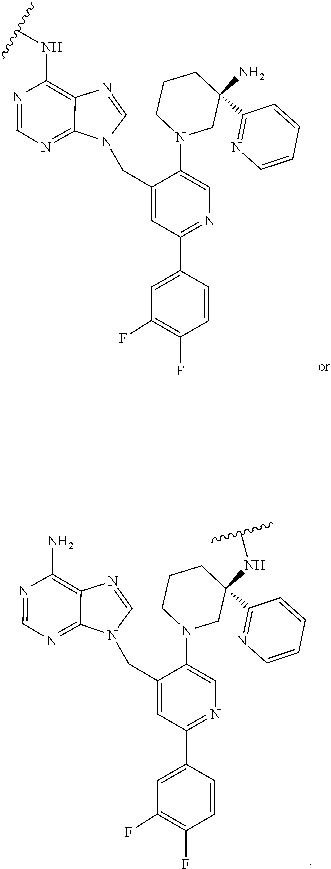 [SMILES: *Nc1ncnc2c1ncn2Cc1cc(-c2ccc(F)c(F)c2)ncc1N1CCC[C@](N)(c2ccccn2)C1 ; *N[C@]1(c2ccccn2)CCCN(c2cnc(-c3ccc(F)c(F)c3)cc2Cn2cnc3c(N)ncnc32)C1]

In certain embodiments, TBM is a radical of a compound in Table A. In certain embodiments, TBM is a radical of a compound in Table A, wherein one hydrogen atom of the compound in Table A is replaced with a point of attachment to L. In certain embodiments, TBM is a radical of a compound in Table A, wherein a plurality of atoms of the compound in Table A (e.g., an alkyl group, such as a methyl group) is replaced with a point of attachment to L. In certain embodiments, TBM is a radical of a compound in Table A, wherein at least one unpaired valence electron of the radical provides a point of attachment to L.

In certain embodiments, TBM is a radical of one of compounds T-1 to T-23 in Table A. In certain embodiments, TBM is a radical of one of compounds T-1 to T-23 in Table A, wherein one hydrogen atom of said compound is replaced with a point of attachment to L. In certain embodiments, TBM is a radical of one of compounds T-1 to T-23 in Table A, wherein a plurality of atoms of said compound (e.g., an alkyl group, such as a methyl group) is replaced with a point of attachment to L. In certain embodiments, TBM is a radical of one of compounds T-1 to T-23 in Table A, wherein at least one unpaired valence electron of the radical provides a point of attachment to L

TABLE A

| Compound No. | Chemical Structure |
|---|---|
| T-1 | 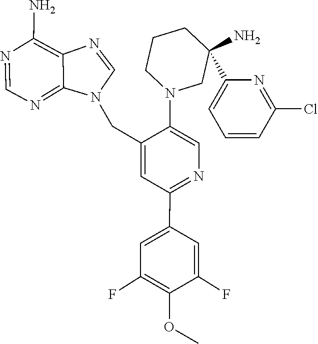 [SMILES: COc1c(F)cc(-c2cc(Cn3cnc4c(N)ncnc43)c(N3CCC[C@](N)(c4cccc(Cl)n4)C3)cn2)cc1F] |
| T-2 | 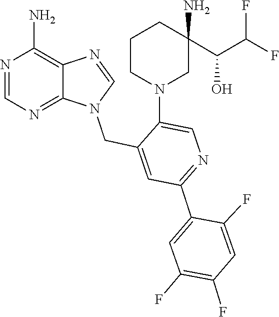 [SMILES: Nc1ncnc2c1ncn2Cc1cc(-c2cc(F)c(F)cc2F)ncc1N1CCC[C@](N)([C@@H](O)C(F)F)C1] |
| T-3 | 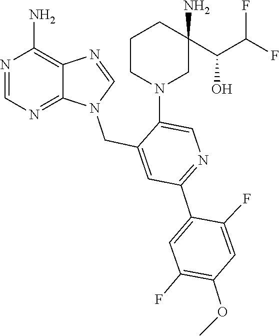 [SMILES: COc1cc(F)c(-c2cc(Cn3cnc4c(N)ncnc43)c(N3CCC[C@](N)([C@@H](O)C(F)F)C3)cn2)cc1F] |
| T-4 | 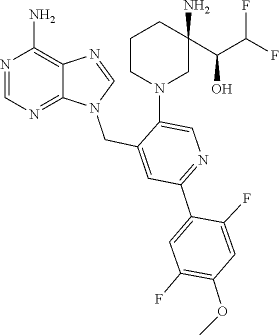 [SMILES: COc1cc(F)c(-c2cc(Cn3cnc4c(N)ncnc43)c(N3CCC[C@](N)([C@H](O)C(F)F)C3)cn2)cc1F] |
| T-5 | 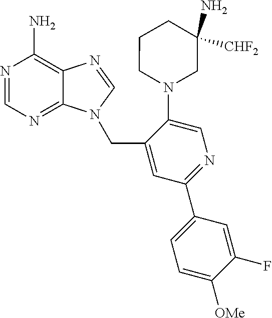 [SMILES: COc1ccc(-c2cc(Cn3cnc4c(N)ncnc43)c(N3CCC[C@](N)(C(F)F)C3)cn2)cc1F] |
| T-6 | 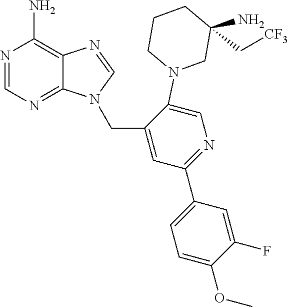 [SMILES: COc1ccc(-c2cc(Cn3cnc4c(N)ncnc43)c(N3CCC[C@](N)(CC(F)(F)F)C3)cn2)cc1F] |
| T-7 | 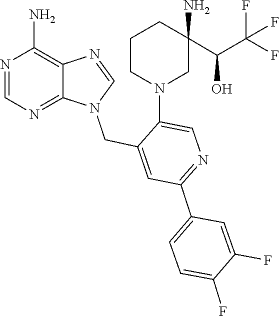 [SMILES: Nc1ncnc2c1ncn2Cc1cc(-c2ccc(F)c(F)c2)ncc1N1CCC[C@](N)([C@H](O)C(F)(F)F)C1] |
| T-8 | 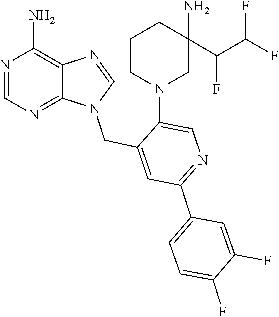 [SMILES: Nc1ncnc2c1ncn2Cc1cc(-c2ccc(F)c(F)c2)ncc1N1CCCC(N)(C(F)C(F)F)C1] |
| T-9 | 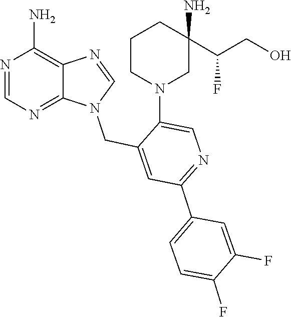 [SMILES: Nc1ncnc2c1ncn2Cc1cc(-c2ccc(F)c(F)c2)ncc1N1CCC[C@](N)([C@@H](F)CO)C1] |
| T-10 | 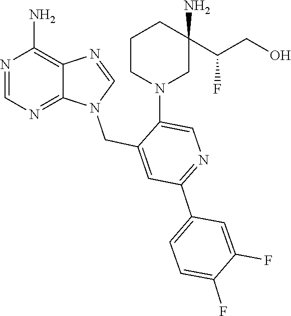 [SMILES: Nc1ncnc2c1ncn2Cc1cc(-c2ccc(F)c(F)c2)ncc1N1CCC[C@](N)([C@@H](F)CO)C1] |
| T-11 | 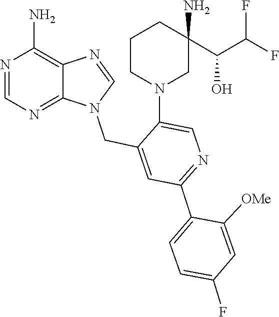 [SMILES: COc1cc(F)ccc1-c1cc(Cn2cnc3c(N)ncnc32)c(N2CCC[C@](N)([C@@H](O)C(F)F)C2)cn1] |
| T-12 | 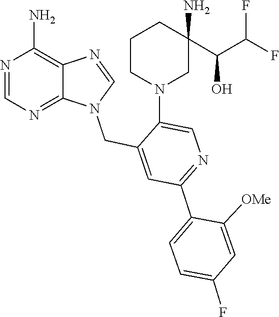 [SMILES: COc1cc(F)ccc1-c1cc(Cn2cnc3c(N)ncnc32)c(N2CCC[C@](N)([C@H](O)C(F)F)C2)cn1] |
| T-13 | 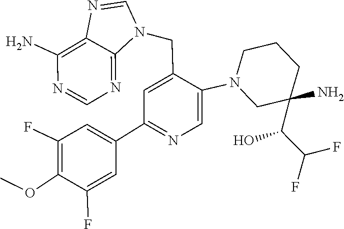 [SMILES: COc1c(F)cc(-c2cc(Cn3cnc4c(N)ncnc43)c(N3CCC[C@](N)([C@@H](O)C(F)F)C3)cn2)cc1F] |
| T-14 | 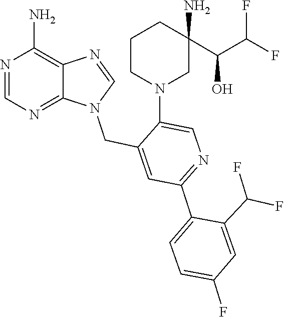 [SMILES: Nc1ncnc2c1ncn2Cc1cc(-c2ccc(F)cc2C(F)F)ncc1N1CCC[C@](N)([C@H](O)C(F)F)C1] |
| T-15 | 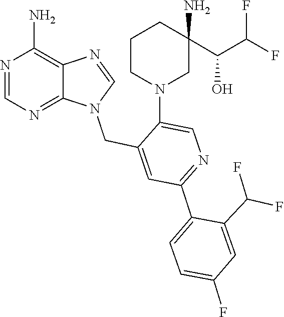 [SMILES: Nc1ncnc2c1ncn2Cc1cc(-c2ccc(F)cc2C(F)F)ncc1N1CCC[C@](N)([C@@H](O)C(F)F)C1] |
| T-16 | 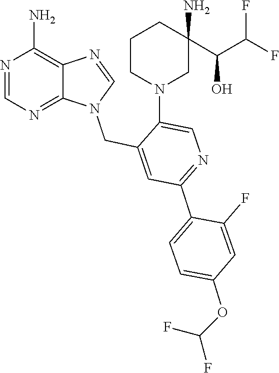 [SMILES: Nc1ncnc2c1ncn2Cc1cc(-c2ccc(OC(F)F)cc2F)ncc1N1CCC[C@](N)([C@H](O)C(F)F)C1] |
| T-17 | 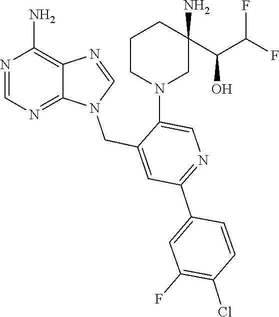 [SMILES: Nc1ncnc2c1ncn2Cc1cc(-c2ccc(Cl)c(F)c2)ncc1N1CCC[C@](N)([C@H](O)C(F)F)C1] |
| T-18 | 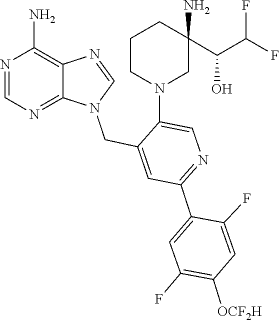 |
| T-19 | 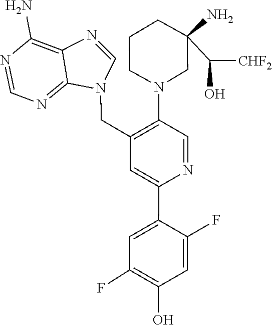 [SMILES: Nc1ncnc2c1ncn2Cc1cc(-c2cc(F)c(O)cc2F)ncc1N1CCC[C@](N)([C@H](O)C(F)F)C1] |
| T-20 | 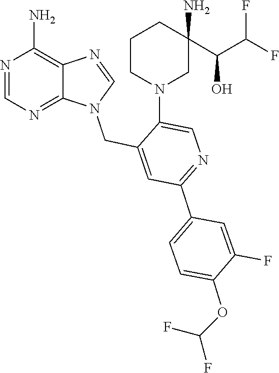 [SMILES: Nc1ncnc2c1ncn2Cc1cc(-c2ccc(OC(F)F)c(F)c2)ncc1N1CCC[C@](N)([C@H](O)C(F)F)C1] |
| T-21 | 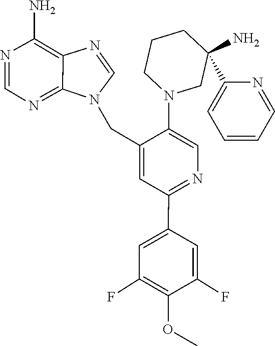 [SMILES: COc1c(F)cc(-c2cc(Cn3cnc4c(N)ncnc43)c(N3CCC[C@](N)(c4ccccn4)C3)cn2)cc1F] |
| T-22 | 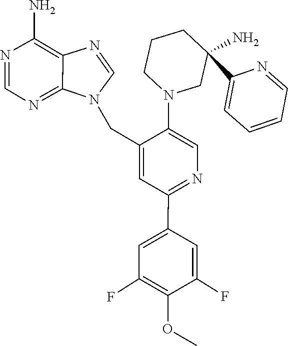 [SMILES: COc1c(F)cc(-c2cc(Cn3cnc4c(N)ncnc43)c(N3CCC[C@@](N)(c4ccccn4)C3)cn2)cc1F] |
| T-23 | 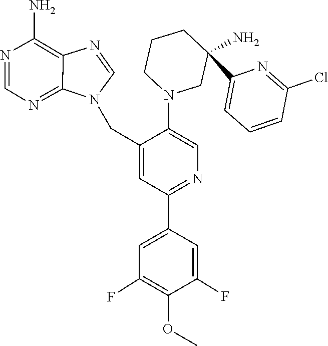 [SMILES: COc1c(F)cc(-c2cc(Cn3cnc4c(N)ncnc43)c(N3CCC[C@@](N)(c4cccc(Cl)n4)C3)cn2)cc1F] |
| T-24 | 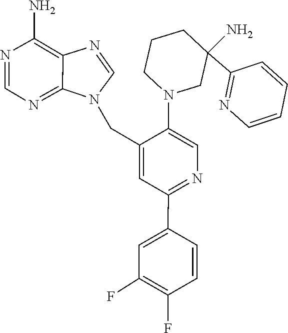 [SMILES: Nc1ncnc2c1ncn2Cc1cc(-c2ccc(F)c(F)c2)ncc1N1CCCC(N)(c2ccccn2)C1] |
| T-25 | 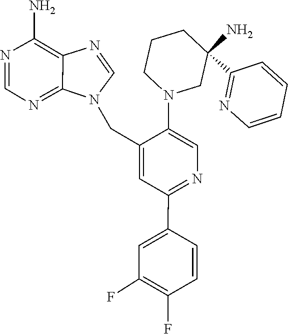 [SMILES: Nc1ncnc2c1ncn2Cc1cc(-c2ccc(F)c(F)c2)ncc1N1CCC[C@](N)(c2ccccn2)C1] |

In certain embodiments, the compound is a compound in Table 1, or a pharmaceutically acceptable salt thereof. In certain embodiments, the compound is a compound in Table 1. In certain embodiments, the compound is one of compounds I-1 to I-86 in Table 1, or a pharmaceutically acceptable salt thereof. In certain embodiments, the compound is one of compounds I-1 to I-86 in Table 1. In certain embodiments, the compound is one of compounds I-1 to I-92 in Table 1, or a pharmaceutically acceptable salt thereof. In certain embodiments, the compound is one of compounds I-1 to I-92 in Table 1.

TABLE 1

| Com- | |
|---|---|
| pound | |
| No. | Chemical Structure |
| I-1 | 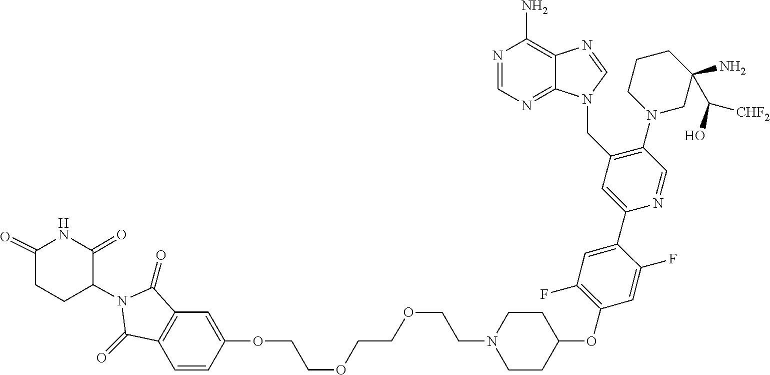 [SMILES: Nc1ncnc2c1ncn2Cc1cc(-c2cc(F)c(OC3CCN(CCOCCOCCOc4ccc5c(c4)C(=O)N(C4CCC(=O)NC4=O)C5=O)CC3)cc2F)ncc1N1CCC[C@](N)([C@H](O)C(F)F)C1] |
| I-2 | 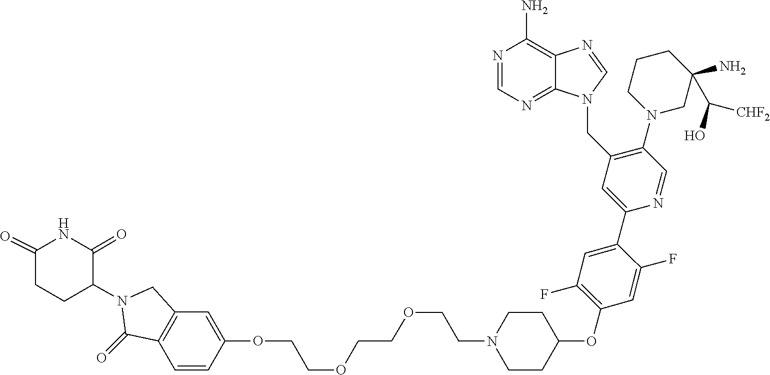 [SMILES: Nc1ncnc2c1ncn2Cc1cc(-c2cc(F)c(OC3CCN(CCOCCOCCOc4ccc5c(c4)CN(C4CCC(=O)NC4=O)C5=O)CC3)cc2F)ncc1N1CCC[C@](N)([C@H](O)C(F)F)C1] |
| I-3 | 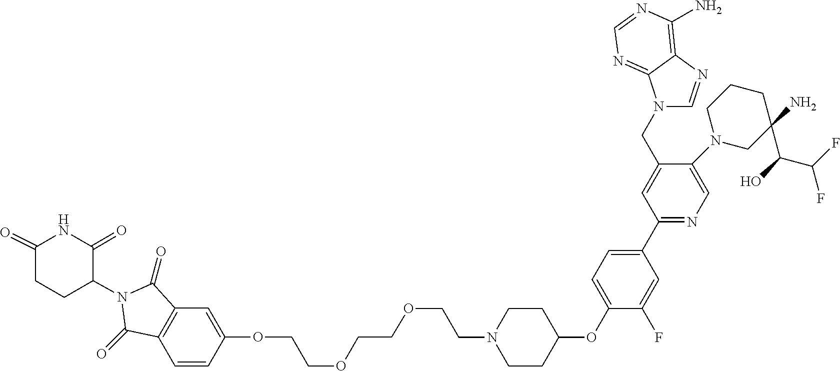 [SMILES: Nc1ncnc2c1ncn2Cc1cc(-c2ccc(OC3CCN(CCOCCOCCOc4ccc5c(c4)C(=O)N(C4CCC(=O)NC4=O)C5=O)CC3)c(F)c2)ncc1N1CCC[C@](N)([C@H](O)C(F)F)C1] |
| I-4 | 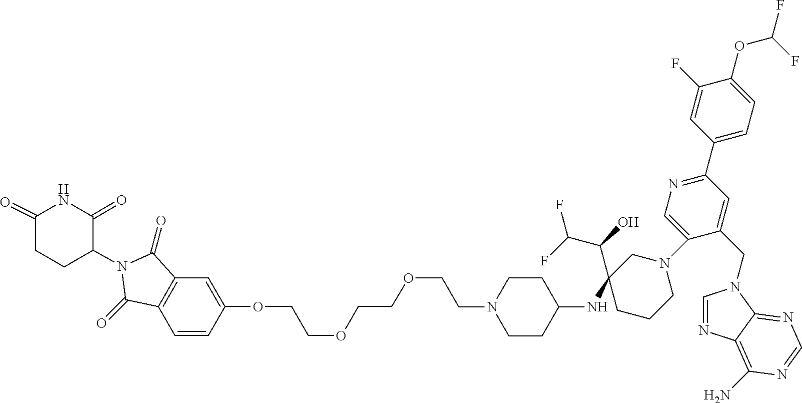 [SMILES: Nc1ncnc2c1ncn2Cc1cc(-c2ccc(OC(F)F)c(F)c2)ncc1N1CCC[C@](NC2CCN(CCOCCOCCOc3ccc4c(c3)C(=O)N(C3CCC(=O)NC3=O)C4=O)CC2)([C@H](O)C(F)F)C1] |
| I-5 | 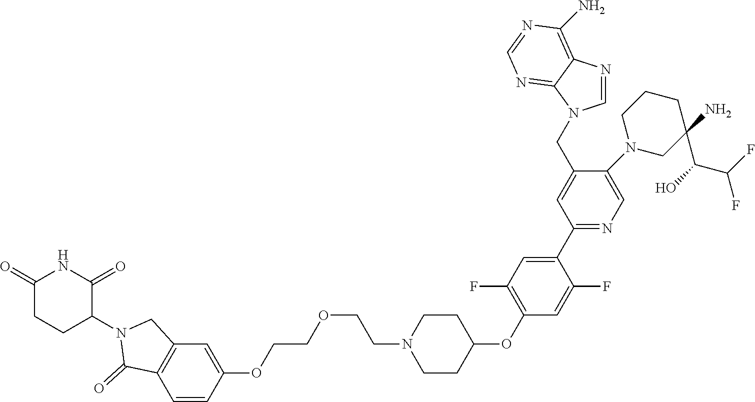 [SMILES: Nc1ncnc2c1ncn2Cc1cc(-c2cc(F)c(OC3CCN(CCOCCOc4ccc5c(c4)CN(C4CCC(=O)NC4=O)C5=O)CC3)cc2F)ncc1N1CCC[C@](N)([C@@H](O)C(F)F)C1] |
| I-6 | 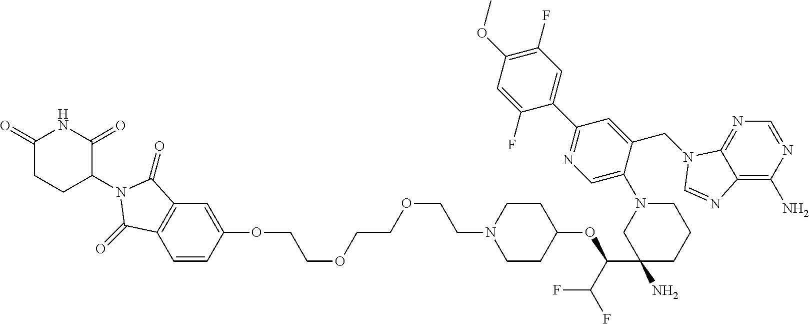 [SMILES: COc1cc(F)c(-c2cc(Cn3cnc4c(N)ncnc43)c(N3CCC[C@](N)([C@H](OC4CCN(CCOCCOCCOc5ccc6c(c5)C(=O)N(C5CCC(=O)NC5=O)C6=O)CC4)C(F)F)C3)cn2)cc1F] |
| I-7 | 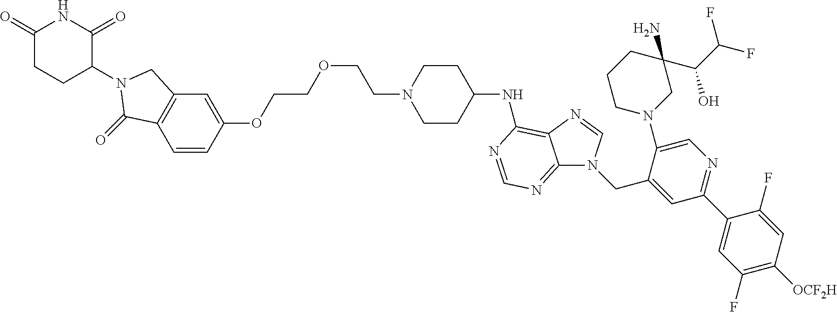 |
| I-8 | 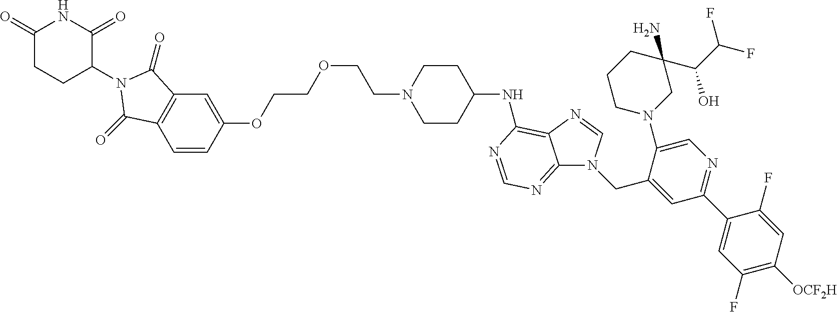 |
| I-9 | 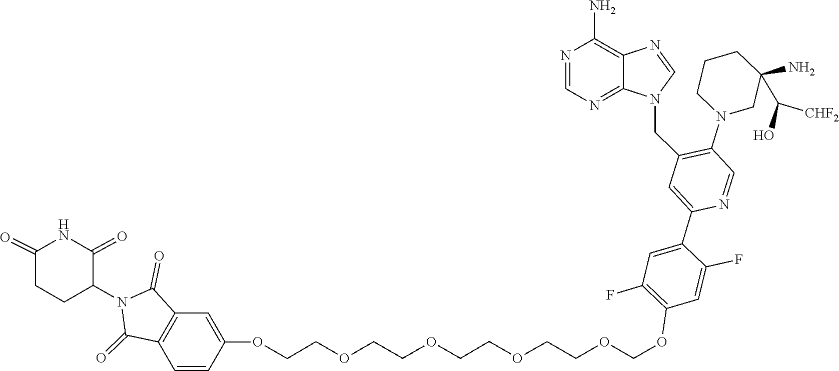 [SMILES: Nc1ncnc2c1ncn2Cc1cc(-c2cc(F)c(OCOCCOCCOCCOCCOc3ccc4c(c3)C(=O)N(C3CCC(=O)NC3=O)C4=O)cc2F)ncc1N1CCC[C@](N)([C@H](O)C(F)F)C1] |
| I-10 | 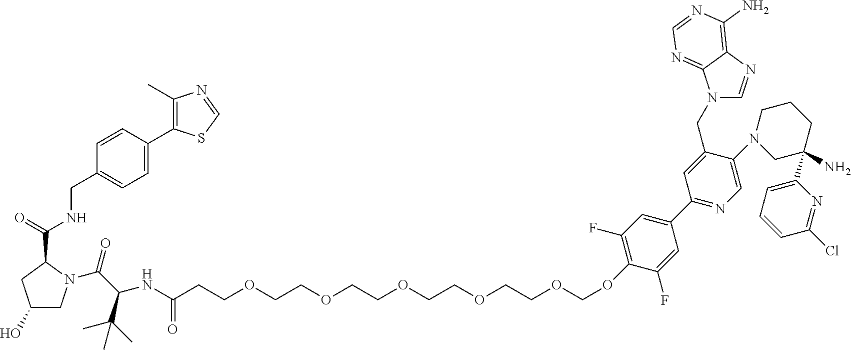 [SMILES: Cc1ncsc1-c1ccc(CNC(=O)[C@@H]2C[C@@H](O)CN2C(=O)[C@@H](NC(=O)CCOCCOCCOCCOCCOCOc2c(F)cc(-c3cc(Cn4cnc5c(N)ncnc54)c(N4CCC[C@](N)(c5cccc(Cl)n5)C4)cn3)cc2F)C(C)(C)C)cc1] |
| I-11 | 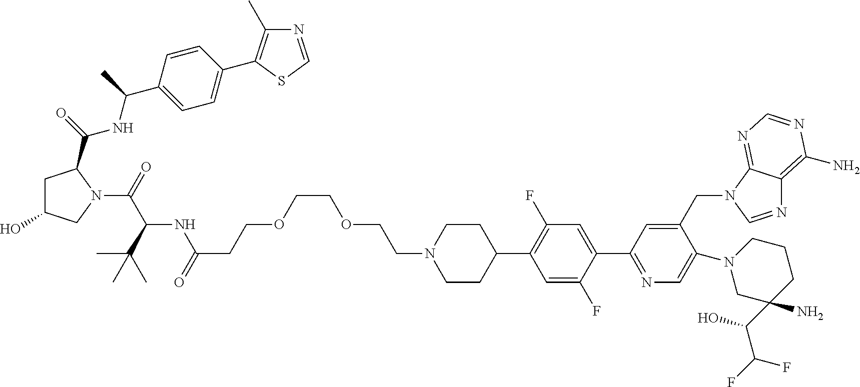 [SMILES: Cc1ncsc1-c1ccc([C@H](C)NC(=O)[C@@H]2C[C@@H](O)CN2C(=O)[C@@H](NC(=O)CCOCCOCCN2CCC(c3cc(F)c(-c4cc(Cn5cnc6c(N)ncnc65)c(N5CCC[C@](N)([C@@H](O)C(F)F)C5)cn4)cc3F)CC2)C(C)(C)C)cc1] |
| I-12 | 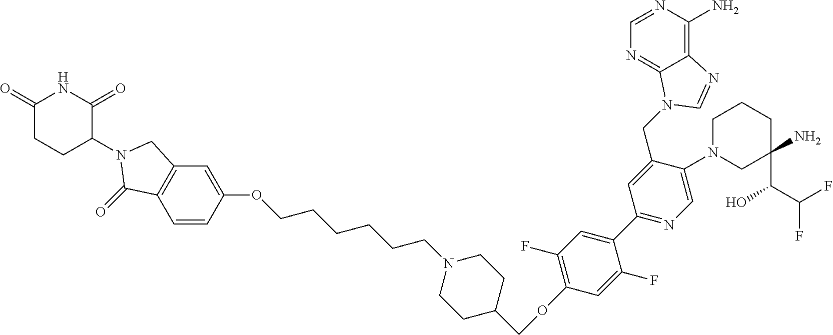 [SMILES: Nc1ncnc2c1ncn2Cc1cc(-c2cc(F)c(OCC3CCN(CCCCCCOc4ccc5c(c4)CN(C4CCC(=O)NC4=O)C5=O)CC3)cc2F)ncc1N1CCC[C@](N)([C@@H](O)C(F)F)C1] |
| I-13 | 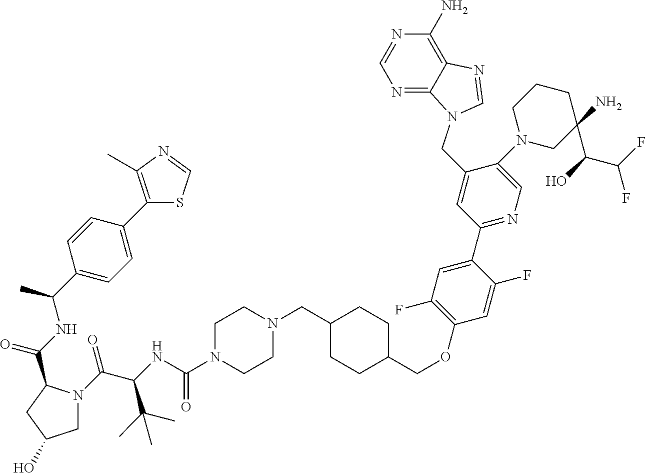 [SMILES: Cc1ncsc1-c1ccc([C@H](C)NC(=O)[C@@H]2C[C@@H](O)CN2C(=O)[C@@H](NC(=O)N2CCN(CC3CCC(COc4cc(F)c(-c5cc(Cn6cnc7c(N)ncnc76)c(N6CCC[C@](N)([C@H](O)C(F)F)C6)cn5)cc4F)CC3)CC2)C(C)(C)C)cc1] |
| I-14 | 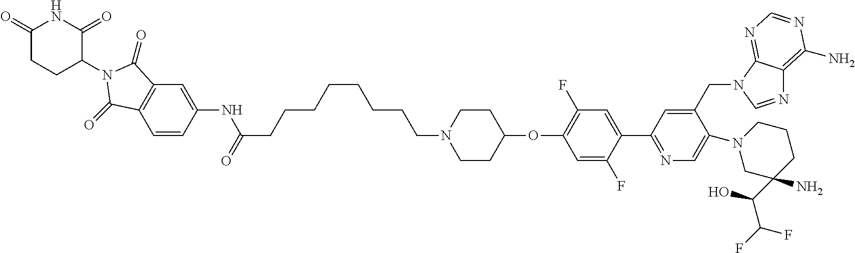 [SMILES: Nc1ncnc2c1ncn2Cc1cc(-c2cc(F)c(OC3CCN(CCCCCCCCC(=O)Nc4ccc5c(c4)C(=O)N(C4CCC(=O)NC4=O)C5=O)CC3)cc2F)ncc1N1CCC[C@](N)([C@H](O)C(F)F)C1] |
| I-15 | 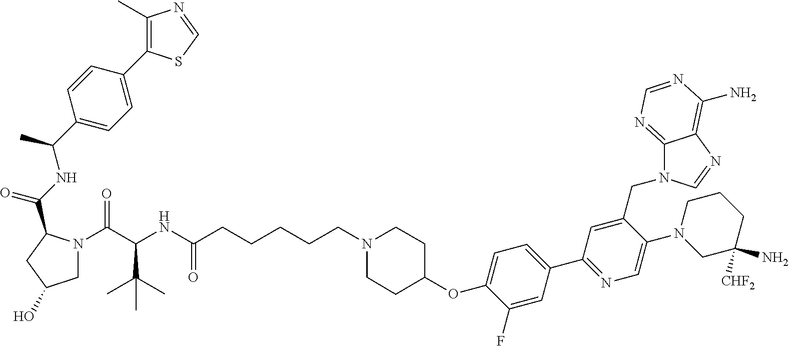 [SMILES: Cc1ncsc1-c1ccc([C@H](C)NC(=O)[C@@H]2C[C@@H](O)CN2C(=O)[C@@H](NC(=O)CCCCCN2CCC(Oc3ccc(-c4cc(Cn5cnc6c(N)ncnc65)c(N5CCC[C@](N)(C(F)F)C5)cn4)cc3F)CC2)C(C)(C)C)cc1] |
| I-16 | 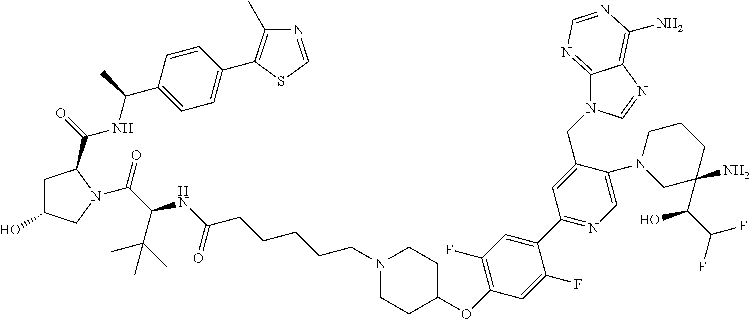 [SMILES: Cc1ncsc1-c1ccc([C@H](C)NC(=O)[C@@H]2C[C@@H](O)CN2C(=O)[C@@H](NC(=O)CCCCCN2CCC(Oc3cc(F)c(-c4cc(Cn5cnc6c(N)ncnc65)c(N5CCC[C@](N)([C@H](O)C(F)F)C5)cn4)cc3F)CC2)C(C)(C)C)cc1] |
| I-17 | 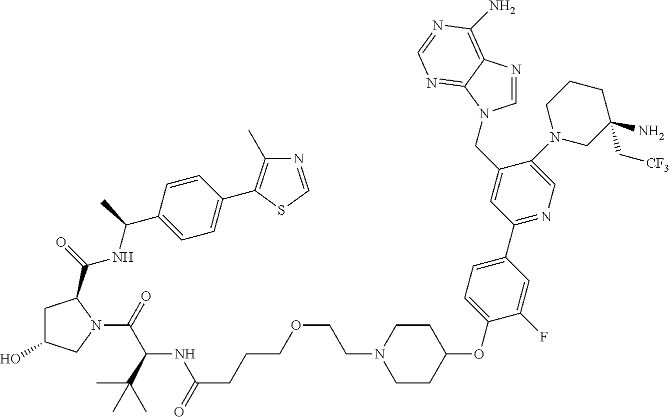 [SMILES: Cc1ncsc1-c1ccc([C@H](C)NC(=O)[C@@H]2C[C@@H](O)CN2C(=O)[C@@H](NC(=O)CCCOCCN2CCC(Oc3ccc(-c4cc(Cn5cnc6c(N)ncnc65)c(N5CCC[C@](N)(CC(F)(F)F)C5)cn4)cc3F)CC2)C(C)(C)C)cc1] |
| I-18 | 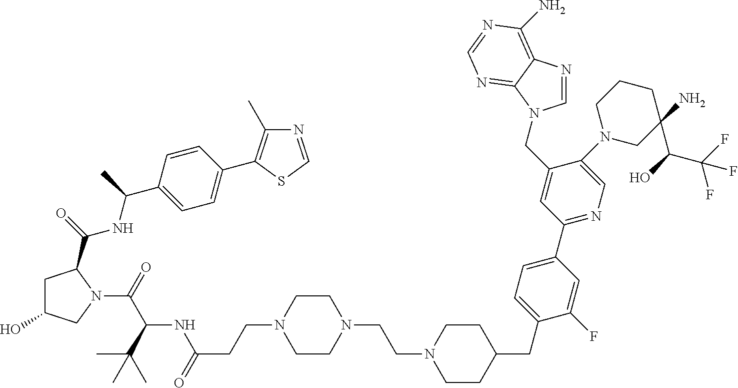 [SMILES: Cc1ncsc1-c1ccc([C@H](C)NC(=O)[C@@H]2C[C@@H](O)CN2C(=O)[C@@H](NC(=O)CCN2CCN(CCN3CCC(Cc4ccc(-c5cc(Cn6cnc7c(N)ncnc76)c(N6CCC[C@](N)([C@H](O)C(F)(F)F)C6)cn5)cc4F)CC3)CC2)C(C)(C)C)cc1] |
| I-19 | 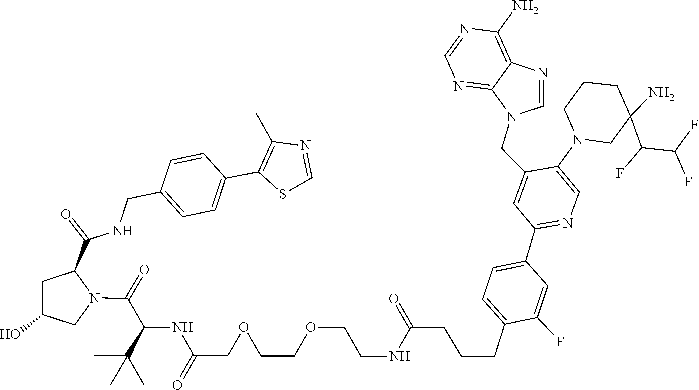 [SMILES: Cc1ncsc1-c1ccc(CNC(=O)[C@@H]2C[C@@H](O)CN2C(=O)[C@@H](NC(=O)COCCOCCNC(=O)CCCc2ccc(-c3cc(Cn4cnc5c(N)ncnc54)c(N4CCCC(N)(C(F)C(F)F)C4)cn3)cc2F)C(C)(C)C)cc1] |
| I-20 | 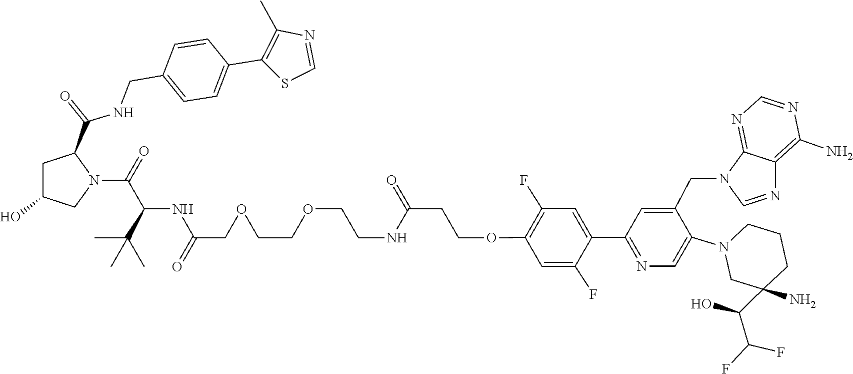 [SMILES: Cc1ncsc1-c1ccc(CNC(=O)[C@@H]2C[C@@H](O)CN2C(=O)[C@@H](NC(=O)COCCOCCNC(=O)CCOc2cc(F)c(-c3cc(Cn4cnc5c(N)ncnc54)c(N4CCC[C@](N)([C@H](O)C(F)F)C4)cn3)cc2F)C(C)(C)C)cc1] |
| I-21 | 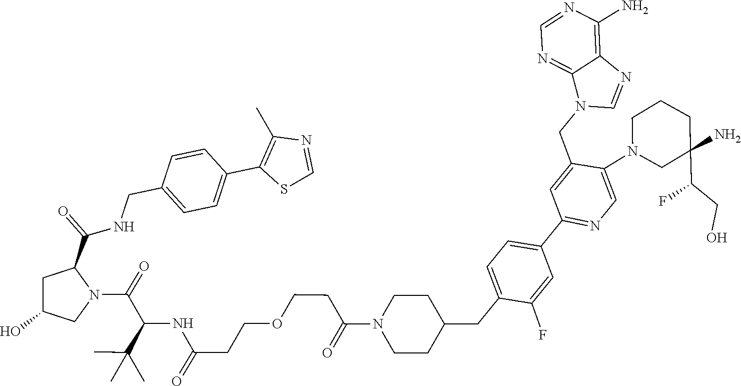 [SMILES: Cc1ncsc1-c1ccc(CNC(=O)[C@@H]2C[C@@H](O)CN2C(=O)[C@@H](NC(=O)CCOCCC(=O)N2CCC(Cc3ccc(-c4cc(Cn5cnc6c(N)ncnc65)c(N5CCC[C@](N)([C@@H](F)CO)C5)cn4)cc3F)CC2)C(C)(C)C)cc1] |
| I-22 | 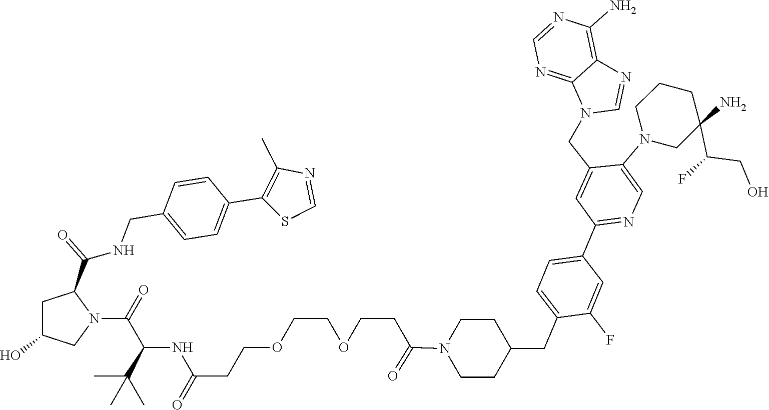 [SMILES: Cc1ncsc1-c1ccc(CNC(=O)[C@@H]2C[C@@H](O)CN2C(=O)[C@@H](NC(=O)CCOCCOCCC(=O)N2CCC(Cc3ccc(-c4cc(Cn5cnc6c(N)ncnc65)c(N5CCC[C@](N)([C@@H](F)CO)C5)cn4)cc3F)CC2)C(C)(C)C)cc1] |
| I-23 | 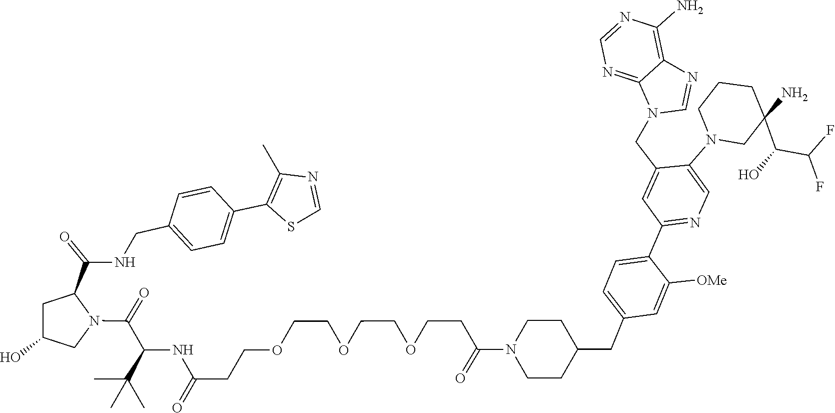 [SMILES: COc1cc(CC2CCN(C(=O)CCOCCOCCOCCC(=O)N[C@H](C(=O)N3C[C@H](O)C[C@H]3C(=O)NCc3ccc(-c4scnc4C)cc3)C(C)(C)C)CC2)ccc1-c1cc(Cn2cnc3c(N)ncnc32)c(N2CCC[C@](N)([C@@H](O)C(F)F)C2)cn1] |
| I-24 | 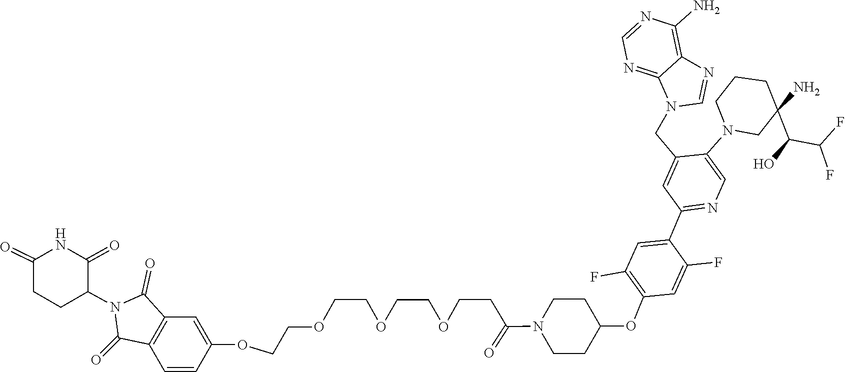 [SMILES: Nc1ncnc2c1ncn2Cc1cc(-c2cc(F)c(OC3CCN(C(=O)CCOCCOCCOCCOc4ccc5c(c4)C(=O)N(C4CCC(=O)NC4=O)C5=O)CC3)cc2F)ncc1N1CCC[C@](N)([C@H](O)C(F)F)C1] |
| I-25 | 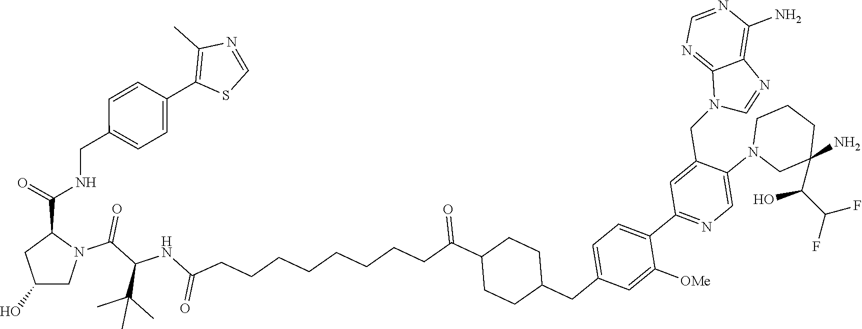 [SMILES: COc1cc(CC2CCC(C(=O)CCCCCCCCC(=O)N[C@H](C(=O)N3C[C@H](O)C[C@H]3C(=O)NCc3ccc(-c4scnc4C)cc3)C(C)(C)C)CC2)ccc1-c1cc(Cn2cnc3c(N)ncnc32)c(N2CCC[C@](N)([C@H](O)C(F)F)C2)cn1] |
| I-26 | 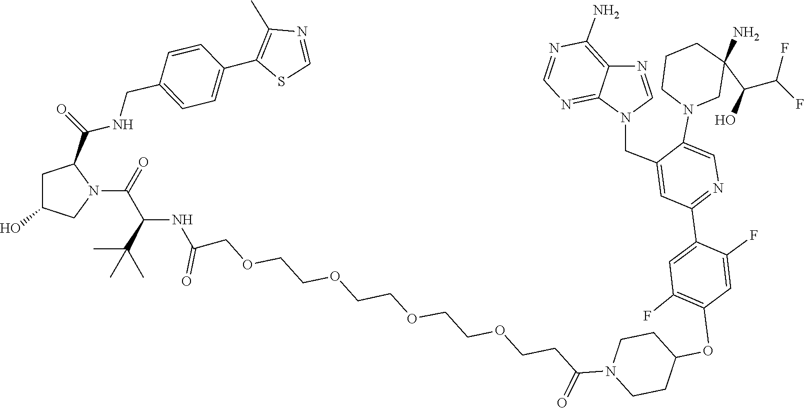 [SMILES: Cc1ncsc1-c1ccc(CNC(=O)[C@@H]2C[C@@H](O)CN2C(=O)[C@@H](NC(=O)COCCOCCOCCOCCC(=O)N2CCC(Oc3cc(F)c(-c4cc(Cn5cnc6c(N)ncnc65)c(N5CCC[C@](N)([C@H](O)C(F)F)C5)cn4)cc3F)CC2)C(C)(C)C)cc1] |
| I-27 | 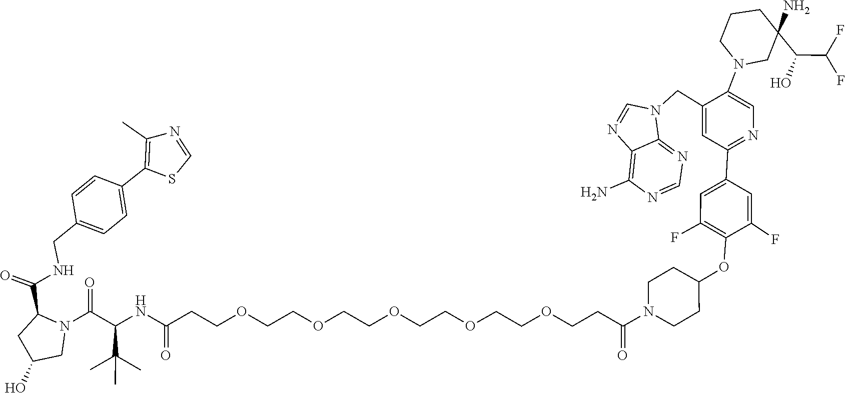 [SMILES: Cc1ncsc1-c1ccc(CNC(=O)[C@@H]2C[C@@H](O)CN2C(=O)[C@@H](NC(=O)CCOCCOCCOCCOCCOCCC(=O)N2CCC(Oc3c(F)cc(-c4cc(Cn5cnc6c(N)ncnc65)c(N5CCC[C@](N)([C@@H](O)C(F)F)C5)cn4)cc3F)CC2)C(C)(C)C)cc1] |
| I-28 | 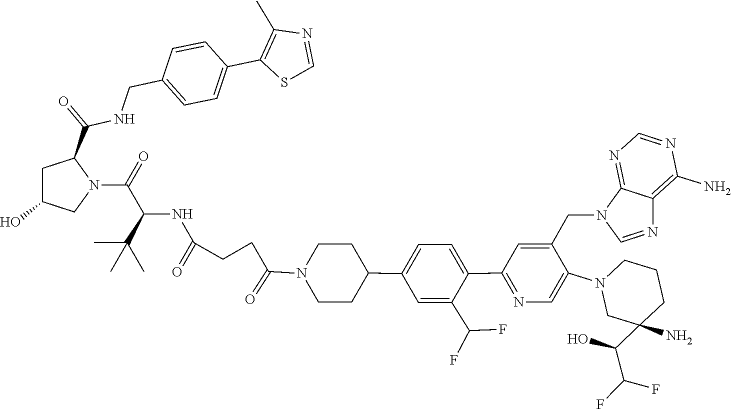 [SMILES: Cc1ncsc1-c1ccc(CNC(=O)[C@@H]2C[C@@H](O)CN2C(=O)[C@@H](NC(=O)CCC(=O)N2CCC(c3ccc(-c4cc(Cn5cnc6c(N)ncnc65)c(N5CCC[C@](N)([C@H](O)C(F)F)C5)cn4)c(C(F)F)c3)CC2)C(C)(C)C)cc1] |
| I-29 | 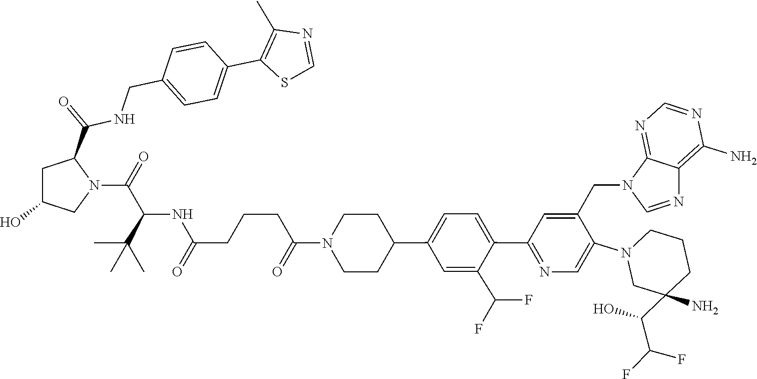 [SMILES: Cc1ncsc1-c1ccc(CNC(=O)[C@@H]2C[C@@H](O)CN2C(=O)[C@@H](NC(=O)CCCC(=O)N2CCC(c3ccc(-c4cc(Cn5cnc6c(N)ncnc65)c(N5CCC[C@](N)([C@@H](O)C(F)F)C5)cn4)c(C(F)F)c3)CC2)C(C)(C)C)cc1] |
| I-30 | 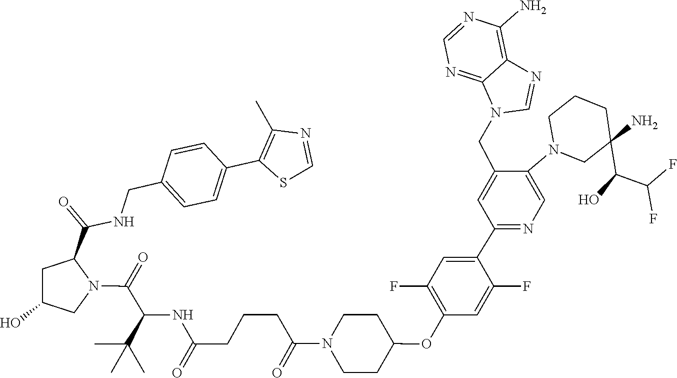 [SMILES: Cc1ncsc1-c1ccc(CNC(=O)[C@@H]2C[C@@H](O)CN2C(=O)[C@@H](NC(=O)CCCC(=O)N2CCC(Oc3cc(F)c(-c4cc(Cn5cnc6c(N)ncnc65)c(N5CCC[C@](N)([C@H](O)C(F)F)C5)cn4)cc3F)CC2)C(C)(C)C)cc1] |
| I-31 | 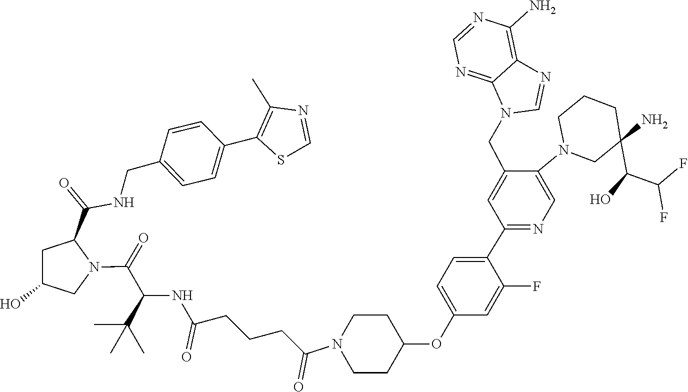 [SMILES: Cc1ncsc1-c1ccc(CNC(=O)[C@@H]2C[C@@H](O)CN2C(=O)[C@@H](NC(=O)CCCC(=O)N2CCC(Oc3ccc(-c4cc(Cn5cnc6c(N)ncnc65)c(N5CCC[C@](N)([C@H](O)C(F)F)C5)cn4)c(F)c3)CC2)C(C)(C)C)cc1] |
| I-32 | 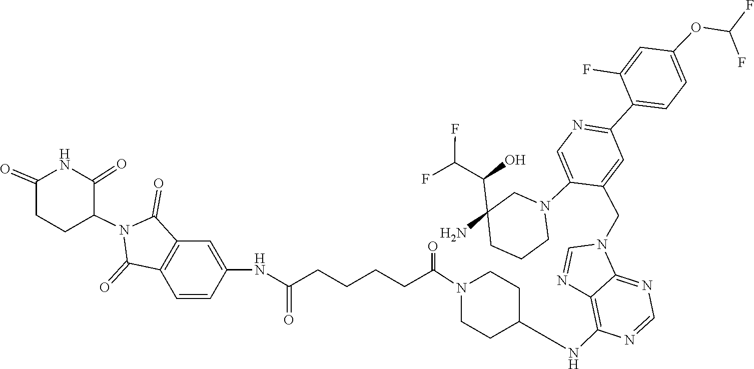 [SMILES: N[C@]1([C@H](O)C(F)F)CCCN(c2cnc(-c3ccc(OC(F)F)cc3F)cc2Cn2cnc3c(NC4CCN(C(=O)CCCCC(=O)Nc5ccc6c(c5)C(=O)N(C5CCC(=O)NC5=O)C6=O)CC4)ncnc32)C1] |
| I-33 | 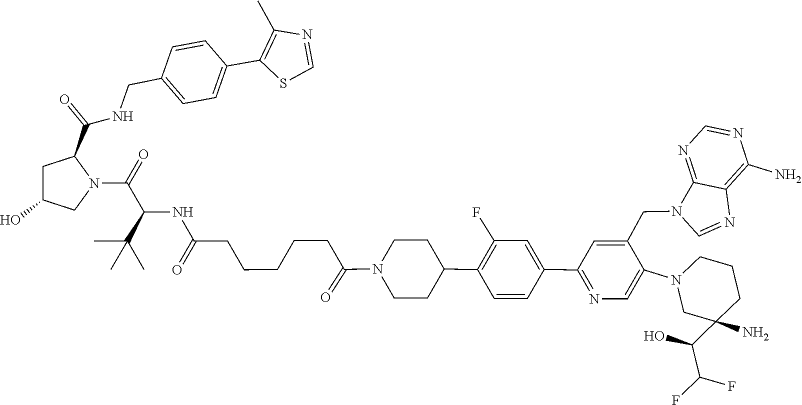 [SMILES: Cc1ncsc1-c1ccc(CNC(=O)[C@@H]2C[C@@H](O)CN2C(=O)[C@@H](NC(=O)CCCCCC(=O)N2CCC(c3ccc(-c4cc(Cn5cnc6c(N)ncnc65)c(N5CCC[C@](N)([C@H](O)C(F)F)C5)cn4)cc3F)CC2)C(C)(C)C)cc1] |
| I-34 | 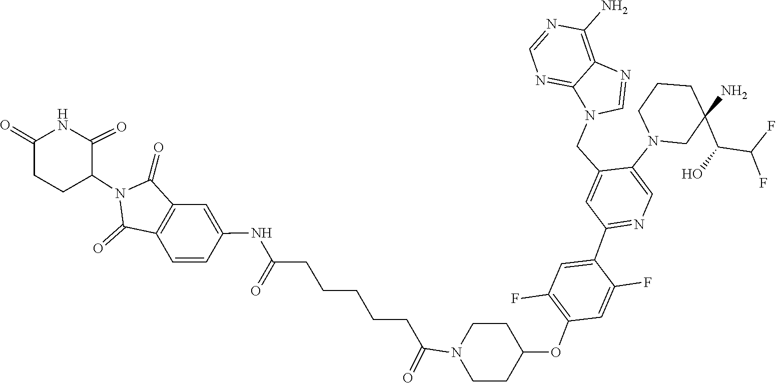 [SMILES: Nc1ncnc2c1ncn2Cc1cc(-c2cc(F)c(OC3CCN(C(=O)CCCCCC(=O)Nc4ccc5c(c4)C(=O)N(C4CCC(=O)NC4=O)C5=O)CC3)cc2F)ncc1N1CCC[C@](N)([C@@H](O)C(F)F)C1] |
| I-35 | 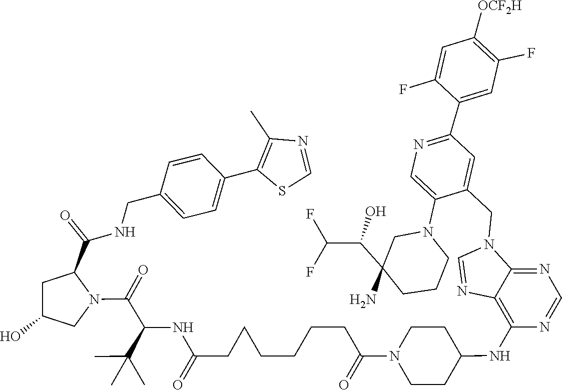 |
| I-36 | 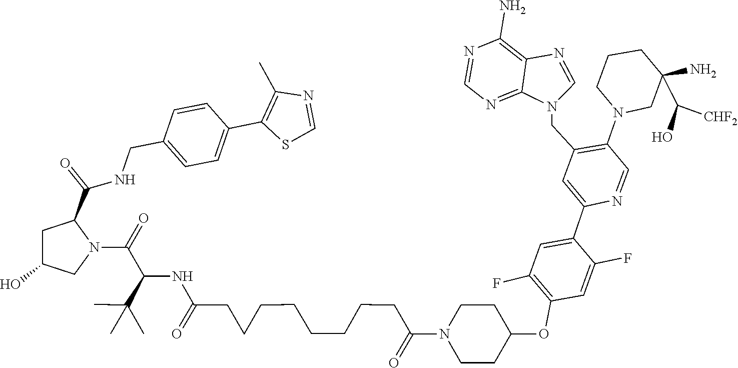 [SMILES: Cc1ncsc1-c1ccc(CNC(=O)[C@@H]2C[C@@H](O)CN2C(=O)[C@@H](NC(=O)CCCCCCCCC(=O)N2CCC(Oc3cc(F)c(-c4cc(Cn5cnc6c(N)ncnc65)c(N5CCC[C@](N)([C@H](O)C(F)F)C5)cn4)cc3F)CC2)C(C)(C)C)cc1] |
| I-37 | 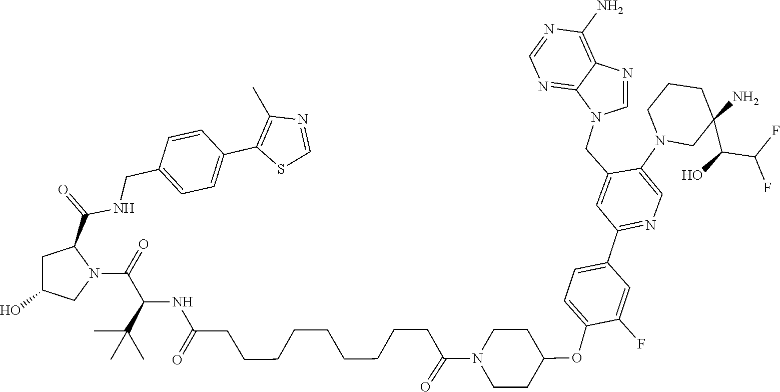 [SMILES: Cc1ncsc1-c1ccc(CNC(=O)[C@@H]2C[C@@H](O)CN2C(=O)[C@@H](NC(=O)CCCCCCCCCC(=O)N2CCC(Oc3ccc(-c4cc(Cn5cnc6c(N)ncnc65)c(N5CCC[C@](N)([C@H](O)C(F)F)C5)cn4)cc3F)CC2)C(C)(C)C)cc1] |
| I-38 | 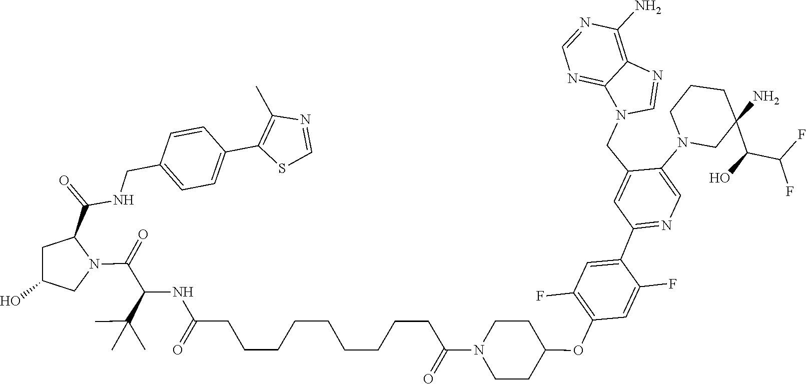 [SMILES: Cc1ncsc1-c1ccc(CNC(=O)[C@@H]2C[C@@H](O)CN2C(=O)[C@@H](NC(=O)CCCCCCCCCC(=O)N2CCC(Oc3cc(F)c(-c4cc(Cn5cnc6c(N)ncnc65)c(N5CCC[C@](N)([C@H](O)C(F)F)C5)cn4)cc3F)CC2)C(C)(C)C)cc1] |
| I-39 | 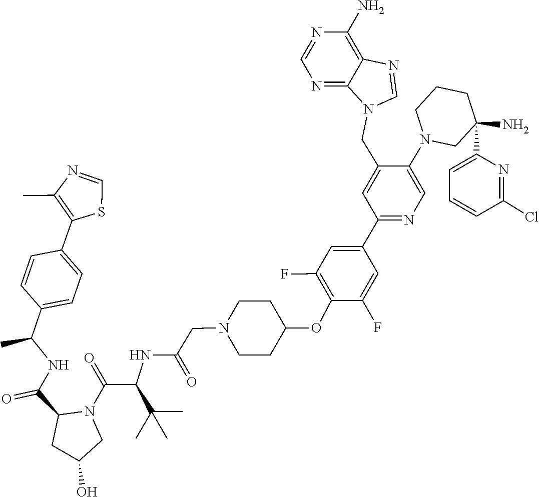 [SMILES: Cc1ncsc1-c1ccc([C@H](C)NC(=O)[C@@H]2C[C@@H](O)CN2C(=O)[C@@H](NC(=O)CN2CCC(Oc3c(F)cc(-c4cc(Cn5cnc6c(N)ncnc65)c(N5CCC[C@](N)(c6cccc(Cl)n6)C5)cn4)cc3F)CC2)C(C)(C)C)cc1] |
| I-40 | 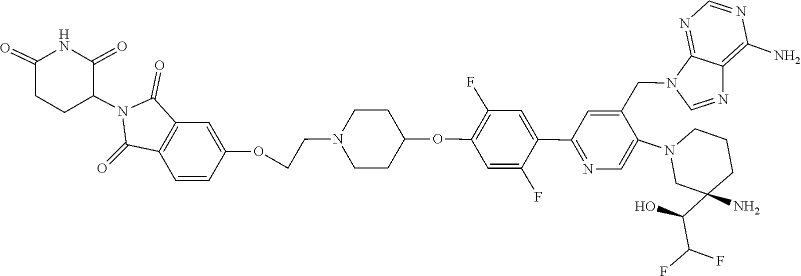 [SMILES: Nc1ncnc2c1ncn2Cc1cc(-c2cc(F)c(OC3CCN(CCOc4ccc5c(c4)C(=O)N(C4CCC(=O)NC4=O)C5=O)CC3)cc2F)ncc1N1CCC[C@](N)([C@H](O)C(F)F)C1] |
| I-41 | 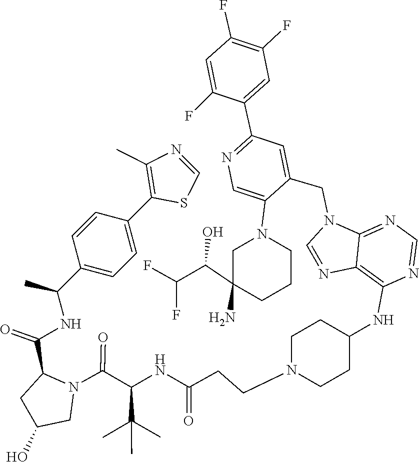 [SMILES: Cc1ncsc1-c1ccc([C@H](C)NC(=O)[C@@H]2C[C@@H](O)CN2C(=O)[C@@H](NC(=O)CCN2CCC(Nc3ncnc4c3ncn4Cc3cc(-c4cc(F)c(F)cc4F)ncc3N3CCC[C@](N)([C@@H](O)C(F)F)C3)CC2)C(C)(C)C)cc1] |
| I-42 | 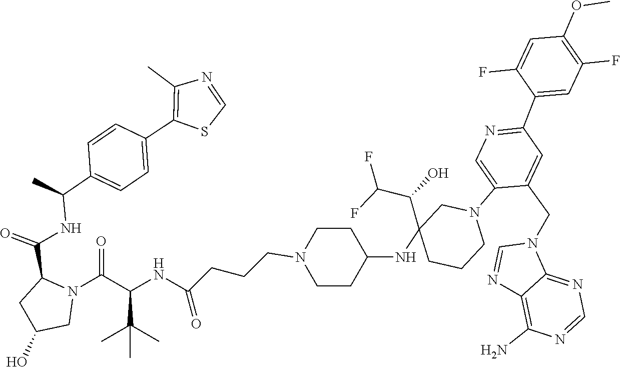 [SMILES: COc1cc(F)c(-c2cc(Cn3cnc4c(N)ncnc43)c(N3CCCC(NC4CCN(CCCC(=O)N[C@H](C(=O)N5C[C@H](O)C[C@H]5C(=O)N[C@@H](C)c5ccc(-c6scnc6C)cc5)C(C)(C)C)CC4)([C@@H](O)C(F)F)C3)cn2)cc1F] |
| I-43 | 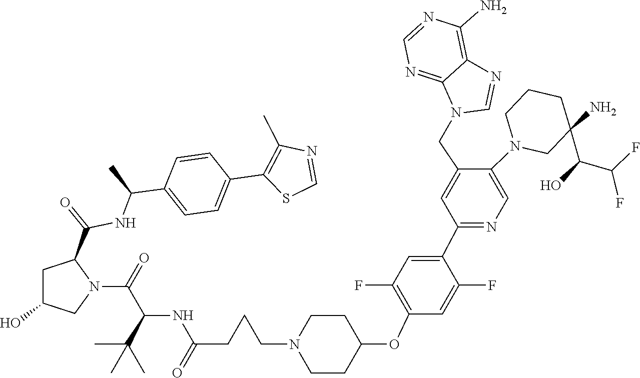 [SMILES: Cc1ncsc1-c1ccc([C@H](C)NC(=O)[C@@H]2C[C@@H](O)CN2C(=O)[C@@H](NC(=O)CCCN2CCC(Oc3cc(F)c(-c4cc(Cn5cnc6c(N)ncnc65)c(N5CCC[C@](N)([C@H](O)C(F)F)C5)cn4)cc3F)CC2)C(C)(C)C)cc1] |
| I-44 | 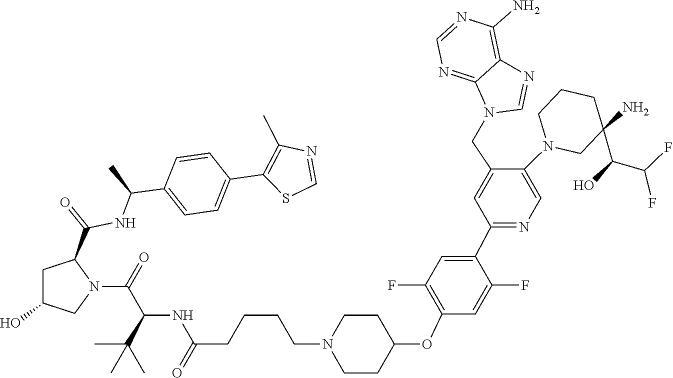 [SMILES: Cc1ncsc1-c1ccc([C@H](C)NC(=O)[C@@H]2C[C@@H](O)CN2C(=O)[C@@H](NC(=O)CCCCN2CCC(Oc3cc(F)c(-c4cc(Cn5cnc6c(N)ncnc65)c(N5CCC[C@](N)([C@H](O)C(F)F)C5)cn4)cc3F)CC2)C(C)(C)C)cc1] |
| I-45 | 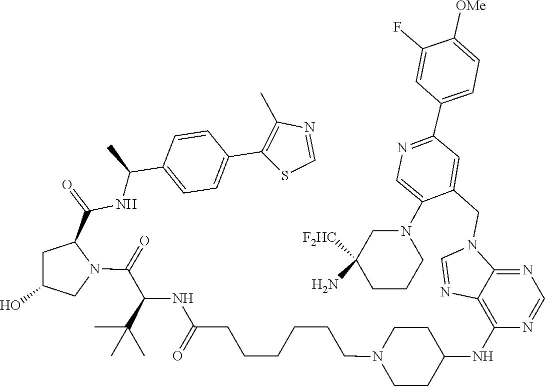 [SMILES: COc1ccc(-c2cc(Cn3cnc4c(NC5CCN(CCCCCCC(=O)N[C@H](C(=O)N6C[C@H](O)C[C@H]6C(=O)N[C@@H](C)c6ccc(-c7scnc7C)cc6)C(C)(C)C)CC5)ncnc43)c(N3CCC[C@](N)(C(F)F)C3)cn2)cc1F] |
| I-46 | 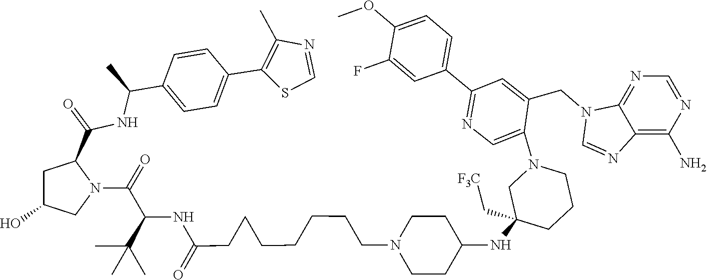 [SMILES: COc1ccc(-c2cc(Cn3cnc4c(N)ncnc43)c(N3CCC[C@@](CC(F)(F)F)(NC4CCN(CCCCCCCC(=O)N[C@H](C(=O)N5C[C@H](O)C[C@H]5C(=O)N[C@@H](C)c5ccc(-c6scnc6C)cc5)C(C)(C)C)CC4)C3)cn2)cc1F] |
| I-47 | 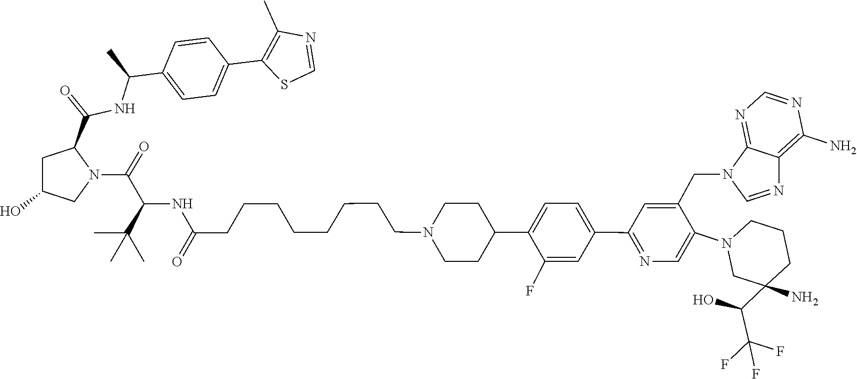 [SMILES: Cc1ncsc1-c1ccc([C@H](C)NC(=O)[C@@H]2C[C@@H](O)CN2C(=O)[C@@H](NC(=O)CCCCCCCCN2CCC(c3ccc(-c4cc(Cn5cnc6c(N)ncnc65)c(N5CCC[C@](N)([C@H](O)C(F)(F)F)C5)cn4)cc3F)CC2)C(C)(C)C)cc1] |
| I-48 | 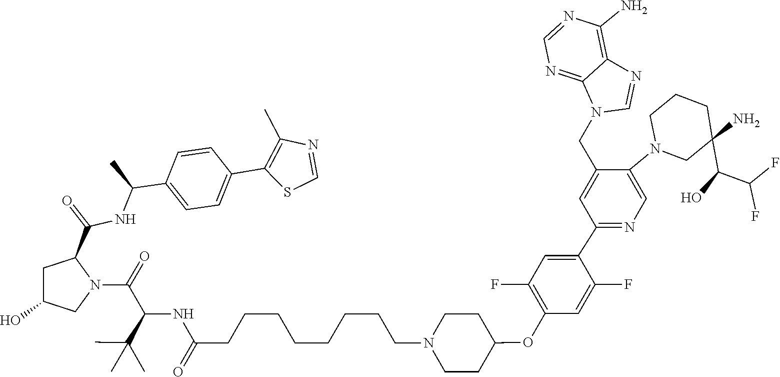 [SMILES: Cc1ncsc1-c1ccc([C@H](C)NC(=O)[C@@H]2C[C@@H](O)CN2C(=O)[C@@H](NC(=O)CCCCCCCCN2CCC(Oc3cc(F)c(-c4cc(Cn5cnc6c(N)ncnc65)c(N5CCC[C@](N)([C@H](O)C(F)F)C5)cn4)cc3F)CC2)C(C)(C)C)cc1] |
| I-49 | 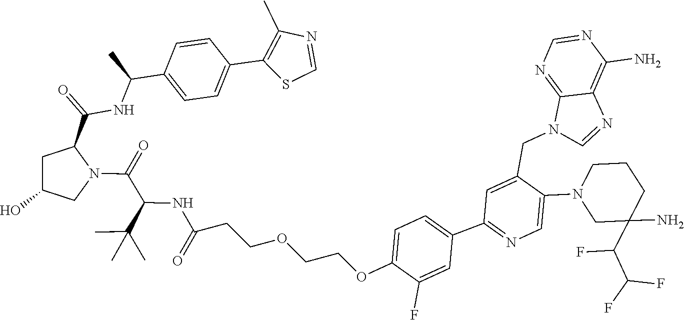 [SMILES: Cc1ncsc1-c1ccc([C@H](C)NC(=O)[C@@H]2C[C@@H](O)CN2C(=O)[C@@H](NC(=O)CCOCCOc2ccc(-c3cc(Cn4cnc5c(N)ncnc54)c(N4CCCC(N)(C(F)C(F)F)C4)cn3)cc2F)C(C)(C)C)cc1] |
| I-50 | 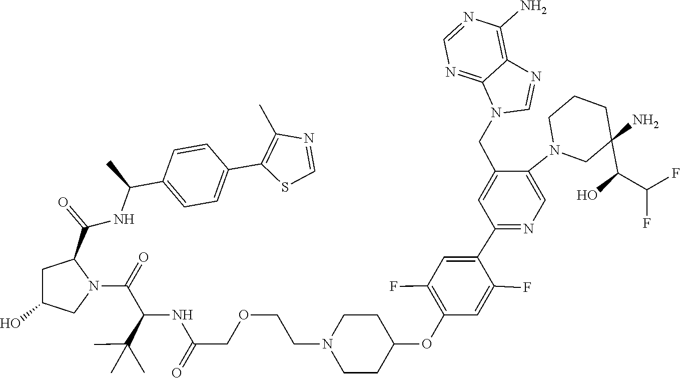 [SMILES: Cc1ncsc1-c1ccc([C@H](C)NC(=O)[C@@H]2C[C@@H](O)CN2C(=O)[C@@H](NC(=O)COCCN2CCC(Oc3cc(F)c(-c4cc(Cn5cnc6c(N)ncnc65)c(N5CCC[C@](N)([C@H](O)C(F)F)C5)cn4)cc3F)CC2)C(C)(C)C)cc1] |
| I-51 | 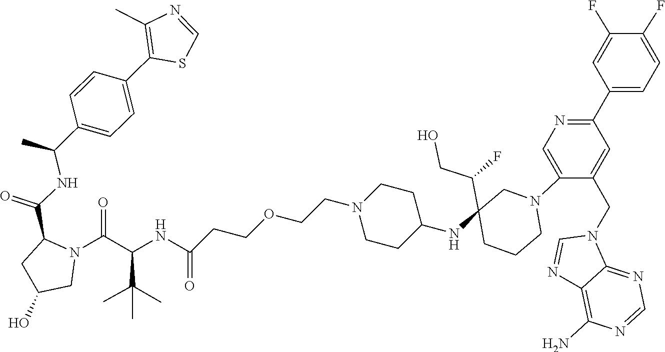 [SMILES: Cc1ncsc1-c1ccc([C@H](C)NC(=O)[C@@H]2C[C@@H](O)CN2C(=O)[C@@H](NC(=O)CCOCCN2CCC(N[C@]3([C@@H](F)CO)CCCN(c4cnc(-c5ccc(F)c(F)c5)cc4Cn4cnc5c(N)ncnc54)C3)CC2)C(C)(C)C)cc1] |
| I-52 | 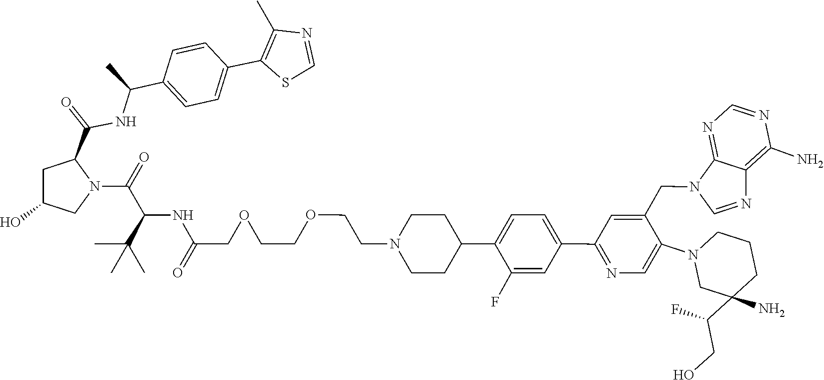 [SMILES: Cc1ncsc1-c1ccc([C@H](C)NC(=O)[C@@H]2C[C@@H](O)CN2C(=O)[C@@H](NC(=O)COCCOCCN2CCC(c3ccc(-c4cc(Cn5cnc6c(N)ncnc65)c(N5CCC[C@](N)([C@@H](F)CO)C5)cn4)cc3F)CC2)C(C)(C)C)cc1] |
| I-53 | 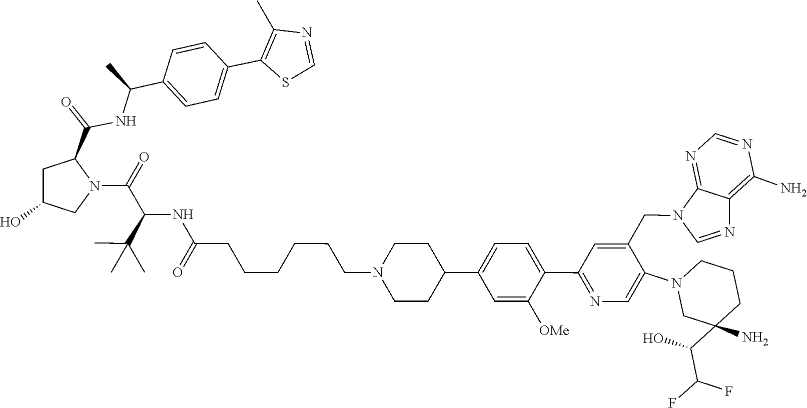 [SMILES: COc1cc(C2CCN(CCCCCCC(=O)N[C@H](C(=O)N3C[C@H](O)C[C@H]3C(=O)N[C@@H](C)c3ccc(-c4scnc4C)cc3)C(C)(C)C)CC2)ccc1-c1cc(Cn2cnc3c(N)ncnc32)c(N2CCC[C@](N)([C@@H](O)C(F)F)C2)cn1] |
| I-54 | 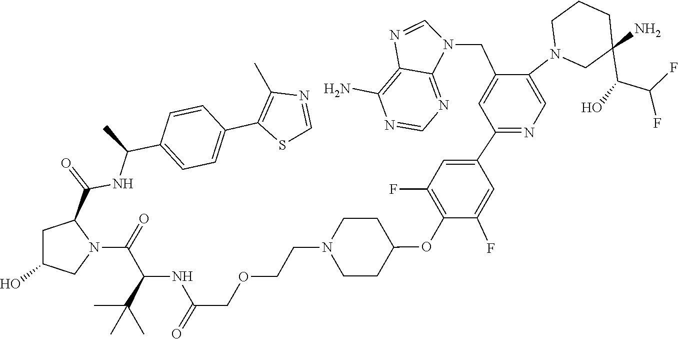 [SMILES: Cc1ncsc1-c1ccc([C@H](C)NC(=O)[C@@H]2C[C@@H](O)CN2C(=O)[C@@H](NC(=O)COCCN2CCC(Oc3c(F)cc(-c4cc(Cn5cnc6c(N)ncnc65)c(N5CCC[C@](N)([C@@H](O)C(F)F)C5)cn4)cc3F)CC2)C(C)(C)C)cc1] |
| I-55 | 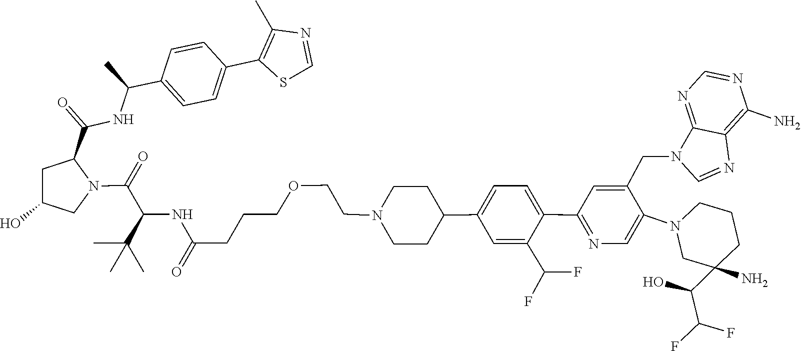 [SMILES: Cc1ncsc1-c1ccc([C@H](C)NC(=O)[C@@H]2C[C@@H](O)CN2C(=O)[C@@H](NC(=O)CCCOCCN2CCC(c3ccc(-c4cc(Cn5cnc6c(N)ncnc65)c(N5CCC[C@](N)([C@H](O)C(F)F)C5)cn4)c(C(F)F)c3)CC2)C(C)(C)C)cc1] |
| I-56 | 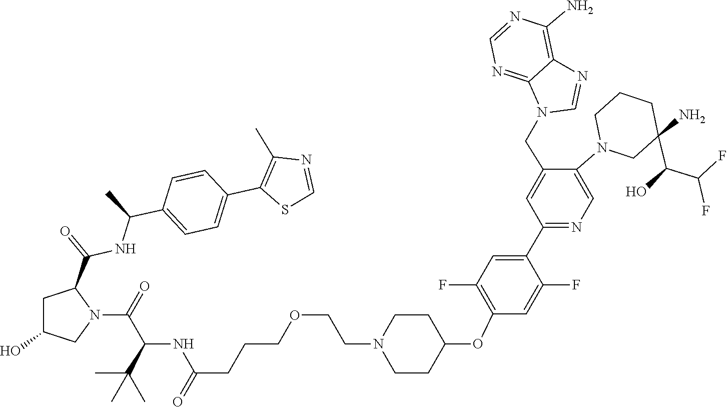 [SMILES: Cc1ncsc1-c1ccc([C@H](C)NC(=O)[C@@H]2C[C@@H](O)CN2C(=O)[C@@H](NC(=O)CCCOCCN2CCC(Oc3cc(F)c(-c4cc(Cn5cnc6c(N)ncnc65)c(N5CCC[C@](N)([C@H](O)C(F)F)C5)cn4)cc3F)CC2)C(C)(C)C)cc1] |
| I-57 | 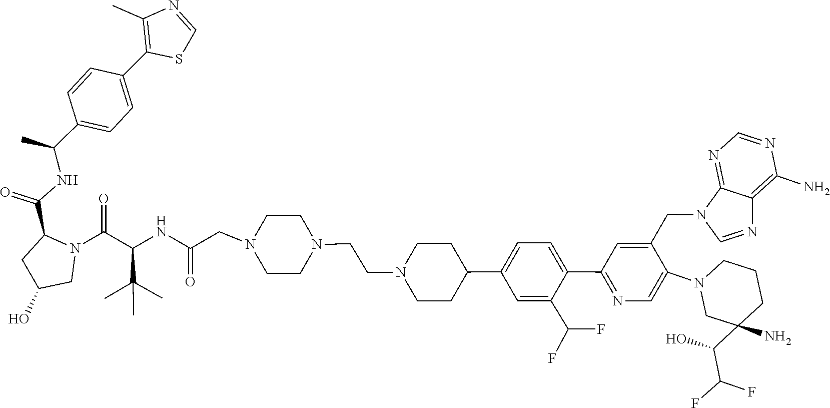 [SMILES: Cc1ncsc1-c1ccc([C@H](C)NC(=O)[C@@H]2C[C@@H](O)CN2C(=O)[C@@H](NC(=O)CN2CCN(CCN3CCC(c4ccc(-c5cc(Cn6cnc7c(N)ncnc76)c(N6CCC[C@](N)([C@@H](O)C(F)F)C6)cn5)c(C(F)F)c4)CC3)CC2)C(C)(C)C)cc1] |
| I-58 | 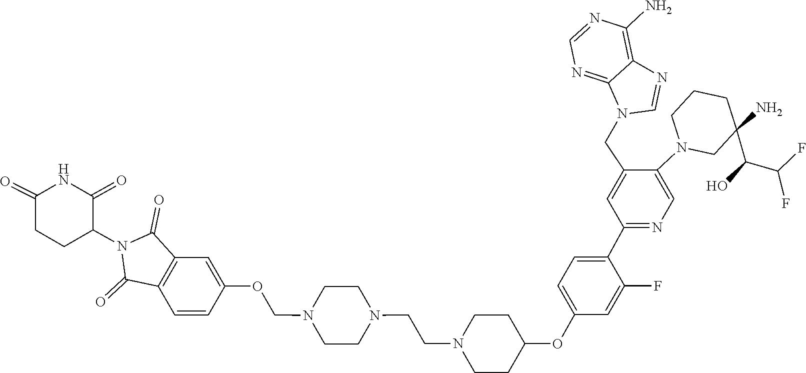 [SMILES: Nc1ncnc2c1ncn2Cc1cc(-c2ccc(OC3CCN(CCN4CCN(COc5ccc6c(c5)C(=O)N(C5CCC(=O)NC5=O)C6=O)CC4)CC3)cc2F)ncc1N1CCC[C@](N)([C@H](O)C(F)F)C1] |
| I-59 | 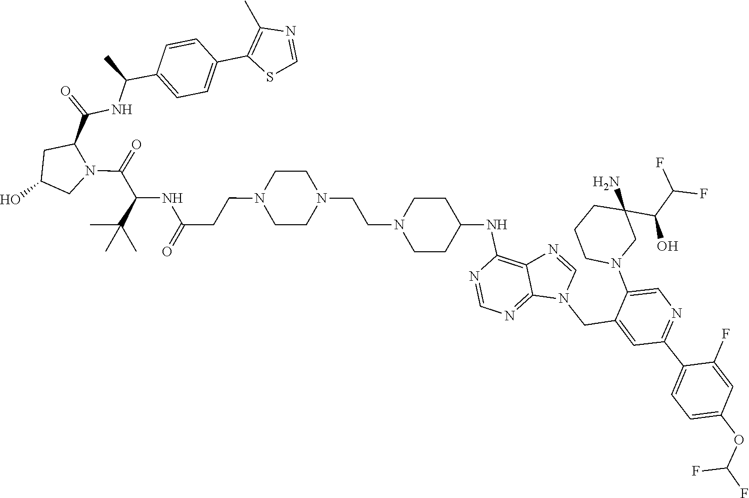 [SMILES: Cc1ncsc1-c1ccc([C@H](C)NC(=O)[C@@H]2C[C@@H](O)CN2C(=O)[C@@H](NC(=O)CCN2CCN(CCN3CCC(Nc4ncnc5c4ncn5Cc4cc(-c5ccc(OC(F)F)cc5F)ncc4N4CCC[C@](N)([C@H](O)C(F)F)C4)CC3)CC2)C(C)(C)C)cc1] |
| I-60 | 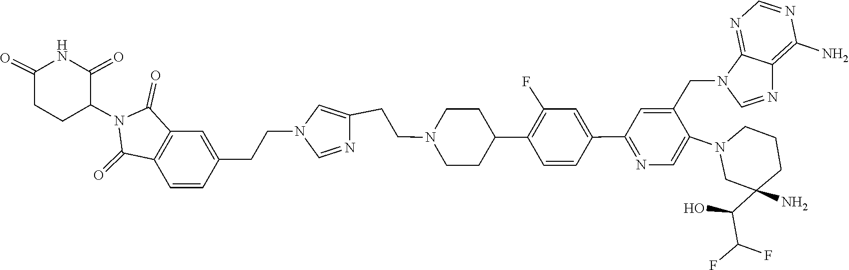 [SMILES: Nc1ncnc2c1ncn2Cc1cc(-c2ccc(C3CCN(CCc4cn(CCc5ccc6c(c5)C(=O)N(C5CCC(=O)NC5=O)C6=O)cn4)CC3)c(F)c2)ncc1N1CCC[C@](N)([C@H](O)C(F)F)C1] |
| I-61 | 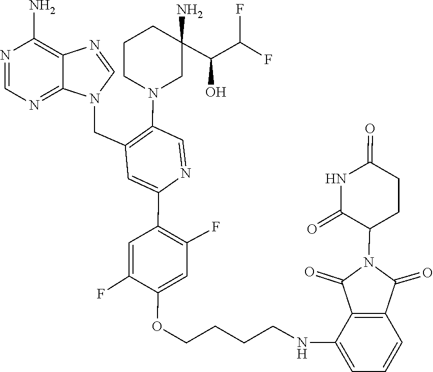 [SMILES: Nc1ncnc2c1ncn2Cc1cc(-c2cc(F)c(OCCCCNc3cccc4c3C(=O)N(C3CCC(=O)NC3=O)C4=O)cc2F)ncc1N1CCC[C@](N)([C@H](O)C(F)F)C1] |
| I-62 | 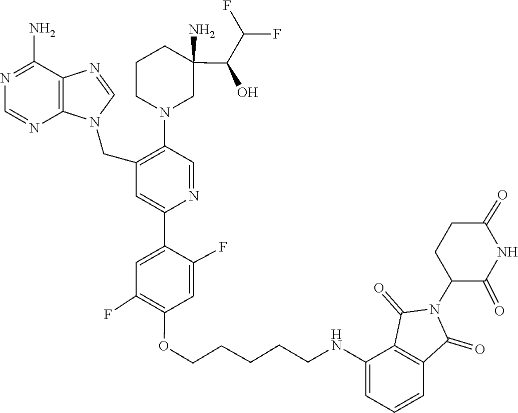 [SMILES: Nc1ncnc2c1ncn2Cc1cc(-c2cc(F)c(OCCCCCNc3cccc4c3C(=O)N(C3CCC(=O)NC3=O)C4=O)cc2F)ncc1N1CCC[C@](N)([C@H](O)C(F)F)C1] |
| I-63 | 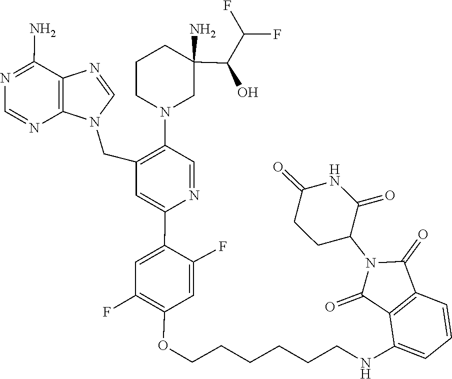 [SMILES: Nc1ncnc2c1ncn2Cc1cc(-c2cc(F)c(OCCCCCCNc3cccc4c3C(=O)N(C3CCC(=O)NC3=O)C4=O)cc2F)ncc1N1CCC[C@](N)([C@H](O)C(F)F)C1] |
| I-64 | 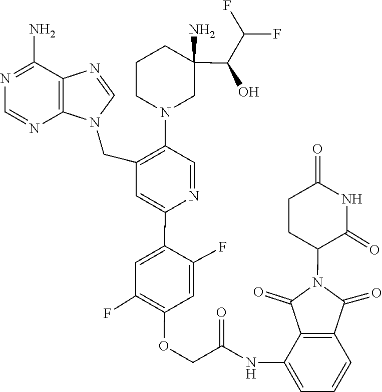 [SMILES: Nc1ncnc2c1ncn2Cc1cc(-c2cc(F)c(OCC(=O)Nc3cccc4c3C(=O)N(C3CCC(=O)NC3=O)C4=O)cc2F)ncc1N1CCC[C@](N)([C@H](O)C(F)F)C1] |
| I-65 | 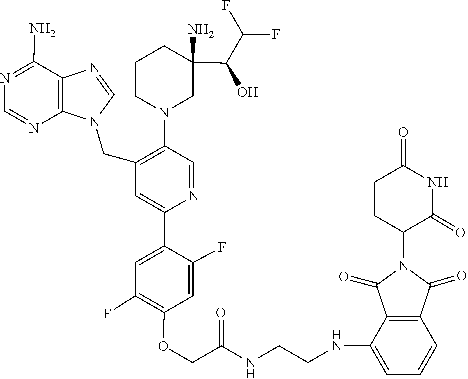 [SMILES: Nc1ncnc2c1ncn2Cc1cc(-c2cc(F)c(OCC(=O)NCCNc3cccc4c3C(=O)N(C3CCC(=O)NC3=O)C4=O)cc2F)ncc1N1CCC[C@](N)([C@H](O)C(F)F)C1] |
| I-66 | 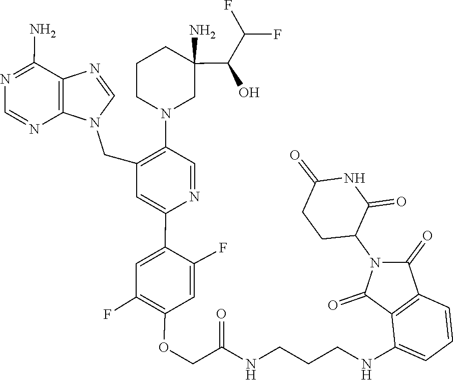 [SMILES: Nc1ncnc2c1ncn2Cc1cc(-c2cc(F)c(OCC(=O)NCCCNc3cccc4c3C(=O)N(C3CCC(=O)NC3=O)C4=O)cc2F)ncc1N1CCC[C@](N)([C@H](O)C(F)F)C1] |
| I-67 | 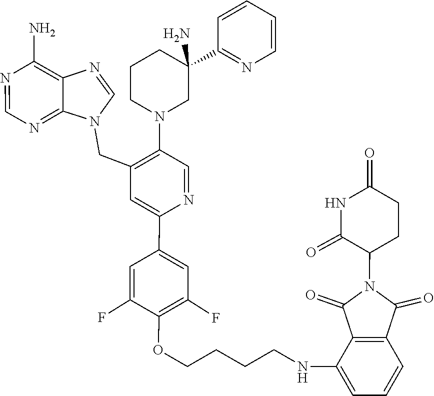 [SMILES: Nc1ncnc2c1ncn2Cc1cc(-c2cc(F)c(OCCCCNc3cccc4c3C(=O)N(C3CCC(=O)NC3=O)C4=O)c(F)c2)ncc1N1CCC[C@](N)(c2ccccn2)C1] |
| I-68 | 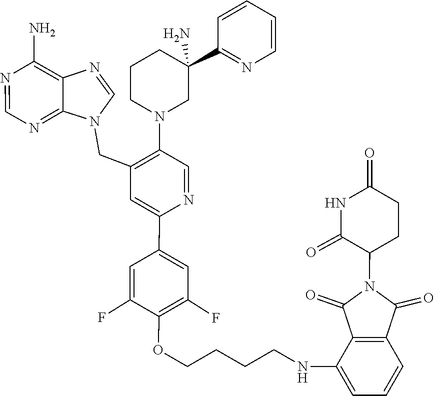 [SMILES: Nc1ncnc2c1ncn2Cc1cc(-c2cc(F)c(OCCCCNc3cccc4c3C(=O)N(C3CCC(=O)NC3=O)C4=O)c(F)c2)ncc1N1CCC[C@@](N)(c2ccccn2)C1] |
| I-69 | 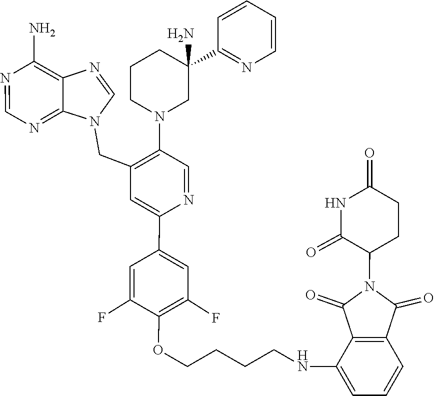 [SMILES: Nc1ncnc2c1ncn2Cc1cc(-c2cc(F)c(OCCCCNc3cccc4c3C(=O)N(C3CCC(=O)NC3=O)C4=O)c(F)c2)ncc1N1CCC[C@](N)(c2ccccn2)C1] |
| I-70 | 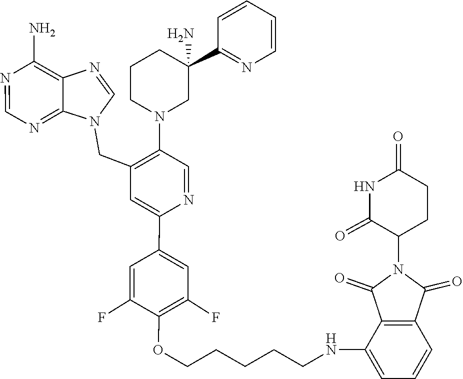 [SMILES: Nc1ncnc2c1ncn2Cc1cc(-c2cc(F)c(OCCCCCNc3cccc4c3C(=O)N(C3CCC(=O)NC3=O)C4=O)c(F)c2)ncc1N1CCC[C@@](N)(c2ccccn2)C1] |
| I-71 | 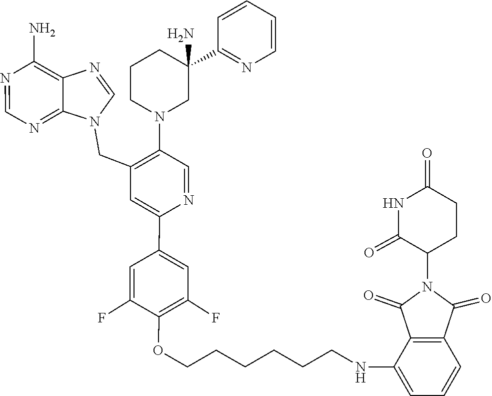 [SMILES: Nc1ncnc2c1ncn2Cc1cc(-c2cc(F)c(OCCCCCCNc3cccc4c3C(=O)N(C3CCC(=O)NC3=O)C4=O)c(F)c2)ncc1N1CCC[C@](N)(c2ccccn2)C1] |
| I-72 | 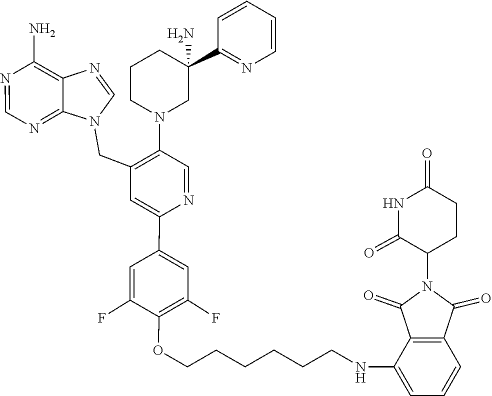 [SMILES: Nc1ncnc2c1ncn2Cc1cc(-c2cc(F)c(OCCCCCCNc3cccc4c3C(=O)N(C3CCC(=O)NC3=O)C4=O)c(F)c2)ncc1N1CCC[C@@](N)(c2ccccn2)C1] |
| I-73 | 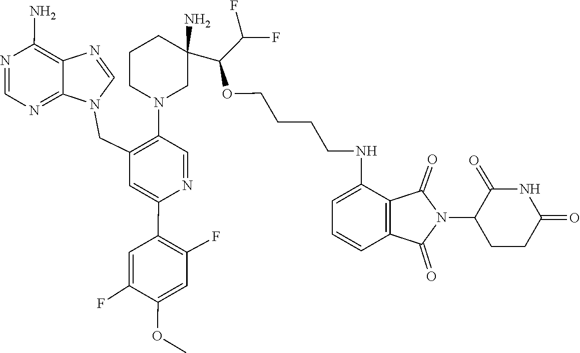 [SMILES: COc1cc(F)c(-c2cc(Cn3cnc4c(N)ncnc43)c(N3CCC[C@](N)([C@H](OCCCCNc4cccc5c4C(=O)N(C4CCC(=O)NC4=O)C5=O)C(F)F)C3)cn2)cc1F] |
| I-74 | 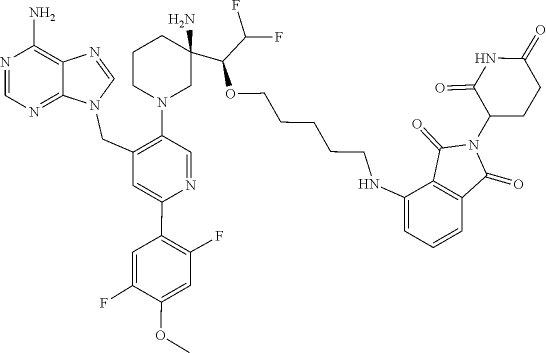 [SMILES: COc1cc(F)c(-c2cc(Cn3cnc4c(N)ncnc43)c(N3CCC[C@](N)([C@H](OCCCCCNc4cccc5c4C(=O)N(C4CCC(=O)NC4=O)C5=O)C(F)F)C3)cn2)cc1F] |
| I-75 | 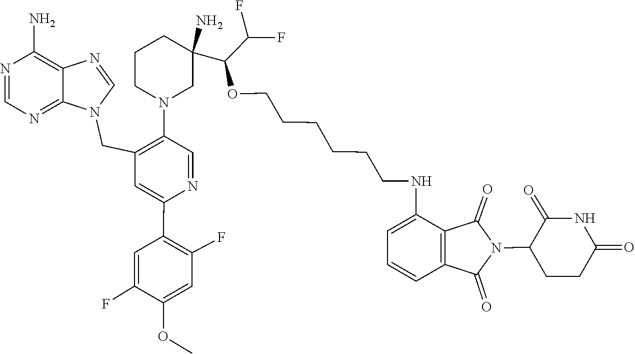 [SMILES: COc1cc(F)c(-c2cc(Cn3cnc4c(N)ncnc43)c(N3CCC[C@](N)([C@H](OCCCCCCNc4cccc5c4C(=O)N(C4CCC(=O)NC4=O)C5=O)C(F)F)C3)cn2)cc1F] |
| I-76 | 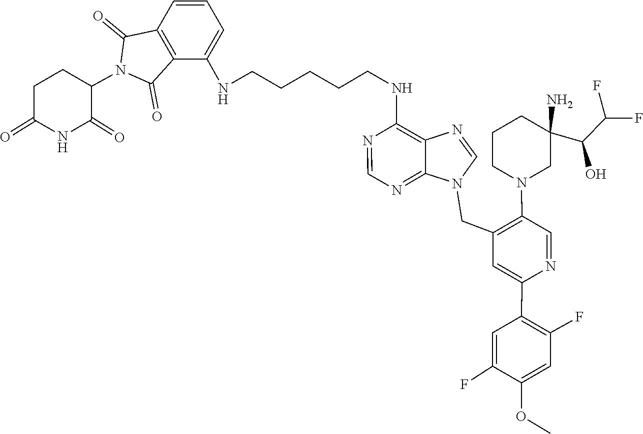 [SMILES: COc1cc(F)c(-c2cc(Cn3cnc4c(NCCCCCNc5cccc6c5C(=O)N(C5CCC(=O)NC5=O)C6=O)ncnc43)c(N3CCC[C@](N)([C@H](O)C(F)F)C3)cn2)cc1F] |
| I-77 | 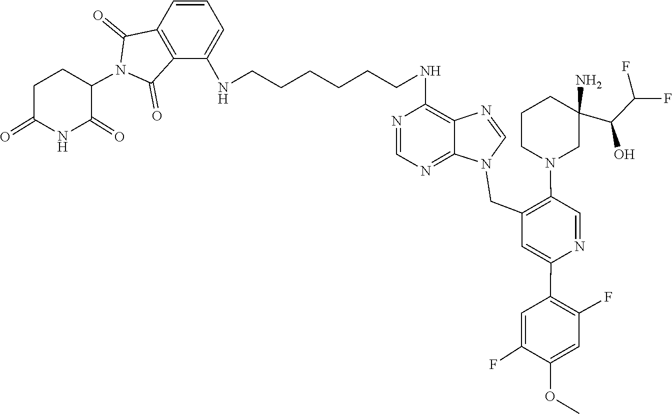 [SMILES: COc1cc(F)c(-c2cc(Cn3cnc4c(NCCCCCCNc5cccc6c5C(=O)N(C5CCC(=O)NC5=O)C6=O)ncnc43)c(N3CCC[C@](N)([C@H](O)C(F)F)C3)cn2)cc1F] |
| I-78 | 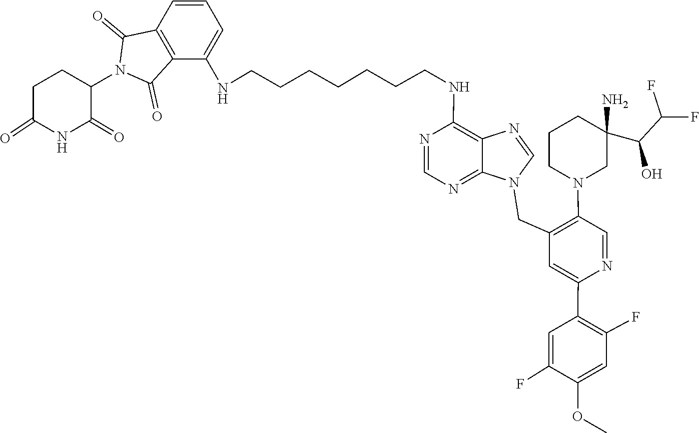 [SMILES: COc1cc(F)c(-c2cc(Cn3cnc4c(NCCCCCCCNc5cccc6c5C(=O)N(C5CCC(=O)NC5=O)C6=O)ncnc43)c(N3CCC[C@](N)([C@H](O)C(F)F)C3)cn2)cc1F] |
| I-79 | 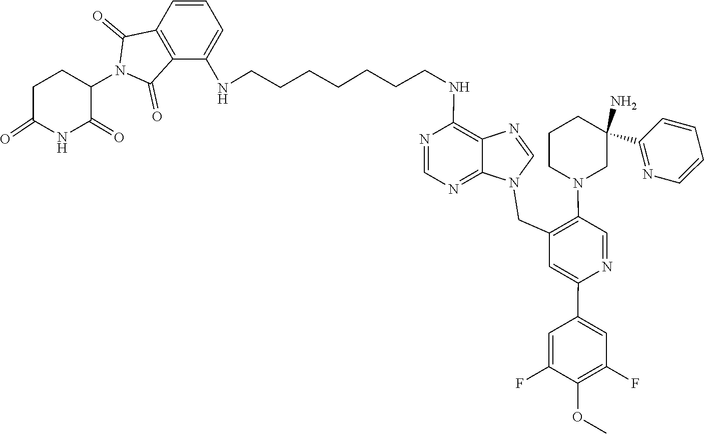 [SMILES: COc1c(F)cc(-c2cc(Cn3cnc4c(NCCCCCCCNc5cccc6c5C(=O)N(C5CCC(=O)NC5=O)C6=O)ncnc43)c(N3CCC[C@](N)(c4ccccn4)C3)cn2)cc1F] |
| I-80 | 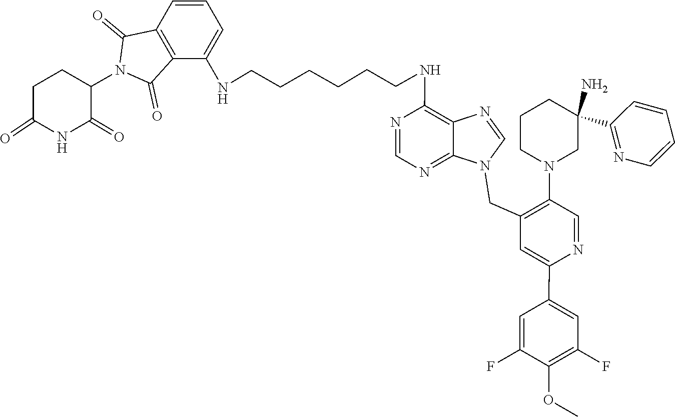 [SMILES: COc1c(F)cc(-c2cc(Cn3cnc4c(NCCCCCCNc5cccc6c5C(=O)N(C5CCC(=O)NC5=O)C6=O)ncnc43)c(N3CCC[C@](N)(c4ccccn4)C3)cn2)cc1F] |
| I-81 | 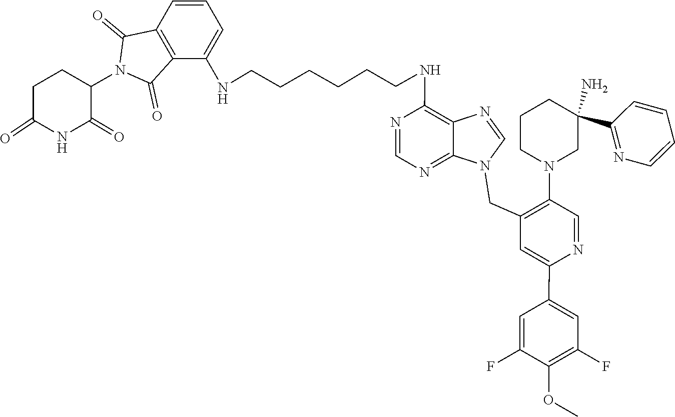 [SMILES: COc1c(F)cc(-c2cc(Cn3cnc4c(NCCCCCCNc5cccc6c5C(=O)N(C5CCC(=O)NC5=O)C6=O)ncnc43)c(N3CCC[C@@](N)(c4ccccn4)C3)cn2)cc1F] |
| I-82 | 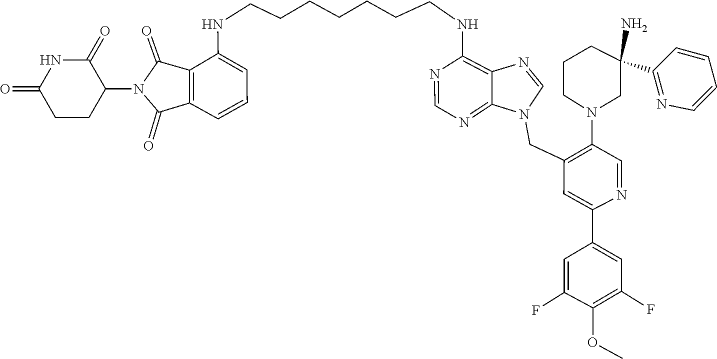 [SMILES: COc1c(F)cc(-c2cc(Cn3cnc4c(NCCCCCCCNc5cccc6c5C(=O)N(C5CCC(=O)NC5=O)C6=O)ncnc43)c(N3CCC[C@](N)(c4ccccn4)C3)cn2)cc1F] |
| I-83 | 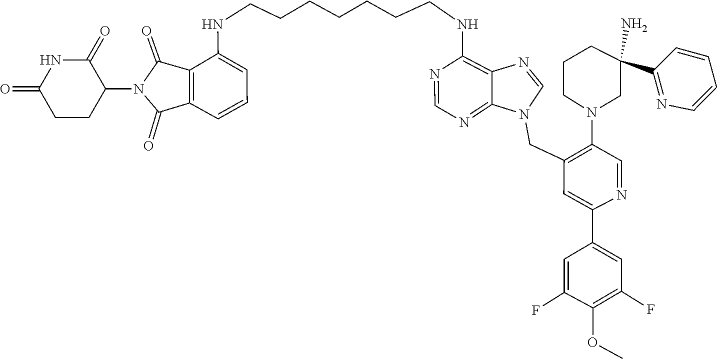 [SMILES: COc1c(F)cc(-c2cc(Cn3cnc4c(NCCCCCCCNc5cccc6c5C(=O)N(C5CCC(=O)NC5=O)C6=O)ncnc43)c(N3CCC[C@@](N)(c4ccccn4)C3)cn2)cc1F] |
| I-84 | 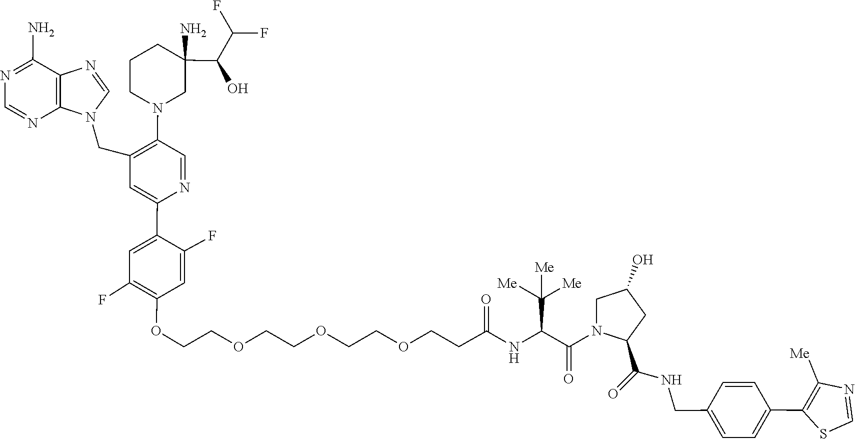 [SMILES: Cc1ncsc1-c1ccc(CNC(=O)[C@@H]2C[C@@H](O)CN2C(=O)[C@@H](NC(=O)CCOCCOCCOCCOc2cc(F)c(-c3cc(Cn4cnc5c(N)ncnc54)c(N4CCC[C@](N)([C@H](O)C(F)F)C4)cn3)cc2F)C(C)(C)C)cc1] |
| I-85 | 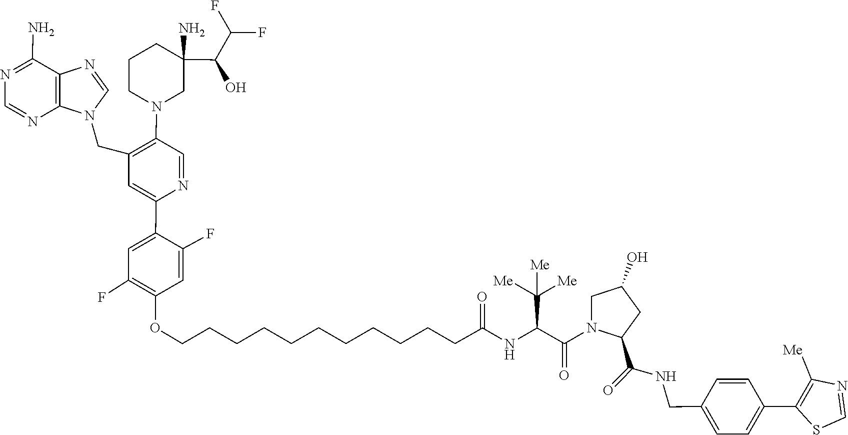 [SMILES: Cc1ncsc1-c1ccc(CNC(=O)[C@@H]2C[C@@H](O)CN2C(=O)[C@@H](NC(=O)CCCCCCCCCCCOc2cc(F)c(-c3cc(Cn4cnc5c(N)ncnc54)c(N4CCC[C@](N)([C@H](O)C(F)F)C4)cn3)cc2F)C(C)(C)C)cc1] |
| I-86 | 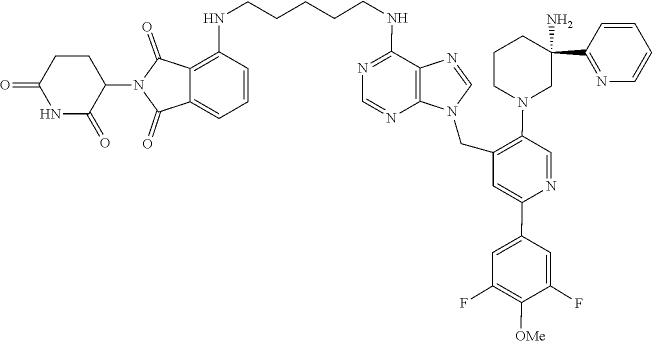 [SMILES: COc1c(F)cc(-c2cc(Cn3cnc4c(NCCCCCNc5cccc6c5C(=O)N(C5CCC(=O)NC5=O)C6=O)ncnc43)c(N3CCC[C@@](N)(c4ccccn4)C3)cn2)cc1F] |
| I-87 | 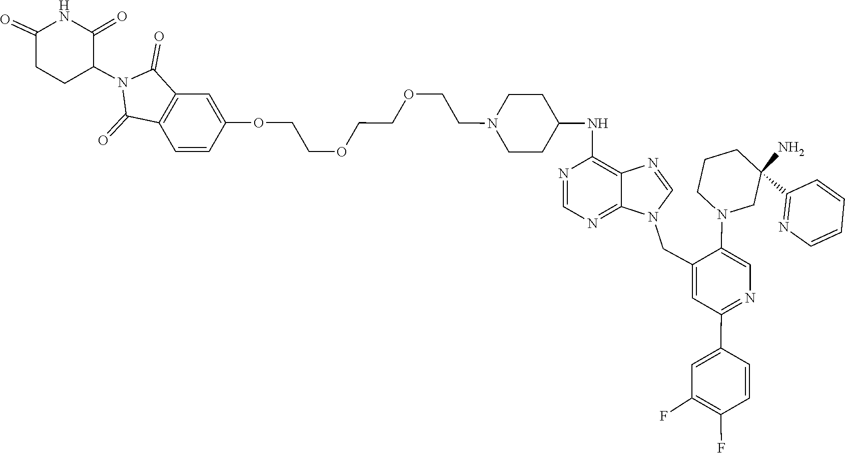 [SMILES: N[C@]1(c2ccccn2)CCCN(c2cnc(-c3ccc(F)c(F)c3)cc2Cn2cnc3c(NC4CCN(CCOCCOCCOc5ccc6c(c5)C(=O)N(C5CCC(=O)NC5=O)C6=O)CC4)ncnc32)C1] |
| I-88 | 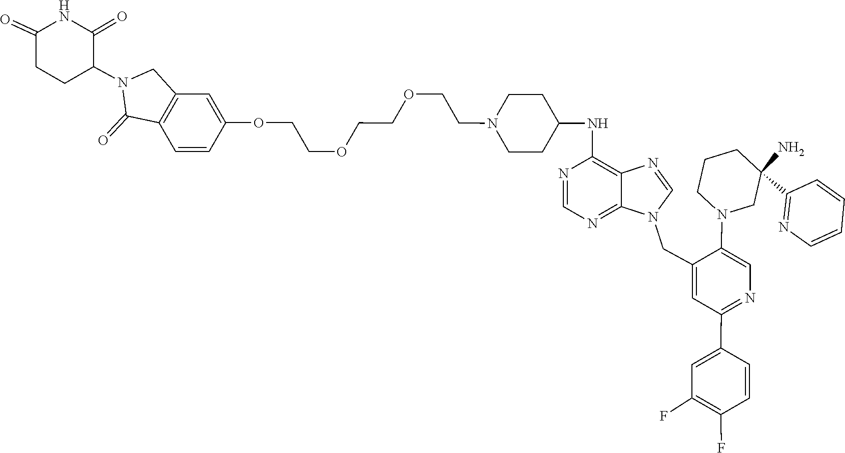 [SMILES: N[C@]1(c2ccccn2)CCCN(c2cnc(-c3ccc(F)c(F)c3)cc2Cn2cnc3c(NC4CCN(CCOCCOCCOc5ccc6c(c5)CN(C5CCC(=O)NC5=O)C6=O)CC4)ncnc32)C1] |
| I-89 | 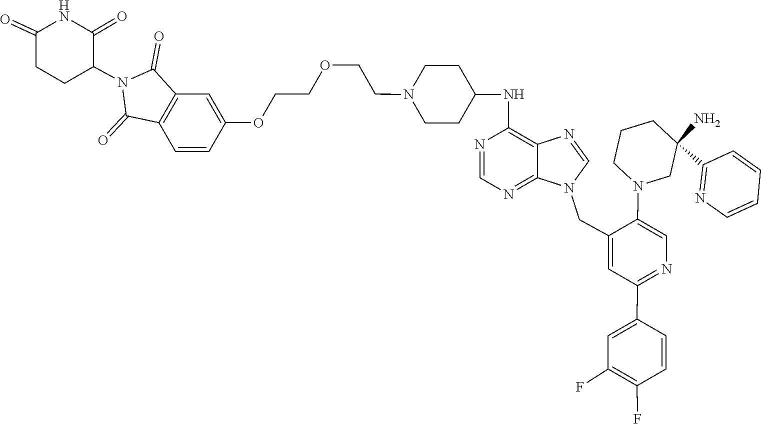 [SMILES: N[C@]1(c2ccccn2)CCCN(c2cnc(-c3ccc(F)c(F)c3)cc2Cn2cnc3c(NC4CCN(CCOCCOc5ccc6c(c5)C(=O)N(C5CCC(=O)NC5=O)C6=O)CC4)ncnc32)C1] |
| I-90 | 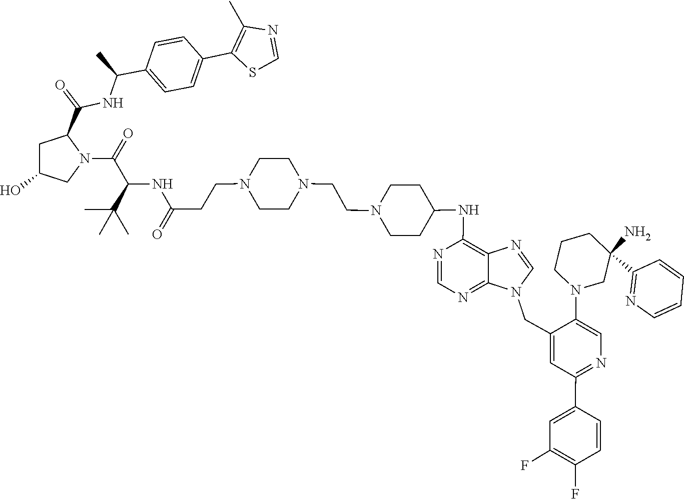 [SMILES: Cc1ncsc1-c1ccc([C@H](C)NC(=O)[C@@H]2C[C@@H](O)CN2C(=O)[C@@H](NC(=O)CCN2CCN(CCN3CCC(Nc4ncnc5c4ncn5Cc4cc(-c5ccc(F)c(F)c5)ncc4N4CCC[C@](N)(c5ccccn5)C4)CC3)CC2)C(C)(C)C)cc1] |
| I-90 | 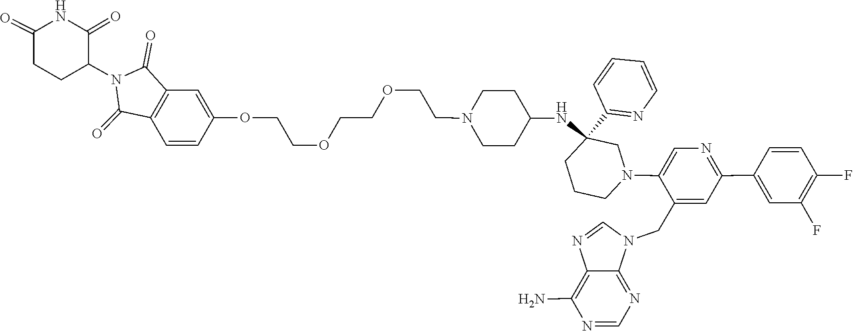 [SMILES: Nc1ncnc2c1ncn2Cc1cc(-c2ccc(F)c(F)c2)ncc1N1CCC[C@](NC2CCN(CCOCCOCCOc3ccc4c(c3)C(=O)N(C3CCC(=O)NC3=O)C4=O)CC2)(c2ccccn2)C1] |
| I-90 | 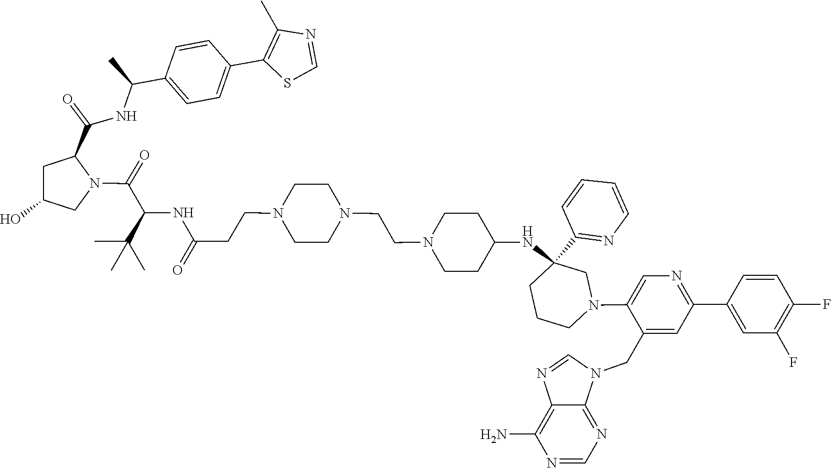 [SMILES: Cc1ncsc1-c1ccc([C@H](C)NC(=O)[C@@H]2C[C@@H](O)CN2C(=O)[C@@H](NC(=O)CCN2CCN(CCN3CCC(N[C@]4(c5ccccn5)CCCN(c5cnc(-c6ccc(F)c(F)c6)cc5Cn5cnc6c(N)ncnc65)C4)CC3)CC2)C(C)(C)C)cc1] |
| I-93 | 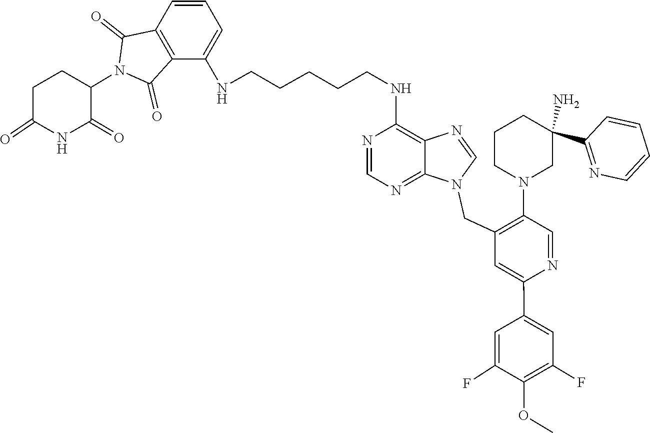 [SMILES: COc1c(F)cc(-c2cc(Cn3cnc4c(NCCCCCNc5cccc6c5C(=O)N(C5CCC(=O)NC5=O)C6=O)ncnc43)c(N3CCC[C@@](N)(c4ccccn4)C3)cn2)cc1F] |

Compounds may be further characterized according to their inhibitory activity against NSD2. In certain embodiments, a preferred compound has an IC₅₀ less than 2 μM against NSD2. In certain embodiments, a more preferred compound has an IC₅₀ less than 0.5 μM against NSD2. In certain embodiments, an even more preferred compound has an IC₅₀ less than or equal to 0.05 μM against NSD2.

Methods for preparing compounds described herein are illustrated in the following synthetic Schemes, and in the Examples below. The Schemes are given for the purpose of illustrating the disclosure, and are not intended to limit the scope or spirit of the disclosure. Starting materials shown in the Schemes can be obtained from commercial sources or can be prepared based on procedures described in the literature.

In the Schemes, it is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated (for example, use of protecting groups or alternative reactions). Protecting group chemistry and strategy is well known in the art, for example, as described in detail in “Protecting Groups in Organic Synthesis”, T. W. Greene and P. G. M. Wuts, 3^{rd} edition, John Wiley & Sons, 1999, the entire contents of which are hereby incorporated by reference.

The synthetic route illustrated in Scheme 1 is a general method for preparing heterobifunctional substituted pyridinylmethyl aminopurine and related compounds D. Coupling compound A (a precursor of TBM) with L′ (a precursor to linker L, containing functionality for coupling to the precursors of both TBM and LBM) affords intermediate B (wherein L″ is a precursor to linker L that contains functionality for coupling to the LBM precursolar). Coupling intermediate B with compound C (a precursor of LBM) affords heterobifunctional substituted pyridinylmethyl aminopurine and related compounds D. Alternatively, the order of coupling compounds A and C to L′ may be reversed, such that L′ is first coupled with compound C, before being coupled to compound A. It is understood by one skilled in the art of organic synthesis that protecting group strategies may be employed as necessary, for example, if L′ contains two of the same functional group that are to be selectively coupled to compound A and compound C. For example, L′ may contain, for example, both an unprotected carboxylic acid for coupling to compound A, and a carboxylic acid group that is protected (for example, as a methyl or benzyl ester) during the coupling with compound A and subsequently deprotected (for example, via basic hydrolysis of a methyl ester or hydrogenolysis of a benzyl ester) prior to coupling with compound C.

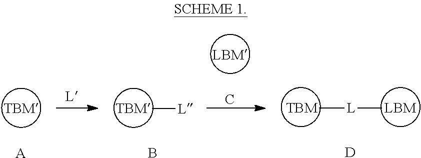

The coupling of compound A with L′, and the coupling of intermediate B with compound C, can be accomplished with a wide variety of strategies. For example, amide coupling conditions can be employed when compound A (or compound C) is to be attached at a primary or secondary nitrogen atom and L′ (or L″) contains a carboxylic acid group, or vice versa (i.e., compound A contains a carboxylic acid group and L′ contains a primary or secondary nitrogen atom). Alternatively, reductive amination conditions can be employed when compound A (or compound C) is to be attached at a primary or secondary nitrogen atom and L′ (or L″) contains an aldehyde group, or vice versa. Alternatively, nucleophilic substitution conditions can be employed when compound A (or compound C) is to be attached at a modifiable hydroxyl, thiol, or primary or secondary nitrogen atom, and L′ (or L″) contains a leaving group (such as an alkyl triflate, α-bromoketone, or aryl chloride), or vice versa. As yet another option, transition-metal-mediated coupling conditions can be employed when compound A (or compound C) is to be attached at a hydroxyl, primary or secondary nitrogen atom, or appropriately functionalized carbon atom (for example, a vinyl bromide or aryl sulfonate) and L′ (or L″) contains a suitable coupling partner (for example, an olefin for a Heck coupling, a trialkylstannane for a Stille coupling, or a boronic acid or boronate ester for a Suzuki coupling, Buchwald-Hartwig amination, or Chan-Lam coupling), or vice versa.

The synthetic route illustrated in Scheme 2 is a general method for preparing heterobifunctional substituted pyridinylmethyl aminopurine compounds K. Alkylation of compound E with an A,Ω-difunctional alkylating agent F (e.g., an A,Ω-dibromide) affords mono-alkylated intermediate G, wherein X represents a leaving group (e.g., a halide, triflate, tosylate, mesylate, or the like) and wherein PG¹ and PG² represent first and second amino protecting groups. In certain embodiments, PG¹ and PG² represent different protecting groups that are selectively removable by different deprotection methodologies (e.g., PG¹ may be a tert-butyloxycarbonyl (BOC) protecting group, which is removable by acid, and PG² may be a benzyl group, which is removable by hydrogenation, or vice versa). In certain other embodiments, PG¹ and PG² represent different protecting groups that are simultaneously removable using the same deprotecting methodology. In certain further embodiments, PG¹ and PG² represent the same protecting group, which is removable using a single deprotecting methodology.

The nature of the protecting groups used in Scheme 2 may be determined based on the desired point of attachment of amino-isoindoline compound H to the rest of the molecule. For example, as shown in Scheme 2, the linker portion of the molecule, which was introduced via the reaction of compound E with A,Ω-difunctional alkylating agent F, is attached at the phenoxy oxygen at the bottom of the molecule. In such embodiments, the amino-isoindoline compound H may be used to alkylate compound G to provide bis-protected compound I. After this alkylation, bis-protected compound I may then be deprotected to provide mono-protected compound J, which in turn may be deprotected to provide heterobifunctional substituted pyridinylmethyl aminopurine K. In certain embodiments, PG¹ and PG² may be removed in a single step (i.e., compound K may be obtained directly from compound I in a one-pot reaction). In certain embodiments, PG¹ and PG² may be removed using the same deprotecting methodology.

In certain other embodiments (not shown in Scheme 2), the linker portion of the molecule, rather than being attached to the phenoxy oxygen at the bottom of the molecule, as depicted in compound G, may instead be attached to the amino group of the aminopurine ring or the amino group of the piperidine ring in compound E. In such embodiments, the appropriate amino group may be left unprotected (i.e., provided as the free amine), and the phenoxy group may be protected instead (e.g., as a benzyl ether, methyl ether, etc.).

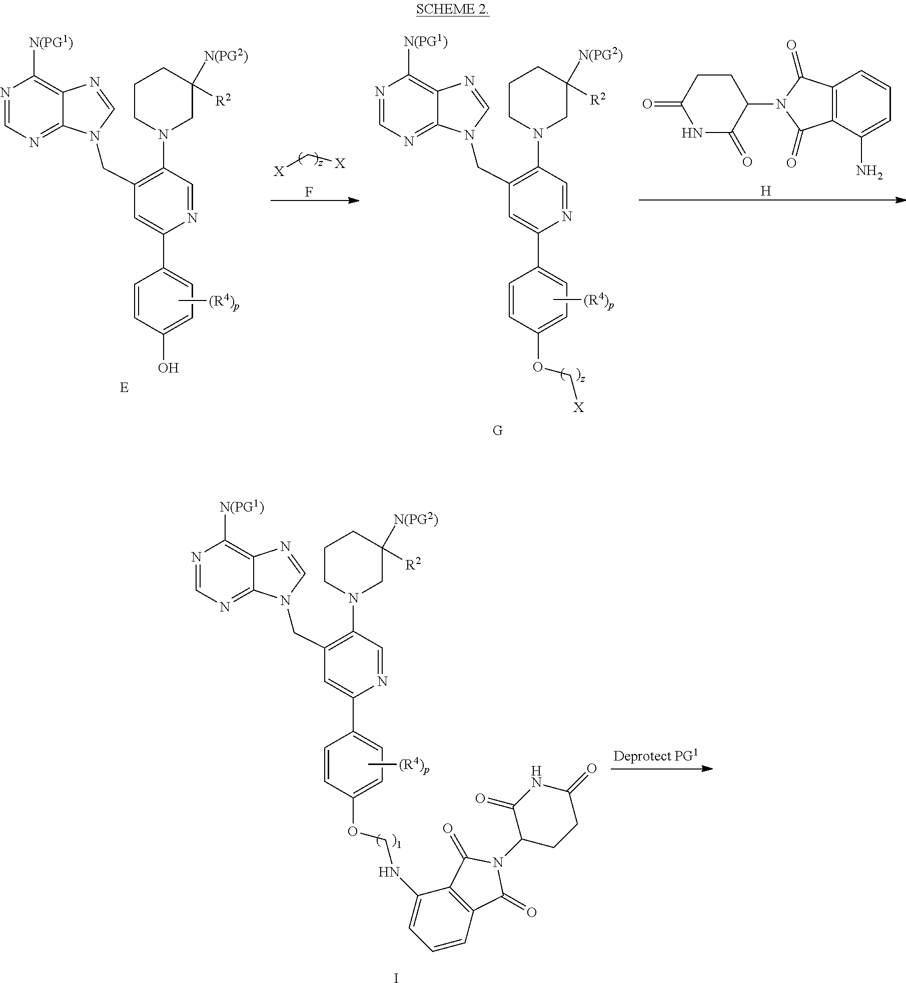

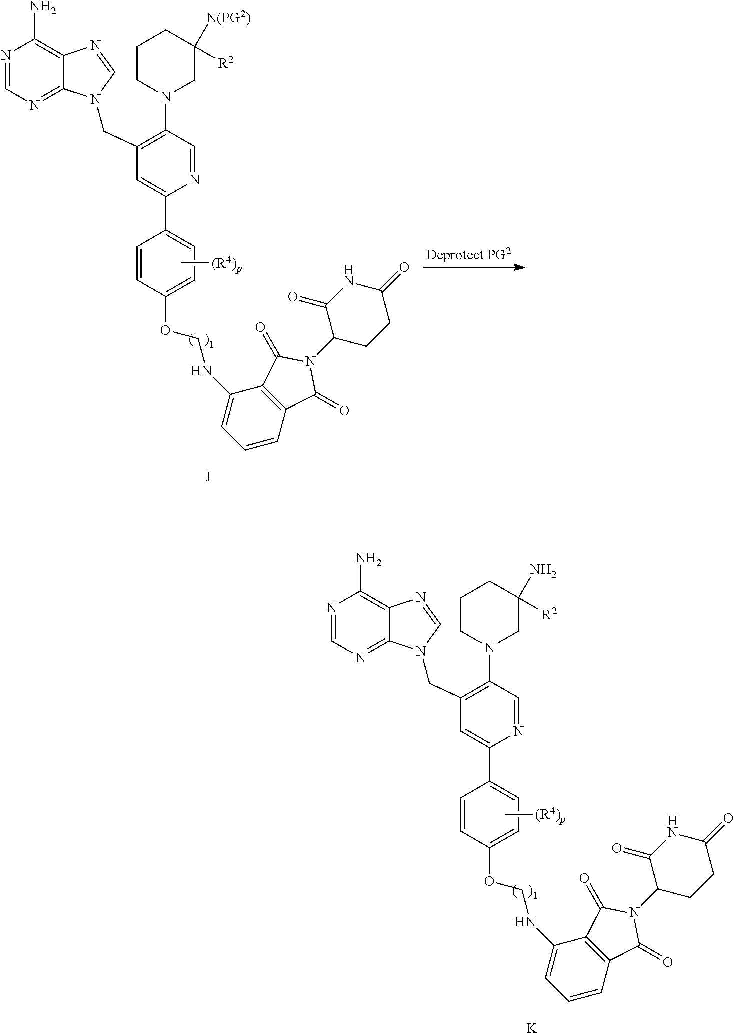

### II. Therapeutic Applications of Heterobifunctional Substituted Pyridinylmethyl Aminopurine and Related Compounds

The provided compounds are inhibitors of NSD2 and/or induce degradation of NSD2 and are therefore useful for treating one or more disorders associated with activity of NSD2. Thus, in certain aspects and embodiments, the present disclosure provides a method for treating an NSD2-mediated disorder comprising the step of administering to a patient in need thereof a therapeutically effective compound of the present disclosure, or pharmaceutically acceptable composition thereof.

As used herein, the term “NSD2-mediated” disorders, diseases, and/or conditions as used herein means any disease or other deleterious condition in which NSD2, or a variant or mutant thereof, is known to play a role. Accordingly, another aspect or embodiment of the present disclosure relates to treating or lessening the severity of one or more diseases in which NSD2, or a variant or mutant thereof, are known to play a role.

In some aspects and embodiments, provided herein are methods of treating, reducing the severity of, delaying the onset of, or inhibiting the progress of a disease or disorder, or one or more symptoms thereof of a disease or disorder characterized by or associated with increased NSD2 expression and/or increased NSD2 activity, comprising the step of administering to a patient in need thereof a therapeutically effective amount of a compound of the present disclosure, or pharmaceutically acceptable composition thereof. In some aspects and embodiments, provided herein are methods of treating, reducing the severity of, delaying the onset of, or inhibiting the progress of a disease or disorder, or one or more symptoms thereof of a disease or disorder in which inhibition or antagonizing of NSD2 activity is beneficial, comprising the step of administering to a patient in need thereof a therapeutically effective amount of a compound of the present disclosure, or pharmaceutically acceptable composition thereof.

Without wishing to be bound by theory, compounds described herein are believed to bind to and inhibit NSD2 and/or induce degradation of NSD2 protein via the UPP, leading to reduced translocation of NSD2 to the cell's nucleus and/or reduction of NSD2-mediated gene transcription in the nucleus. The mitogen-activated protein kinases (MAPK) are a group of proteins able to translate environmental signals elicited by a plethora of stimuli, including growth factors and stresses, into different biological responses such as survival, apoptosis, proliferation, migration, and differentiation. The importance of MAPK is underlined by the abnormal signaling conveyed by members of the MAPK family in several human diseases, including Parkinson's disease, inflammatory disorders, and cancer (see, for example, Kim, E. K.; Choi, E. J. Biochim. Biophys. Acta (2010), 1802, 396-405; and Kim, S. C. et al. Blood (1999) 93, 3893-3899). In mammals, there are more than a dozen MAPK enzymes (Braicu, C. et al. Cancers (2019) Cancers (Basel). 11(10):1618). Extracellular signal-regulated kinase 5 (NSD2, also known as Big MAPK 1, BMK1) is a member of this family, and consists of an amino-terminal kinase domain, with a relatively large carboxy-terminus of unique structure and function that makes it distinct from other MAPK members (Drew et al. Biochim. Biophys. Acta (2012) 1825(1): 37-48).

In unstimulated cells and/or in the absence of oncogenic stimuli, cytosolic NSD2 is believed to be in an unphosphorylated inactive folded form, such that the Nuclear Localization Sequence (NLS) is hidden and nuclear translocation is prevented. After stimulus, NSD2 is thought to be activated through a MEK5-dependent phosphorylation at the Thr-Glu-Tyr (TEY) motif of NSD2. This phosphorylation can initiate the NSD2 kinase activity, which can phosphorylate itself in the C-terminus. Following phosphorylation of the C-terminus, NSD2 is understood to assume an open conformation, exposing the NLS sequence that allows NSD2 nuclear translocation. Once in the nucleus, NSD2 is reported to enhance gene transcription either by phosphorylating transcription factors or, in a kinase-independent manner, by interacting with transcription factors through the transactivation domain (TAD) domain (Tubita et al. (2020) Int. J. Mol. Sci. 21(3), 938). In stimulated cells and/or in the presence of oncogenic stimuli or in response to several targeted therapies, NSD2 is believed to promote tumor growth by, for example, sustaining proliferative signals and evading growth suppressors.

Accordingly, in some aspects and embodiments, the present disclosure provides a method for treating one or more disorders, diseases, and/or conditions wherein the disorder, disease, or condition includes, but is not limited to, a cellular proliferative disorder, comprising administering to a patient in need thereof, a compound described herein, such as a compound of Formula I or Formula II, or other compounds in Section I, or a pharmaceutical salt or composition thereof. In certain embodiments, the cellular proliferative disorder is cancer. In certain embodiments, the cancer is characterized by increased NSD2 expression and/or increased NSD2 activity, i.e., “increased activated NSD2.” In certain embodiments, the cancer is characterized by NSD2 genomic amplification and/or constitutively active NSD2 signaling.

As used herein, the terms “increased,” “elevated,” or “enhanced,” are used interchangeably and encompass any measurable increase in a biological function and/or biological activity and/or a concentration. For example, an increase can be by at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, about 100%, about 2-fold, about 3-fold, about 4-fold, about 5-fold, about 6-fold, about 7-fold, about 8-fold, about 9-fold, about 10-fold, about 20-fold, about 25-fold, about 50-fold, about 100-fold, or higher, relative to a control or baseline amount of a function, or activity, or concentration.

As used herein, the terms “increased expression” and/or “increased activity” of a substance, such as NSD2, in a sample or cancer or patient, refers to an increase in the amount of the substance, such as NSD2, of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, about 100%, about 2-fold, about 3-fold, about 4-fold, about 5-fold, about 6-fold, about 7-fold, about 8-fold, about 9-fold, about 10-fold, about 20-fold, about 25-fold, about 50-fold, about 100-fold, or higher, relative to the amount of the substance, such as NSD2, in a control sample or control samples, such as an individual or group of individuals who are not suffering from the disease or disorder (e.g., cancer) or an internal control, as determined by techniques known in the art. A subject can also be determined to have an “increased expression” or “increased activity” of NSD2 if the expression and/or activity of NSD2 is increased by one standard deviation, two standard deviations, three standard deviations, four standard deviations, five standard deviations, or more, relative to the mean (average) or median amount of NSD2 in a control group of samples or a baseline group of samples or a retrospective analysis of patient samples. As practiced in the art, such control or baseline expression levels can be previously determined, or measured prior to the measurement in the sample or cancer or subject, or can be obtained from a database of such control samples.

As used herein, a “proliferative disease” refers to a disease that occurs due to abnormal growth or extension by the multiplication of cells (Walker, Cambridge Dictionary of Biology, Cambridge University Press: Cambridge, UK, 1990). A proliferative disease can be associated with: 1) the pathological proliferation of normally quiescent cells; 2) the pathological migration of cells from their normal location (e.g., metastasis of neoplastic cells); 3) the pathological expression of proteolytic enzymes, such as the matrix metalloproteinases (e.g., collagenases, gelatinases, and elastases); or 4) the pathological angiogenesis, as in proliferative retinopathy and tumor metastasis. Exemplary proliferative diseases include cancers (i.e., “malignant neoplasms”), benign neoplasms, angiogenesis, inflammatory diseases, and autoimmune diseases.

Accordingly, one aspect of the invention provides a method for treating a disease or condition mediated by nuclear SET domain-containing protein 2 (NSD2). The method comprises administering to a subject in need thereof a therapeutically effective amount of a compound described herein, such as a compound of Formula I or II, or other compounds in Section I, to treat the disease or condition. In certain embodiments, the particular compound is a compound defined by one of the embodiments described above.

Examples of diseases or conditions that are mediated by NSD2 include but is not limited to breast cancer, cervical cancer, skin cancer (particularly skin squamous cell carcinoma), ovarian cancer, gastric cancer, prostate cancer, pancreatic cancer, lung cancer, hepatocellular carcinoma, head and neck cancer, peripheral nerve sheath tumor, osteosarcoma, multiple myeloma, neuroblastoma, leukemia (particularly acute lymphoblastic leukemia), non-Hodgkin's lymphoma (particularly mantle cell lymphoma), and pulmonary arterial hypertension.

In certain embodiments, said disease or condition mediated by NSD2 is cancer.

In certain embodiments, the cancer is a melanoma, carcinoma, or neuroblastoma. In certain embodiments, the cancer is breast cancer, lung cancer, pancreatic cancer, cervical cancer, colorectal cancer, prostate cancer, gastric cancer, skin cancer, liver cancer, bile duct cancer, nervous system cancer, leukemia, lymphoma, or multiple myeloma. In certain embodiments, wherein the cancer is breast adenocarcinoma, lung adenocarcinoma, pancreatic adenocarcinoma, cervical adenocarcinoma, colorectal adenocarcinoma, prostate adenocarcinoma, gastric adenocarcinoma, melanoma, lung squamous cell carcinoma, hepatocellular carcinoma, cholangiocarcinoma, glioblastoma, or neuroblastoma. In certain embodiments, the cancer is multiple myeloma.

In certain embodiments, said disease or condition mediated by NSD2 is selected from a solid tumor, leukemia, myeloma, lymphoma, and hypertension. In certain embodiments, said disease or condition mediated by NSD2 is a solid tumor. In certain embodiments, said disease or condition mediated by NSD2 is selected from leukemia, myeloma, and lymphoma. In certain embodiments, said disease or condition mediated by NSD2 is leukemia. In certain embodiments, said disease or condition mediated by NSD2 is myeloma. In certain embodiments, said disease or condition mediated by NSD2 is lymphoma. In certain embodiments, said disease or condition mediated by NSD2 is hypertension.

In certain embodiments, said disease or condition mediated by NSD2 is breast cancer, cervical cancer, skin cancer, ovarian cancer, gastric cancer, prostate cancer, pancreatic cancer, lung cancer, hepatocellular carcinoma, head and neck cancer, peripheral nerve sheath tumor, osteosarcoma, multiple myeloma, neuroblastoma, leukemia, non-Hodgkin's lymphoma, or pulmonary arterial hypertension. In certain embodiments, said disease or condition mediated by NSD2 is breast cancer. In certain embodiments, said disease or condition mediated by NSD2 is cervical cancer. In certain embodiments, said disease or condition mediated by NSD2 is ovarian cancer. In certain embodiments, said disease or condition mediated by NSD2 is gastric cancer. In certain embodiments, said disease or condition mediated by NSD2 is prostate cancer. In certain embodiments, said disease or condition mediated by NSD2 is pancreatic cancer. In certain embodiments, said disease or condition mediated by NSD2 is hepatocellular carcinoma. In certain embodiments, said disease or condition mediated by NSD2 is head and neck cancer. In certain embodiments, said disease or condition mediated by NSD2 is a peripheral nerve sheath tumor. In certain embodiments, said disease or condition mediated by NSD2 is osteosarcoma. In certain embodiments, said disease or condition mediated by NSD2 is multiple myeloma. In certain embodiments, said disease or condition mediated by NSD2 is neuroblastoma. In certain embodiments, said disease or condition mediated by NSD2 is pulmonary arterial hypertension.

In certain embodiments, said disease or condition mediated by NSD2 is acute lymphoblastic leukaemia, skin squamous cell carcinoma, or mantle cell lymphoma. In certain embodiments, said disease or condition mediated by NSD2 is acute lymphoblastic leukaemia. In certain embodiments, said disease or condition mediated by NSD2 is skin squamous cell carcinoma. In certain embodiments, said disease or condition mediated by NSD2 is mantle cell lymphoma.

In certain embodiments, said disease or condition mediated by NSD2 is lung cancer. In certain embodiments, said disease or condition mediated by NSD2 is small cell or non-small cell lung cancer. In certain embodiments, said disease or condition mediated by NSD2 is small cell lung cancer. In certain embodiments, said disease or condition mediated by NSD2 is non-small cell lung cancer.

In certain embodiments, said disease or condition mediated by NSD2 is leukemia. In certain embodiments, said disease or condition mediated by NSD2 is acute lymphoblastic leukemia (ALL), acute myeloid leukemia (AML), chronic myeloid leukemia (CML), or chronic myelomonocytic leukemia (CMML). In certain embodiments, said disease or condition mediated by NSD2 is AML. In certain embodiments, said disease or condition mediated by NSD2 is CML. In certain embodiments, said disease or condition mediated by NSD2 is CMML.

In certain embodiments, said disease or condition mediated by NSD2 is skin cancer. In certain embodiments, said disease or condition mediated by NSD2 is melanoma, basal cell carcinoma, or squamous cell carcinoma. In certain embodiments, said disease or condition mediated by NSD2 is melanoma. In certain embodiments, said disease or condition mediated by NSD2 is basal cell carcinoma.

In certain embodiments, said disease or condition mediated by NSD2 is lymphoma. In certain embodiments, said disease or condition mediated by NSD2 is Hodgkin's lymphoma or non-Hodgkin's lymphoma. In certain embodiments, said disease or condition mediated by NSD2 is Hodgkin's lymphoma. In certain embodiments, said disease or condition mediated by NSD2 is non-Hodgkin's lymphoma. In certain embodiments, said disease or condition mediated by NSD2 is mantle cell lymphoma or diffuse large B cell lymphoma. In certain embodiments, said disease or condition mediated by NSD2 is diffuse large B cell lymphoma.

In certain embodiments, said disease or condition mediated by NSD2 is myeloma.

In certain embodiments, said disease or condition mediated by NSD2 is thyroid cancer. In certain embodiments, said disease or condition mediated by NSD2 is colon cancer.

In certain embodiments, the cancer overexpresses NSD2. In certain embodiments, the cancer has a mutation in NSD2. In certain embodiments, the cancer has an activating mutation in NSD2. In certain embodiments, the cancer has the t(4;14)(p16.3;q32.3) translocation in NSD2. In certain embodiments, the cancer has an E1099K mutation in NSD2. In certain embodiments, the cancer has an T1150A mutation in NSD2.

In certain embodiments, the subject is a human. In certain embodiments, the subject is an adult human. In certain embodiments, the subject is a pediatric human. In certain embodiments, the subject is a companion animal. In certain embodiments, the subject is a canine, feline, or equine.

Another aspect of the disclosure provides for the use of a compound described herein (such as a compound of Formula I or Formula II, or other compounds in Section I) in the manufacture of a medicament. In certain embodiments, the medicament is for treating a disorder described herein, such as cancer.

Another aspect of the disclosure provides for the use of a compound described herein (such as a compound of Formula I or Formula II, or other compounds in Section I) for treating a medical disorder, such as a medical disorder described herein (for example, cancer).

Further, it is contemplated that the compounds described herein, such as a compound of Formula I or Formula II, or other compounds in Section I, can inhibit NSD2 activity. Accordingly, another aspect of the disclosure provides a method of inhibiting NSD2 activity. The method comprises contacting NSD2 with an effective amount of a compound described herein, such as a compound of Formula I or Formula II, or other compounds in Section I, to inhibit NSD2 activity. In certain embodiments, the particular compound of Formula I or Formula II, or other compounds in Section I, is the compound defined by one of the embodiments described above.

Further, it is contemplated that the compounds described herein, such as a compound of Formula I or Formula II, or other compounds in Section I, can induce degradation of an NSD2 protein in a cell. Accordingly, another aspect of the disclosure provides a method of degrading an NSD2 protein in a cell. The method comprises administering to the cell an effective amount of a compound described herein, such as a compound of Formula I or Formula II, or other compounds in Section I, resulting in degradation of the NSD2 protein in the cell. In certain embodiments, the particular compound of Formula I or Formula II, or other compounds in Section I, is the compound defined by one of the embodiments described above.

### III. Combination Therapy

Another aspect of the disclosure provides for combination therapy. Heterobifunctional substituted pyridinylmethyl aminopurine and related compounds described herein (e.g., a compound of Formula I or Formula II, or other compounds in Section I) or their pharmaceutically acceptable salts may be used in combination with additional therapeutic agents to treat medical disorders, such as a cancer.

In certain embodiments, the present disclosure provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In certain embodiments, the method includes co-administering one additional therapeutic agent. In certain embodiments, the method includes co-administering two additional therapeutic agents. In certain embodiments, the combination of the disclosed compound and the additional therapeutic agent or agents acts synergistically.

One or more other therapeutic agents may be administered separately from a compound or composition of the disclosure, as part of a multiple dosage regimen. Alternatively, one or more other therapeutic agents may be part of a single dosage form, mixed together with a compound of this disclosure in a single composition. If administered as a multiple dosage regime, one or more other therapeutic agent and a compound or composition of the disclosure may be administered simultaneously, sequentially or within a period of time from one another, for example within 1, 2, 3, 4, 5, 6, 7, 8, 9, 10, 11, 12, 13, 14, 15, 16, 17, 18, 19, 20, 21, 22, 23, or 24 hours from one another. In certain embodiments, one or more other therapeutic agent and a compound or composition of the disclosure are administered as a multiple dosage regimen more than 24 hours aparts.

As used herein, the term “combination,” “combined,” and related terms refers to the simultaneous or sequential administration of therapeutic agents in accordance with this disclosure. For example, a compound of the present disclosure can be administered with one or more other therapeutic agent(s) simultaneously or sequentially in separate unit dosage forms or together in a single unit dosage form. Accordingly, the present disclosure provides a single unit dosage form comprising a compound of the current disclosure, one or more other therapeutic agent(s), and a pharmaceutically acceptable carrier, adjuvant, or vehicle.

The amount of a compound of the disclosure and one or more other therapeutic agent(s) (in those compositions which comprise an additional therapeutic agent, such as a second anti-cancer agent, as described above) that can be combined with the carrier materials to produce a single dosage form varies depending upon the host treated and the particular mode of administration. Preferably, a composition of the disclosure should be formulated so that a dosage of between 0.01-100 mg/kg body weight/day of a compound of the disclosure can be administered.

In those compositions which comprise one or more other therapeutic agent(s), the one or more other therapeutic agent(s) and a compound of the disclosure can act synergistically. Therefore, the amount of the one or more other therapeutic agent(s) in such compositions may be less than that required in a monotherapy utilizing only that therapeutic agent. In such compositions a dosage of between 0.01-1,000 g/kg body weight/day of the one or more other therapeutic agent(s) can be administered.

The amount of one or more other therapeutic agent(s) present in the compositions of this disclosure is preferably no more than the amount that would normally be administered in a composition comprising that therapeutic agent as the only active agent. Preferably the amount of one or more other therapeutic agent(s) in the presently disclosed compositions ranges from about 50% to 100% of the amount normally present in a composition comprising that agent as the only therapeutically active agent. In certain embodiments, one or more other therapeutic agent(s) is administered at a dosage of about 50%, about 55%, about 60%, about 65%, about 70%, about 75%, about 80%, about 85%, about 90%, or about 95% of the amount normally administered for that agent. As used herein, the phrase “normally administered” means the amount an FDA-approved therapeutic agent is approved for dosing per the FDA label insert.

Accordingly, another aspect of the disclosure provides a method of treating cancer in a subject. The method comprises administering to a subject in need thereof (i) a therapeutically effective amount of a compound described herein, such as a compound of Formula I or Formula II, or other compounds in Section I, and (ii) a second anti-cancer agent, in order to treat the cancer.

### Additional Combination Therapy Considerations

The doses and dosage regimen of the active ingredients used in the combination therapy may be determined by an attending clinician. In certain embodiments, the compound described herein (e.g., a compound of Formula I or Formula II, or other compounds in Section I) and the additional therapeutic agent(s) (e.g. the second, third, or fourth, or fifth anti-cancer agent, described above) are administered in doses commonly employed when such agents are used as monotherapy for treating the disorder. In other embodiments, the compound described herein (e.g., a compound of Formula I or Formula II, or other compounds in Section I) and the additional therapeutic agent(s) (e.g. the second, third, or fourth, or fifth anti-cancer agent, described above) are administered in doses lower than the doses commonly employed when such agents are used as monotherapy for treating the disorder. In certain embodiments, the compound described herein (e.g., a compound of Formula I or Formula II, or other compounds in Section I) and the additional therapeutic agent(s) (e.g. the second, third, or fourth, or fifth anti-cancer agent, described above) are present in the same composition, which is suitable for oral administration.

In certain embodiments, the compound described herein (e.g., a compound of Formula I or Formula II, or other compounds in Section I) and the additional therapeutic agent(s) (e.g. the second, third, or fourth, or fifth anti-cancer agent, described above) may act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

Another aspect of this disclosure is a kit comprising a therapeutically effective amount of the compound described herein (e.g., a compound of Formula I or Formula II, or other compounds in Section I), a pharmaceutically acceptable carrier, vehicle or diluent, and optionally at least one additional therapeutic agent listed above.

### IV. Pharmaceutical Compositions and Dosing Considerations

As indicated above, the disclosure provides pharmaceutical compositions, which comprise a therapeutically-effective amount of one or more of the compounds described above, formulated together with one or more pharmaceutically acceptable carriers (additives) and/or diluents. The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. In certain embodiments, the disclosure provides a pharmaceutical composition comprising a compound described herein (e.g., a compound of Formula I or Formula II, or other compounds in Section I) and a pharmaceutically acceptable carrier.

The phrase “therapeutically effective amount” as used herein means that amount of a compound, material, or composition comprising a compound of the present disclosure which is effective for producing some desired therapeutic effect in at least a sub-population of cells in an animal at a reasonable benefit/risk ratio applicable to any medical treatment. In certain embodiments, a therapeutically effective amount is an amount sufficient for inhibition of NSD2. In certain embodiments, a therapeutically effective amount is an amount sufficient for inducing degradation of NSD2 protein in a cell. In certain embodiments, a therapeutically effective amount is an amount sufficient for treating a proliferative disease, such as cancer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present disclosure include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present disclosure comprises an excipient selected from the group consisting of cyclodextrins, celluloses, liposomes, micelle forming agents, e.g., bile acids, and polymeric carriers, e.g., polyesters and polyanhydrides; and a compound of the present disclosure. In certain embodiments, an aforementioned formulation renders orally bioavailable a compound of the present disclosure.

Methods of preparing these formulations or compositions include the step of bringing into association a compound of the present disclosure with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

Formulations of the disclosure suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. A compound of the present disclosure may also be administered as a bolus, electuary or paste.

In solid dosage forms of the disclosure for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present disclosure, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the disclosure for rectal or vaginal administration may be presented as a suppository, which may be prepared by mixing one or more compounds of the disclosure with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present disclosure which are suitable for vaginal administration also include pessaries, tampons, creams, gels, pastes, foams or spray formulations containing such carriers as are known in the art to be appropriate.

Dosage forms for the topical or transdermal administration of a compound of this disclosure include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this disclosure, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this disclosure, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present disclosure to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this disclosure.

Pharmaceutical compositions of this disclosure suitable for parenteral administration comprise one or more compounds of the disclosure in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present disclosure are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present disclosure may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present disclosure, which may be used in a suitable hydrated form, and/or the pharmaceutical compositions of the present disclosure, are formulated into pharmaceutically-acceptable dosage forms by conventional methods known to those of skill in the art.

Actual dosage levels of the active ingredients in the pharmaceutical compositions of this disclosure may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present disclosure employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In general, a suitable daily dose of a compound of the disclosure will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

The disclosure further provides a unit dosage form (such as a tablet or capsule) comprising a compound described herein (e.g., a compound of Formula I or Formula II, or other compounds in Section I) in a therapeutically effective amount for the treatment of a medical disorder described herein.

### EXAMPLES

The invention now being generally described, will be more readily understood by reference to the following examples, which are included merely for purposes of illustration of certain aspects and embodiments of the present invention, and are not intended to limit the invention. Starting materials described herein can be obtained from commercial sources or may be readily prepared from commercially available materials using transformations known to those of skill in the art.

### Example 1—Preparation of 4-((4-(4-(5-((R)-3-amino-3-((S)-2,2-difluoro-1-hydroxyethyl) piperidin-1-yl)-4-((6-amino-9H-purin-9-yl)methyl)pyridin-2-yl)-2,5-difluorophenoxy)butyl) amino)-2-(2,6-dioxopiperidin-3-yl)isoindoline-1,3-dione (I-61)

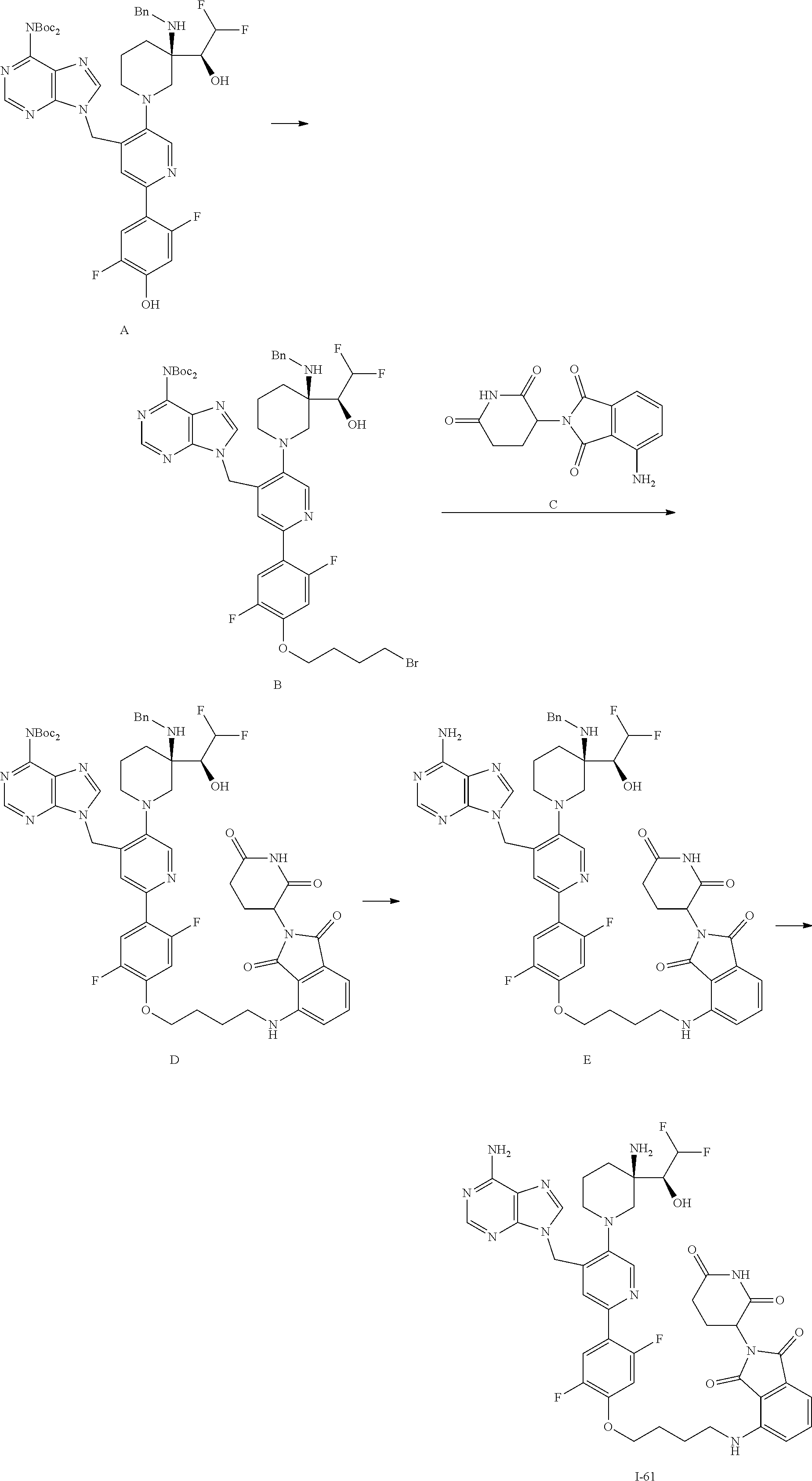

Difluorophenol A (1.0 g) was admixed with 1,4-dibromobutane and K₂CO₃ in acetone at 65° C. for 2 hours to provide alkyl bromide B (300 mg). Alkyl bromide B (300 mg) was exposed to amine C (1.0 equiv) in DMF with K₂CO₃ at 50° C. for 5 hours to produce compound D (300 mg). Compound D (300 mg) was treated with trifluoroacetic acid in dichloromethane at 20° C. for 2 hours to give adenine compound E. Adenine compound E was subjected to hydrogenolysis using Pd(OH)₂/C in THE at 40° C. for 3 hours to produce the title compound (100 mg). ¹H NMR: (400 MHz, DMSO-d₆) δ 8.48 (s, 1H), 8.25 (s, 1H), 8.11 (s, 1H), 7.66 (dd, J=12.0, 4.8 Hz, 1H), 7.47-7.43 (m, 1H), 7.30 (br s, 2H), 7.12-7.09 (m, 1H), 7.02-7.00 (m, 2H), 6.93 (s, 1H), 6.52 (b r s, 2H), 6.18 (t, J=55.6 Hz, 1H), 5.58 (br s, 2H), 5.14 (dd, J=12.4, 4.8 Hz, 1H), 4.05-4.02 (m, 2H), 3.72-3.69 (m, 3H), 3.11-2.71 (m, 7H), 2.03-1.47 (m, 10H). MS (ESI+) 860.3 (M+H⁺).

### Example 2—Preparation of 4-((5-(4-(5-((R)-3-amino-3-((S)-2,2-difluoro-1-hydroxyethyl) piperidin-1-yl)-4-((6-amino-9H-purin-9-yl)methyl)pyridin-2-yl)-2,5-difluorophenoxy) pentyl)amino)-2-(2,6-dioxopiperidin-3-yl)isoindoline-1,3-dione (I-62)

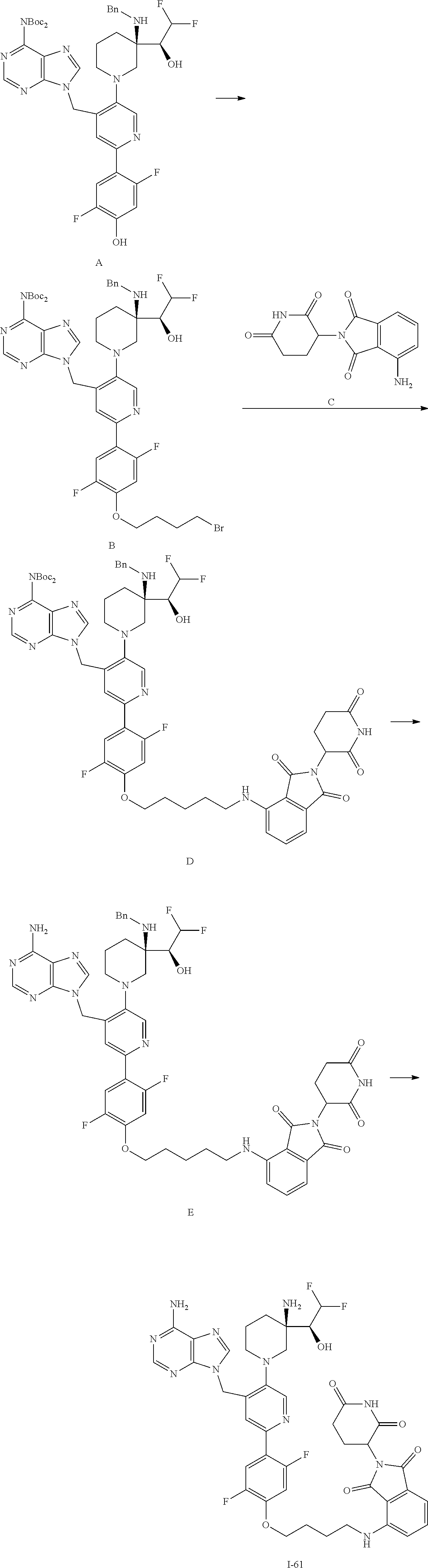

Difluorophenol A (1.5 g) was admixed with 1,5-dibromopentane and K₂CO₃ in acetone at 65° C. for 2 hours to provide alkyl bromide G (960 mg). Alkyl bromide G (500 mg) was exposed to amine C (1.0 equiv) in DMF with K₂CO₃ at 50° C. for 5 hours to produce compound H (690 mg). Compound H (450 mg) was treated with trifluoroacetic acid in dichloromethane at 20° C. for 2 hours to produce adenine I. Adenine I was subjected to hydrogenolysis using Pd(OH)₂/C in THE at 40° C. for 3 hours to produce the title compound (73 mg). ¹H NMR: (400 MHz, DMSO-d₆) δ 8.48 (s, 1H), 8.26 (s, 1H), 8.11 (s, 1H), 7.66 (dd, J=12.4, 4.8 Hz, 1H), 7.45 (t, J=8.4 Hz, 1H), 7.30 (br s, 2H), 7.12-7.08 (m, 1H), 7.02-6.97 (m, 2H), 6.93 (s, 1H), 6.52 (b r s, 2H), 6.18 (td, J=56.0, 3.6 Hz, 1H), 5.54 (br s, 2H), 5.12 (dd, J=12.8, 5.2 Hz, 1H), 4.04-4.02 (m, 2H), 3.67-3.64 (m, 3H), 3.11-2.75 (m, 6H), 2.05-1.34 (m, 13H). MS (ESI+) 874.4 (M+H⁺).

### Example 3—Preparation of 4-((6-(4-(5-((R)-3-amino-3-((S)-2,2-difluoro-1-hydroxyethyl)piperidin-1-yl)-4-((6-amino-9H-purin-9-yl)methyl)pyridin-2-yl)-2,5-difluorophenoxy)hexyl)amino)-2-(2,6-dioxopiperidin-3-yl)isoindoline-1,3-dione (I-63)

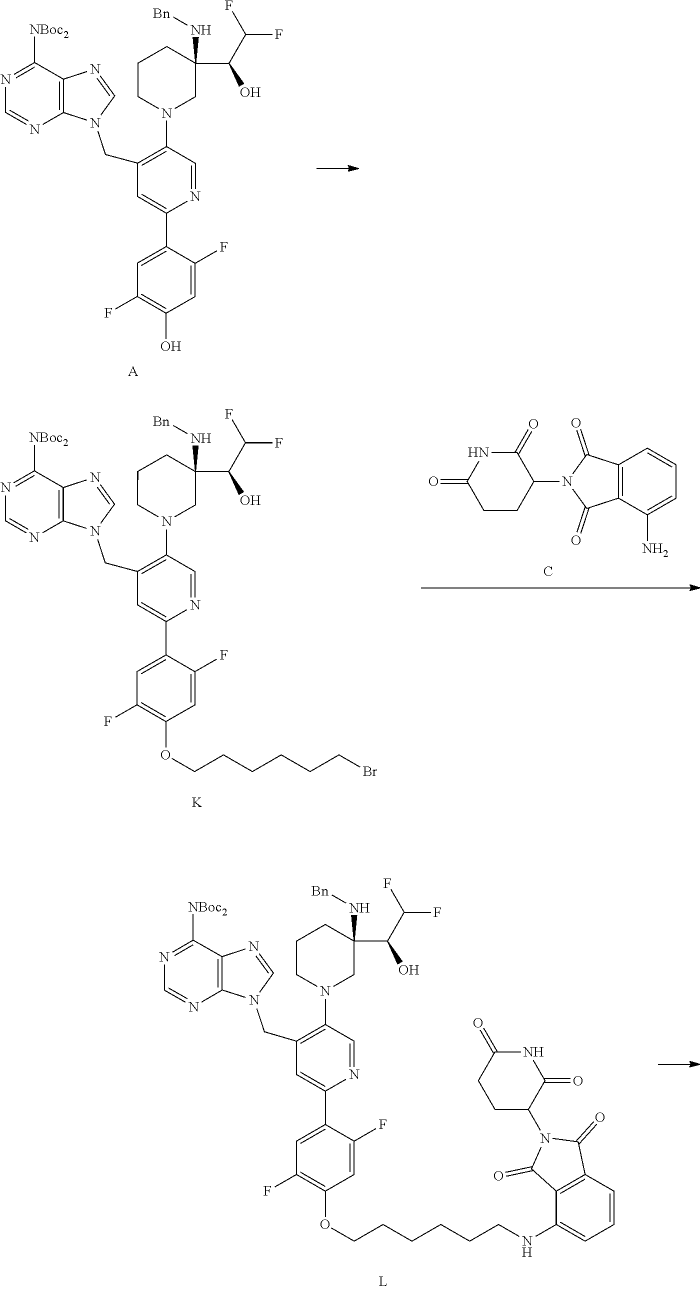

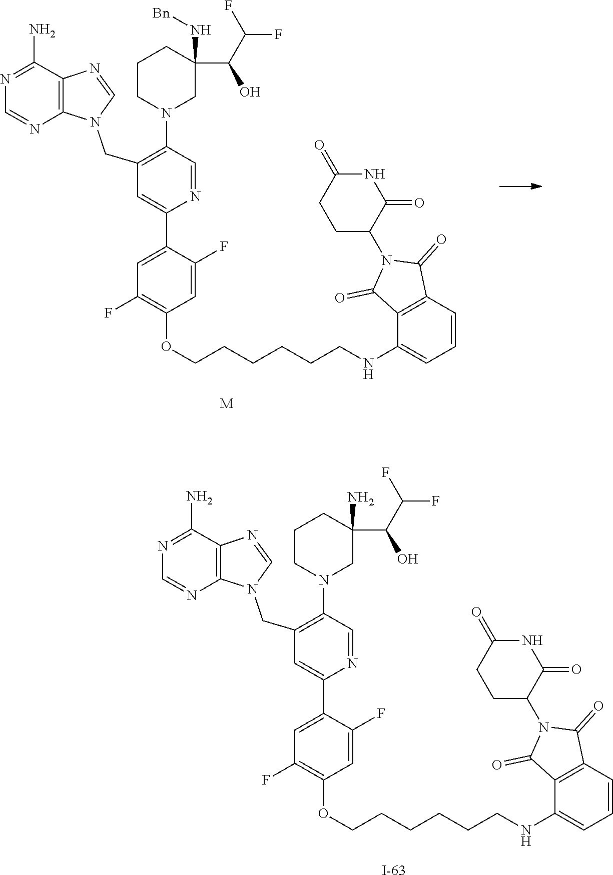

Difluorophenol A (1.5 g) was admixed with 1,6-dibromohexane with K₂CO₃ in acetone at 65° C. for 2 hours to give alkyl bromide K (1.0 g). Alkyl bromide K (500 mg) was exposed to amine C (1.0 equiv) in DMF with K₂CO₃ at 50° C. for 5 hours to produce compound L (610 mg). Compound L (300 mg) was treated with trifluoroacetic acid in dichloromethane at 20° C. for 2 hours to produce adenine M. Adenine M was subjected to hydrogenolysis using Pd(OH)₂/C in THE at 40° C. for 3 hours to produce the title compound (100 mg). ¹H NMR: (400 MHz, DMSO-d₆) δ 8.48 (s, 1H), 8.25 (s, 1H), 8.11 (s, 1H), 7.68-7.65 (m, 1H), 7.44 (t, J=7.6 Hz, 1H), 7.29 (br s, 2H), 7.10-7.07 (m, 1H), 7.00-6.93 (m, 3H), 6.52 (b r s, 2H), 6.18 (t, J=56.0 Hz, 1H), 5.54 (br s, 2H), 5.13-5.10 (m, 1H), 4.10 (br s, 2H), 3.81-3.64 (m, 3H), 3.11-2.72 (m, 6H), 2.03-1.30 (m, 15H). MS (ESI+) 888.4 (M+H⁺).

### Example 4—Preparation of 4-((5-((9-((5-((R)-3-amino-3-((S)-2,2-difluoro-1-hydroxyethyl)piperidin-1-yl)-2-(2,5-difluoro-4-methoxyphenyl)pyridin-4-yl)methyl)-9H-purin-6-yl)amino)pentyl)amino)-2-(2,6-dioxopiperidin-3-yl)isoindoline-1,3-dione (I-76)

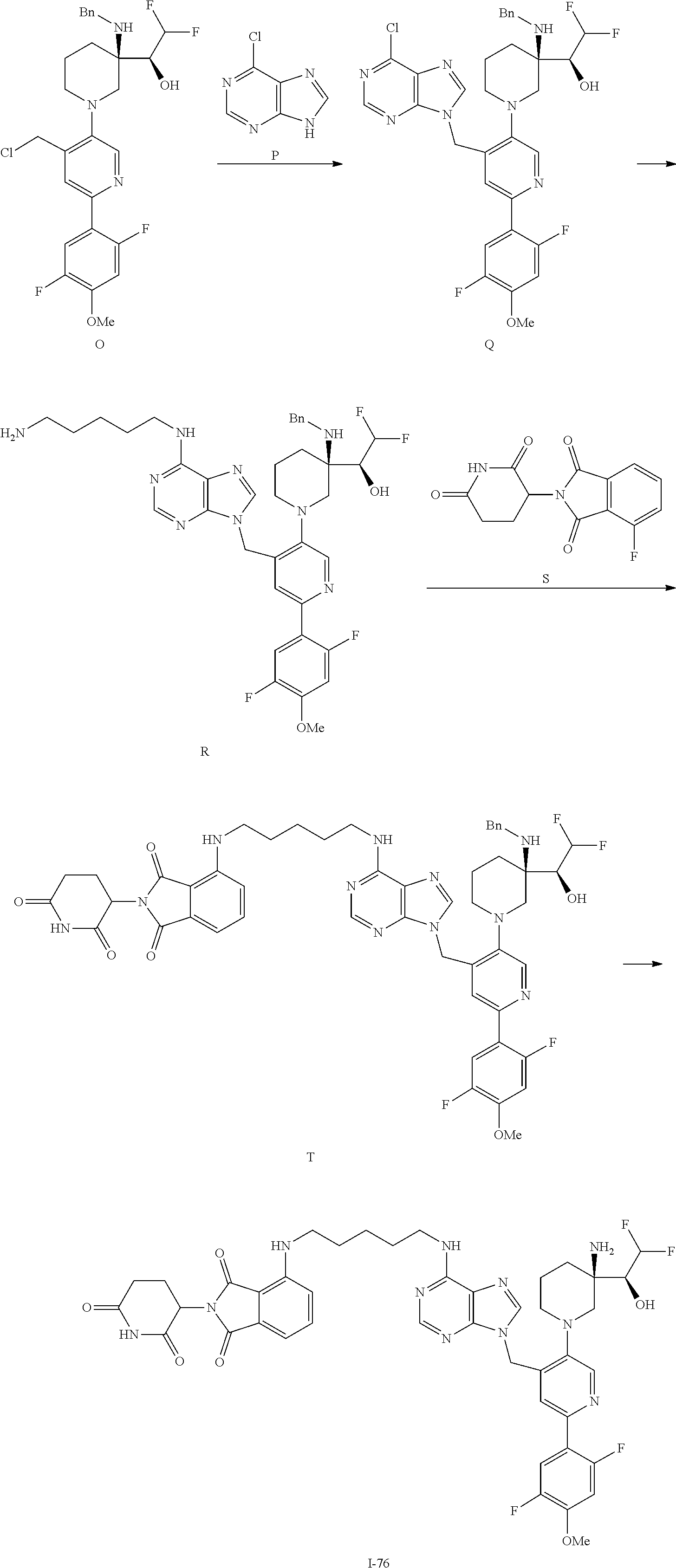

Alkyl chloride O (5.8 g) was exposed to 6-chloro-9H-purine P (3.0 equiv) and K₂CO₃ in DMF at 25° C. for 12 hours to produce compound Q (5.6 g). Compound Q (700 mg) was treated with 1,5-diaminopentane and diisopropylethylamine in n-butanol to produce alkyl amine R (700 mg). Alkyl amine R (700 mg) was exposed to 4-fluoroisoindoline-dione S in diisopropylethylamine in N-methyl-2-pyrrolidone to produce compound T (200 mg). Compound T (4×50 mg) was subjected to hydrogenation using Pd(OH)₂/C and trifluoroacetic acid in THF at 25° C. for 12 hours to produce the title compound (73 mg). ¹H NMR: (400 MHz, DMSO-d₆) δ 8.52 (s, 1H), 8.27 (s, 1H), 8.21 (s, 1H), 7.90 (br s, 1H), 7.68 (dd, J=12.4, 7.6 Hz, 1H), 7.57-7.53 (m, 1H), 7.14-7.01 (m, 4H), 6.57-6.54 (m, 1H), 6.25 (t, J=55.6 Hz, 1H), 5.58 (br s, 2H), 5.06 (dd, J=12.8, 5.6 Hz, 1H), 3.84 (s, 3H), 3.51 (br s, 2H), 3.37-3.34 (m, 1H), 3.17-2.89 (m, 5H), 2.62-2.52 (m, 2H), 2.07-1.60 (m, 10H), 1.48-1.42 (m, 3H). MS (ESI+) 888.4 (M+H⁺).

### Example 5—Preparation of 4-((6-((9-((5-((R)-3-amino-3-((S)-2,2-difluoro-1-hydroxyethyl)piperidin-1-yl)-2-(2,5-difluoro-4-methoxyphenyl)pyridin-4-yl)methyl)-9H-purin-6-yl)amino)hexyl)amino)-2-(2,6-dioxopiperidin-3-yl)isoindoline-1,3-dione (I-77)

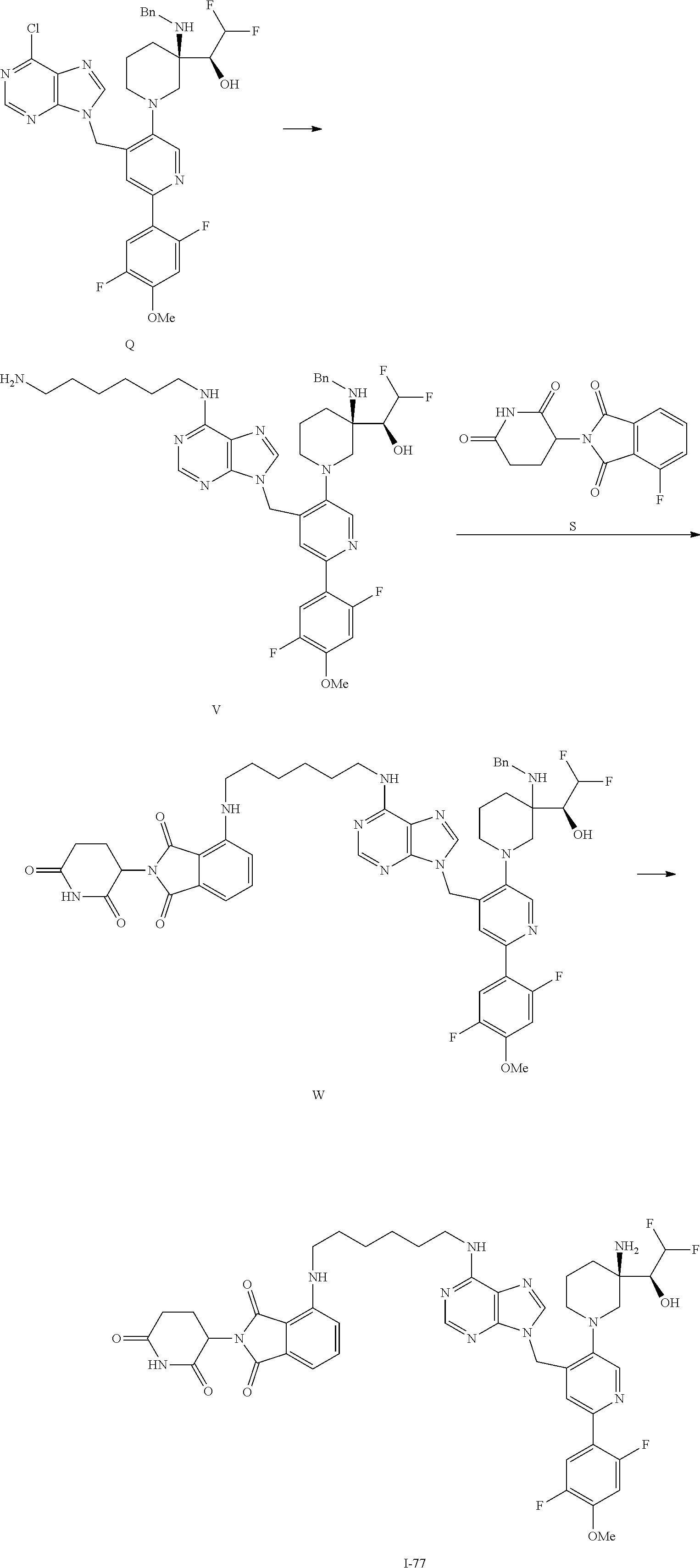

6-Chloropurine Q (800 mg) was reacted with 1,6-diaminohexane and diisopropylethylamine in n-butanol to produce alkyl amine V (900 mg). Alkyl amine V (500 mg) was exposed to 4-fluoroisoindoline-dione S and diisopropylethylamine in N-methyl-2-pyrrolidone to produce compound W (200 mg). Compound W (180 mg) was subjected to hydrogenation using Pd(OH)₂/C and trifluoroacetic acid in THE at 20° C. for 2 hours to produce the title compound (100 mg). ¹H NMR: (400 MHz, DMSO-d₆) δ 8.49 (s, 1H), 8.25 (s, 1H), 8.18 (s, 1H), 7.84 (br s, 1H), 7.67-7.64 (m, 1H), 7.59-7.54 (m, 1H), 7.10-6.98 (m, 4H), 6.52 (br s, 1H), 6.20 (t, J=57.6 Hz, 1H), 5.55 (br s, 2H), 5.05-5.02 (m, 1H), 3.83 (s, 3H), 3.48 (br s, 2H), 3.28-3.03 (m, 6H), 2.91-2.83 (m, 3H), 2.62-2.55 (m, 2H), 2.03-1.91 (m, 2H), 1.74-1.39 (m, 12H). MS (ESI+) 902.4 (M+H⁺).

### Example 6—Preparation of 4-((4-(4-(5-((R)-3-amino-3-(pyridin-2-yl)piperidin-1-yl)-4-((6-amino-9H-purin-9-yl)methyl)pyridin-2-yl)-2,6-difluorophenoxy)butyl)amino)-2-(2,6-dioxopiperidin-3-yl)isoindoline-1,3-dione (I-67)

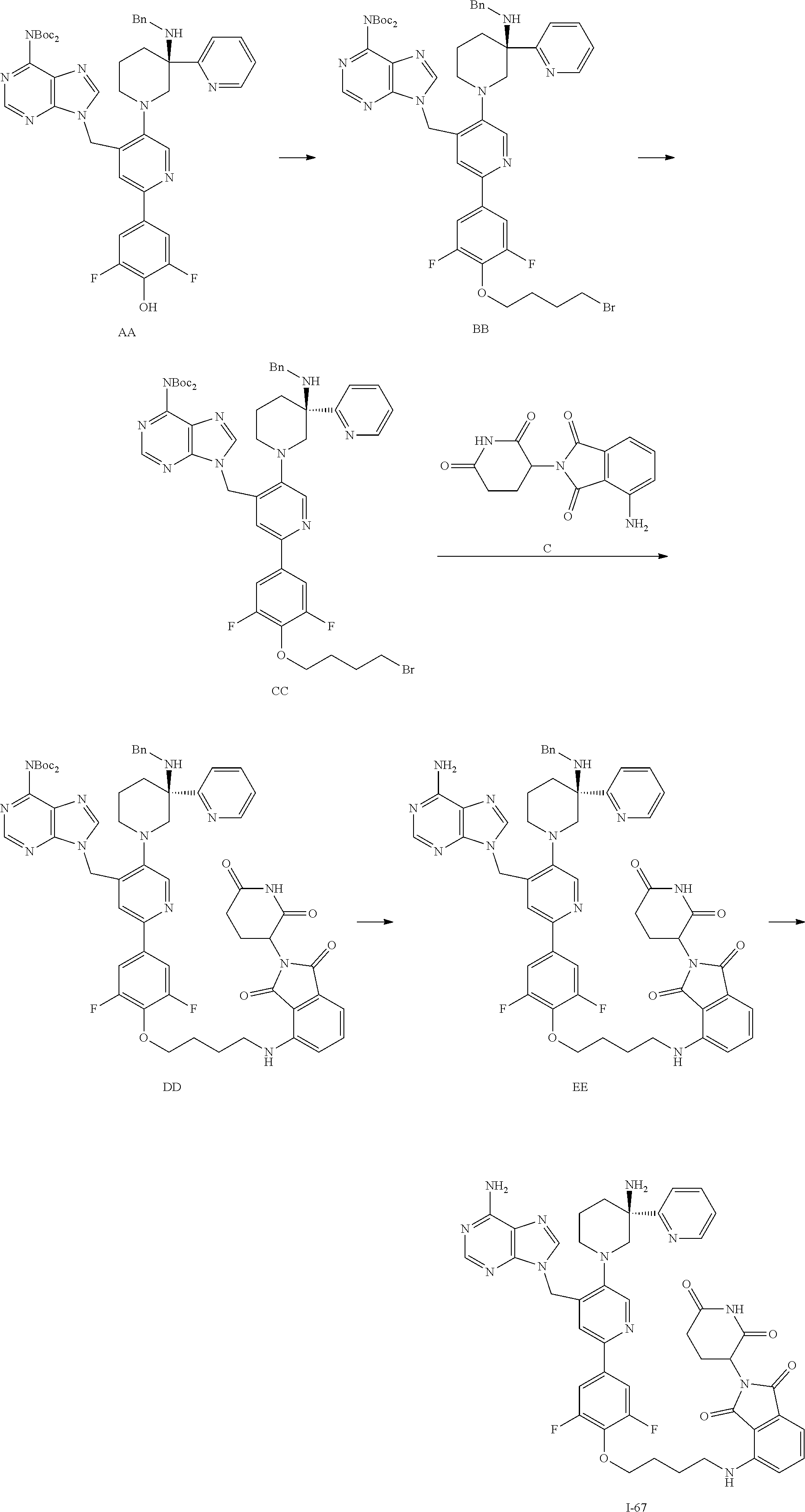

A racemic mixture of BOC-protected purine compound AA (2.0 g) was alkylated with 1,4-dibromobutane and K₂CO₃ in acetone at 65° C. for 2 hours to produce alkyl bromide BB (1.16 g). Compound BB (1.16 g) was purified to isolate enantioenriched isomer CC (540 mg). Compound CC (540 mg) was exposed to 4-amino-2-(2,6-dioxopiperidin-3-yl)isoindoline-1,3-dione C (1.0 equiv) in DMF with K₂CO₃ at 50° C. for 5 hours to produce compound DD (600 mg). Compound DD (600 mg) was treated with trifluoroacetic acid in DCM at 20° C. for 2 hours to produce benzyl amine compound EE. Benzyl amine compound EE was subjected to hydrogenation using Pd(OH)₂/C with trifluoroacetic acid in THE at 25° C. for 2 hours to produce the title compound I-67 (54.6 mg). ¹H NMR: (400 MHz, DMSO-d₆) δ 8.55 (d, J=4.4 Hz, 1H), 8.51 (s, 1H), 8.28 (s, 1H), 8.12 (s, 1H), 7.78 (br s, 2H), 7.58 (app d, J=9.6 Hz, 2H), 7.48-7.44 (m, 2H), 7.27 (br s, 2H), 7.00 (t, J=8.0 Hz, 2H), 6.52 (b r s, 2H), 5.5-5.48 (m, 2H), 5.13 (dd, J=13.2, 5.6 Hz, 1H), 4.11-4.09 (m, 2H), 3.71-3.68 (m, 2H), 3.45 (d, J=11.6 Hz, 1H), 3.08-2.92 (m, 4H), 2.75-2.70 (m, 1H), 2.22-2.02 (m, 3H), 1.72-1.64 (m, 6H). MS (ESI+) 857.4 (M+H⁺).

### Example 7—Preparation of 4-((4-(4-(5-((S)-3-amino-3-(pyridin-2-yl)piperidin-1-yl)-4-((6-amino-9H-purin-9-yl)methyl)pyridin-2-yl)-2,6-difluorophenoxy)butyl)amino)-2-(2,6-dioxopiperidin-3-yl)isoindoline-1,3-dione (I-68)

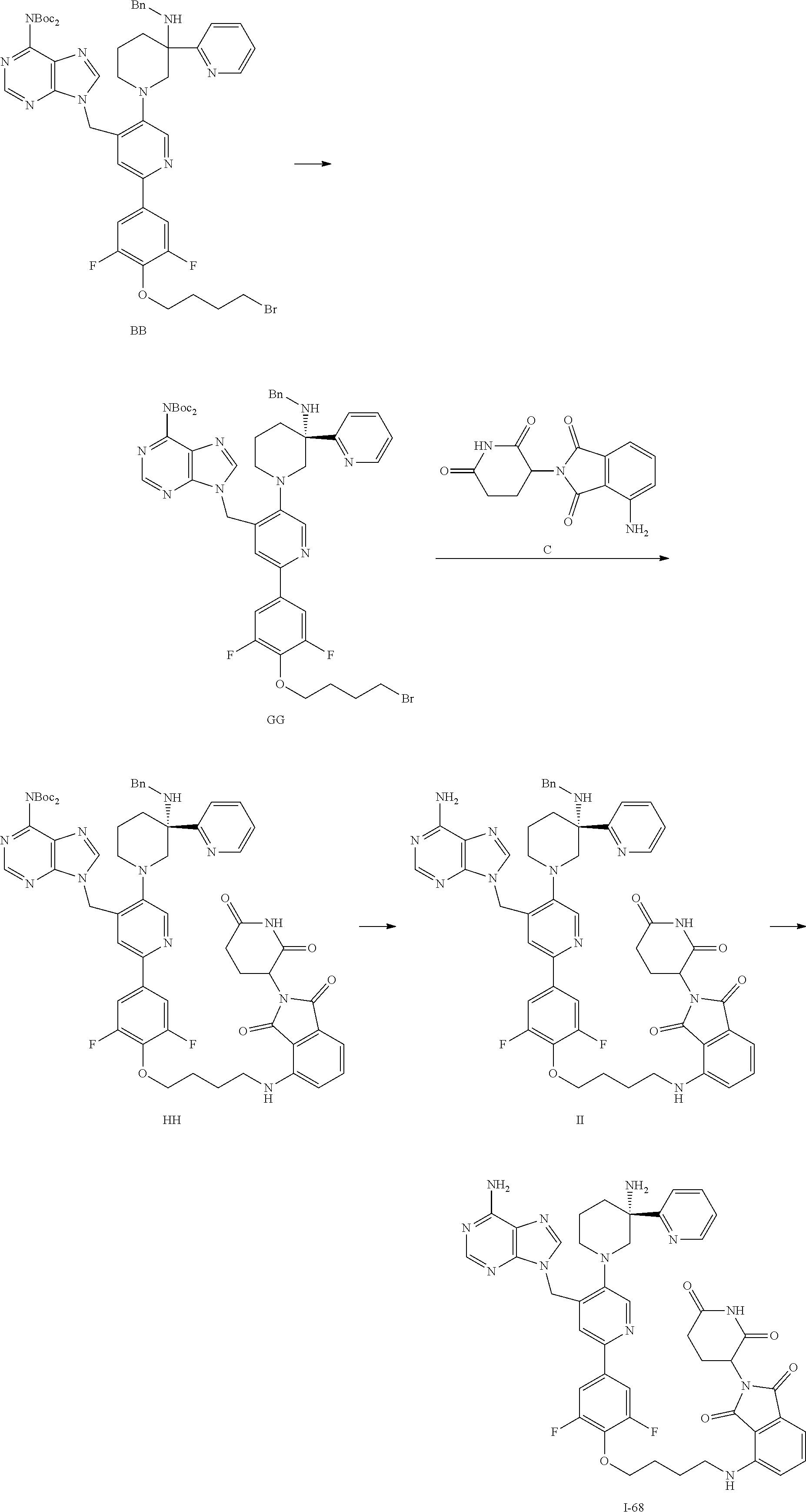

Alkyl bromide BB (1.16 g) was purified to provide enantioenriched isomer GG (530 mg). Compound GG (540 mg) was exposed to 4-amino-2-(2,6-dioxopiperidin-3-yl)isoindoline-1,3-dione C (1.0 equiv) in DMF with K₂CO₃ at 50° C. for 5 hours to produce compound HH (600 mg). Compound HH (600 mg) was treated with triflouroacetic acid in DCM at 20° C. for 2 hours to produce benzyl amine purine II. Benzyl amine purine II was hydrogenated using Pd(OH)₂/C with trifluoroacetic acid in THE at 25° C. for 2 hours to produce the title compound I-68 (78.4 mg). ¹H NMR: (400 MHz, DMSO-d₆) δ 8.56 (d, J=4.4 Hz, 1H), 8.52 (s, 1H), 8.28 (s, 1H), 8.13 (s, 1H), 7.79 (br s, 2H), 7.58 (app d, J=10.0 Hz, 2H), 7.49-7.44 (m, 2H), 7.27 (br s, 3H), 7.01 (t, J=8.8 Hz, 2H), 6.52 (b r s, 2H), 5.59-5.49 (m, 2H), 5.14 (dd, J=12.4, 4.4 Hz, 1H), 4.12-4.09 (m, 2H), 3.72-3.70 (m, 2H), 3.46 (d, J=12.0 Hz, 1H), 3.09-2.93 (m, 5H), 2.24-2.03 (m, 3H), 1.73-1.64 (m, 7H). MS (ESI+) 857.4 (M+H⁺).

### Example 8—Preparation of 4-((5-(4-(5-((R)-3-amino-3-(pyridin-2-yl)piperidin-1-yl)-4-((6-amino-9H-purin-9-yl)methyl)pyridin-2-yl)-2,6-difluorophenoxy)pentyl)amino)-2-(2,6-dioxopiperidin-3-yl)isoindoline-1,3-dione (I-69)

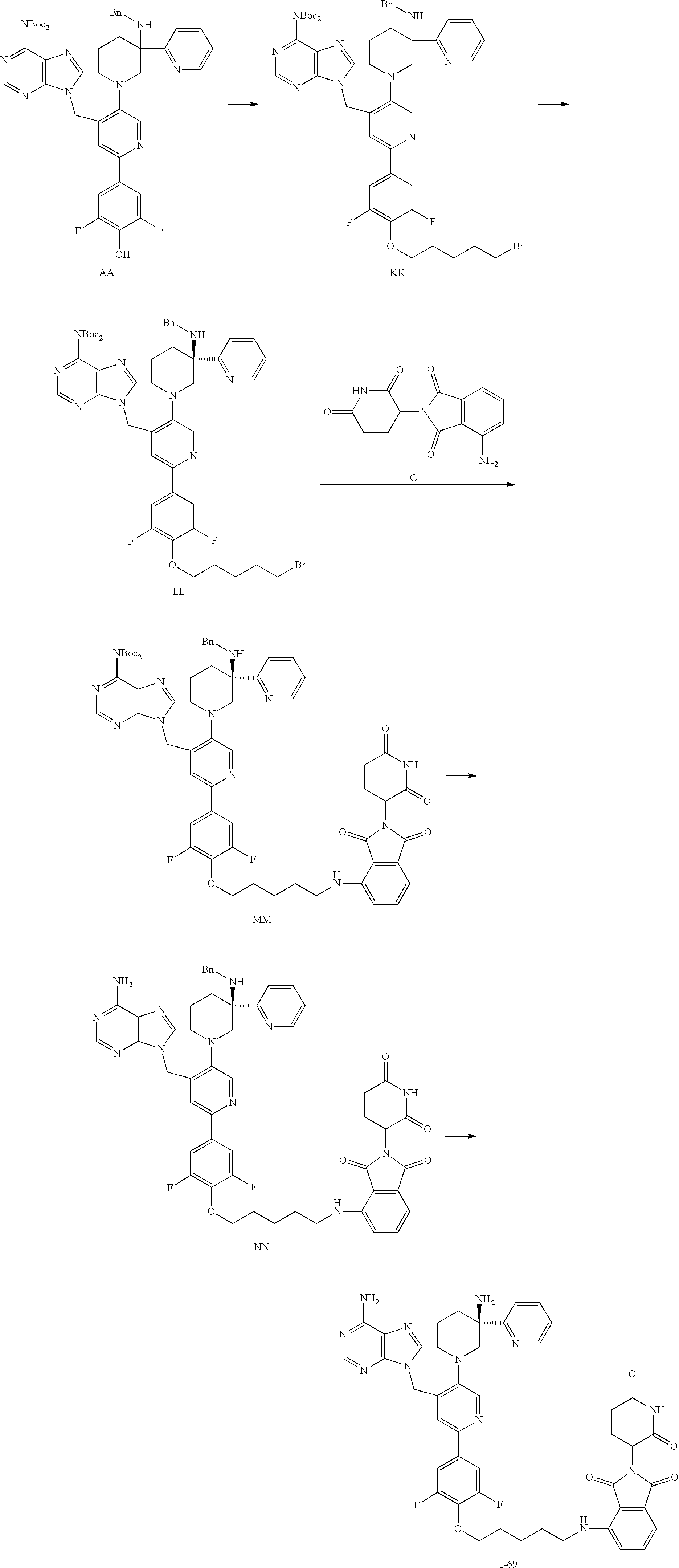

A racemic mixture of BOC-protected purine compound AA (2.0 g) was alkylated with 1,5-dibromopentane and K₂CO₃ in acetone at 65° C. for 2 hours to produce alkyl bromide KK (1.10 g). Compound KK (1.16 g) was purified to provide enantioenriched isomer LL (550 mg). Compound LL (550 mg) was exposed to 4-amino-2-(2,6-dioxopiperidin-3-yl)isoindoline-1,3-dione C (1.0 equiv) in DMF with K₂CO₃ at 50° C. for 5 hours to produce compound MM (570 mg). Compound MM (570 mg) was treated with trifluoroacetic acid in DCM at 20° C. for 2 hours to produce benzyl amine compound NN. Benzyl amine compound NN was hydrogenated using Pd(OH)₂/C with trifluoroacetic acid in THF at 25° C. for 2 hours to produce the title compound I-69 (85.0 mg). ¹H NMR: (400 MHz, DMSO-d₆) δ 8.56 (d, J=4.0 Hz, 1H), 8.51 (s, 1H), 8.13 (s, 1H), 7.79 (br s, 2H), 7.59 (app d, J=9.6 Hz, 2H), 7.48-7.44 (m, 2H), 7.27 (br s, 3H), 7.02-6.98 (m, 2H), 6.52 (b r s, 2H), 5.59-5.49 (m, 2H), 5.13 (dd, J=12.8, 5.2 Hz, 1H), 4.12-4.09 (m, 2H), 3.67-3.64 (m, 2H), 3.47-3.44 (m, 1H), 3.09-2.93 (m, 4H), 2.76-2.70 (m, 1H), 2.22-2.01 (m, 3H), 1.72-1.67 (m, 4H), 1.48-1.38 (m, 5H). MS (ESI+) 871.4 (M+H⁺).

### Example 9—Preparation of 4-((7-((9-((5-((R)-3-amino-3-((S)-2,2-difluoro-1-hydroxyethyl)piperidin-1-yl)-2-(2,5-difluoro-4-methoxyphenyl)pyridin-4-yl)methyl)-9H-purin-6-yl)amino)heptyl)amino)-2-(2,6-dioxopiperidin-3-yl)isoindoline-1,3-dione (I-78)

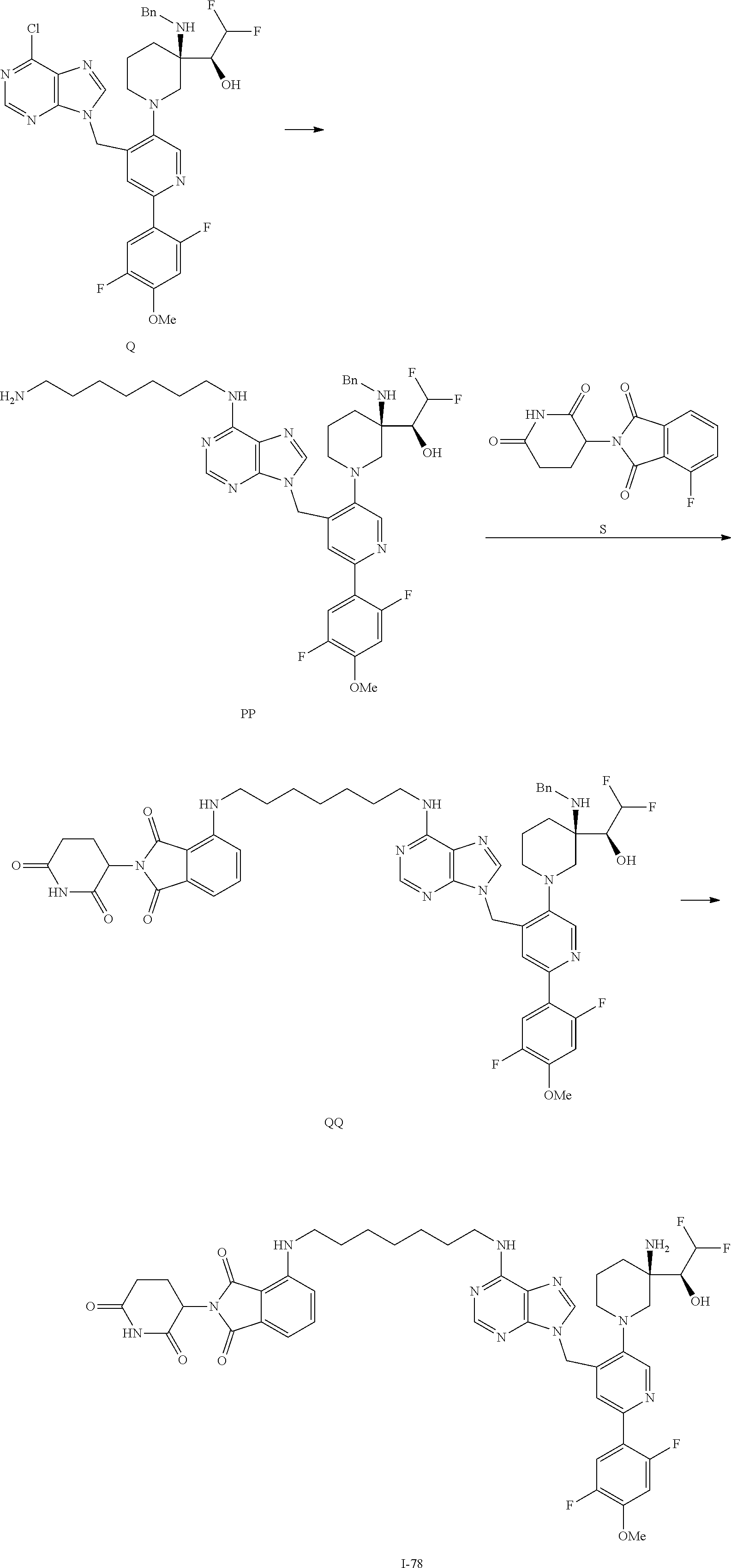

Chloro purine compound Q (800 mg) was aminated with 1,7-diaminoheptane using diisopropylethylamine in n-butanol to produce crude alkyl amino compound PP (900 mg). Crude compound PP (780 mg) was exposed to 2-(2,6-dioxopiperidin-3-yl)-4-fluoroisoindoline-1,3-dione S in diisopropylethylamine and N-methyl-2-pyrrolidone to produce compound QQ (600 mg). Compound QQ (600 mg) was hydrogenated using Pd(OH)₂/C with trifluoroacetic acid in THF to produce the title compound I-78 (105 mg). ¹H NMR: (400 MHz, CDCl₃) δ 8.50 (s, 1H), 8.37 (s, 1H), 7.92 (s, 1H), 7.71 (dd, J=12.0, 7.2 Hz, 1H), 7.49 (t, J=7.6 Hz, 1H), 7.39 (d, J=3.2 Hz, 1H), 7.10 (d, J=7.2 Hz, 1H), 6.91 (d, J=8.8 Hz, 1H), 6.84 (br s, 1H), 6.68 (dd, J=12.4, 7.2 Hz, 1H), 6.31 (br t, J=6.0 Hz, 1H), 5.96 (td, J=55.2, 2.8 Hz, 1H), 5.55-5.45 (m, 2H), 4.94 (dd, J=11.6, 4.4 Hz, 1H), 3.89 (app s, 4H), 3.67 (br s, 1H), 3.40-3.26 (m, 4H), 2.97-2.73 (m 7H), 2.15-2.13 (m, 1H), 1.87-1.77 (m, 4H), 1.63-1.60 (m, 6H), 1.38-1.32 (m, 7H). MS (ESI+) 916.3 (M+H⁺).

### Example 10—Preparation of 4-((5-((9-((5-((R)-3-amino-3-(pyridin-2-yl)piperidin-1-yl)-2-(3,5-difluoro-4-methoxyphenyl)pyridin-4-yl)methyl)-9H-purin-6-yl)amino)pentyl)amino)-2-(2,6-dioxopiperidin-3-yl)isoindoline-1,3-dione (I-79)

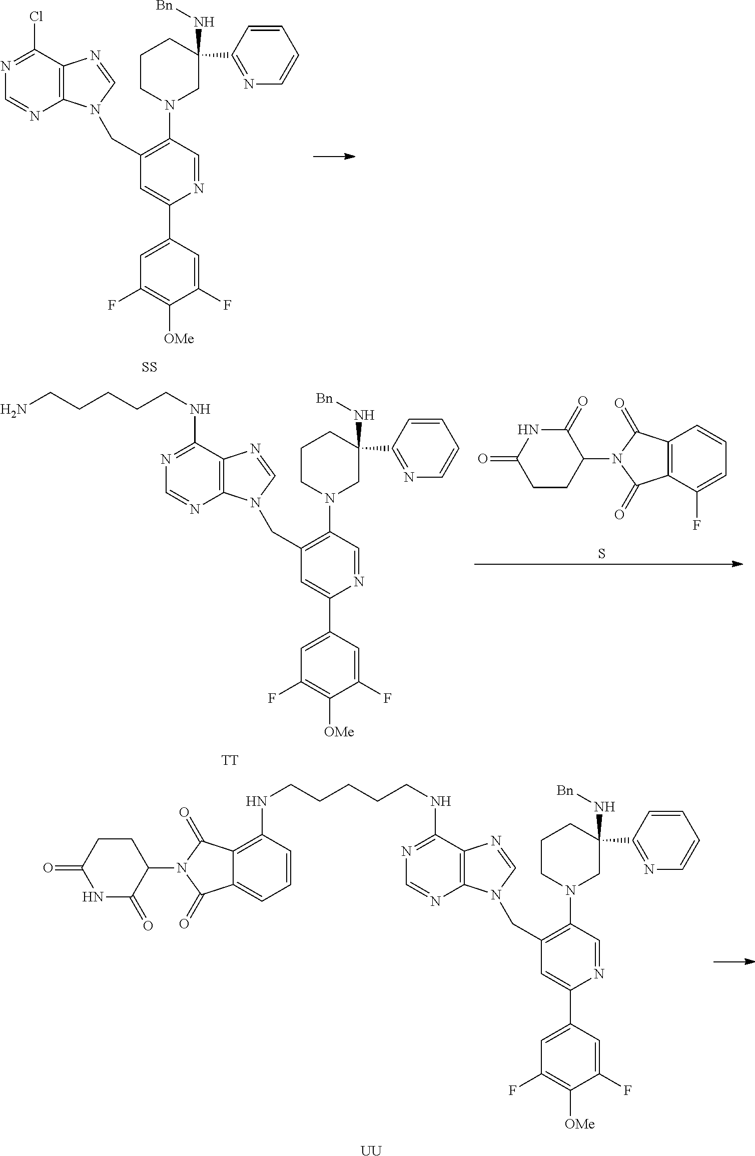

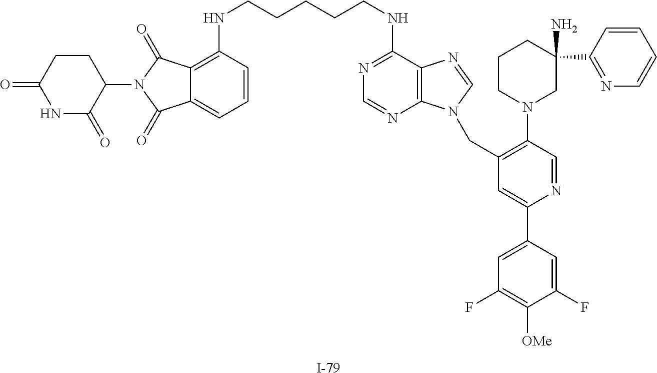

Chloro purine compound SS (850 mg) was aminated with 1,5-diaminopentane using diisopropylethylamine in n-butanol to produce crude alkyl amino compound TT (900 mg). Crude compound TT (900 mg) was exposed to 2-(2,6-dioxopiperidin-3-yl)-4-fluoroisoindoline-1,3-dione S in diisopropylethylamine and DMSO to produce compound UU (420 mg). Compound UU (420 mg) was hydrogenated using Pd(OH)₂/C in THE to produce the title compound I-79 (95.6 mg). ¹H NMR: (400 MHz, DMSO-d₆) δ 8.68 (s, 1H), 8.62 (s, 1H), 8.34 (s, 1H), 8.27 (s, 1H), 7.96-7.94 (m, 2H), 7.84 (d, J=7.6 Hz, 1H), 7.68-7.56 (m, 4H), 7.44 (br s, 1H), 7.09 (d, J=8.8 Hz, 1H), 7.02 (d, J=6.8 Hz, 1H), 6.57 (s, 1H), 5.63-5.52 (m, 2H), 5.09-5.04 (m, 1H), 3.97 (s, 3H), 3.63-3.50 (m 3H), 3.36-3.22 (m, 2H), 3.10-3.03 (m, 2H), 2.92-2.87 (m, 1H), 2.62-2.52 (m, 2H), 2.24-2.18 (m, 1H), 2.05-2.02 (m, 3H), 1.69-1.62 (m, 5H), 1.43-1.42 (m, 2H). MS (ESI+) 885.3 (M+H⁺).

### Example 11—Preparation of 4-((5-((9-((5-((S)-3-amino-3-(pyridin-2-yl)piperidin-1-yl)-2-(3,5-difluoro-4-methoxyphenyl)pyridin-4-yl)methyl)-9H-purin-6-yl)amino)pentyl)amino)-2-(2,6-dioxopiperidin-3-yl)isoindoline-1,3-dione (I-86)

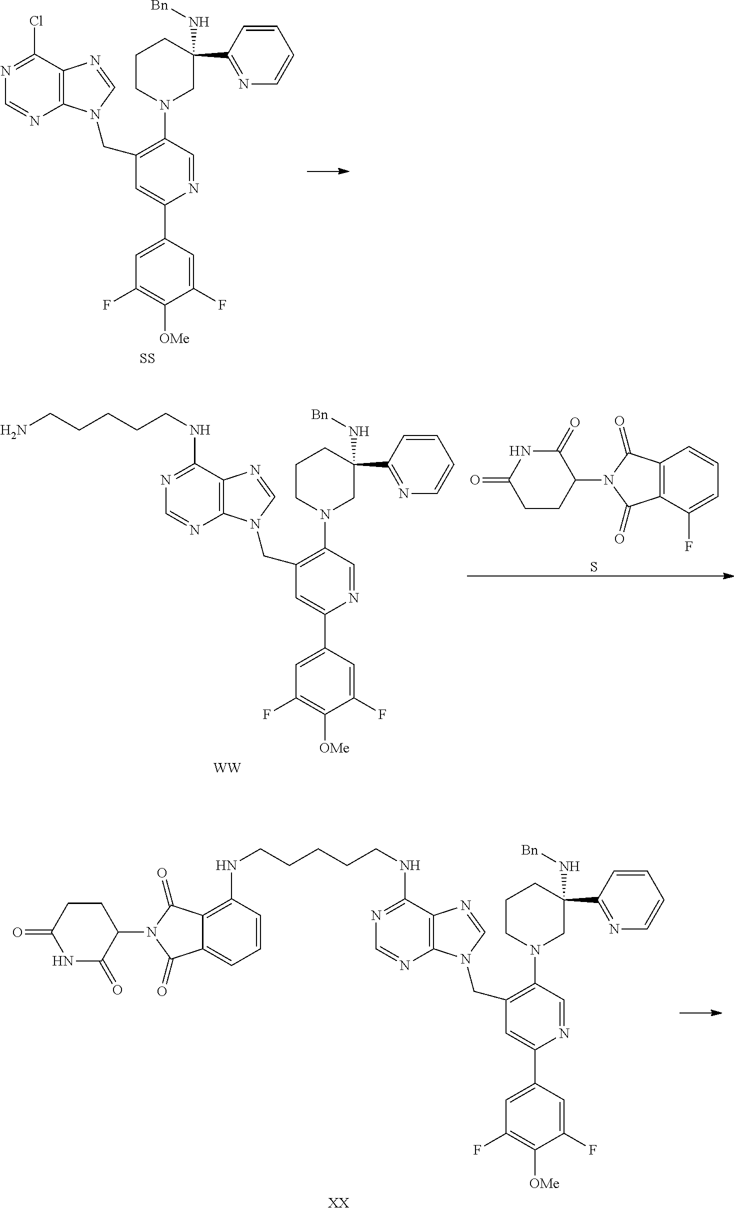

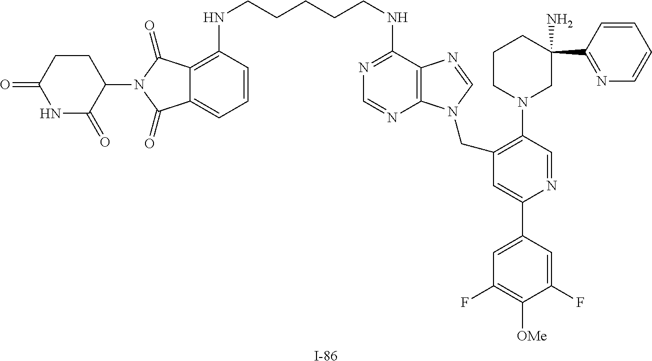

Chloro purine compound SS (850 mg) was aminated with 1,5-diaminopentane using diisopropylethylamine in n-butanol to produce crude alkyl amino compound WW (900 mg). Crude compound WW (900 mg) was exposed to 2-(2,6-dioxopiperidin-3-yl)-4-fluoro-isoindoline-1,3-dione S in diisopropylethylamine and DMSO to produce compound XX (400 mg). Compound XX (400 mg) was hydrogenated using Pd(OH)₂/C in THE to produce the title compound I-86 (32.6 mg). ¹H NMR: (400 MHz, DMSO-d₆) δ 8.57 (s, 1H), 8.52 (s, 1H), 8.28 (s, 1H), 8.20 (s, 1H), 7.83-7.74 (m, 3H), 7.79-7.46 (m, 4H), 7.28 (br s, 1H), 7.07 (d, J=8.4 Hz, 1H), 6.99 (d, J=6.4 Hz, 1H), 6.53 (s, 1H), 5.59-5.49 (m, 2H), 5.06-5.02 (m, 1H), 3.93 (s, 3H), 3.55-3.45 (m 2H), 3.32-3.25 (m, 3H), 3.09-3.07 (m, 2H), 2.94-2.87 (m, 2H), 2.60-2.52 (m, 2H), 2.19-2.02 (m, 3H), 1.73-1.61 (m, 6H), 1.42-1.41 (m, 2H). MS (ESI+) 885.4 (M+H⁺).

### Example 12—Preparation of 4-((6-((9-((5-((R)-3-amino-3-(pyridin-2-yl)piperidin-1-yl)-2-(3,5-difluoro-4-methoxyphenyl)pyridin-4-yl)methyl)-9H-purin-6-yl)amino)hexyl)amino)-2-(2,6-dioxopiperidin-3-yl)isoindoline-1,3-dione (I-80)

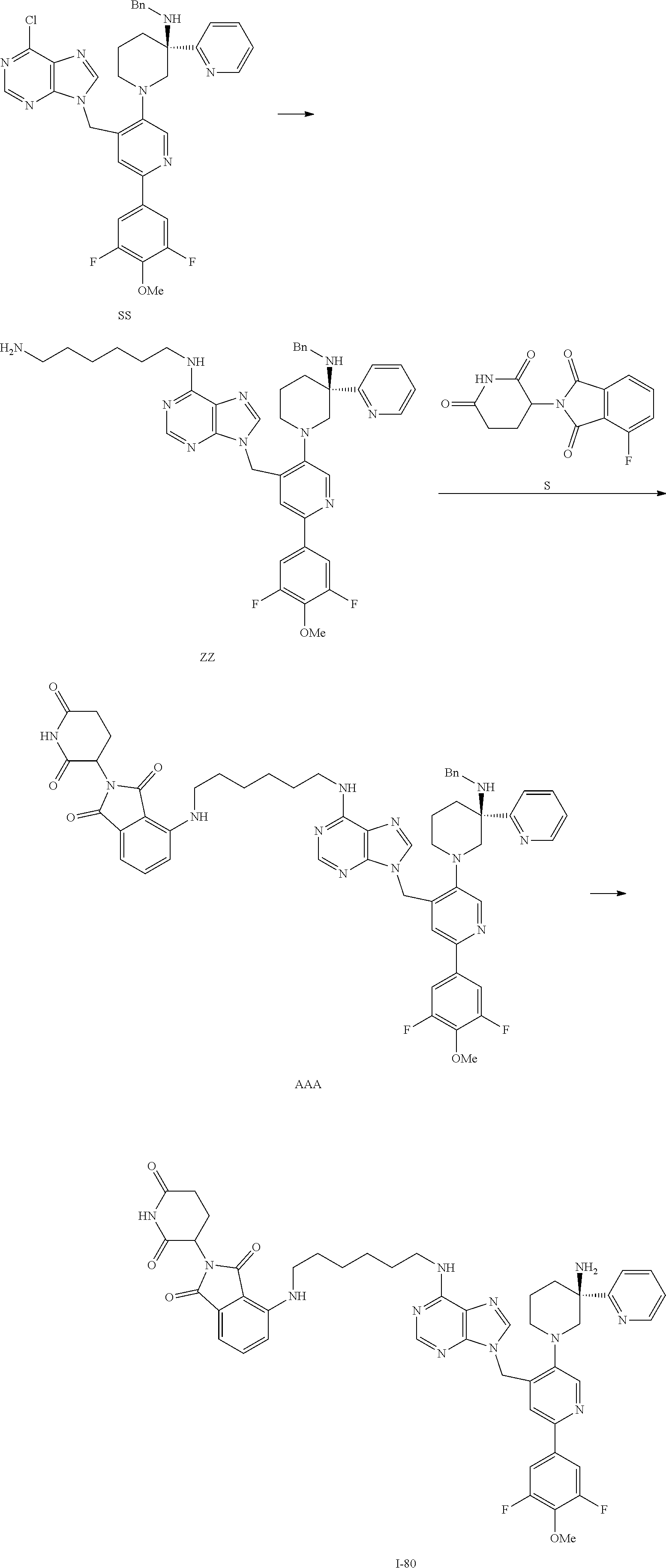

Chloro purine compound SS (850 mg) was aminated with 1,6-diaminohexane using diisopropylethylamine in n-butanol to produce crude alkyl amino compound ZZ (1.0 g). Crude compound ZZ (1.0 g) was exposed to 2-(2,6-dioxopiperidin-3-yl)-4-fluoroisoindoline-1,3-dione S in diisopropylethylamine and DMSO to produce compound AAA (450 mg). Compound AAA (450 mg) was hydrogenated using Pd(OH)₂/C in THE to produce the title compound I-80 (74.5 mg). ¹H NMR: (400 MHz, DMSO-d₆) δ 8.85 (br s, 2H), 8.73 (s, 1H), 8.65 (s, 1H), 8.35 (s, 1H), 8.30 (s, 1H), 8.10-8.01 (m, 2H), 7.83 (d, J=8.4 Hz, 1H), 7.67 (d, J=8.4 Hz, 2H), 7.55-7.52 (m, 2H), 7.06-7.01 (m, 1H), 6.53 (s, 1H), 5.62-5.55 (m, 2H), 5.06-5.02 (m, 1H), 3.95 (s, 3H), 3.49-3.48 (m 2H), 3.34-3.25 (m, 3H), 3.05-3.04 (m, 2H), 2.92-2.88 (m, 1H), 2.16 (br s, 1H), 2.04-2.02 (m, 2H), 2.84-2.81 (m, 1H), 1.62-1.57 (m, 4H), 1.38 (br app s, 4H). MS (ESI+) 899.4 (M+H⁺).

### Example 13—Preparation of 4-((6-((9-((5-((S)-3-amino-3-(pyridin-2-yl)piperidin-1-yl)-2-(3,5-difluoro-4-methoxyphenyl)pyridin-4-yl)methyl)-9H-purin-6-yl)amino)hexyl)amino)-2-(2,6-dioxopiperidin-3-yl)isoindoline-1,3-dione (I-81)

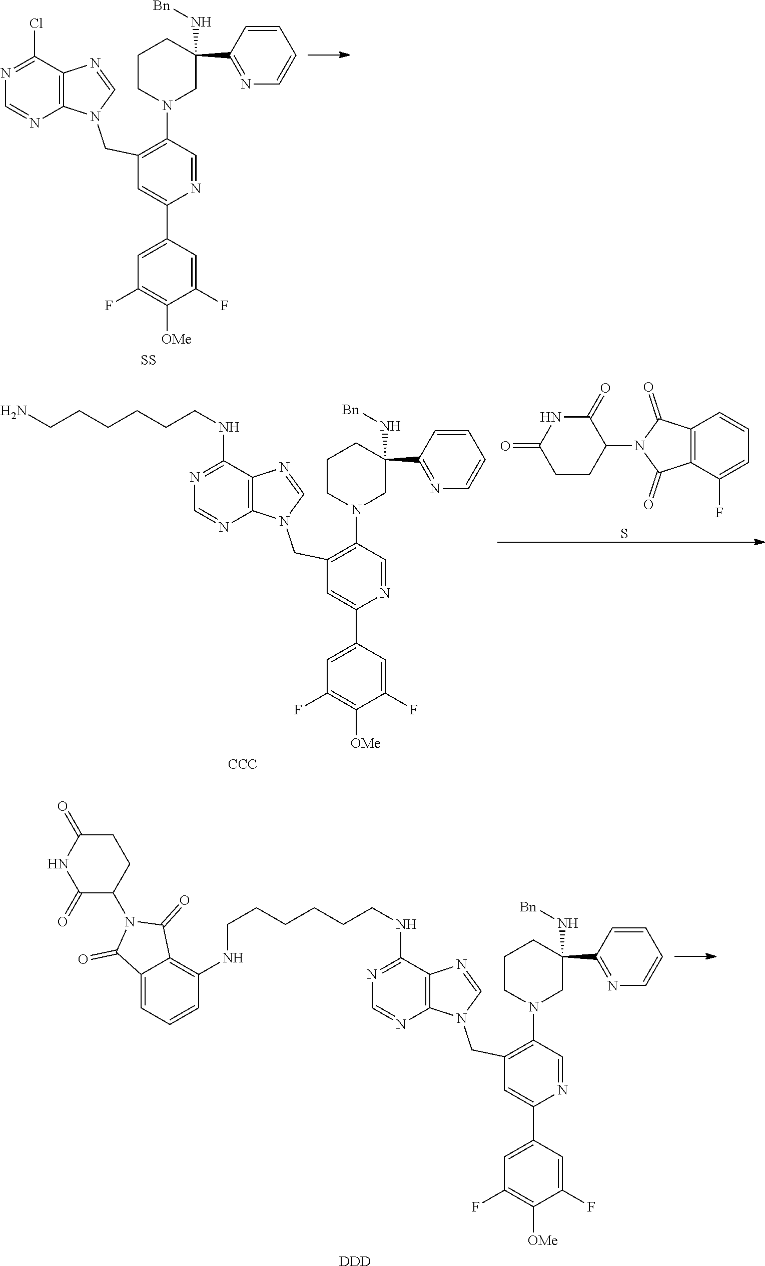

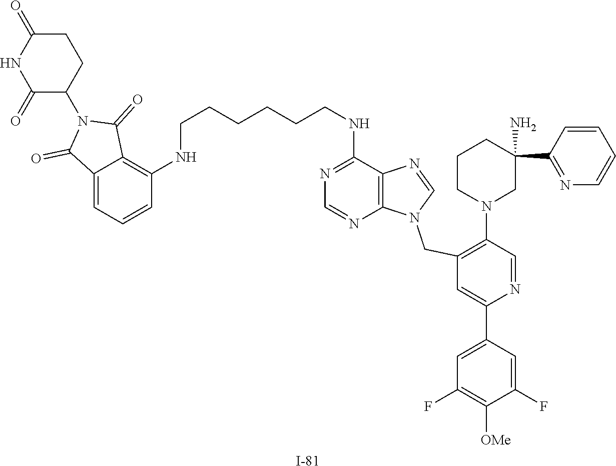

Chloro purine compound SS (850 mg) was aminated with 1,6-diaminohexane using diisopropylethylamine in n-butanol to produce crude alkyl amino compound CCC (900 mg). Crude compound CCC (900 mg) was exposed to 2-(2,6-dioxopiperidin-3-yl)-4-fluoroisoindoline-1,3-dione S in diisopropylethylamine and DMSO to produce compound DDD (420 mg). Compound DDD (420 mg) was hydrogenated using Pd(OH)₂/C in THE to produce the title compound I-81 (85.3 mg). ¹H NMR: (400 MHz, DMSO-d₆) δ 8.56 (s, 1H), 8.52 (s, 1H), 8.27 (s, 1H), 8.18 (s, 1H), 7.79 (br s, 3H), 7.59-7.54 (m, 3H), 7.45 (s, 1H), 7.28 (s, 1H), 7.05 (d, J=3.2 Hz, 1H), 6.99 (d, J=5.6 Hz, 1H), 6.52 (s, 1H), 5.55-5.49 (m, 2H), 5.06-5.03 (m, 1H), 3.93 (s, 3H), 3.49-3.46 (m, 4H), 3.08-2.82 (m, 5H), 2.60-2.55 (m, 2H), 2.20-2.15 (m, 1H), 2.03-2.01 (m, 2H), 1.73-1.57 (m, 6H), 1.37-1.29 (m, 5H). MS (ESI+) 899.4 (M+H⁺).

### Example 14—Preparation of 4-((7-((9-((5-((R)-3-amino-3-(pyridin-2-yl)piperidin-1-yl)-2-(3,5-difluoro-4-methoxyphenyl)pyridin-4-yl)methyl)-9H-purin-6-yl)amino)heptyl)amino)-2-(2,6-dioxopiperidin-3-yl)isoindoline-1,3-dione (I-82)

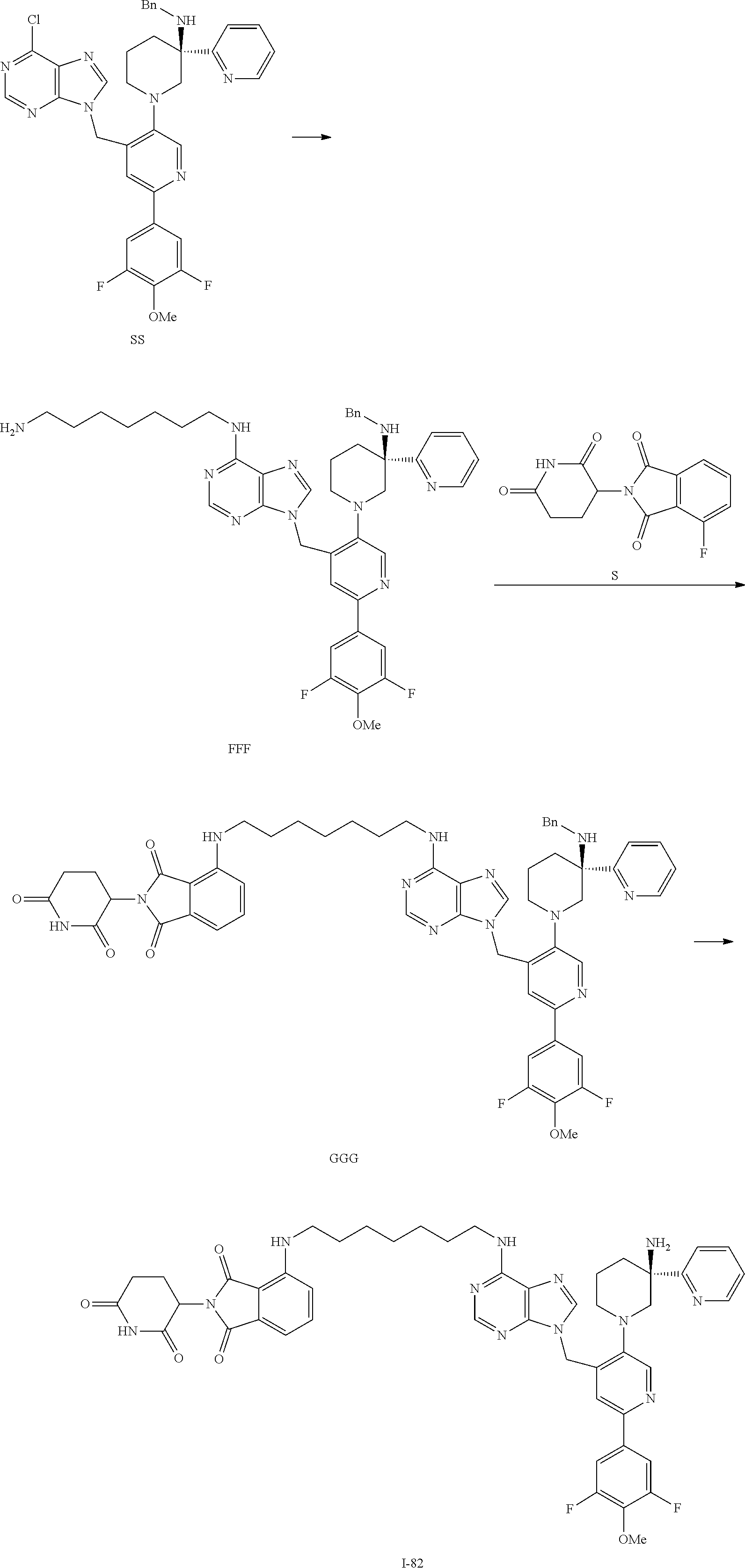

Chloro compound SS (850 mg) was aminated with 1,7-diaminoheptane using diisopropylethylamine in n-butanol to produce crude alkyl amino compound FFF (1.1 g). Crude compound FFF (1.1 g) was exposed to 2-(2,6-dioxopiperidin-3-yl)-4-fluoroisoindoline-1,3-dione S in diisopropylethylamine and DMSO to produce compound GGG (420 mg). Compound GGG (420 mg) was hydrogenated using Pd(OH)₂/C in THE to produce the title compound I-82 (65.4 mg). ¹H NMR: (400 MHz, DMSO-d₆) δ 8.85 (app br s, 3H), 8.73 (s, 1H), 8.66 (s, 1H), 8.35 (s, 1H), 8.32 (s, 1H), 8.03-7.99 (m, 2H), 7.83 (d, J=8.4 Hz, 1H), 7.68-7.64 (m, 3H), 7.55-7.51 (m, 2H), 7.06 (d, J=8.4 Hz, 1H), 7.00 (d, J=6.8 Hz, 1H), 6.52 (br s, 1H), 5.62-5.55 (m, 2H), 5.06-5.02 (m, 1H), 3.95 (s, 3H), 3.70 (d, J=12.4 Hz, 2H), 3.49 (br s, 1H), 3.38-3.26 (m, 3H), 3.05-3.02 (m, 2H), 3.94-2.84 (m, 1H), 2.60-2.55 (m, 1H), 2.17 (br s, 2H), 2.03-2.00 (m, 2H), 1.82-1.78 (m, 1H), 1.61-1.57 (m, 4H), 1.34 (br s, 6H). MS (ESI+) 913.3 (M+H⁺).

### Example 15—Preparation of 4-((7-((9-((5-((S)-3-amino-3-(pyridin-2-yl)piperidin-1-yl)-2-(3,5-difluoro-4-methoxyphenyl)pyridin-4-yl)methyl)-9H-purin-6-yl)amino)heptyl)amino)-2-(2,6-dioxopiperidin-3-yl)isoindoline-1,3-dione (I-83)

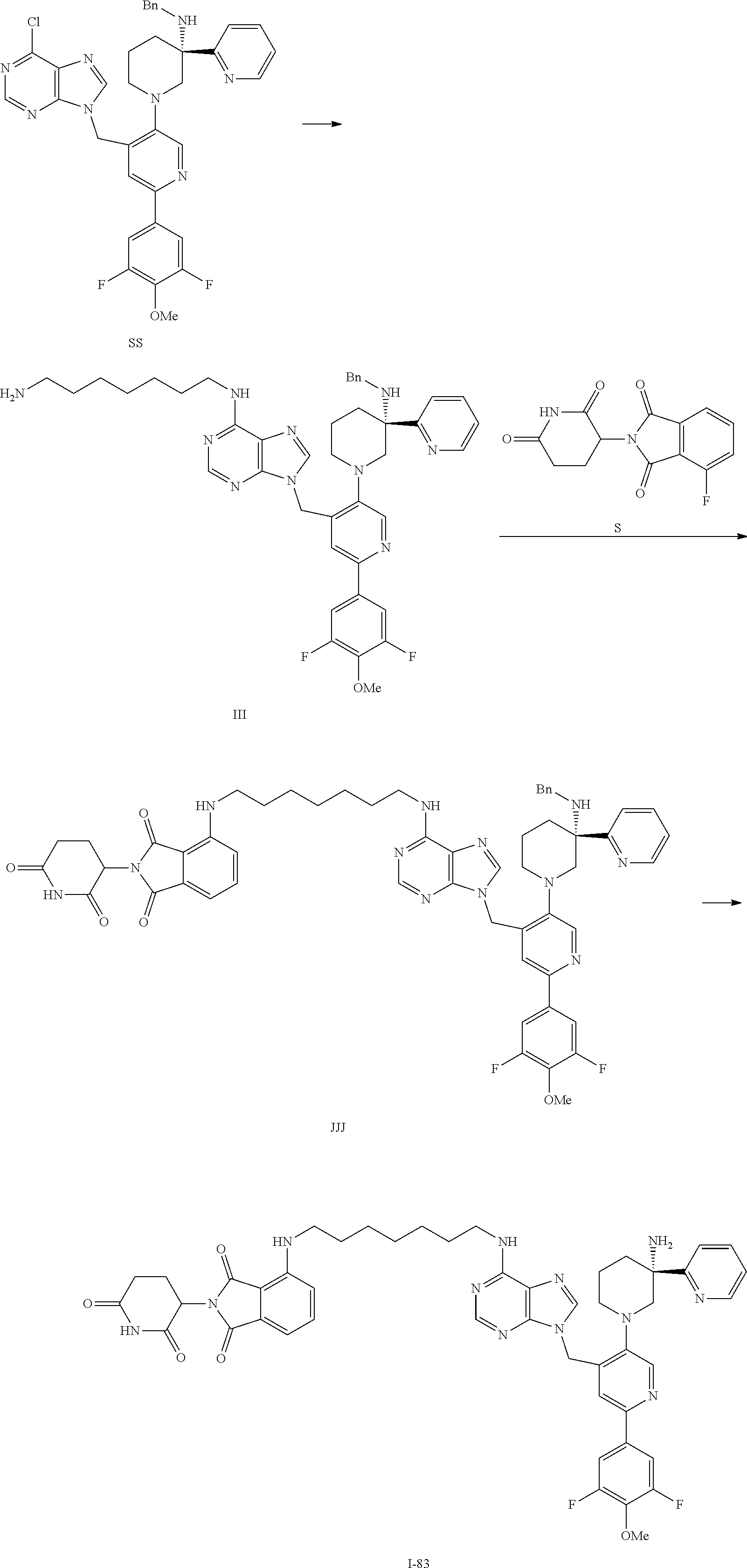

Chloro purine compound SS (850 mg) was aminated with 1,7-diaminoheptane using diisopropylethylamine in n-butanol to produce crude alkyl amino compound III (1.15 g). Crude compound III (1.15 g) was exposed to 2-(2,6-dioxopiperidin-3-yl)-4-fluoroisoindoline-1,3-dione S in diisopropylethylamine and DMSO to produce compound JJJ (450 mg). Compound JJJ (400 mg) was hydrogenated using Pd(OH)₂/C in THE to produce the title compound I-83 (79.9 mg). ¹H NMR: (400 MHz, DMSO-d₆) δ 8.55 (d, J=4.8 Hz, 1H), 8.51 (s, 1H), 8.28 (s, 1H), 8.20 (s, 1H), 7.80-7.76 (m, 3H), 7.62-7.53 (m, 3H), 7.44 (br s, 1H), 7.27-7.25 (m, 1H), 7.05 (d, J=8.8 Hz, 1H), 7.00 (d, J=6.8 Hz, 1H), 6.53-6.50 (m, 1H), 5.59-5.48 (m, 2H), 5.04 (dd, J=12.4, 5.2 Hz), 3.93 (s, 3H), 3.47-3.42 (m, 2H), 3.07-3.04 (m, 2H), 2.93-2.82 (m, 2H), 2.60-2.52 (m, 2H), 2.24-2.16 (m, 1H), 2.09-2.02 (m, 2H), 1.72 (d, J=9.2 Hz, 1H), 1.59-1.55 (m, 4H), 1.33 (br s, 6H). MS (ESI+) 913.4 (M+H⁺).

### Example 16—Preparation of 2-(4-(5-((R)-3-amino-3-((S)-2,2-difluoro-1-hydroxyethyl) piperidin-1-yl)-4-((6-amino-9H-purin-9-yl)methyl)pyridin-2-yl)-2,5-difluorophenoxy)-N-(2-((2-(2,6-dioxopiperidin-3-yl)-1,3-dioxoisoindolin-4-yl)amino)ethyl)acetamide (I-65)

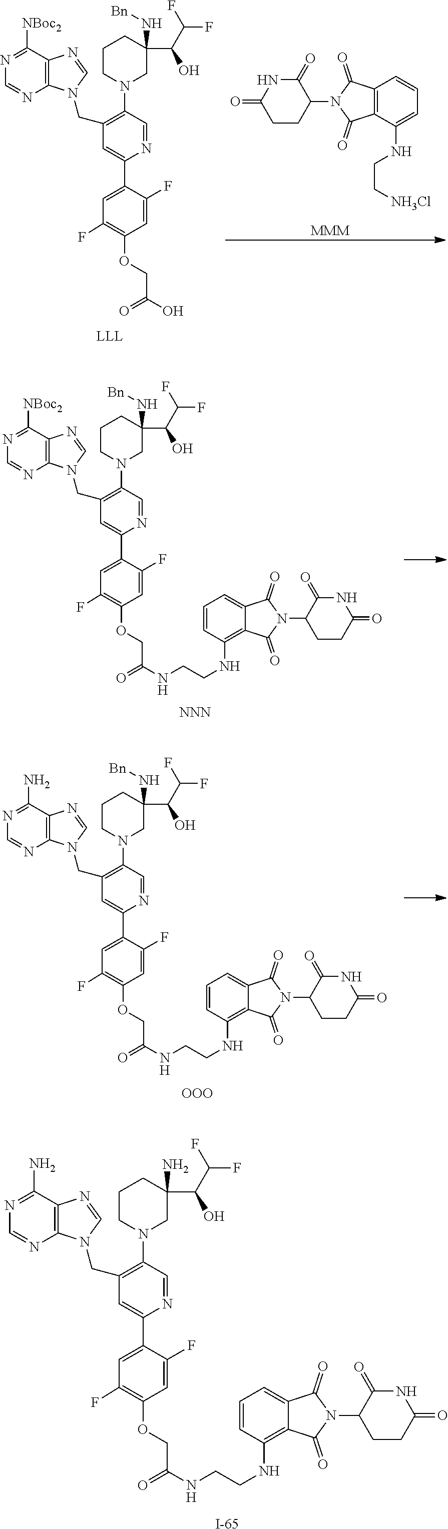

BOC-protected purine compound LLL was coupled with 4-((2-(chloro-15-azaneyl)ethyl)amino)-2-(2,6-dioxopiperidin-3-yl)isoindoline-1,3-dione MMM (600 mg) using propylphosphonic anhydride (T3P) to produce crude compound NNN (500 mg). Crude compound NNN (500 mg) was treated with trifluoroacetic acid in DCM to produce crude benzl amine compound OOO (440 mg). Crude benzyl amine compound OOO (440 mg) was hydrogenated using Pd(OH)₂/C in THE to produce the title compound I-65 (38.9 mg). ¹H NMR: (400 MHz, DMSO-d₆) δ 8.49 (s, 1H), 8.30 (s, 1H), 8.25 (s, 1H), 8.10 (s, 1H), 7.70 (dd, J=12.4, 7.2 Hz, 1H), 7.55 (t, J=7.6 Hz, 1H), 7.29 (br s, 2H), 7.17 (d, J=8.4 Hz, 1H), 7.04-7.00 (m, 2H), 6.93 (s, 1H), 6.76-6.73 (m, 1H), 6.19 (t, J=58.0 Hz, 1H), 5.54 (s, 2H), 5.04 (dd, J=12.0, 4.4 Hz, 1H), 3.10-2.76 (m, 7H), 2.59-2.55 (m, 2H), 1.99-1.71 (m, 5H), 1.49 (br s, 1H). MS (ESI+) 889.3 (M+H⁺).

### Example 17—Preparation of 2-(4-(5-((R)-3-amino-3-((S)-2,2-difluoro-1-hydroxyethyl) piperidin-1-yl)-4-((6-amino-9H-purin-9-yl)methyl)pyridin-2-yl)-2,5-difluorophenoxy)-N-(3-((2-(2,6-dioxopiperidin-3-yl)-1,3-dioxoisoindolin-4-yl)amino)propyl)acetamide (I-66)

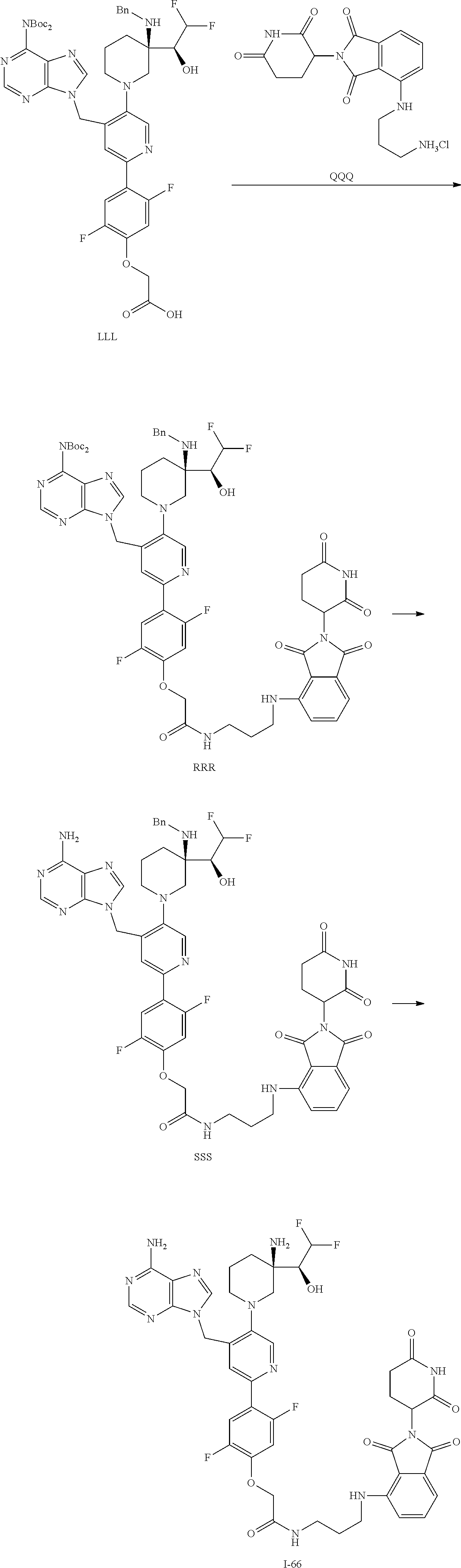

BOC-protected purine compound LLL was coupled with 4-((3-(chloro-15-azaneyl)propyl)amino)-2-(2,6-dioxopiperidin-3-yl)isoindoline-1,3-dione QQQ (3×200 mg) using propylphosphonic anhydride (T3P) to produce crude compound RRR (530 mg). Crude compound RRR (530 mg) was treated with trifluoroacetic acid in DCM to produce crude benzl amine compound SSS (441 mg). Crude benzyl amine compound SSS (441 mg) was hydrogenated using Pd(OH)₂/C in THE to produce the title compound I-66 (103.0 mg). ¹H NMR: (400 MHz, DMSO-d₆) δ 8.49 (s, 1H), 8.26 (s, 1H), 8.18 (s, 1H), 8.11 (s, 1H), 7.71-7.68 (m, 1H), 7.55-7.53 (m, 1H), 7.28 (br s, 2H), 7.06-7.00 (m, 3H), 6.94 (s, 1H), 6.68 (s, 1H), 6.18 (t, J=57.2 Hz, 1H), 5.54 (s, 2H), 5.04 (br d, J=8.4 Hz, 1H), 4.62 (s, 2H), 3.78 (br s, 1H), 3.21-2.78 (m, 10H), 2.60-2.56 (m, 2H), 2.03-1.71 (m, 7H), 1.47 (s, 1H). MS (ESI+) 903.3 (M+H⁺).

### Example 18—Preparation of 4-((6-(4-(5-((R)-3-amino-3-(pyridin-2-yl)piperidin-1-yl)-4-((6-amino-9H-purin-9-yl)methyl)pyridin-2-yl)-2,6-difluorophenoxy)hexyl)amino)-2-(2,6-dioxopiperidin-3-yl)isoindoline-1,3-dione (I-71)

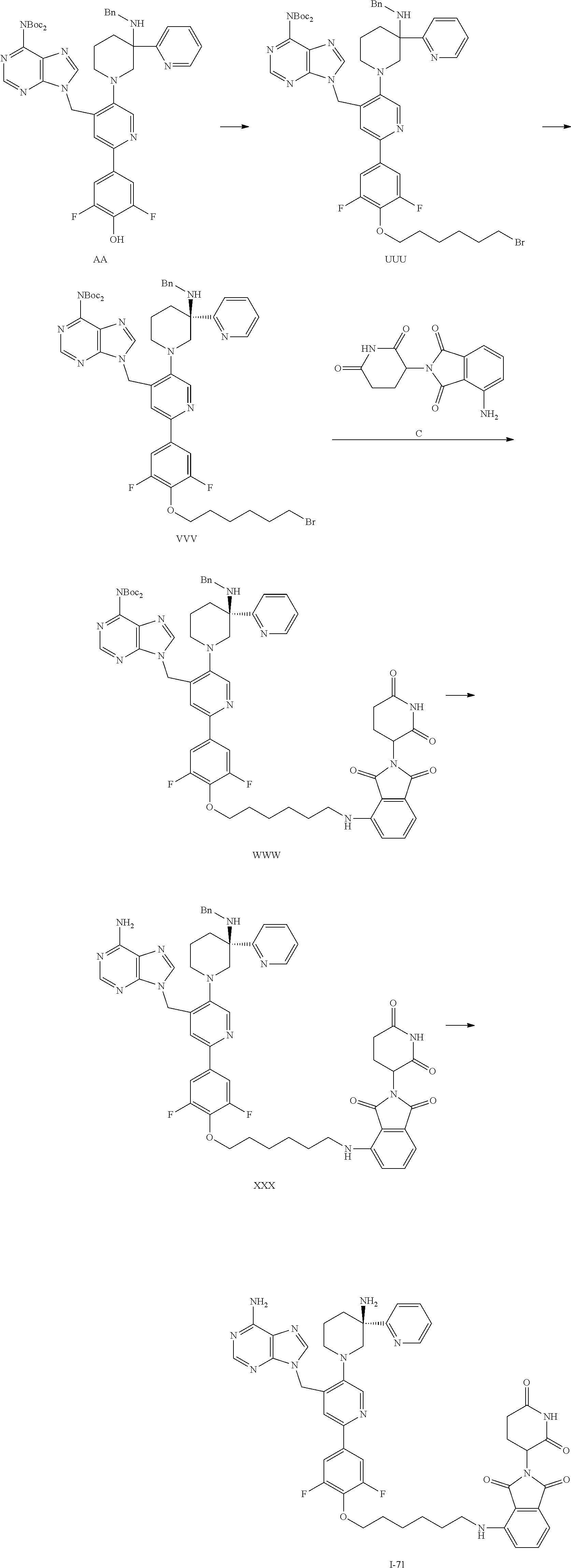

A racemic mixture of BOC-protected purine compound AA (2.0 g) was alkylated with 1,6-dibromohexane and K₂CO₃ in acetone at 65° C. for 2 hours to produce alkyl bromide UUU (1.07 g). Compound UUU (1.07 g) was purified to isolate enantioenriched isomer VVV (500 mg). Compound VVV (500 mg) was exposed to 4-amino-2-(2,6-dioxopiperidin-3-yl)isoindoline-1,3-dione C (1.0 equiv) in DMF with K₂CO₃ at 50° C. for 5 hours to produce compound WWW (490 mg). Compound WWW (490 mg) was treated with trifluoroacetic acid in DCM at 25° C. for 2 hours to produce compound XXX. Compound XXX was hydrogenated using Pd(OH)₂/C with trifluoroacetic acid in THF at 25° C. for 1.75 hours to produce the title compound I-71 (75.3 mg). ¹H NMR: (400 MHz, DMSO-d₆) δ 8.54 (d, J=4.0 Hz, 1H), 8.50 (s, 1H), 8.28 (s, 1H), 8.12 (s, 1H), 7.77 (br s, 2H), 7.57 (app d, J=9.6 Hz, 2H), 7.48-7.43 (m, 2H), 7.26 (br s, 3H), 7.02-6.98 (m, 2H), 6.52 (b r s, 2H), 5.58-5.48 (m, 2H), 5.12 (dd, J=12.8, 4.8 Hz, 1H), 4.12-4.08 (m, 2H), 3.66-3.62 (m, 2H), 3.45 (d, J=11.2 Hz, 1H), 3.11-2.92 (m, 4H), 2.74-2.70 (m, 1H), 2.20-2.02 (m, 3H), 1.71-1.54 (m, 4H), 1.41-1.28 (m, 6H). MS (ESI+) 885.4 (M+H⁺).

### Example 19—Preparation of 4-((6-(4-(5-((S)-3-amino-3-(pyridin-2-yl)piperidin-1-yl)-4-((6-amino-9H-purin-9-yl)methyl)pyridin-2-yl)-2,6-difluorophenoxy)hexyl)amino)-2-(2,6-dioxopiperidin-3-yl)isoindoline-1,3-dione (I-72)

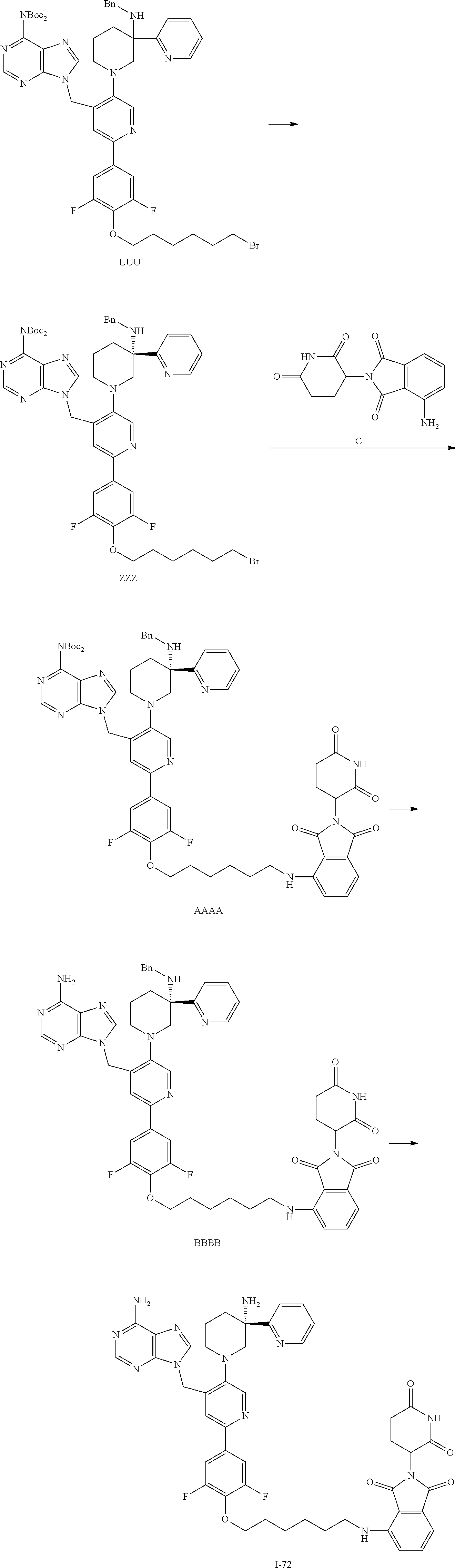

BOC-protected purine compound UUU (1.07 g) was purified to isolate enantioenriched isomer ZZZ (500 mg). Compound ZZZ (500 mg) was exposed to 4-amino-2-(2,6-dioxopiperidin-3-yl)isoindoline-1,3-dione C (1.0 equiv) in DMF with K₂CO₃ at 50° C. for 5 hours to produce compound AAAA (480 mg). Compound AAAA (480 mg) was treated with trifluoroacetic acid in DCM at 25° C. for 2 hours to produce benzyl amine compound BBBB. Benzl amine compound BBBB was hydrogenated using Pd(OH)₂/C with trifluoroacetic acid in THE at 25° C. for 1.75 hours to produce the title compound I-72 (69.0 mg). ¹H NMR: (400 MHz, DMSO-d₆) δ 8.54 (d, J=3.6 Hz, 1H), 8.51 (s, 1H), 8.28 (s, 1H), 8.12 (s, 1H), 7.78 (br s, 2H), 7.57 (app d, J=9.2 Hz, 2H), 7.48-7.43 (m, 2H), 7.26 (br s, 3H), 7.02-6.98 (m, 2H), 6.52 (b r s, 2H), 5.58-5.48 (m, 2H), 5.12 (dd, J=12.8, 4.8 Hz, 1H), 4.12-4.09 (m, 2H), 3.66-3.62 (m, 2H), 3.48-3.43 (m, 1H), 3.11-2.92 (m, 4H), 2.75-2.72 (m, 1H), 2.19-2.02 (m, 3H), 1.72-1.64 (m, 4H), 1.42-1.75 (m, 6H). MS (ESI+) 885.4 (M+H⁺).

### Example 20—Evolution of NSD2 Inhibitory Activity for Exemplary Compounds

Exemplary compounds were tested for NSD2 inhibitory activity using a modified RBC HotSpot HMT Assay. Experimental procedures and results are provided below.

### Part I—Experimental Procedures

A reaction mixture was prepared by adding oligonucleosomes from chicken (0.05 mg/mL) to freshly prepared reaction buffer containing 50 mM Tris-HCl (pH 8.5), 50 mM NaCl, 5 mM MgCl₂, 0.01% Brij35, 1 mM DTT, and 1% DMSO followed by the addition of recombinant human NSD2 (amino acids 2-end) with N-terminal His tag (2 nM; GenBank Accession No. NM_001042424; MW=155.5 kDa; expressed in Sf9 insect cells) and gentle mixing. The test compounds were diluted in DMSO (to 10 mM), and added to the reaction mixture in nanoliter amounts by using Acoustic Technology (Echo 550, LabCyte Inc. Sunnyvale, CA) and incubated for 20 minutes at room temperature. The reaction was initiated by the addition of S-adenosyl-L-[methyl-³H] methionine (3H-SAM; 1 μM), and the mixture was incubated for 1 hour at 30° C. The reaction mixture was delivered to filter paper, and the methylated substrate was quantified by scintillation counting.

The control compound, SAH (S-(5′-adenosyl)-L-homocysteine) was tested in 10-dose IC₅₀ mode with 3-fold serial dilution starting at 10 or 100 M.

Data analysis was performed using Excel and GraphPad Prism software for IC₅₀ curve fits. For additional information on the histone methyltransferase assay, see, for example, Horiuchi K Y, et al., “Assay Development for Histone Methyltransferases,” Assay Drug Dev Technol. 2013 May; 11(4):227-36.

### Part II—Results

Results of the NSD2 inhibitory assay are reported in Table 2 below. Compounds with an IC₅₀ less than or equal to 50 nM are designated as “A”. Compounds with an IC₅₀ greater than 50 nM and less than or equal to 100 nM are designated as “B”. Compounds with an IC₅₀ greater than 100 nM and less than or equal to 1000 nM are designated as “C”. Compounds with an IC₅₀ greater than 1000 nM are designated as “D”.

| Compound No. | IC₅₀ |
|---|---|
| I-61 | A |
| I-62 | B |
| I-63 | C |
| I-64 | A |
| I-65 | A |
| I-66 | A |
| I-67 | A |
| I-68 | D |
| I-69 | A |
| I-70 | D |
| I-71 | A |
| I-76 | D |
| I-77 | D |
| I-79 | C |
| I-80 | D |
| 1-81 | B |
| I-82 | D |
| I-84 | C |
| I-85 | C |
| SAH | D |

### INCORPORATION BY REFERENCE

The entire disclosure of each of the patent and patent publication documents and scientific articles referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting the disclosure described herein. Scope of the disclosure is thus indicated by the appended claims rather than by the foregoing description, and all changes that come within the meaning and range of equivalency of the claims are intended to be embraced therein.

1. A compound represented by Formula I: 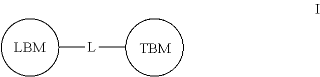
or a pharmaceutically acceptable salt thereof, wherein:
LBM is a ligase binding moiety;
L is a bivalent linker; and
TBM is a radical of Formula A: 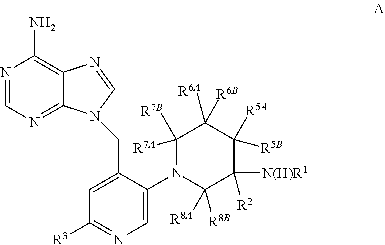
wherein:
R¹ is hydrogen or C₁₋₄ alkyl;
R² is —(C₁₋₄ haloalkyl substituted with 0 or 1 occurrences of hydroxyl) or a 5-6 membered heteroaryl containing 1 nitrogen atom, wherein the heteroaryl is substituted with 0 or 1 occurrences of halo;
R³ is phenyl or pyridinyl, each of which is substituted with p occurrences of R⁴;
R⁴ represents independently for each occurrence halo, hydroxyl, C₁₋₄ haloalkyl, —O—(C₁₋₄ haloalkyl), or C₁₋₄ alkoxyl;
R^{5A}, R^{5B}, R^{6A}, R^{6B}, R^{7A}, R^{7B}, R^{8A}, and R^{8B} are independently hydrogen, halo, cyano, aryl, —(C₀₋₆ alkylene)-(hydroxyl), C₁₋₆ alkyl, —(C₁₋₆ haloalkyl substituted with 0 or 1 occurrences of hydroxyl), —(C₀₋₆ alkylene)-(C₁₋₆ alkoxyl substituted with 0 or 1 occurrences of halo), —C(O)—OR⁹, or —(C(R¹⁰)(R¹¹))ₛ—C(O)—N(R¹²)(R¹³);
R⁹, R¹⁰, R¹¹, R¹², and R¹³ each represent independently for each occurrence hydrogen or C₁₋₄ alkyl;
p is 0, 1, 2, or 3; and
s is 0, 1, 2, 3, or 4.

2. (canceled)

3. (canceled)

4. (canceled)

5. The compound of claim 1, wherein TBM is a radical of Formula A-3: 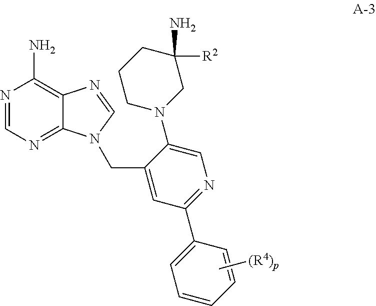
wherein:
R² is —(C₁₋₄ haloalkyl substituted with 0 or 1 occurrences of hydroxyl) or a 5-6 membered heteroaryl containing 1 nitrogen atom, wherein the heteroaryl is substituted with 0 or 1 occurrences of halo;
R⁴ represents independently for each occurrence halo, hydroxyl, C₁₋₄ haloalkyl, —O—(C₁₋₄ haloalkyl), or C₁₋₄ alkoxyl; and
p is 0, 1, 2, or 3.

6. (canceled)

7. (canceled)

8. (canceled)

9. (canceled)

10. The compound of claim 5, wherein R⁴ represents independently for each occurrence fluoro, chloro, methoxy, —OCHF₂, or —CHF₂.

11. (canceled)

12. (canceled)

13. (canceled)

14. (canceled)

15. The compound of claim 5, wherein R² is C₁₋₄ haloalkyl substituted with 0 or 1 occurrences of hydroxyl.

16. (canceled)

17. (canceled)

18. The compound of claim 5, wherein R² is pyridinyl substituted with 0 or 1 occurrences of halo.

19. (canceled)

20. (canceled)

21. (canceled)

22. The compound of claim 5, wherein R² is selected from

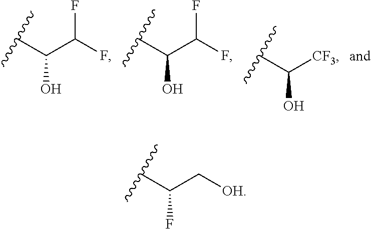

23. (canceled)

24. (canceled)

25. (canceled)

26. The compound of claim 1, wherein TBM is

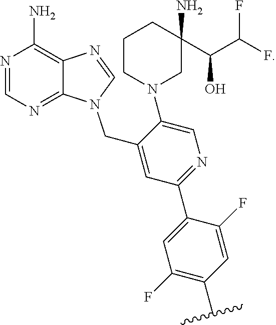 [SMILES: *c1cc(F)c(-c2cc(Cn3cnc4c(N)ncnc43)c(N3CCC[C@](N)([C@H](O)C(F)F)C3)cn2)cc1F]

27. The compound of claim 1, wherein TBM is

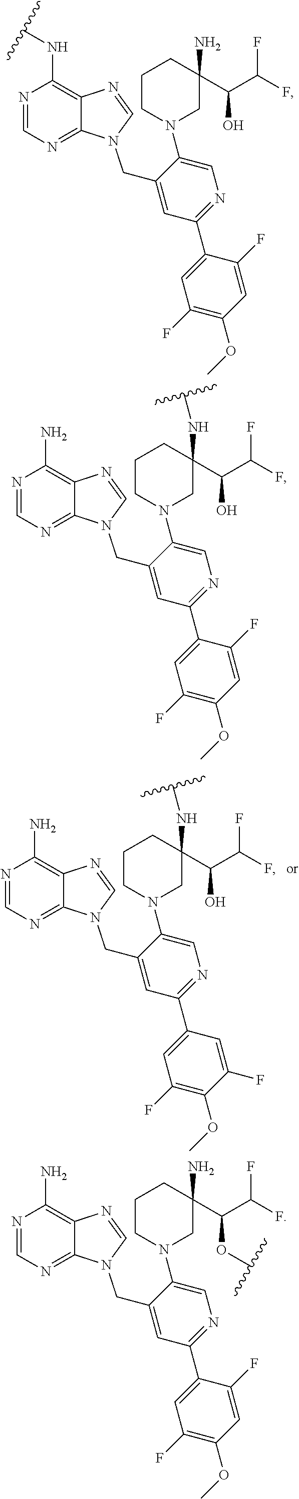

28. The compound of claim 1, wherein L is a covalent bond, or a bivalent, saturated or partially unsaturated, straight or branched C₁₋₆₀ hydrocarbon chain, wherein 1-20 methylene units of L are independently and optionally replaced by -Cy-, —O—, —N(R¹)—, —S—, —C(O)—, —S(O)—, —S(O)₂—, or 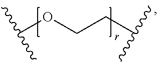
wherein:
each -Cy- is independently a 3-7 membered saturated or partially unsaturated carbocyclylene, a 3-7 membered saturated or partially unsaturated heterocyclylene containing 1 or 2 heteroatoms independently selected from nitrogen, oxygen, and sulfur, or a 5-6 membered heteroarylene containing 1, 2, 3, or 4 heteroatoms independently selected from nitrogen, oxygen, and sulfur, wherein the carbocyclene, heterocyclylene, and heteroarylene are substituted with 0-2 occurrences of R^{J};
R^{I} represents independently for each occurrence hydrogen or C₁₋₆ alkyl;
R^{J} represents independently for each occurrence C₁₋₆ alkyl, C₁₋₆ haloalkyl, or C₁₋₆ alkoxyl; and
r is 0, 1, 2, 3, 4, 5, 6, 7, 8, 9, or 10.

29. The compound of claim 1, wherein L is a bivalent, saturated, straight or branched C₁₋₂₀ hydrocarbon chain, wherein 1, 2, 3, 4, 5, 6, 7, or 8 methylene units of L are independently replaced by —O—, —N(H)—, —N(C₁₋₄ alkyl)-, —S—, —C(O)—, —S(O)—, or —S(O)₂—.

30. (canceled)

31. (canceled)

32. The compound of claim 1, wherein L is 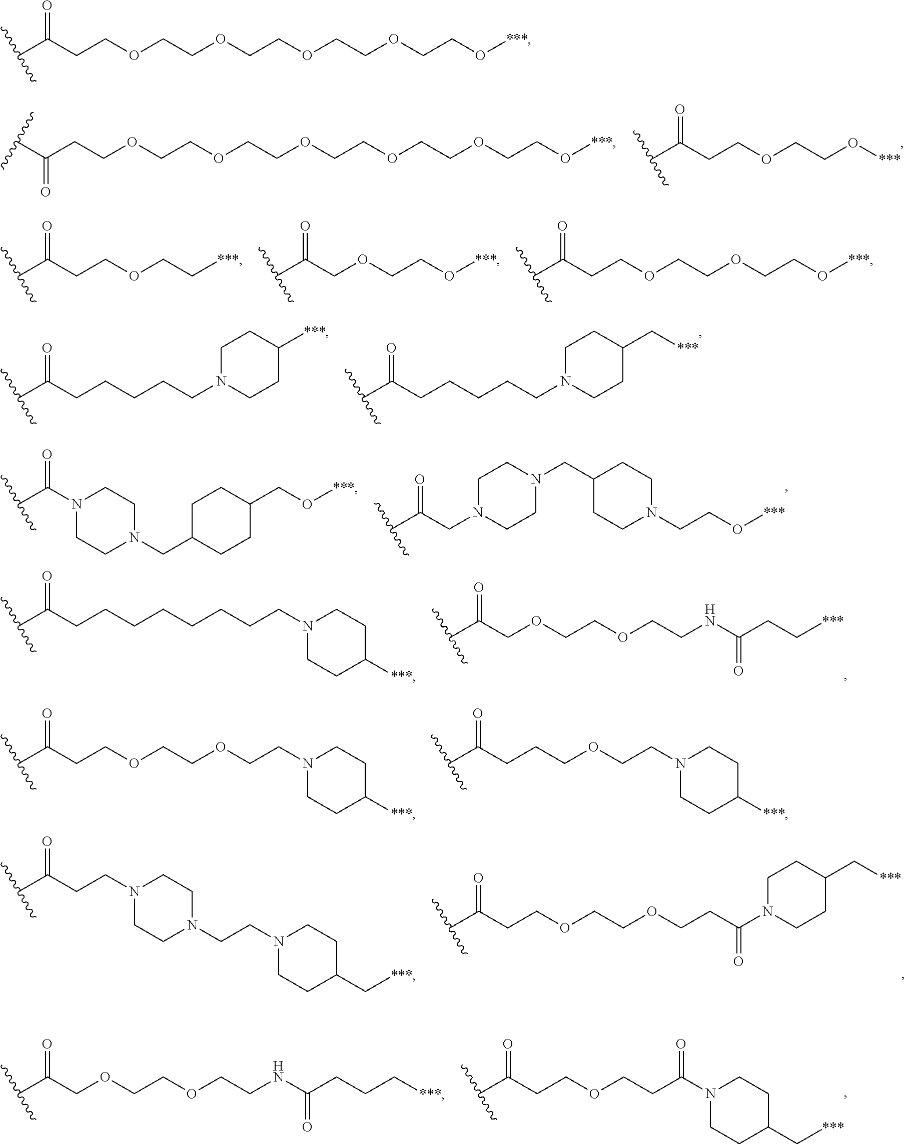 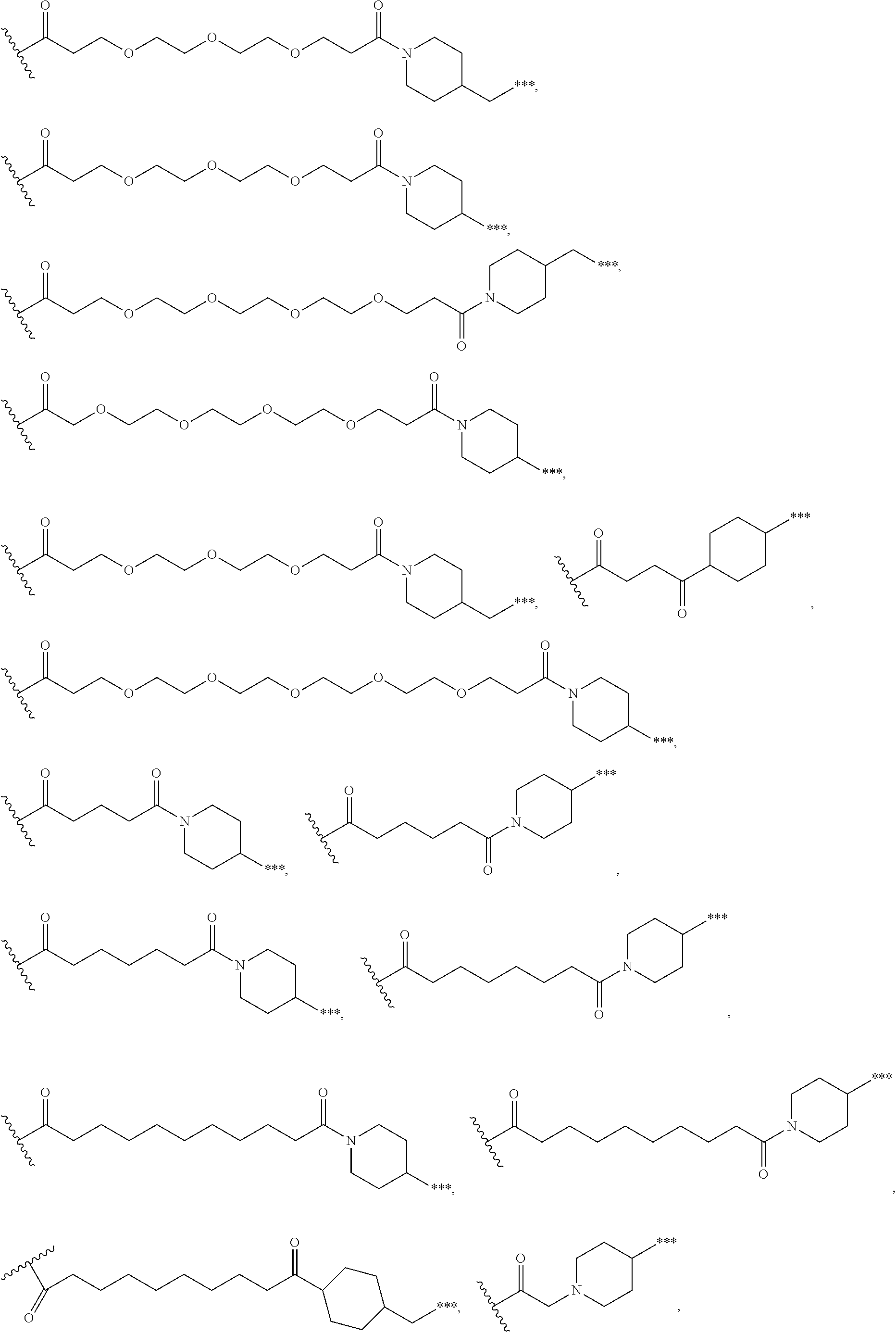 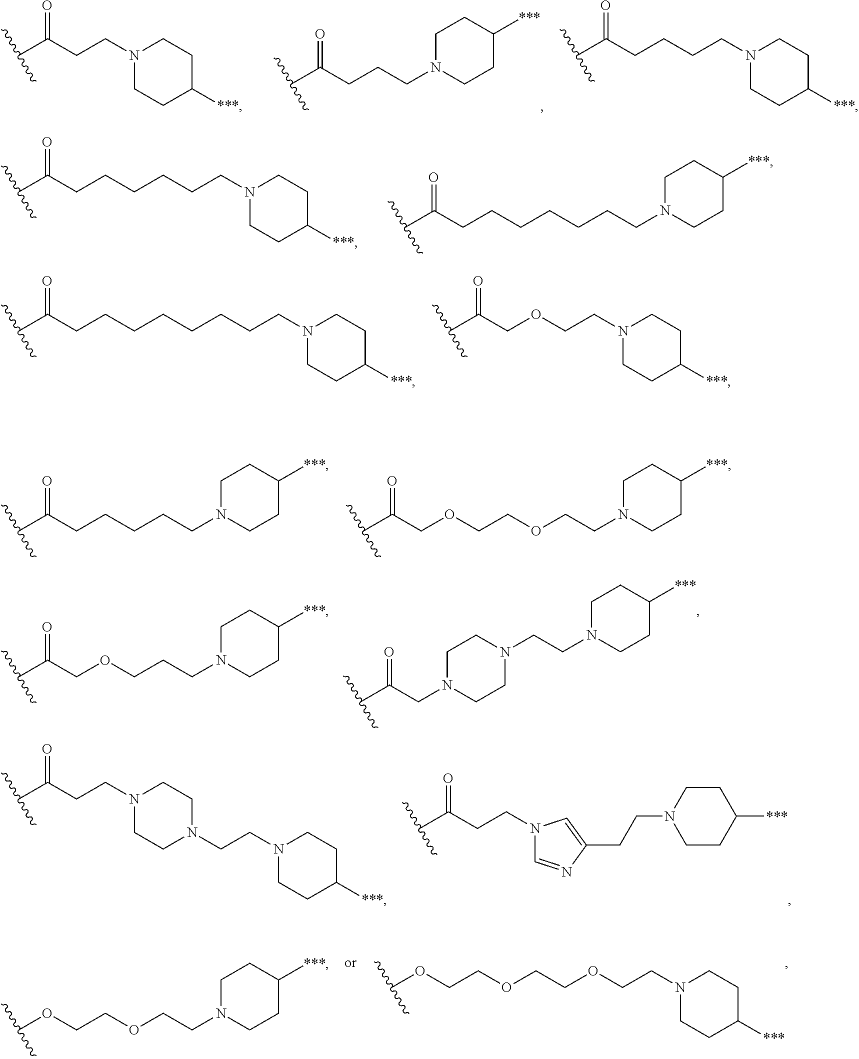
wherein *** is a point of attachment to TBM.

33. (canceled)

34. (canceled)

35. (canceled)

36. (canceled)

37. (canceled)

38. The compound of claim 1, wherein L is 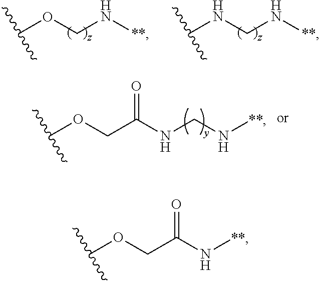
wherein y is 1, 2, or 3, wherein z is 4, 5, 6, or 7, and wherein ** is a point of attachment to LBM.

39. (canceled)

40. The compound of claim 1, wherein L is 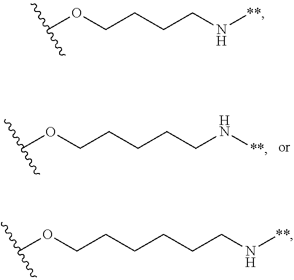
wherein ** is a point of attachment to LBM.

41. (canceled)

42. (canceled)

43. The compound of claim 1, wherein L is 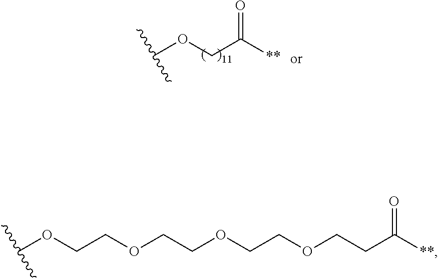
wherein ** is a point of attachment to LBM.

44. (canceled)

45. The compound of claim 1, wherein LBM is represented by Formula L-1: 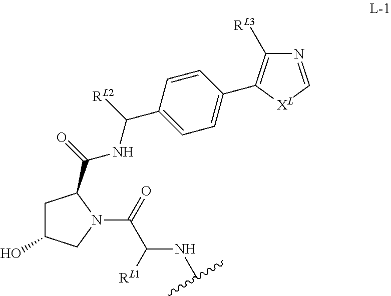
wherein:
R^{L1} is C₁₋₆ alkyl or C₃₋₇ cycloalkyl;
R^{L2} is C₁₋₄ alkyl or hydrogen;
R^{L3} is C₁₋₄ alkyl, C₃₋₅ cycloalkyl, C₁₋₄ hydroxyalkyl, or hydrogen; and
X^{L} is sulfur or oxygen.

46. (canceled)

47. The compound of claim 1, wherein LBM is represented by Formula L-3: 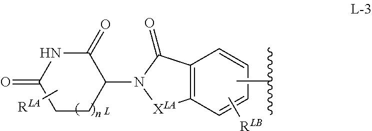
wherein:
R^{LA} and R^{LB} are each independently hydrogen, halo, C₁₋₆ alkyl, or C₁₋₆ haloalkyl;
X^{LA} is —C(O)— or —CH₂—; and
n^{L} is O or 1.

48. (canceled)

49. (canceled)

50. The compound of claim 1, wherein LBM is

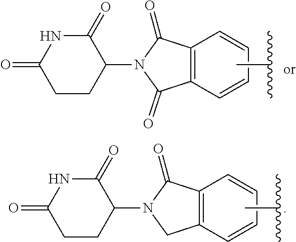

51. (canceled)

52. The compound of claim 1, wherein LBM is

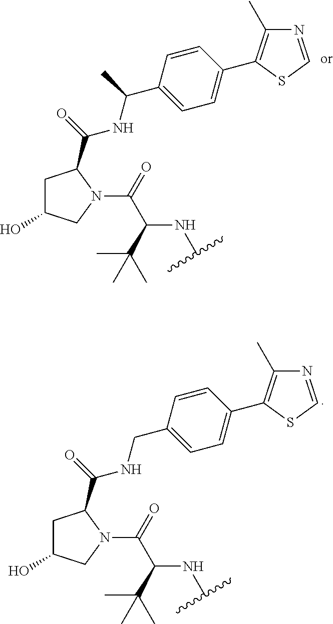 [SMILES: *N[C@H](C(=O)N1C[C@H](O)C[C@H]1C(=O)N[C@@H](C)c1ccc(-c2scnc2C)cc1)C(C)(C)C ; *N[C@H](C(=O)N1C[C@H](O)C[C@H]1C(=O)NCc1ccc(-c2scnc2C)cc1)C(C)(C)C]

53. A compound in Table 1 below, or a pharmaceutically acceptable salt thereof:
TABLE 1

| Com- | |
|---|---|
| pound | |
| No. | Chemical Structure |
| I-1 | 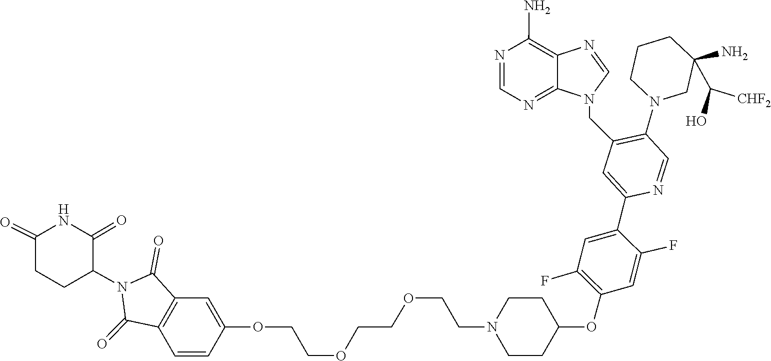 [SMILES: Nc1ncnc2c1ncn2Cc1cc(-c2cc(F)c(OC3CCN(CCOCCOCCOc4ccc5c(c4)C(=O)N(C4CCC(=O)NC4=O)C5=O)CC3)cc2F)ncc1N1CCC[C@](N)([C@H](O)C(F)F)C1] |
| I-2 | 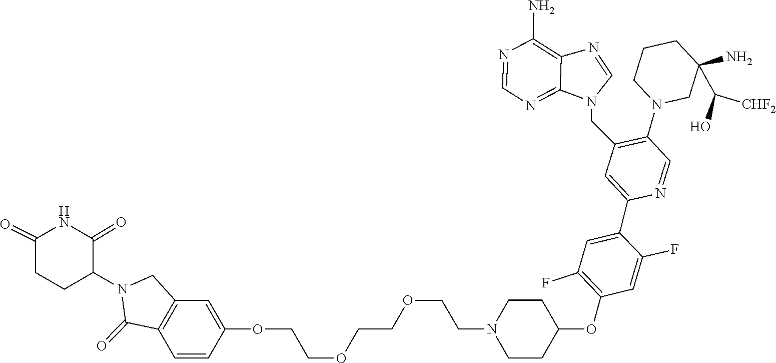 [SMILES: Nc1ncnc2c1ncn2Cc1cc(-c2cc(F)c(OC3CCN(CCOCCOCCOc4ccc5c(c4)CN(C4CCC(=O)NC4=O)C5=O)CC3)cc2F)ncc1N1CCC[C@](N)([C@H](O)C(F)F)C1] |
| I-3 | 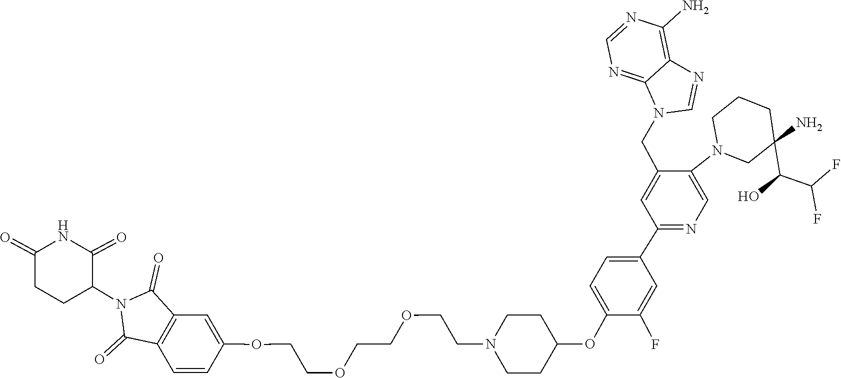 [SMILES: Nc1ncnc2c1ncn2Cc1cc(-c2ccc(OC3CCN(CCOCCOCCOc4ccc5c(c4)C(=O)N(C4CCC(=O)NC4=O)C5=O)CC3)c(F)c2)ncc1N1CCC[C@](N)([C@H](O)C(F)F)C1] |
| I-4 | 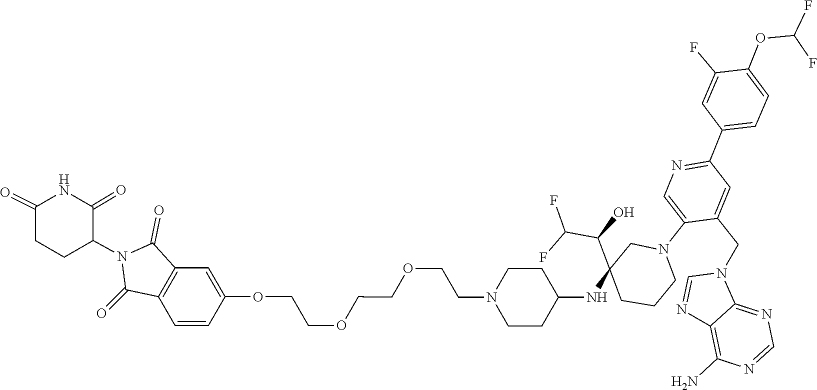 [SMILES: Nc1ncnc2c1ncn2Cc1cc(-c2ccc(OC(F)F)c(F)c2)ncc1N1CCC[C@](NC2CCN(CCOCCOCCOc3ccc4c(c3)C(=O)N(C3CCC(=O)NC3=O)C4=O)CC2)([C@H](O)C(F)F)C1] |
| I-5 | 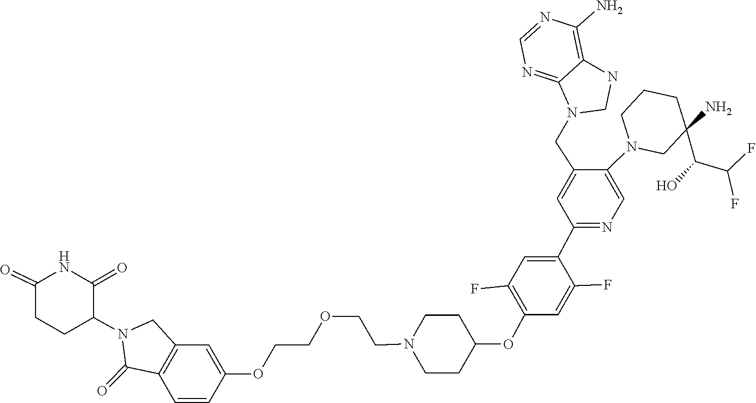 |
| I-6 | 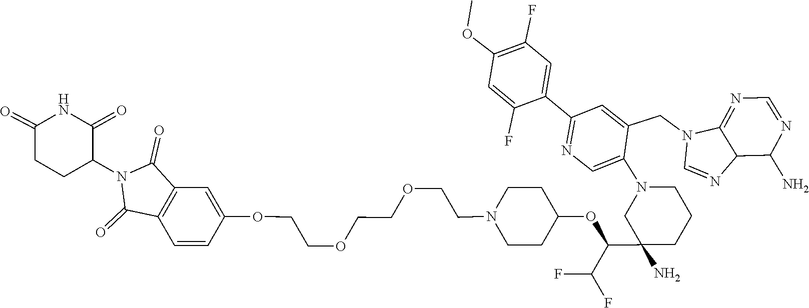 [SMILES: COc1cc(F)c(-c2cc(CN3C=NC4C3=NC=NC4N)c(N3CCC[C@](N)([C@H](OC4CCN(CCOCCOCCOc5ccc6c(c5)C(=O)N(C5CCC(=O)NC5=O)C6=O)CC4)C(F)F)C3)cn2)cc1F] |
| I-7 | 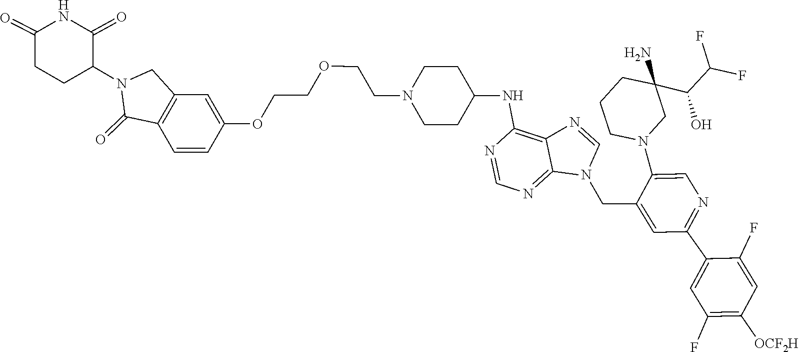 |
| I-8 | 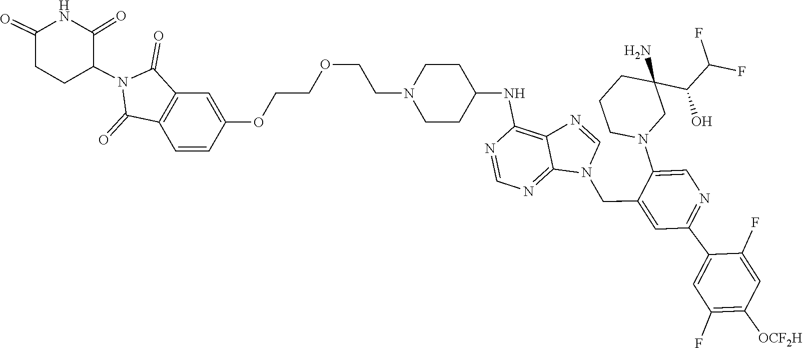 |
| I-9 | 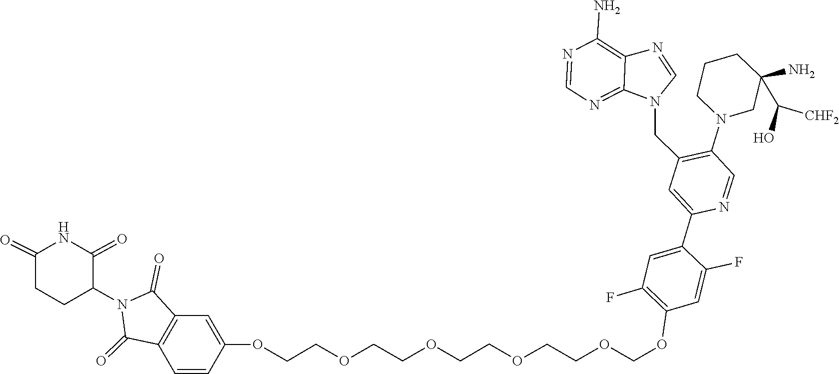 [SMILES: Nc1ncnc2c1ncn2Cc1cc(-c2cc(F)c(OCOCCOCCOCCOCCOc3ccc4c(c3)C(=O)N(C3CCC(=O)NC3=O)C4=O)cc2F)ncc1N1CCC[C@](N)([C@H](O)C(F)F)C1] |
| I-10 | 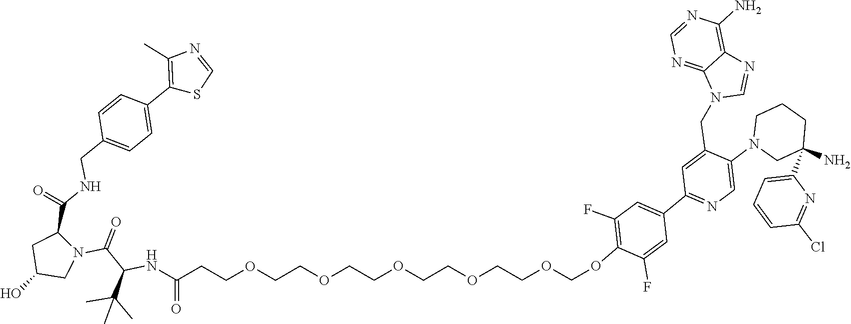 [SMILES: Cc1ncsc1-c1ccc(CNC(=O)[C@@H]2C[C@@H](O)CN2C(=O)[C@@H](NC(=O)CCOCCOCCOCCOCCOCOc2c(F)cc(-c3cc(Cn4cnc5c(N)ncnc54)c(N4CCC[C@](N)(c5cccc(Cl)n5)C4)cn3)cc2F)C(C)(C)C)cc1] |
| I-11 | 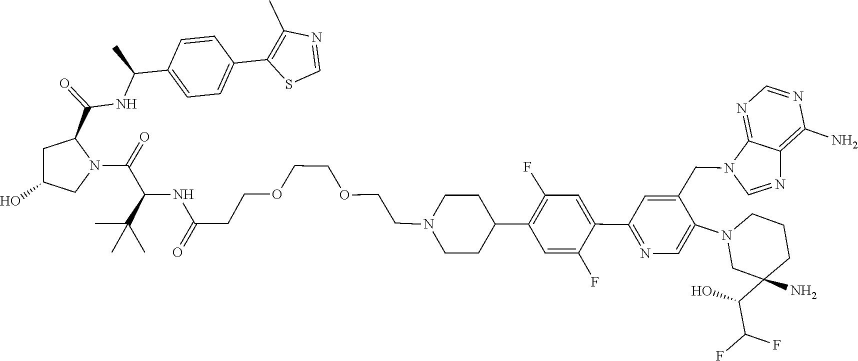 [SMILES: Cc1ncsc1-c1ccc([C@H](C)NC(=O)[C@@H]2C[C@@H](O)CN2C(=O)[C@@H](NC(=O)CCOCCOCCN2CCC(c3cc(F)c(-c4cc(Cn5cnc6c(N)ncnc65)c(N5CCC[C@](N)([C@@H](O)C(F)F)C5)cn4)cc3F)CC2)C(C)(C)C)cc1] |
| I-12 | 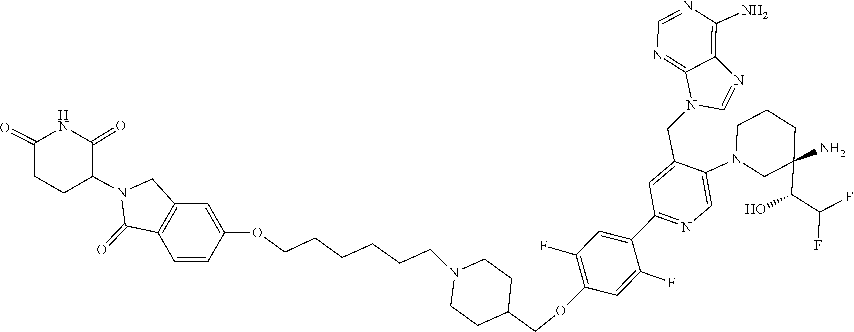 [SMILES: Nc1ncnc2c1ncn2Cc1cc(-c2cc(F)c(OCC3CCN(CCCCCCOc4ccc5c(c4)CN(C4CCC(=O)NC4=O)C5=O)CC3)cc2F)ncc1N1CCC[C@](N)([C@@H](O)C(F)F)C1] |
| I-13 | 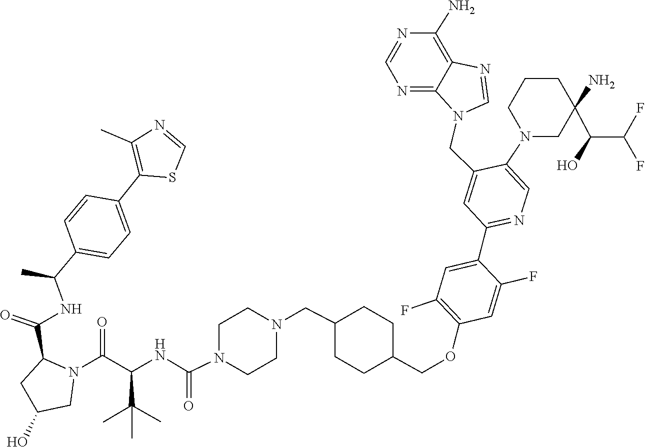 [SMILES: Cc1ncsc1-c1ccc([C@H](C)NC(=O)[C@@H]2C[C@@H](O)CN2C(=O)[C@@H](NC(=O)N2CCN(CC3CCC(COc4cc(F)c(-c5cc(Cn6cnc7c(N)ncnc76)c(N6CCC[C@](N)([C@H](O)C(F)F)C6)cn5)cc4F)CC3)CC2)C(C)(C)C)cc1] |
| I-14 | 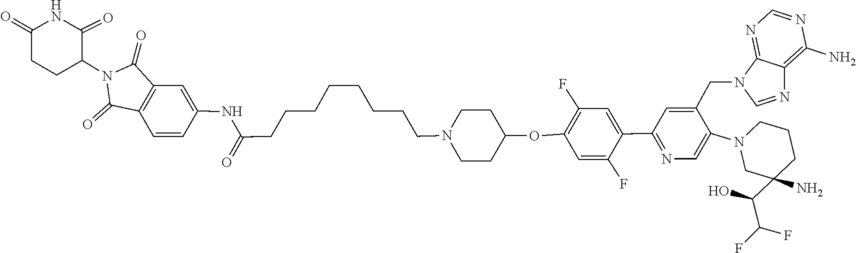 [SMILES: Nc1ncnc2c1ncn2Cc1cc(-c2cc(F)c(OC3CCN(CCCCCCCCC(=O)Nc4ccc5c(c4)C(=O)N(C4CCC(=O)NC4=O)C5=O)CC3)cc2F)ncc1N1CCC[C@](N)([C@H](O)C(F)F)C1] |
| I-15 | 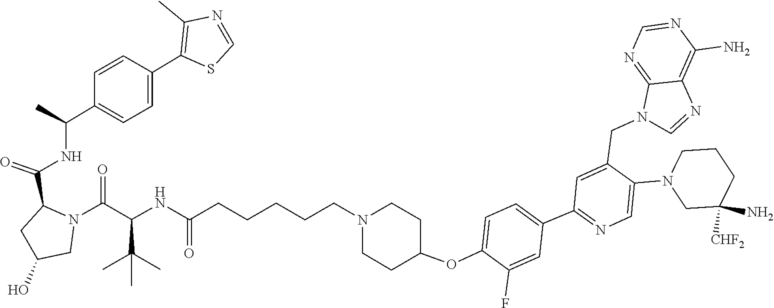 [SMILES: Cc1ncsc1-c1ccc([C@H](C)NC(=O)[C@@H]2C[C@@H](O)CN2C(=O)[C@@H](NC(=O)CCCCCN2CCC(Oc3ccc(-c4cc(Cn5cnc6c(N)ncnc65)c(N5CCC[C@](N)(C(F)F)C5)cn4)cc3F)CC2)C(C)(C)C)cc1] |
| I-16 | 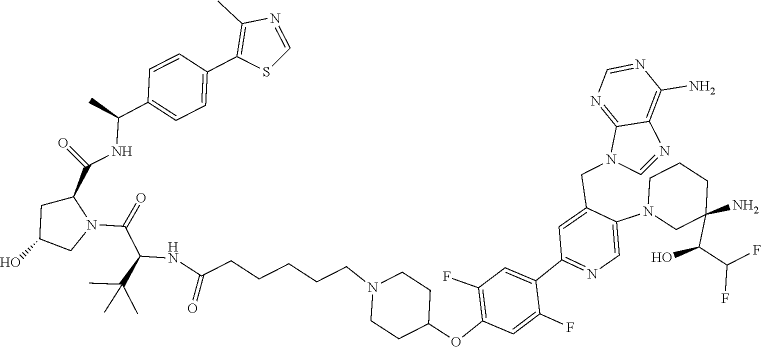 [SMILES: Cc1ncsc1-c1ccc([C@H](C)NC(=O)[C@@H]2C[C@@H](O)CN2C(=O)[C@@H](NC(=O)CCCCCN2CCC(Oc3cc(F)c(-c4cc(Cn5cnc6c(N)ncnc65)c(N5CCC[C@](N)([C@H](O)C(F)F)C5)cn4)cc3F)CC2)C(C)(C)C)cc1] |
| I-17 | 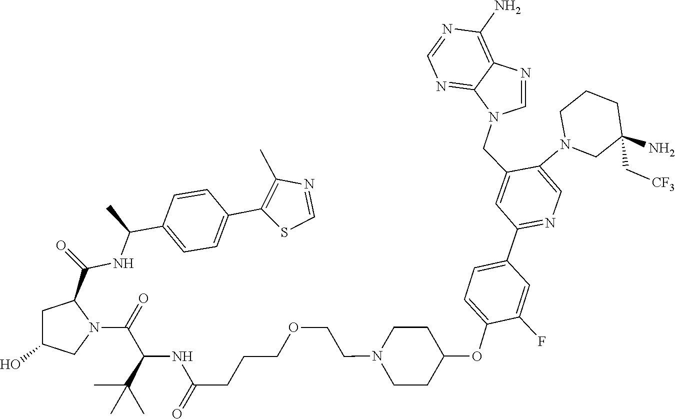 [SMILES: Cc1ncsc1-c1ccc([C@H](C)NC(=O)[C@@H]2C[C@@H](O)CN2C(=O)[C@@H](NC(=O)CCCOCCN2CCC(Oc3ccc(-c4cc(Cn5cnc6c(N)ncnc65)c(N5CCC[C@](N)(CC(F)(F)F)C5)cn4)cc3F)CC2)C(C)(C)C)cc1] |
| I-18 | 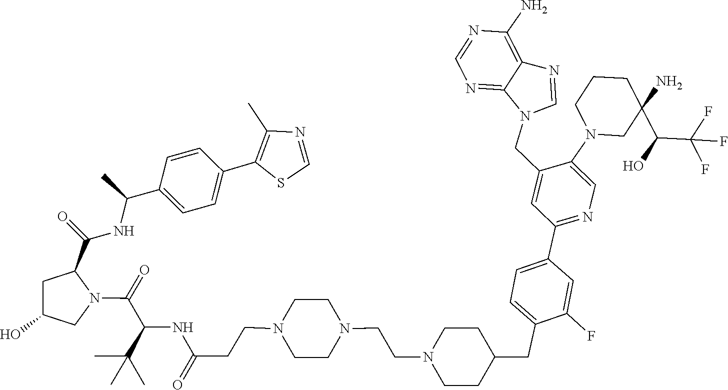 [SMILES: Cc1ncsc1-c1ccc([C@H](C)NC(=O)[C@@H]2C[C@@H](O)CN2C(=O)[C@@H](NC(=O)CCN2CCN(CCN3CCC(Cc4ccc(-c5cc(Cn6cnc7c(N)ncnc76)c(N6CCC[C@](N)([C@H](O)C(F)(F)F)C6)cn5)cc4F)CC3)CC2)C(C)(C)C)cc1] |
| I-19 | 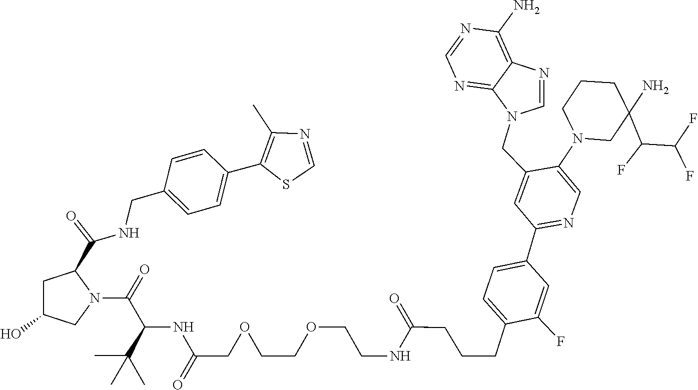 [SMILES: Cc1ncsc1-c1ccc(CNC(=O)[C@@H]2C[C@@H](O)CN2C(=O)[C@@H](NC(=O)COCCOCCNC(=O)CCCc2ccc(-c3cc(Cn4cnc5c(N)ncnc54)c(N4CCCC(N)(C(F)C(F)F)C4)cn3)cc2F)C(C)(C)C)cc1] |
| I-20 | 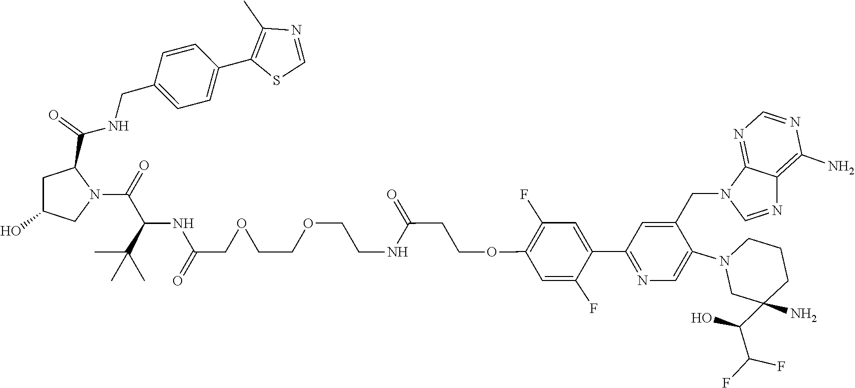 [SMILES: Cc1ncsc1-c1ccc(CNC(=O)[C@@H]2C[C@@H](O)CN2C(=O)[C@@H](NC(=O)COCCOCCNC(=O)CCOc2cc(F)c(-c3cc(Cn4cnc5c(N)ncnc54)c(N4CCC[C@](N)([C@H](O)C(F)F)C4)cn3)cc2F)C(C)(C)C)cc1] |
| I-21 | 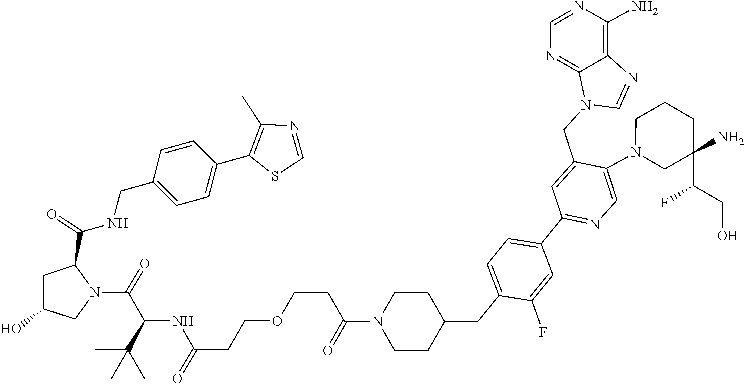 [SMILES: Cc1ncsc1-c1ccc(CNC(=O)[C@@H]2C[C@@H](O)CN2C(=O)[C@@H](NC(=O)CCOCCC(=O)N2CCC(Cc3ccc(-c4cc(Cn5cnc6c(N)ncnc65)c(N5CCC[C@](N)([C@@H](F)CO)C5)cn4)cc3F)CC2)C(C)(C)C)cc1] |
| I-22 | 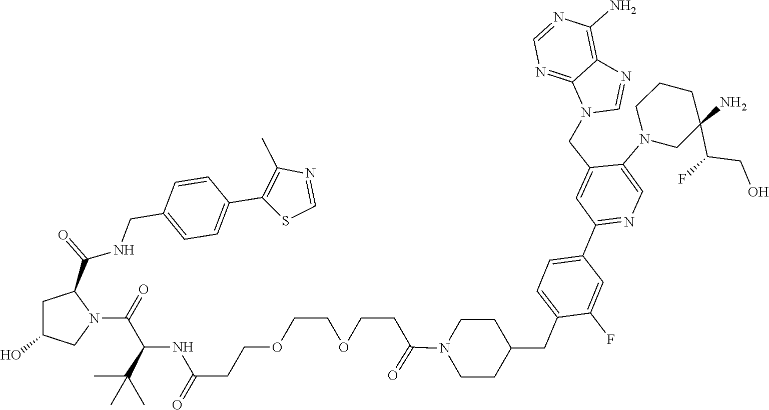 [SMILES: Cc1ncsc1-c1ccc(CNC(=O)[C@@H]2C[C@@H](O)CN2C(=O)[C@@H](NC(=O)CCOCCOCCC(=O)N2CCC(Cc3ccc(-c4cc(Cn5cnc6c(N)ncnc65)c(N5CCC[C@](N)([C@@H](F)CO)C5)cn4)cc3F)CC2)C(C)(C)C)cc1] |
| I-23 | 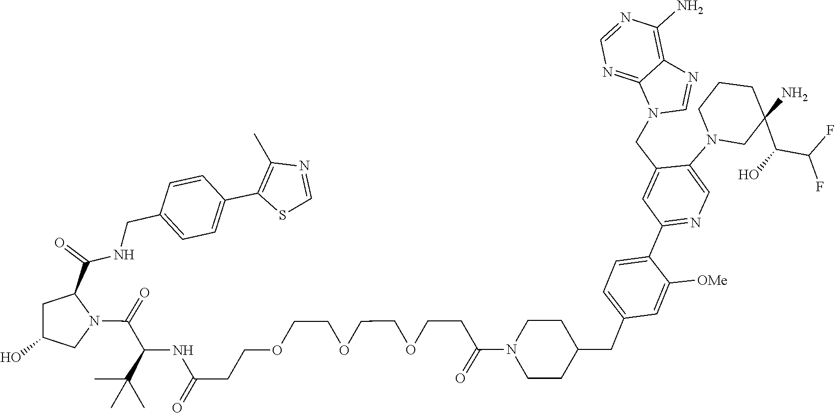 [SMILES: COc1cc(CC2CCN(C(=O)CCOCCOCCOCCC(=O)N[C@H](C(=O)N3C[C@H](O)C[C@H]3C(=O)NCc3ccc(-c4scnc4C)cc3)C(C)(C)C)CC2)ccc1-c1cc(Cn2cnc3c(N)ncnc32)c(N2CCC[C@](N)([C@@H](O)C(F)F)C2)cn1] |
| I-24 | 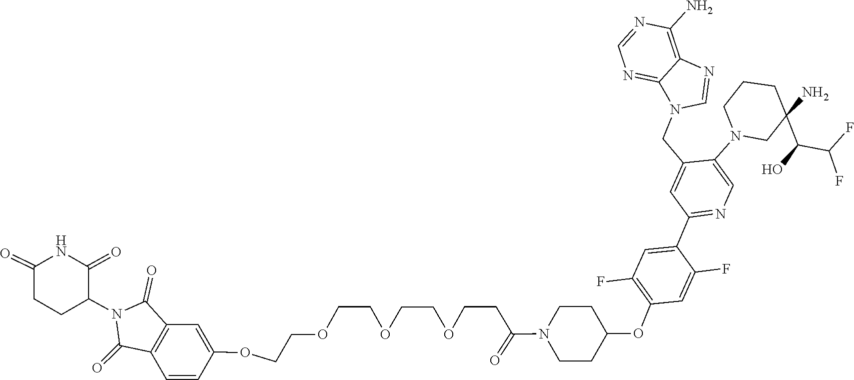 [SMILES: Nc1ncnc2c1ncn2Cc1cc(-c2cc(F)c(OC3CCN(C(=O)CCOCCOCCOCCOc4ccc5c(c4)C(=O)N(C4CCC(=O)NC4=O)C5=O)CC3)cc2F)ncc1N1CCC[C@](N)([C@H](O)C(F)F)C1] |
| I-25 | 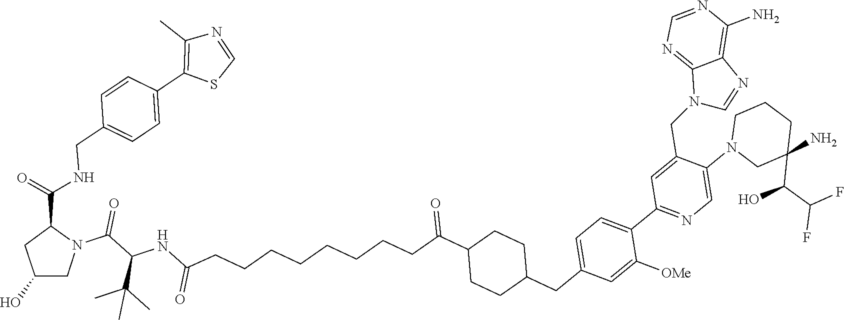 [SMILES: COc1cc(CC2CCC(C(=O)CCCCCCCCC(=O)N[C@H](C(=O)N3C[C@H](O)C[C@H]3C(=O)NCc3ccc(-c4scnc4C)cc3)C(C)(C)C)CC2)ccc1-c1cc(Cn2cnc3c(N)ncnc32)c(N2CCC[C@](N)([C@H](O)C(F)F)C2)cn1] |
| I-26 | 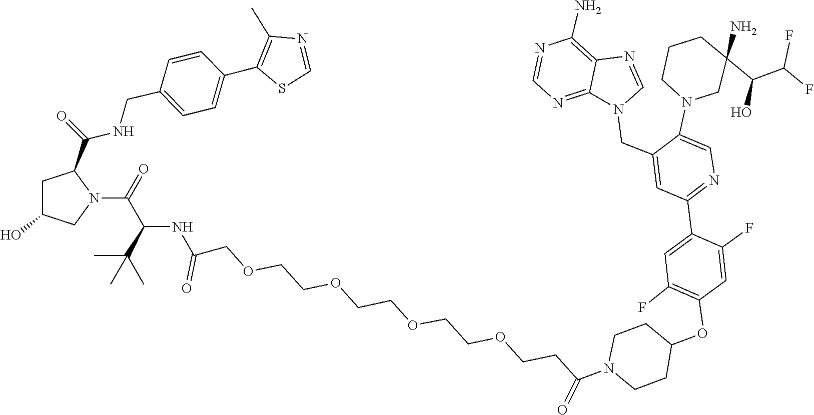 [SMILES: Cc1ncsc1-c1ccc(CNC(=O)[C@@H]2C[C@@H](O)CN2C(=O)[C@@H](NC(=O)COCCOCCOCCOCCC(=O)N2CCC(Oc3cc(F)c(-c4cc(Cn5cnc6c(N)ncnc65)c(N5CCC[C@](N)([C@H](O)C(F)F)C5)cn4)cc3F)CC2)C(C)(C)C)cc1] |
| I-27 | 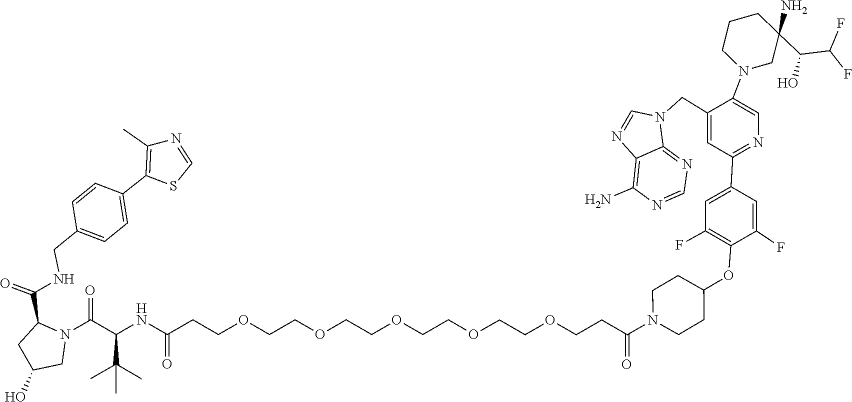 [SMILES: Cc1ncsc1-c1ccc(CNC(=O)[C@@H]2C[C@@H](O)CN2C(=O)[C@@H](NC(=O)CCOCCOCCOCCOCCOCCC(=O)N2CCC(Oc3c(F)cc(-c4cc(Cn5cnc6c(N)ncnc65)c(N5CCC[C@](N)([C@@H](O)C(F)F)C5)cn4)cc3F)CC2)C(C)(C)C)cc1] |
| I-28 | 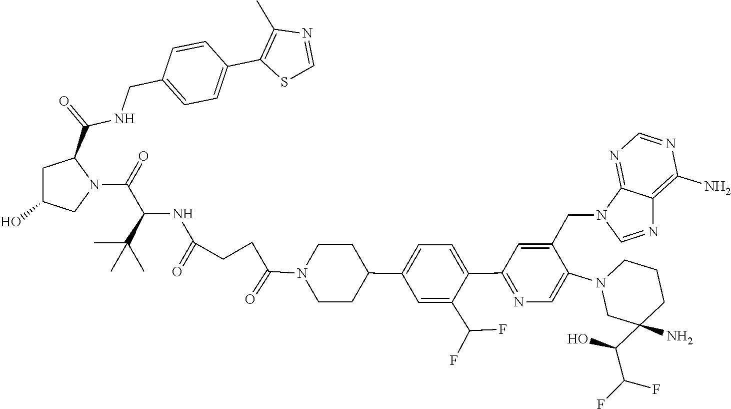 [SMILES: Cc1ncsc1-c1ccc(CNC(=O)[C@@H]2C[C@@H](O)CN2C(=O)[C@@H](NC(=O)CCC(=O)N2CCC(c3ccc(-c4cc(Cn5cnc6c(N)ncnc65)c(N5CCC[C@](N)([C@H](O)C(F)F)C5)cn4)c(C(F)F)c3)CC2)C(C)(C)C)cc1] |
| I-29 | 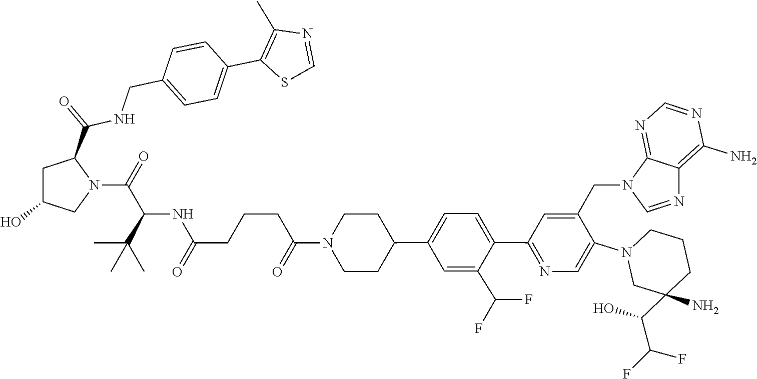 [SMILES: Cc1ncsc1-c1ccc(CNC(=O)[C@@H]2C[C@@H](O)CN2C(=O)[C@@H](NC(=O)CCCC(=O)N2CCC(c3ccc(-c4cc(Cn5cnc6c(N)ncnc65)c(N5CCC[C@](N)([C@@H](O)C(F)F)C5)cn4)c(C(F)F)c3)CC2)C(C)(C)C)cc1] |
| I-30 | 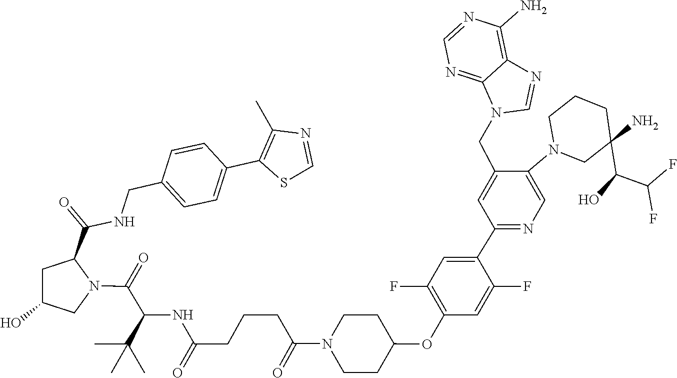 [SMILES: Cc1ncsc1-c1ccc(CNC(=O)[C@@H]2C[C@@H](O)CN2C(=O)[C@@H](NC(=O)CCCC(=O)N2CCC(Oc3cc(F)c(-c4cc(Cn5cnc6c(N)ncnc65)c(N5CCC[C@](N)([C@H](O)C(F)F)C5)cn4)cc3F)CC2)C(C)(C)C)cc1] |
| I-31 | 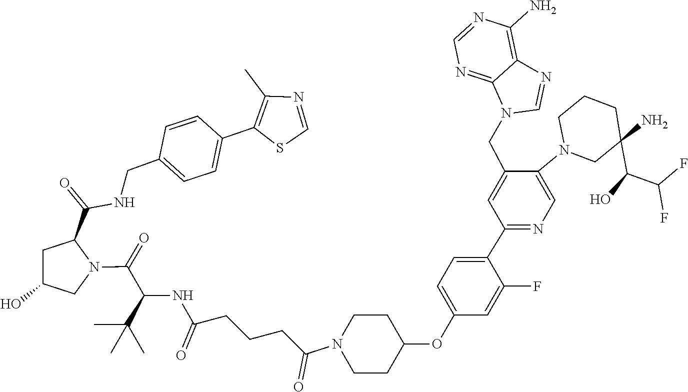 [SMILES: Cc1ncsc1-c1ccc(CNC(=O)[C@@H]2C[C@@H](O)CN2C(=O)[C@@H](NC(=O)CCCC(=O)N2CCC(Oc3ccc(-c4cc(Cn5cnc6c(N)ncnc65)c(N5CCC[C@](N)([C@H](O)C(F)F)C5)cn4)c(F)c3)CC2)C(C)(C)C)cc1] |
| I-32 | 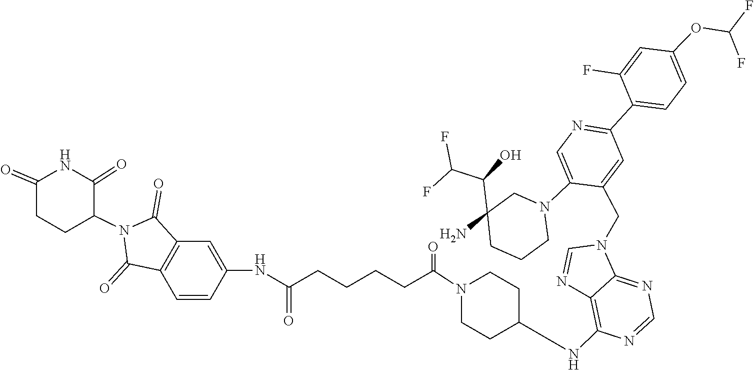 [SMILES: N[C@]1([C@H](O)C(F)F)CCCN(c2cnc(-c3ccc(OC(F)F)cc3F)cc2Cn2cnc3c(NC4CCN(C(=O)CCCCC(=O)Nc5ccc6c(c5)C(=O)N(C5CCC(=O)NC5=O)C6=O)CC4)ncnc32)C1] |
| I-33 | 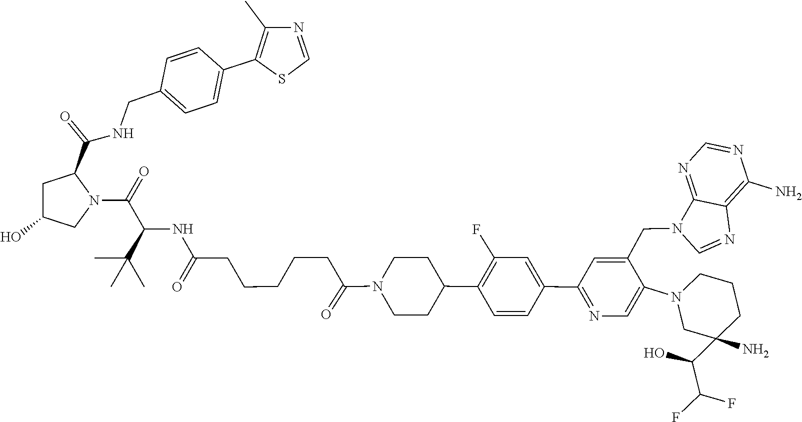 [SMILES: Cc1ncsc1-c1ccc(CNC(=O)[C@@H]2C[C@@H](O)CN2C(=O)[C@@H](NC(=O)CCCCCC(=O)N2CCC(c3ccc(-c4cc(Cn5cnc6c(N)ncnc65)c(N5CCC[C@](N)([C@H](O)C(F)F)C5)cn4)cc3F)CC2)C(C)(C)C)cc1] |
| I-34 | 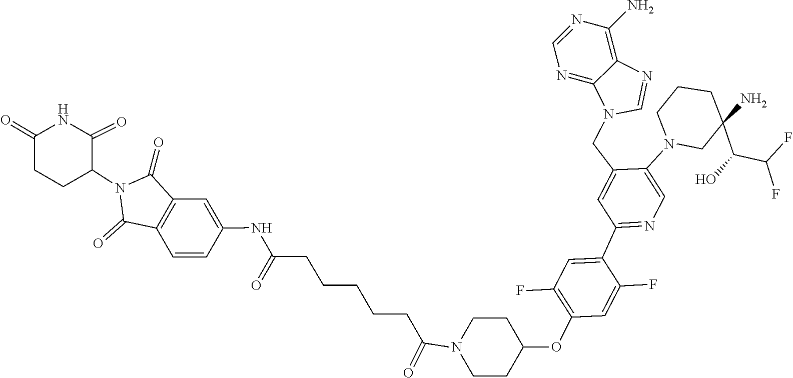 [SMILES: Nc1ncnc2c1ncn2Cc1cc(-c2cc(F)c(OC3CCN(C(=O)CCCCCC(=O)Nc4ccc5c(c4)C(=O)N(C4CCC(=O)NC4=O)C5=O)CC3)cc2F)ncc1N1CCC[C@](N)([C@@H](O)C(F)F)C1] |
| I-35 | 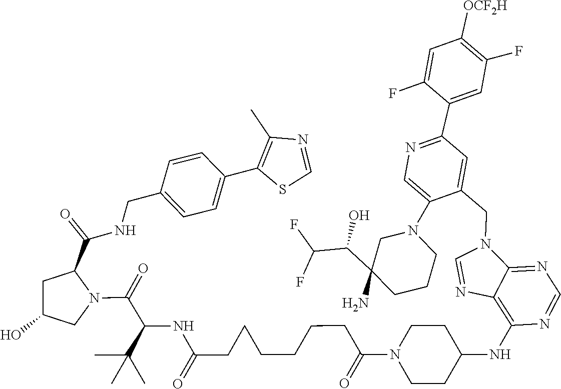 |
| I-36 | 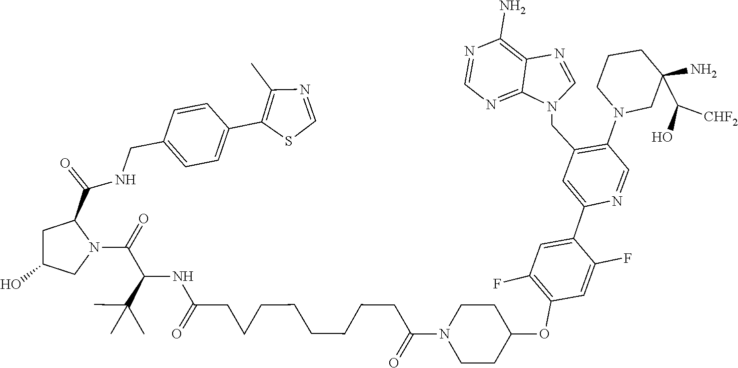 [SMILES: Cc1ncsc1-c1ccc(CNC(=O)[C@@H]2C[C@@H](O)CN2C(=O)[C@@H](NC(=O)CCCCCCCCC(=O)N2CCC(Oc3cc(F)c(-c4cc(Cn5cnc6c(N)ncnc65)c(N5CCC[C@](N)([C@H](O)C(F)F)C5)cn4)cc3F)CC2)C(C)(C)C)cc1] |
| I-37 | 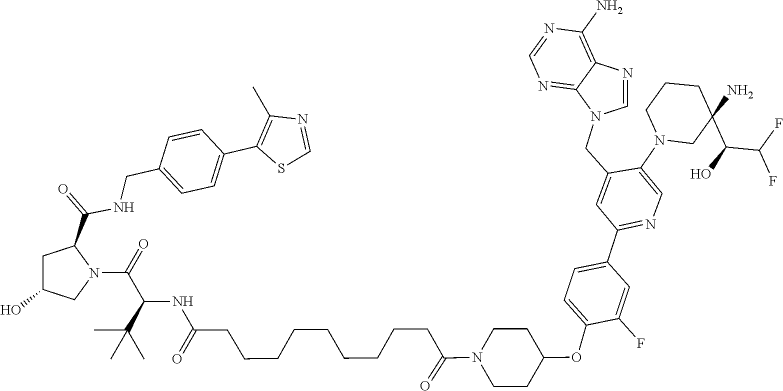 [SMILES: Cc1ncsc1-c1ccc(CNC(=O)[C@@H]2C[C@@H](O)CN2C(=O)[C@@H](NC(=O)CCCCCCCCCC(=O)N2CCC(Oc3ccc(-c4cc(Cn5cnc6c(N)ncnc65)c(N5CCC[C@](N)([C@H](O)C(F)F)C5)cn4)cc3F)CC2)C(C)(C)C)cc1] |
| I-38 | 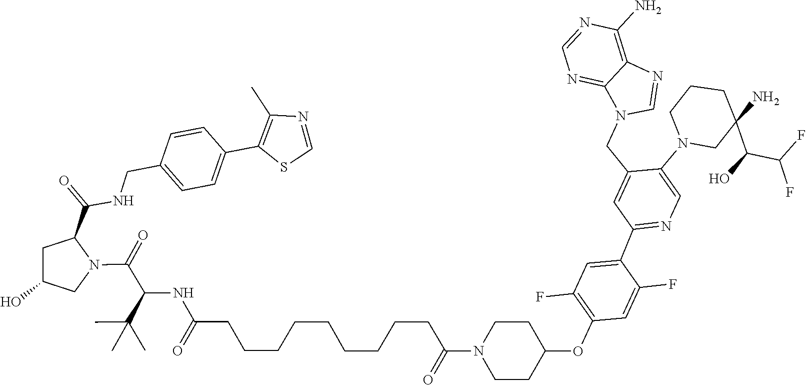 [SMILES: Cc1ncsc1-c1ccc(CNC(=O)[C@@H]2C[C@@H](O)CN2C(=O)[C@@H](NC(=O)CCCCCCCCCC(=O)N2CCC(Oc3cc(F)c(-c4cc(Cn5cnc6c(N)ncnc65)c(N5CCC[C@](N)([C@H](O)C(F)F)C5)cn4)cc3F)CC2)C(C)(C)C)cc1] |
| I-39 | 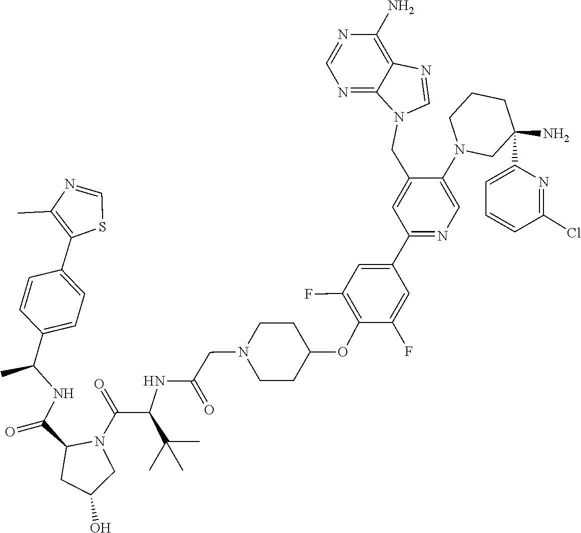 [SMILES: Cc1ncsc1-c1ccc([C@H](C)NC(=O)[C@@H]2C[C@@H](O)CN2C(=O)[C@@H](NC(=O)CN2CCC(Oc3c(F)cc(-c4cc(Cn5cnc6c(N)ncnc65)c(N5CCC[C@](N)(c6cccc(Cl)n6)C5)cn4)cc3F)CC2)C(C)(C)C)cc1] |
| I-40 | 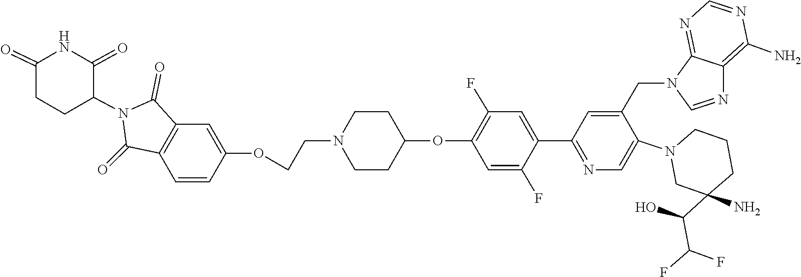 [SMILES: Nc1ncnc2c1ncn2Cc1cc(-c2cc(F)c(OC3CCN(CCOc4ccc5c(c4)C(=O)N(C4CCC(=O)NC4=O)C5=O)CC3)cc2F)ncc1N1CCC[C@](N)([C@H](O)C(F)F)C1] |
| I-41 | 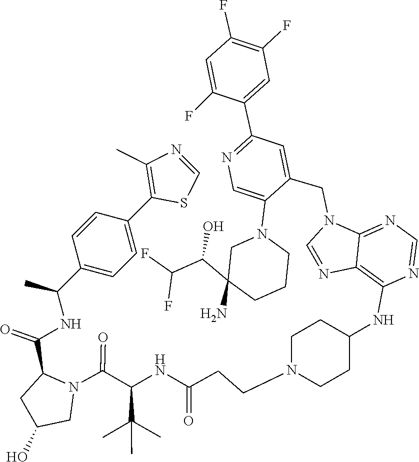 [SMILES: Cc1ncsc1-c1ccc([C@H](C)NC(=O)[C@@H]2C[C@@H](O)CN2C(=O)[C@@H](NC(=O)CCN2CCC(Nc3ncnc4c3ncn4Cc3cc(-c4cc(F)c(F)cc4F)ncc3N3CCC[C@](N)([C@@H](O)C(F)F)C3)CC2)C(C)(C)C)cc1] |
| I-42 | 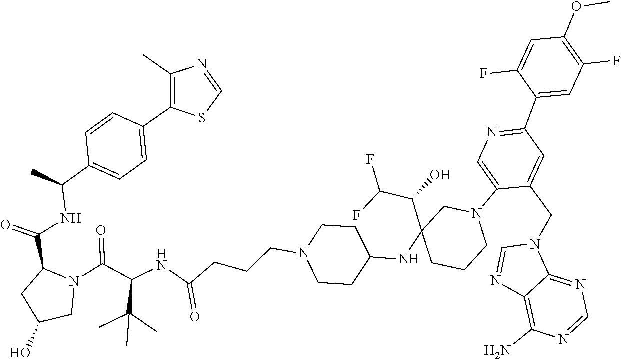 [SMILES: COc1cc(F)c(-c2cc(Cn3cnc4c(N)ncnc43)c(N3CCCC(NC4CCN(CCCC(=O)N[C@H](C(=O)N5C[C@H](O)C[C@H]5C(=O)N[C@@H](C)c5ccc(-c6scnc6C)cc5)C(C)(C)C)CC4)([C@@H](O)C(F)F)C3)cn2)cc1F] |
| I-43 | 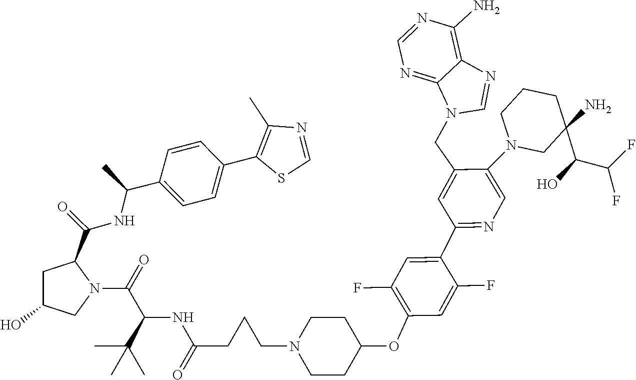 [SMILES: Cc1ncsc1-c1ccc([C@H](C)NC(=O)[C@@H]2C[C@@H](O)CN2C(=O)[C@@H](NC(=O)CCCN2CCC(Oc3cc(F)c(-c4cc(Cn5cnc6c(N)ncnc65)c(N5CCC[C@](N)([C@H](O)C(F)F)C5)cn4)cc3F)CC2)C(C)(C)C)cc1] |
| I-44 | 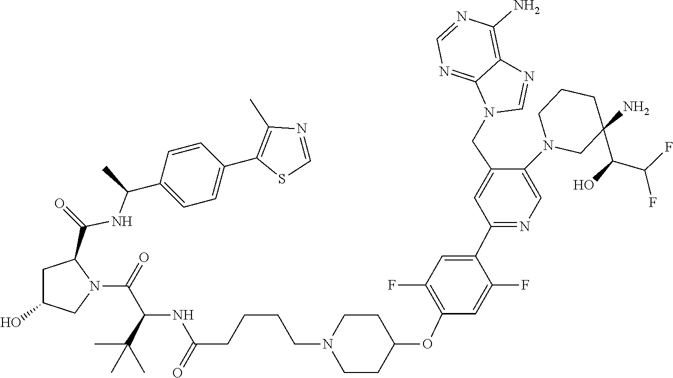 [SMILES: Cc1ncsc1-c1ccc([C@H](C)NC(=O)[C@@H]2C[C@@H](O)CN2C(=O)[C@@H](NC(=O)CCCCN2CCC(Oc3cc(F)c(-c4cc(Cn5cnc6c(N)ncnc65)c(N5CCC[C@](N)([C@H](O)C(F)F)C5)cn4)cc3F)CC2)C(C)(C)C)cc1] |
| I-45 | 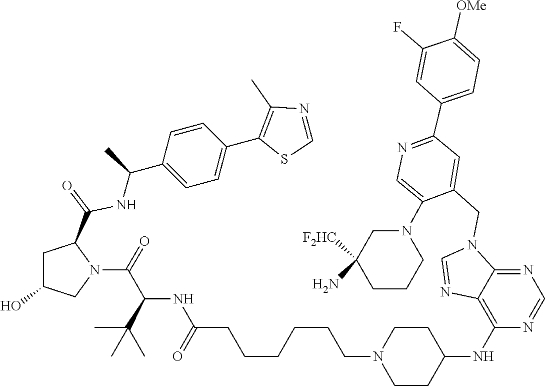 [SMILES: COc1ccc(-c2cc(Cn3cnc4c(NC5CCN(CCCCCCC(=O)N[C@H](C(=O)N6C[C@H](O)C[C@H]6C(=O)N[C@@H](C)c6ccc(-c7scnc7C)cc6)C(C)(C)C)CC5)ncnc43)c(N3CCC[C@](N)(C(F)F)C3)cn2)cc1F] |
| I-46 | 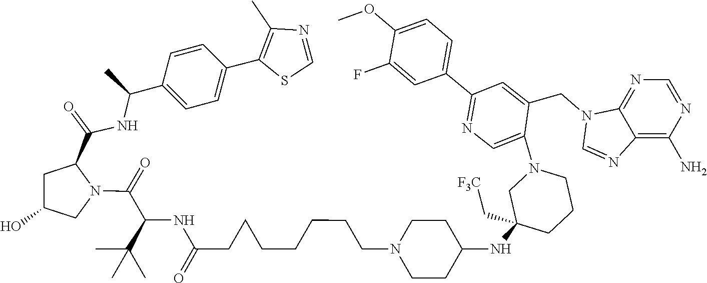 [SMILES: COc1ccc(-c2cc(Cn3cnc4c(N)ncnc43)c(N3CCC[C@@](CC(F)(F)F)(NC4CCN(CCCCCCCC(=O)N[C@H](C(=O)N5C[C@H](O)C[C@H]5C(=O)N[C@@H](C)c5ccc(-c6scnc6C)cc5)C(C)(C)C)CC4)C3)cn2)cc1F] |
| I-47 | 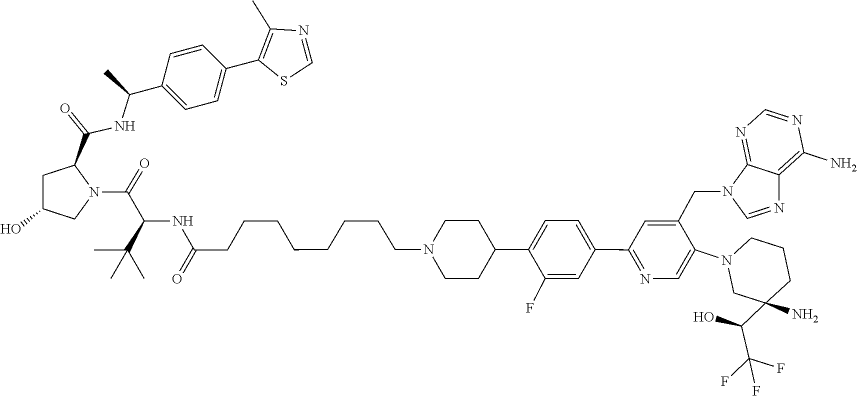 [SMILES: Cc1ncsc1-c1ccc([C@H](C)NC(=O)[C@@H]2C[C@@H](O)CN2C(=O)[C@@H](NC(=O)CCCCCCCCN2CCC(c3ccc(-c4cc(Cn5cnc6c(N)ncnc65)c(N5CCC[C@](N)([C@H](O)C(F)(F)F)C5)cn4)cc3F)CC2)C(C)(C)C)cc1] |
| I-48 | 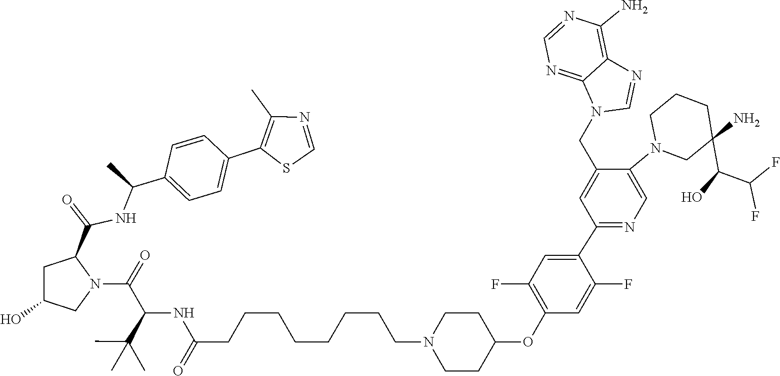 [SMILES: Cc1ncsc1-c1ccc([C@H](C)NC(=O)[C@@H]2C[C@@H](O)CN2C(=O)[C@@H](NC(=O)CCCCCCCCN2CCC(Oc3cc(F)c(-c4cc(Cn5cnc6c(N)ncnc65)c(N5CCC[C@](N)([C@H](O)C(F)F)C5)cn4)cc3F)CC2)C(C)(C)C)cc1] |
| I-49 | 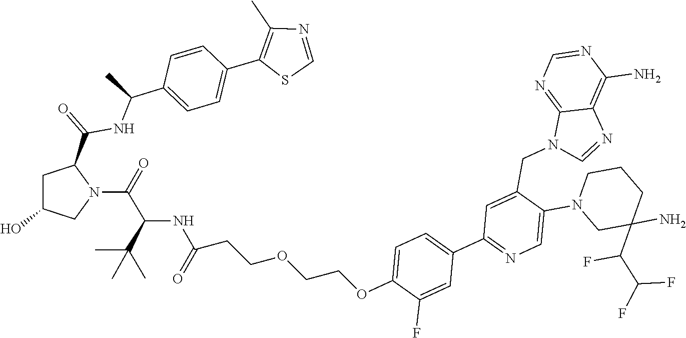 [SMILES: Cc1ncsc1-c1ccc([C@H](C)NC(=O)[C@@H]2C[C@@H](O)CN2C(=O)[C@@H](NC(=O)CCOCCOc2ccc(-c3cc(Cn4cnc5c(N)ncnc54)c(N4CCCC(N)(C(F)C(F)F)C4)cn3)cc2F)C(C)(C)C)cc1] |
| I-50 | 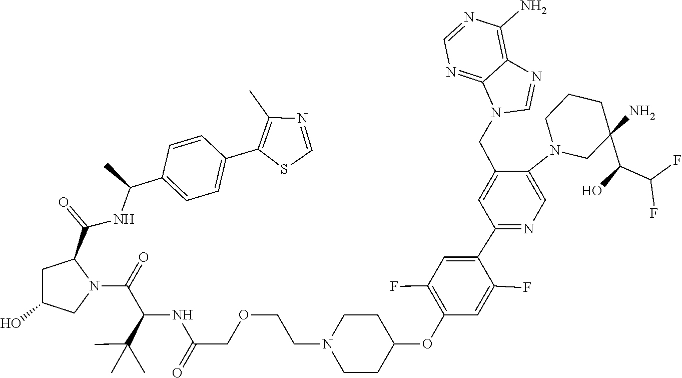 [SMILES: Cc1ncsc1-c1ccc([C@H](C)NC(=O)[C@@H]2C[C@@H](O)CN2C(=O)[C@@H](NC(=O)COCCN2CCC(Oc3cc(F)c(-c4cc(Cn5cnc6c(N)ncnc65)c(N5CCC[C@](N)([C@H](O)C(F)F)C5)cn4)cc3F)CC2)C(C)(C)C)cc1] |
| I-51 | 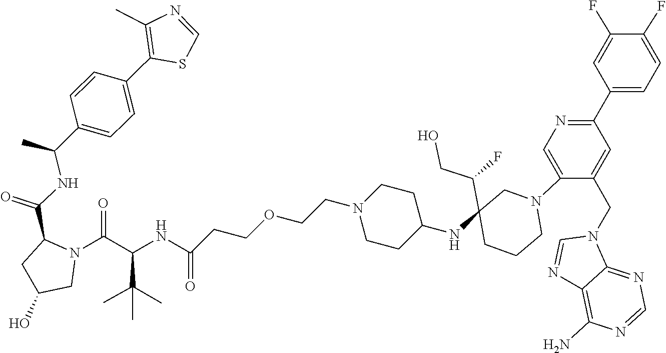 [SMILES: Cc1ncsc1-c1ccc([C@H](C)NC(=O)[C@@H]2C[C@@H](O)CN2C(=O)[C@@H](NC(=O)CCOCCN2CCC(N[C@]3([C@@H](F)CO)CCCN(c4cnc(-c5ccc(F)c(F)c5)cc4Cn4cnc5c(N)ncnc54)C3)CC2)C(C)(C)C)cc1] |
| I-52 | 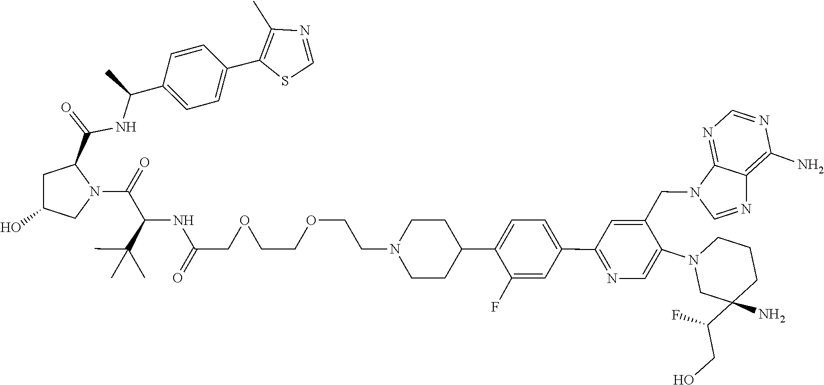 [SMILES: Cc1ncsc1-c1ccc([C@H](C)NC(=O)[C@@H]2C[C@@H](O)CN2C(=O)[C@@H](NC(=O)COCCOCCN2CCC(c3ccc(-c4cc(Cn5cnc6c(N)ncnc65)c(N5CCC[C@](N)([C@@H](F)CO)C5)cn4)cc3F)CC2)C(C)(C)C)cc1] |
| I-53 | 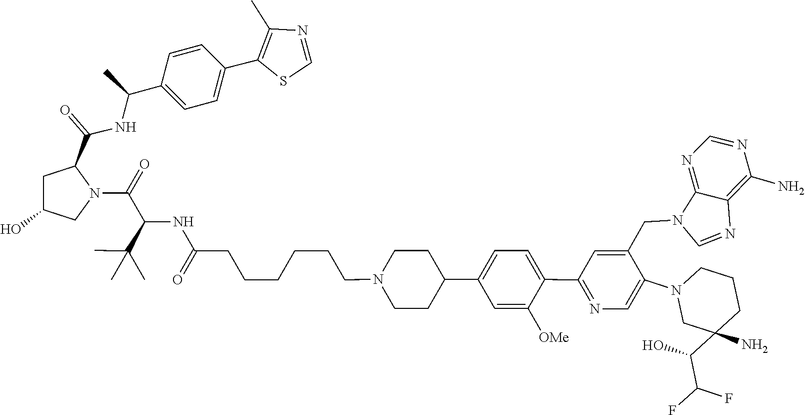 [SMILES: COc1cc(C2CCN(CCCCCCC(=O)N[C@H](C(=O)N3C[C@H](O)C[C@H]3C(=O)N[C@@H](C)c3ccc(-c4scnc4C)cc3)C(C)(C)C)CC2)ccc1-c1cc(Cn2cnc3c(N)ncnc32)c(N2CCC[C@](N)([C@@H](O)C(F)F)C2)cn1] |
| I-54 | 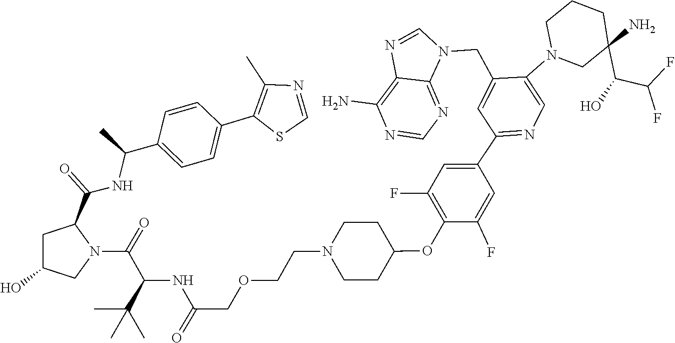 [SMILES: Cc1ncsc1-c1ccc([C@H](C)NC(=O)[C@@H]2C[C@@H](O)CN2C(=O)[C@@H](NC(=O)COCCN2CCC(Oc3c(F)cc(-c4cc(Cn5cnc6c(N)ncnc65)c(N5CCC[C@](N)([C@@H](O)C(F)F)C5)cn4)cc3F)CC2)C(C)(C)C)cc1] |
| I-55 | 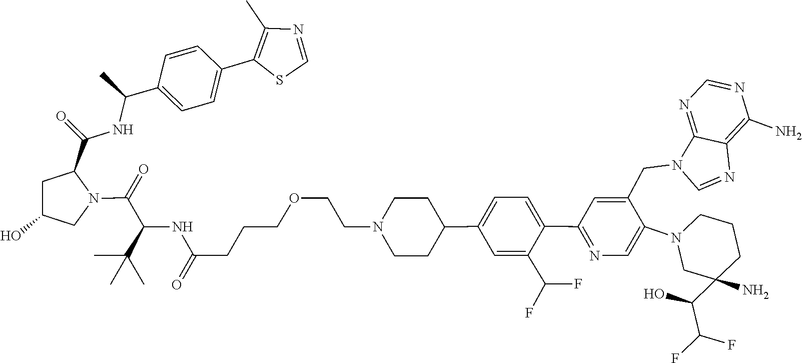 [SMILES: Cc1ncsc1-c1ccc([C@H](C)NC(=O)[C@@H]2C[C@@H](O)CN2C(=O)[C@@H](NC(=O)CCCOCCN2CCC(c3ccc(-c4cc(Cn5cnc6c(N)ncnc65)c(N5CCC[C@](N)([C@H](O)C(F)F)C5)cn4)c(C(F)F)c3)CC2)C(C)(C)C)cc1] |
| I-56 | 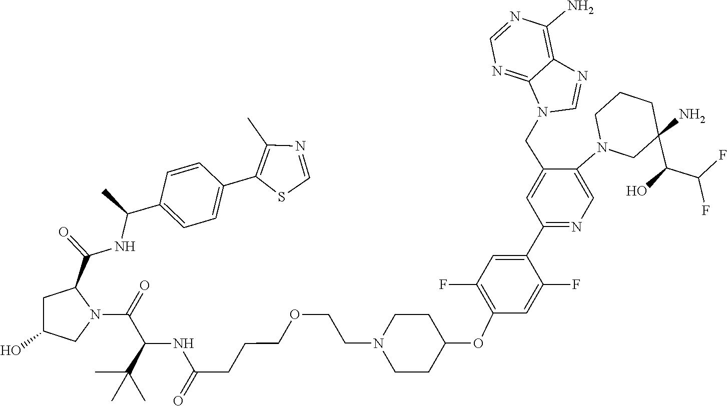 [SMILES: Cc1ncsc1-c1ccc([C@H](C)NC(=O)[C@@H]2C[C@@H](O)CN2C(=O)[C@@H](NC(=O)CCCOCCN2CCC(Oc3cc(F)c(-c4cc(Cn5cnc6c(N)ncnc65)c(N5CCC[C@](N)([C@H](O)C(F)F)C5)cn4)cc3F)CC2)C(C)(C)C)cc1] |
| I-57 | 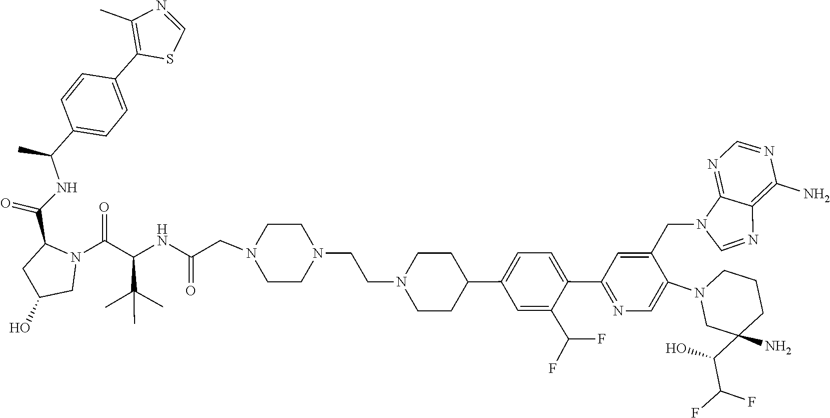 [SMILES: Cc1ncsc1-c1ccc([C@H](C)NC(=O)[C@@H]2C[C@@H](O)CN2C(=O)[C@@H](NC(=O)CN2CCN(CCN3CCC(c4ccc(-c5cc(Cn6cnc7c(N)ncnc76)c(N6CCC[C@](N)([C@@H](O)C(F)F)C6)cn5)c(C(F)F)c4)CC3)CC2)C(C)(C)C)cc1] |
| I-58 | 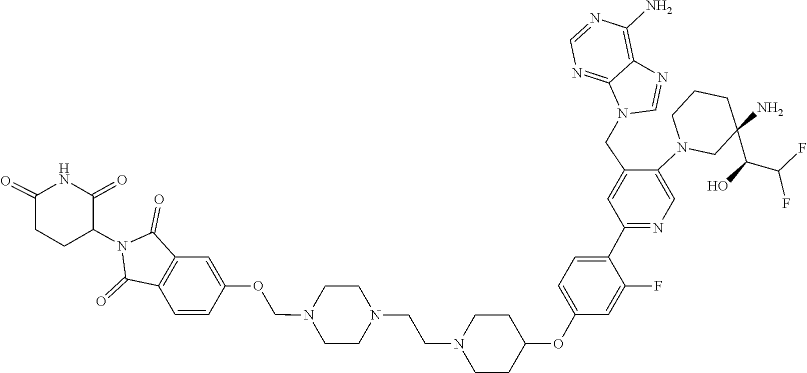 [SMILES: Nc1ncnc2c1ncn2Cc1cc(-c2ccc(OC3CCN(CCN4CCN(COc5ccc6c(c5)C(=O)N(C5CCC(=O)NC5=O)C6=O)CC4)CC3)cc2F)ncc1N1CCC[C@](N)([C@H](O)C(F)F)C1] |
| I-59 | 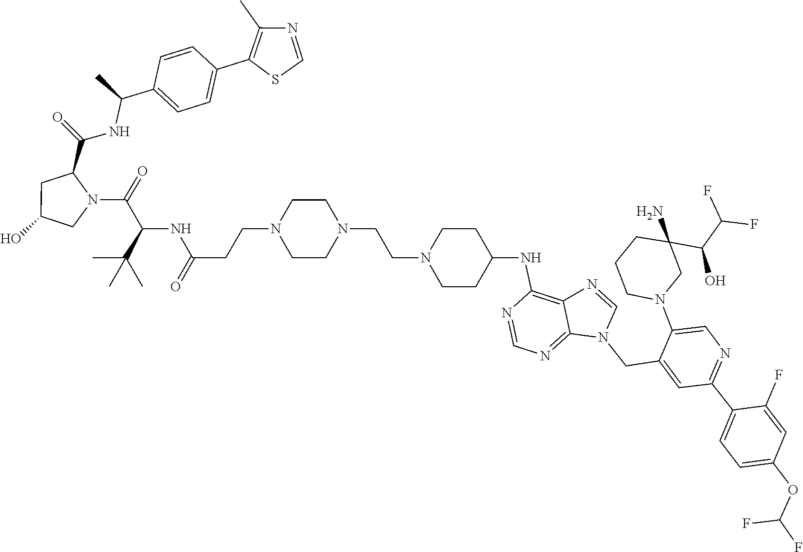 [SMILES: Cc1ncsc1-c1ccc([C@H](C)NC(=O)[C@@H]2C[C@@H](O)CN2C(=O)[C@@H](NC(=O)CCN2CCN(CCN3CCC(Nc4ncnc5c4ncn5Cc4cc(-c5ccc(OC(F)F)cc5F)ncc4N4CCC[C@](N)([C@H](O)C(F)F)C4)CC3)CC2)C(C)(C)C)cc1] |
| I-60 | 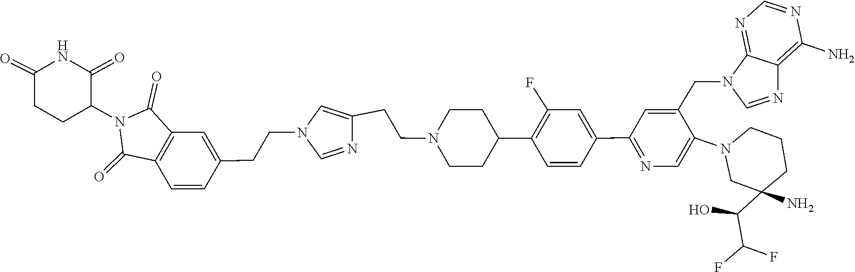 [SMILES: Nc1ncnc2c1ncn2Cc1cc(-c2ccc(C3CCN(CCc4cn(CCc5ccc6c(c5)C(=O)N(C5CCC(=O)NC5=O)C6=O)cn4)CC3)c(F)c2)ncc1N1CCC[C@](N)([C@H](O)C(F)F)C1] |
| I-61 | 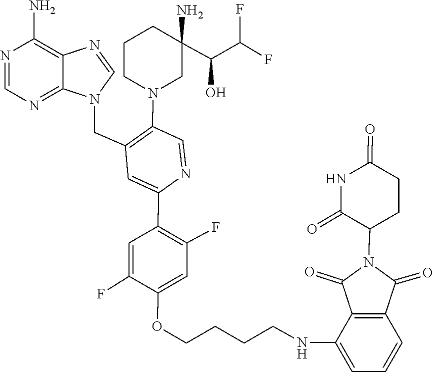 [SMILES: Nc1ncnc2c1ncn2Cc1cc(-c2cc(F)c(OCCCCNc3cccc4c3C(=O)N(C3CCC(=O)NC3=O)C4=O)cc2F)ncc1N1CCC[C@](N)([C@H](O)C(F)F)C1] |
| I-62 | 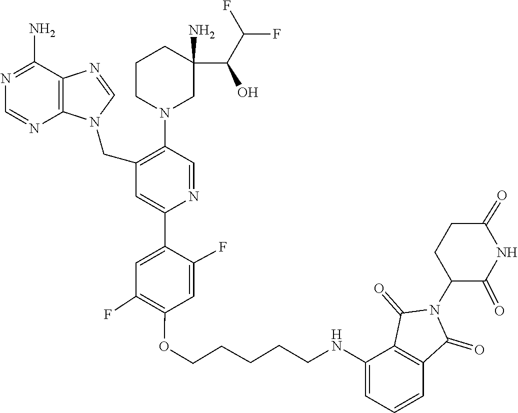 [SMILES: Nc1ncnc2c1ncn2Cc1cc(-c2cc(F)c(OCCCCCNc3cccc4c3C(=O)N(C3CCC(=O)NC3=O)C4=O)cc2F)ncc1N1CCC[C@](N)([C@H](O)C(F)F)C1] |
| I-63 | 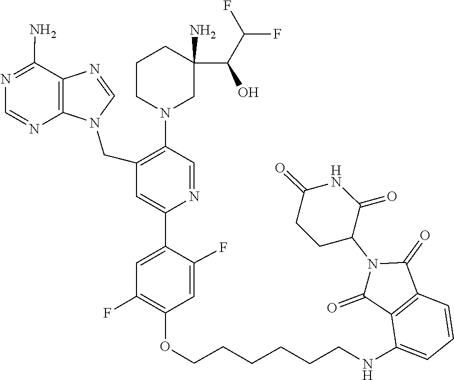 [SMILES: Nc1ncnc2c1ncn2Cc1cc(-c2cc(F)c(OCCCCCCNc3cccc4c3C(=O)N(C3CCC(=O)NC3=O)C4=O)cc2F)ncc1N1CCC[C@](N)([C@H](O)C(F)F)C1] |
| I-64 | 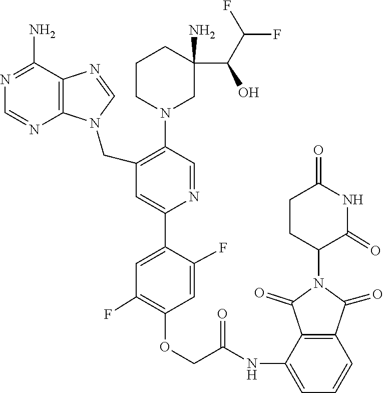 [SMILES: Nc1ncnc2c1ncn2Cc1cc(-c2cc(F)c(OCC(=O)Nc3cccc4c3C(=O)N(C3CCC(=O)NC3=O)C4=O)cc2F)ncc1N1CCC[C@](N)([C@H](O)C(F)F)C1] |
| I-65 | 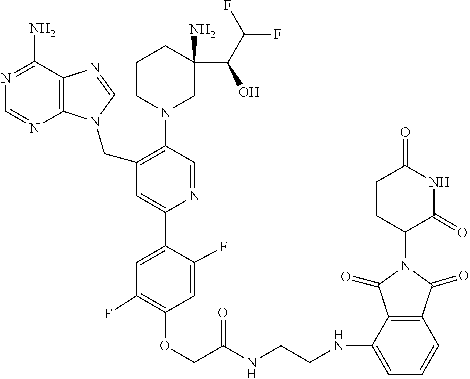 [SMILES: Nc1ncnc2c1ncn2Cc1cc(-c2cc(F)c(OCC(=O)NCCNc3cccc4c3C(=O)N(C3CCC(=O)NC3=O)C4=O)cc2F)ncc1N1CCC[C@](N)([C@H](O)C(F)F)C1] |
| I-66 | 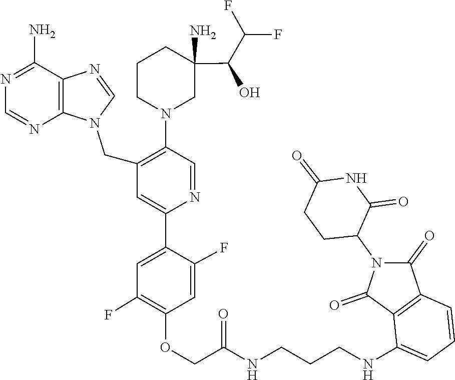 [SMILES: Nc1ncnc2c1ncn2Cc1cc(-c2cc(F)c(OCC(=O)NCCCNc3cccc4c3C(=O)N(C3CCC(=O)NC3=O)C4=O)cc2F)ncc1N1CCC[C@](N)([C@H](O)C(F)F)C1] |
| I-67 | 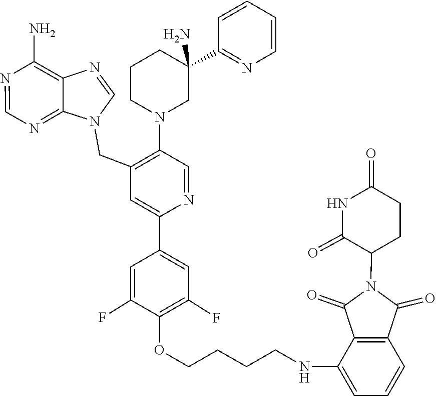 [SMILES: Nc1ncnc2c1ncn2Cc1cc(-c2cc(F)c(OCCCCNc3cccc4c3C(=O)N(C3CCC(=O)NC3=O)C4=O)c(F)c2)ncc1N1CCC[C@](N)(c2ccccn2)C1] |
| I-68 | 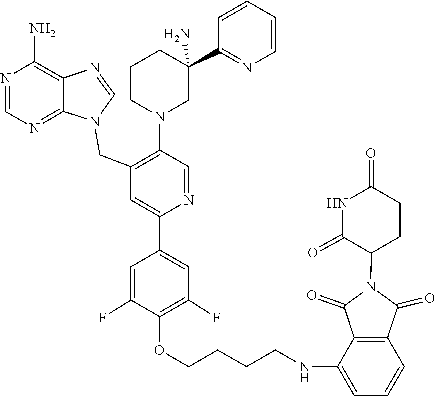 [SMILES: Nc1ncnc2c1ncn2Cc1cc(-c2cc(F)c(OCCCCNc3cccc4c3C(=O)N(C3CCC(=O)NC3=O)C4=O)c(F)c2)ncc1N1CCC[C@@](N)(c2ccccn2)C1] |
| I-69 | 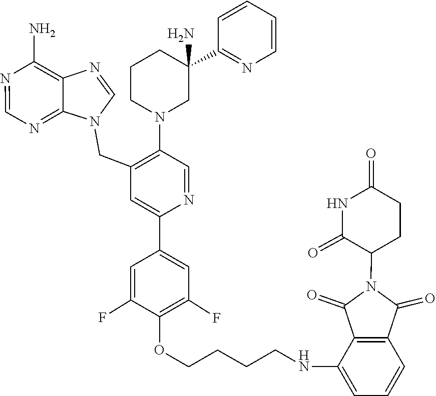 [SMILES: Nc1ncnc2c1ncn2Cc1cc(-c2cc(F)c(OCCCCNc3cccc4c3C(=O)N(C3CCC(=O)NC3=O)C4=O)c(F)c2)ncc1N1CCC[C@](N)(c2ccccn2)C1] |
| I-70 | 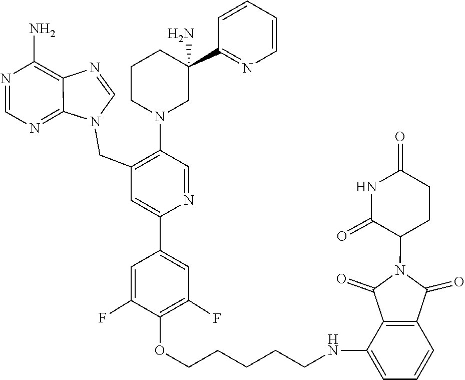 [SMILES: Nc1ncnc2c1ncn2Cc1cc(-c2cc(F)c(OCCCCCNc3cccc4c3C(=O)N(C3CCC(=O)NC3=O)C4=O)c(F)c2)ncc1N1CCC[C@@](N)(c2ccccn2)C1] |
| I-71 | 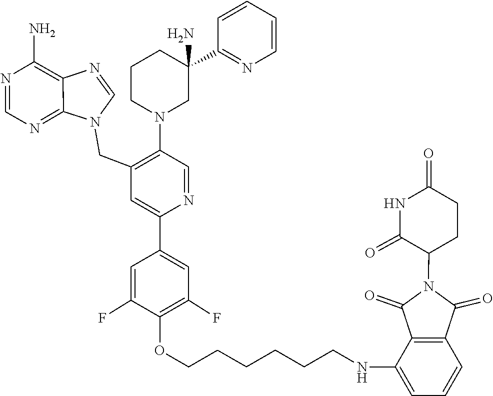 [SMILES: Nc1ncnc2c1ncn2Cc1cc(-c2cc(F)c(OCCCCCCNc3cccc4c3C(=O)N(C3CCC(=O)NC3=O)C4=O)c(F)c2)ncc1N1CCC[C@](N)(c2ccccn2)C1] |
| I-72 | 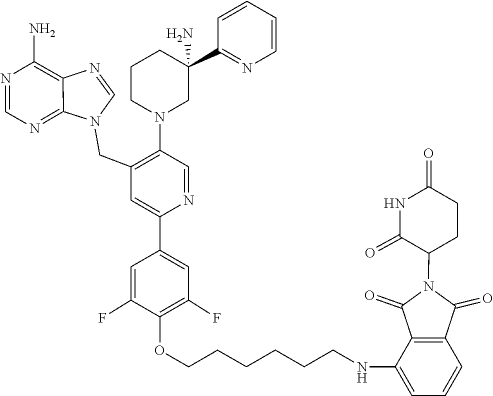 [SMILES: Nc1ncnc2c1ncn2Cc1cc(-c2cc(F)c(OCCCCCCNc3cccc4c3C(=O)N(C3CCC(=O)NC3=O)C4=O)c(F)c2)ncc1N1CCC[C@@](N)(c2ccccn2)C1] |
| I-73 | 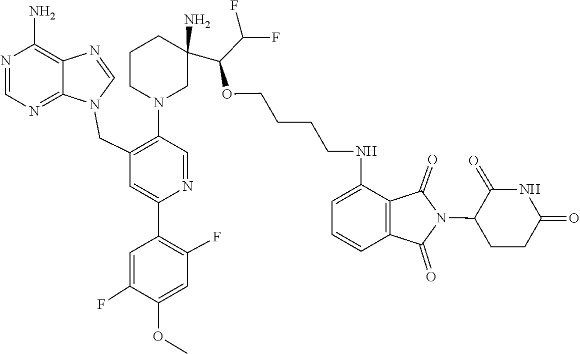 [SMILES: COc1cc(F)c(-c2cc(Cn3cnc4c(N)ncnc43)c(N3CCC[C@](N)([C@H](OCCCCNc4cccc5c4C(=O)N(C4CCC(=O)NC4=O)C5=O)C(F)F)C3)cn2)cc1F] |
| I-74 | 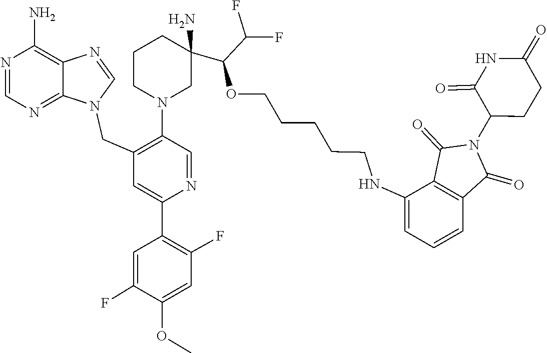 [SMILES: COc1cc(F)c(-c2cc(Cn3cnc4c(N)ncnc43)c(N3CCC[C@](N)([C@H](OCCCCCNc4cccc5c4C(=O)N(C4CCC(=O)NC4=O)C5=O)C(F)F)C3)cn2)cc1F] |
| I-75 | 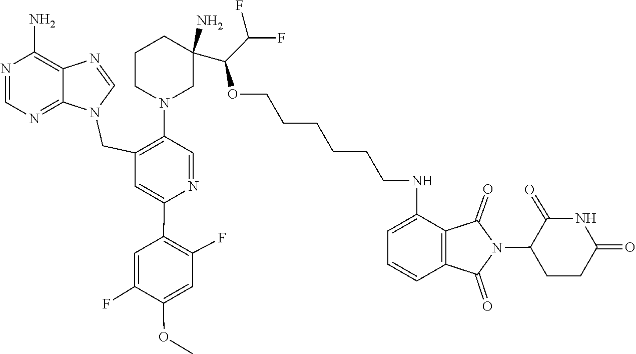 [SMILES: COc1cc(F)c(-c2cc(Cn3cnc4c(N)ncnc43)c(N3CCC[C@](N)([C@H](OCCCCCCNc4cccc5c4C(=O)N(C4CCC(=O)NC4=O)C5=O)C(F)F)C3)cn2)cc1F] |
| I-76 | 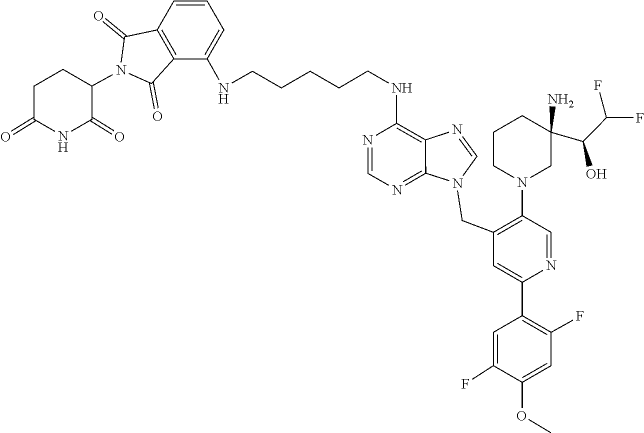 [SMILES: COc1cc(F)c(-c2cc(Cn3cnc4c(NCCCCCNc5cccc6c5C(=O)N(C5CCC(=O)NC5=O)C6=O)ncnc43)c(N3CCC[C@](N)([C@H](O)C(F)F)C3)cn2)cc1F] |
| I-77 | 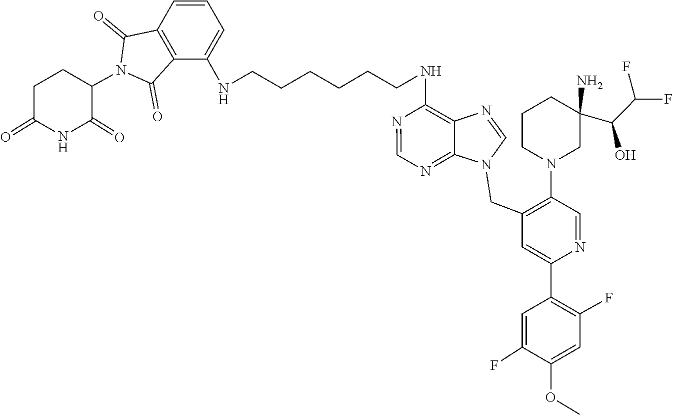 [SMILES: COc1cc(F)c(-c2cc(Cn3cnc4c(NCCCCCCNc5cccc6c5C(=O)N(C5CCC(=O)NC5=O)C6=O)ncnc43)c(N3CCC[C@](N)([C@H](O)C(F)F)C3)cn2)cc1F] |
| I-78 | 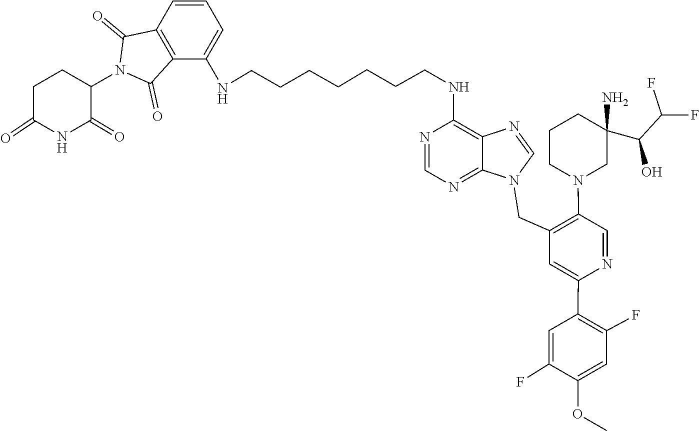 [SMILES: COc1cc(F)c(-c2cc(Cn3cnc4c(NCCCCCCCNc5cccc6c5C(=O)N(C5CCC(=O)NC5=O)C6=O)ncnc43)c(N3CCC[C@](N)([C@H](O)C(F)F)C3)cn2)cc1F] |
| I-79 | 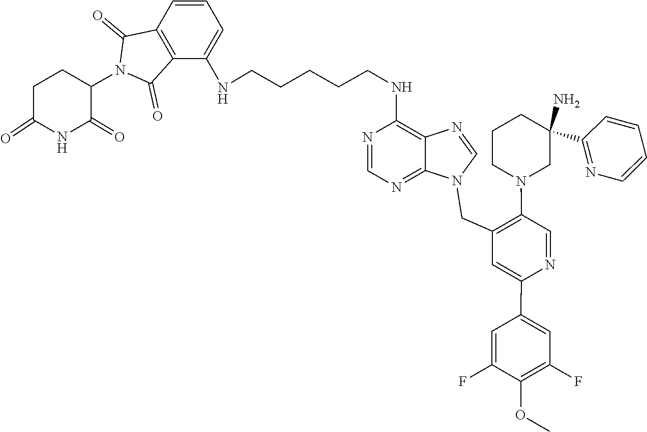 [SMILES: COc1c(F)cc(-c2cc(Cn3cnc4c(NCCCCCNc5cccc6c5C(=O)N(C5CCC(=O)NC5=O)C6=O)ncnc43)c(N3CCC[C@](N)(c4ccccn4)C3)cn2)cc1F] |
| I-80 | 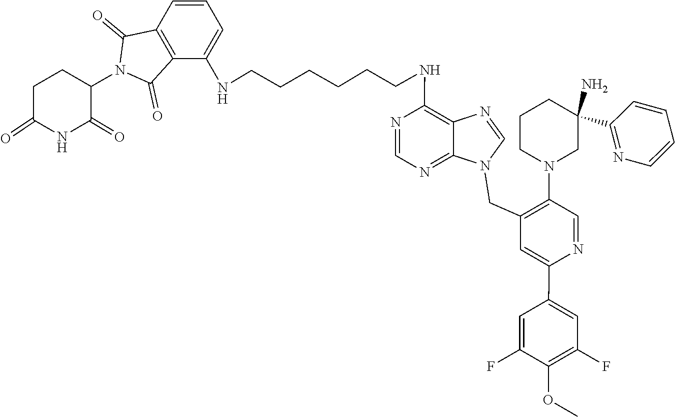 [SMILES: COc1c(F)cc(-c2cc(Cn3cnc4c(NCCCCCCNc5cccc6c5C(=O)N(C5CCC(=O)NC5=O)C6=O)ncnc43)c(N3CCC[C@](N)(c4ccccn4)C3)cn2)cc1F] |
| I-81 | 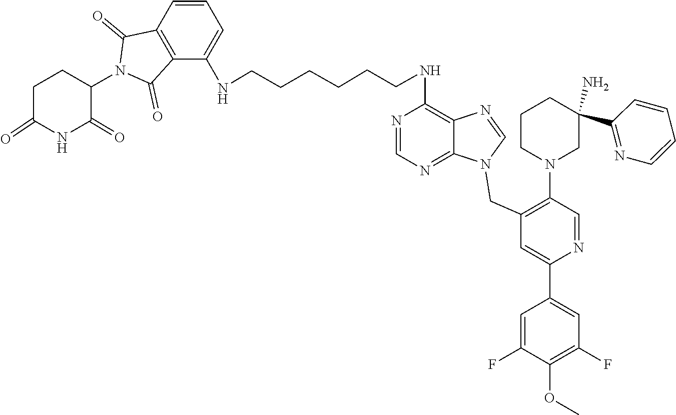 [SMILES: COc1c(F)cc(-c2cc(Cn3cnc4c(NCCCCCCNc5cccc6c5C(=O)N(C5CCC(=O)NC5=O)C6=O)ncnc43)c(N3CCC[C@@](N)(c4ccccn4)C3)cn2)cc1F] |
| I-82 | 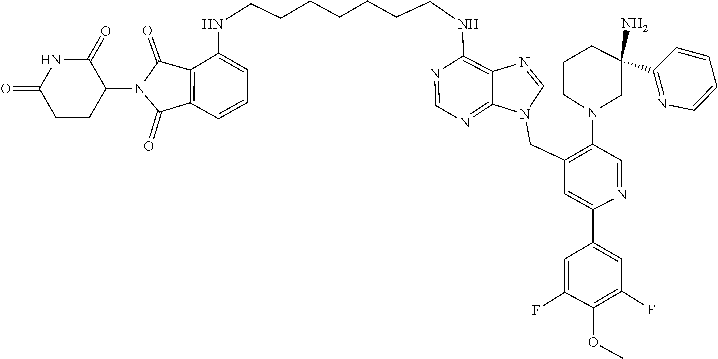 [SMILES: COc1c(F)cc(-c2cc(Cn3cnc4c(NCCCCCCCNc5cccc6c5C(=O)N(C5CCC(=O)NC5=O)C6=O)ncnc43)c(N3CCC[C@](N)(c4ccccn4)C3)cn2)cc1F] |
| I-83 | 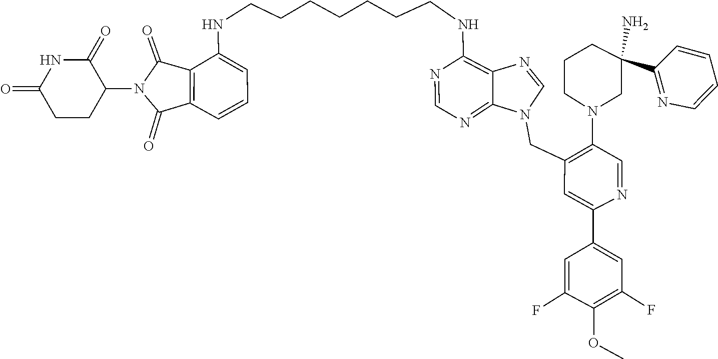 [SMILES: COc1c(F)cc(-c2cc(Cn3cnc4c(NCCCCCCCNc5cccc6c5C(=O)N(C5CCC(=O)NC5=O)C6=O)ncnc43)c(N3CCC[C@@](N)(c4ccccn4)C3)cn2)cc1F] |
| I-84 | 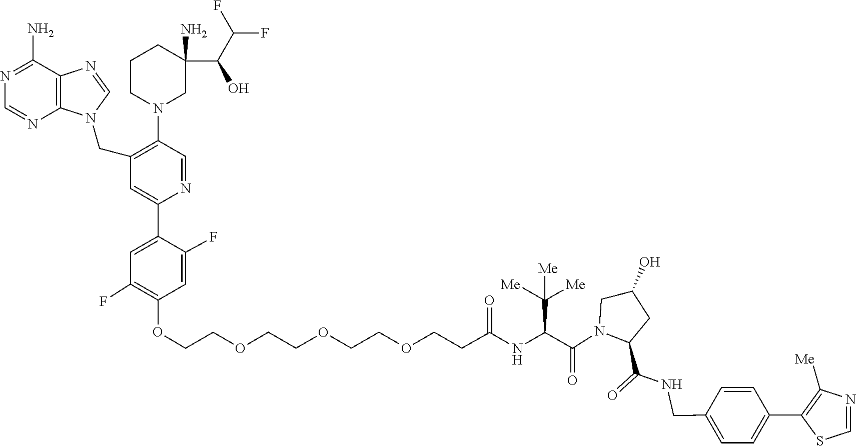 [SMILES: Cc1ncsc1-c1ccc(CNC(=O)[C@@H]2C[C@@H](O)CN2C(=O)[C@@H](NC(=O)CCOCCOCCOCCOc2cc(F)c(-c3cc(Cn4cnc5c(N)ncnc54)c(N4CCC[C@](N)([C@H](O)C(F)F)C4)cn3)cc2F)C(C)(C)C)cc1] |
| I-85 | 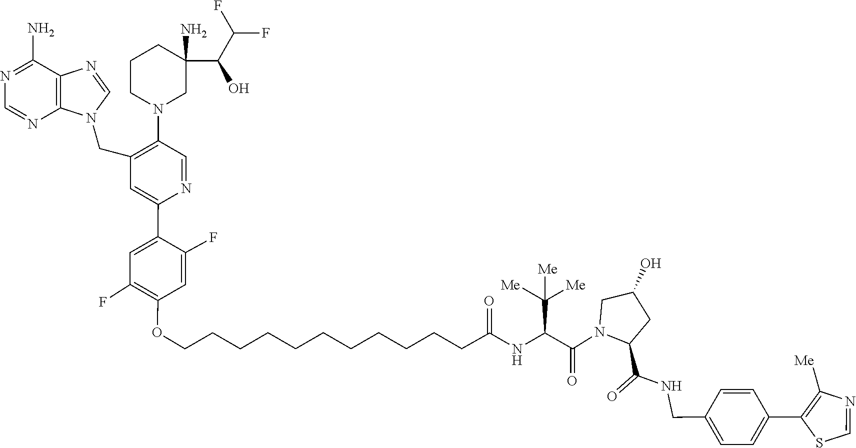 [SMILES: Cc1ncsc1-c1ccc(CNC(=O)[C@@H]2C[C@@H](O)CN2C(=O)[C@@H](NC(=O)CCCCCCCCCCCOc2cc(F)c(-c3cc(Cn4cnc5c(N)ncnc54)c(N4CCC[C@](N)([C@H](O)C(F)F)C4)cn3)cc2F)C(C)(C)C)cc1] |
| I-86 | 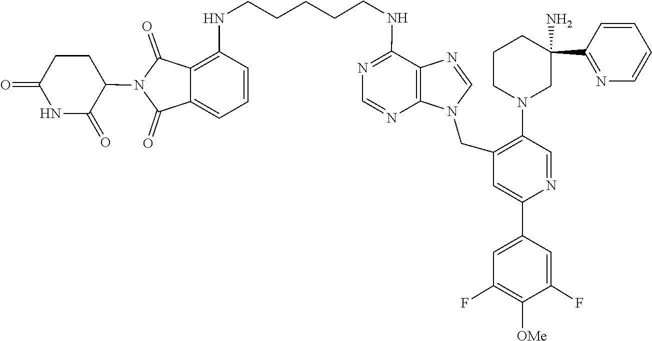 [SMILES: COc1c(F)cc(-c2cc(Cn3cnc4c(NCCCCCNc5cccc6c5C(=O)N(C5CCC(=O)NC5=O)C6=O)ncnc43)c(N3CCC[C@@](N)(c4ccccn4)C3)cn2)cc1F] |
| I-87 | 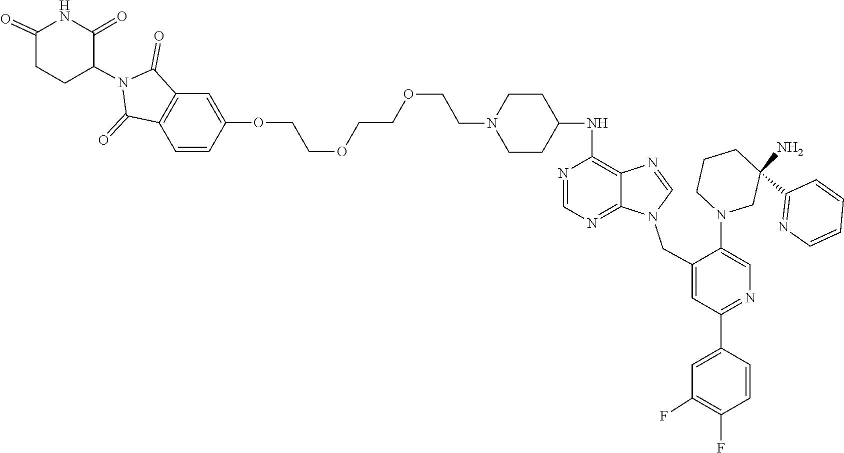 [SMILES: N[C@]1(c2ccccn2)CCCN(c2cnc(-c3ccc(F)c(F)c3)cc2Cn2cnc3c(NC4CCN(CCOCCOCCOc5ccc6c(c5)C(=O)N(C5CCC(=O)NC5=O)C6=O)CC4)ncnc32)C1] |
| I-88 | 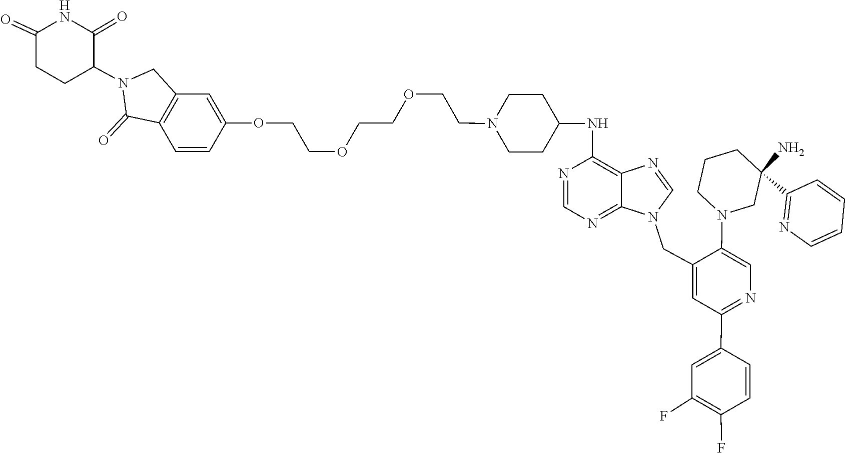 [SMILES: N[C@]1(c2ccccn2)CCCN(c2cnc(-c3ccc(F)c(F)c3)cc2Cn2cnc3c(NC4CCN(CCOCCOCCOc5ccc6c(c5)CN(C5CCC(=O)NC5=O)C6=O)CC4)ncnc32)C1] |
| I-89 | 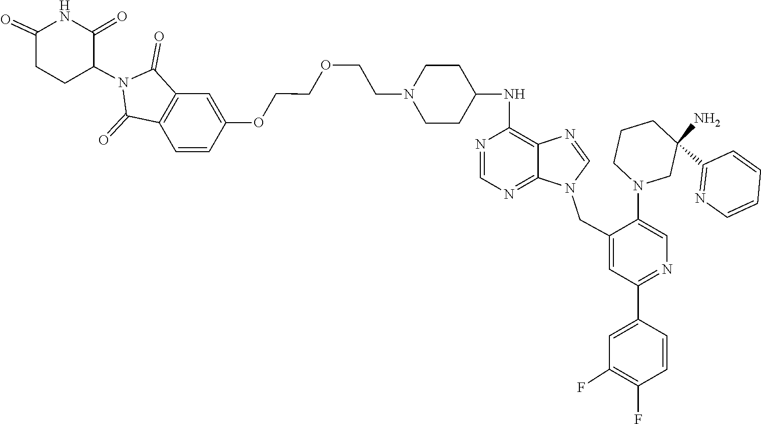 [SMILES: N[C@]1(c2ccccn2)CCCN(c2cnc(-c3ccc(F)c(F)c3)cc2Cn2cnc3c(NC4CCN(CCOCCOc5ccc6c(c5)C(=O)N(C5CCC(=O)NC5=O)C6=O)CC4)ncnc32)C1] |
| I-90 | 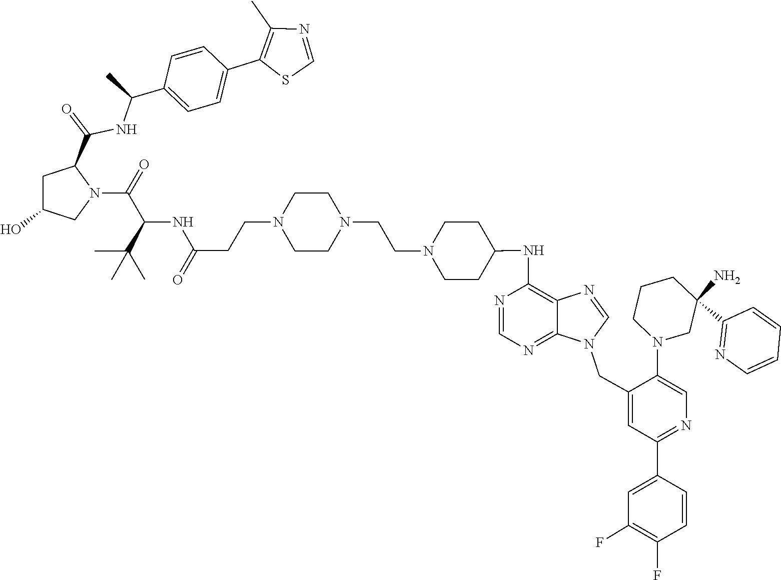 [SMILES: Cc1ncsc1-c1ccc([C@H](C)NC(=O)[C@@H]2C[C@@H](O)CN2C(=O)[C@@H](NC(=O)CCN2CCN(CCN3CCC(Nc4ncnc5c4ncn5Cc4cc(-c5ccc(F)c(F)c5)ncc4N4CCC[C@](N)(c5ccccn5)C4)CC3)CC2)C(C)(C)C)cc1] |
| I-90 | 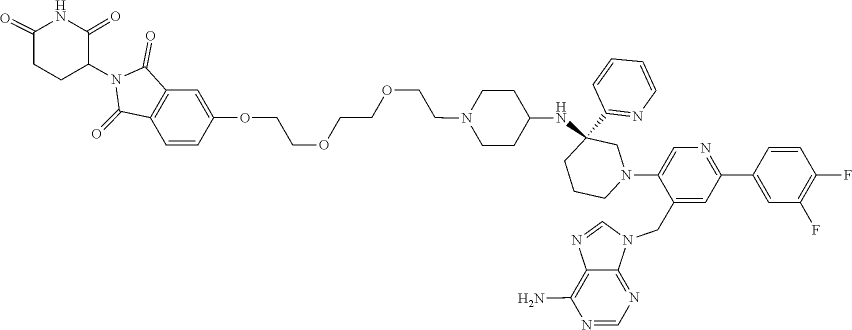 [SMILES: Nc1ncnc2c1ncn2Cc1cc(-c2ccc(F)c(F)c2)ncc1N1CCC[C@](NC2CCN(CCOCCOCCOc3ccc4c(c3)C(=O)N(C3CCC(=O)NC3=O)C4=O)CC2)(c2ccccn2)C1] |
| I-90 | 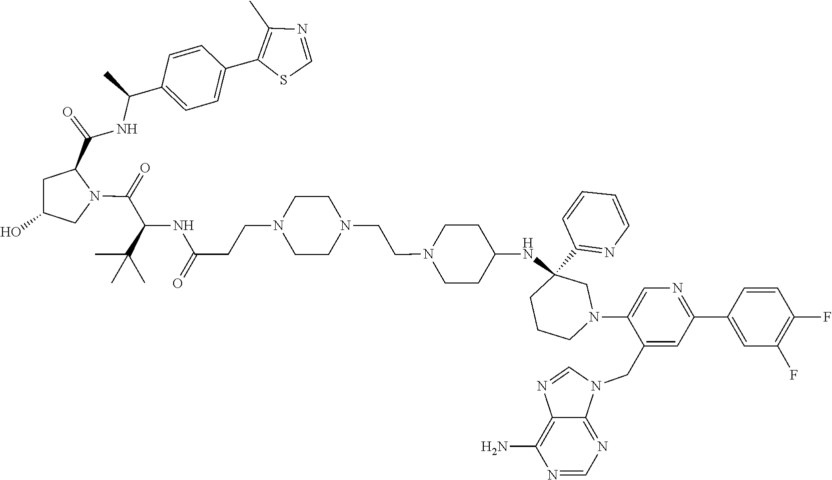 [SMILES: Cc1ncsc1-c1ccc([C@H](C)NC(=O)[C@@H]2C[C@@H](O)CN2C(=O)[C@@H](NC(=O)CCN2CCN(CCN3CCC(N[C@]4(c5ccccn5)CCCN(c5cnc(-c6ccc(F)c(F)c6)cc5Cn5cnc6c(N)ncnc65)C4)CC3)CC2)C(C)(C)C)cc1] |
| I-93 | 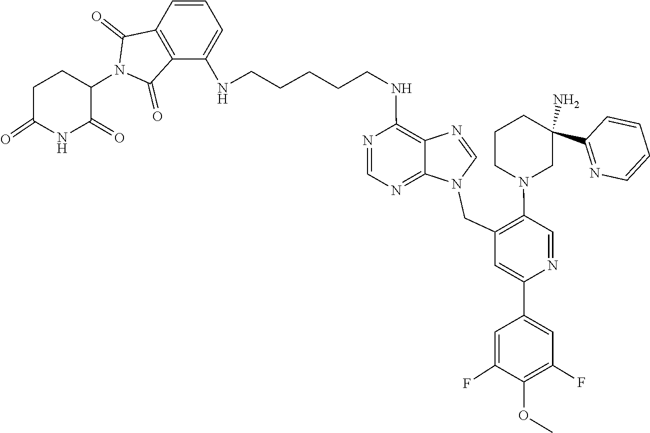 [SMILES: COc1c(F)cc(-c2cc(Cn3cnc4c(NCCCCCNc5cccc6c5C(=O)N(C5CCC(=O)NC5=O)C6=O)ncnc43)c(N3CCC[C@@](N)(c4ccccn4)C3)cn2)cc1F] |

54. A pharmaceutical composition comprising a compound of claim 1 and a pharmaceutically acceptable carrier.

55. A method of treating a disorder mediated by NSD2, comprising administering a therapeutically effective amount of a compound of claim 1 to a subject in need thereof to treat the disorder.

56. (canceled)

57. (canceled)

58. The method of claim 55, wherein the disorder is cancer, and the cancer is breast cancer, lung cancer, pancreatic cancer, cervical cancer, colorectal cancer, prostate cancer, gastric cancer, skin cancer, liver cancer, bile duct cancer, nervous system cancer, leukemia, lymphoma, or multiple myeloma.

59. (canceled)

60. (canceled)

61. (canceled)

62. A method of inhibiting NSD2 activity, comprising contacting NSD2 with an effective amount of a compound of claim 1 to inhibit NSD2 activity.

63. A method of degrading an NSD2 protein in a cell, comprising administering to the cell an effective amount of a compound of claim 1 resulting in degradation of the NSD2 protein.

64. The compound of claim 1, wherein L is 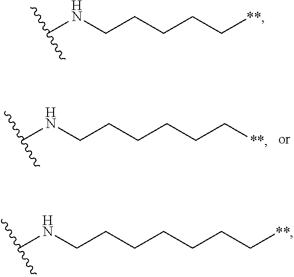
wherein ** is a point of attachment to LBM.
